(19) United States
(12) Patent Application Publication
(10) Pub. No.: US 2022/0096606 A1
(43) Pub. Date: Mar. 31, 2022

(54) Compositions and Methods for Treatment of Duchenne Muscular Dystrophy

(71) Applicant: Vertex Pharmaceuticals Incorporated, Boston, MA (US)

(72) Inventors: Jesper Gromada, Boston, MA (US); Tudor Fulga, Boston, MA (US); Alison McVie-Wylie, Boston, MA (US); Giselle Dominguez Gutierrez, Boston, MA (US); Yurong Xin, Boston, MA (US); Fatih Bolukbasi, Arlington, MA (US); Yi-Li Min, Boston, MA (US); Eric Anderson, Cambridge, MA (US)

(73) Assignee: Vertex Pharmaceuticals Incorporated, Boston, MA (US)

(21) Appl. No.: 17/469,665

(22) Filed: Sep. 8, 2021

Related U.S. Application Data
(60) Provisional application No. 63/179,850, filed on Apr. 26, 2021.
(60) Provisional application No. 63/166,174, filed on Mar. 25, 2021.
(60) Provisional application No. 63/152,114, filed on Feb. 22, 2021.
(60) Provisional application No. 63/076,250, filed on Sep. 9, 2020.

(51) Int. Cl. A61K 38/46; A61K 31/7105; A61K 38/16

(52) U.S. Cl. CPC A61K 38/465; A61K 38/164; A61K 31/7105

(57) ABSTRACT

Compositions and methods for treating Duchenne Muscular Dystrophy (DMD) are encompassed.

This application claims the benefit of priority to U.S. Provisional Application No. 63/076,250, filed Sep. 9, 2020; U.S. Provisional Application No. 63/152,114, filed Feb. 22, 2021; U.S. Provisional Patent Application No. 63/166,174, filed Mar. 25, 2021; and U.S. Provisional Application No. 63/179,850, filed Apr. 26, 2021; all of which are incorporated by reference in their entirety.

The instant application contains a Sequence Listing which has been submitted electronically in ASCII format and is hereby incorporated by reference in its entirety. Said ASCII copy, created on Sep. 7, 2021, is named 2021-09-07_01245-0024-00US_ST25.txt and is 646,734 bytes in size.

### INTRODUCTION AND SUMMARY

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), which result in loss of expression of dystrophin, causing muscle membrane fragility and progressive muscle wasting.

CRISPR-based genome editing can provide sequence-specific cleavage of genomic DNA using a Cas9 and a guide RNA. For example, a nucleic acid encoding the Cas9 enzyme and a nucleic acid encoding for the appropriate guide RNA can be provided on separate vectors or together on a single vector and administered in vivo or in vitro to knockout or correct a genetic mutation, for example. The approximately 20 nucleotides at the 5′ end of the guide RNA serves as the guide or spacer sequence that can be any sequence complementary to one strand of a genomic target location that has an adjacent protospacer adjacent motif (PAM). The PAM sequence is a short sequence adjacent to the Cas9 nuclease cut site that the Cas9 molecule requires for appropriate binding. The nucleotides 3′ of the guide or spacer sequence of the guide RNA serve as a scaffold sequence for interacting with Cas9. When a guide RNA and a Cas9 are expressed, the guide RNA will bind to Cas9 and direct it to the sequence complementary to the guide sequence, where it will then initiate a double-stranded break (DSB). To repair these breaks, cells typically use an error prone mechanism of non-homologous end joining (NHEJ) which can lead to disruption of function in the target gene through insertions or deletion of codons, shifts in the reading frame, or result in a premature stop codon triggering nonsense-mediated decay. See, e.g., Kumar et al. (2018) Front. Mol. Neurosci. Vol. 11, Article 413.

While gene editing strategies using systems (e.g., CRISPR) for treating DMD have been previously explored, these strategies have focused primarily on either: a) cutting at multiple different sites to excise large portions (e.g., one or more exons) of the dystrophin gene (see, e.g., Ousterout et al., 2015, Nat Commun 6:6244); or b) cutting in a single site to introduce indels that either result in a frame-shifting mutation and/or that destroy a splice acceptor/donor site in the dystrophin gene (see, e.g., Amoassi et al., 2018, Science, 362(6410):86-91). However, there remains a need for additional alternative and effective gene editing strategies for treating diseases like DMD.

In order for gene editing systems like CRISPR to be effective in treating diseases and disorders like DMD in a patient, effective delivery vectors of the CRISPR system components are necessary. Adeno-associated virus (AAV) administration of the CRISPR-Cas components in vivo or in vitro is attractive due to the early and ongoing successes of AAV vector design, manufacturing, and clinical stage administration for gene therapy. See, e.g., Wang et al. (2019) Nature Reviews Drug Discovery 18:358-378; Ran et al. (2015a) Nature 520: 186-101. However, the commonly used Streptococcus pyogenes (spCas9) is very large, and when used in AAV-based CRISPR/Cas systems, requires two AAV vectors—one vector carrying the nucleic acid encoding the spCas9, and the other carrying the nucleic acid encoding the guide RNA. One possible way to overcome this technical hurdle is to take advantage of the smaller orthologs of Cas9 derived from different prokaryotic species. Smaller Cas9's such as Staphylococcus aureus (SaCas9) and Staphylococcus lugdunensis (SluCas9) may be able to be manufactured on a single AAV vector together with a nucleic acid encoding one or more guide RNAs. One advantage of incorporating one or more guide RNAs on a single vector together with the smaller SaCas9 or SluCas9 is that doing so allows extreme design flexibility in situations where more than one guide RNA is desired for optimal performance. For example, one vector may be utilized to express SaCas9 or SluCas9 and one or more guide RNAs targeting a first genomic target (e.g., a pair of guide RNAs that together bind regions flanking a genomic target), and a second vector may be utilized to express multiple copies of the same (e.g., the same pair of guides in the first vector) or different guide RNAs targeting the same or a different genomic target. As another example, multiple copies of the same guide RNA may be beneficial, and the use of smaller Cas9's allow for multiple copies of guide RNA to be incorporated on the same vector as the Cas9, and also for even more copies of guide RNA when combined with a second vector. Compositions and methods utilizing these configurations have the benefit of reducing manufacturing costs, reducing complexity of administration routes and protocols, and allowing maximum flexibility with regard to using multiple copies of the same or different guide RNAs targeting the same or different genomic target sequences. In some instances, providing multiple copies of the same guide RNA improves the efficiency of the guide, improving an already successful system.

Provided herein are compositions and methods for treating DMD utilizing the smaller Cas9s from Staphylococcus aureus (SaCas9) and Staphylococcus lugdunensis (SluCas9). Compositions comprising a single AAV vector comprising a nucleic acid molecule encoding SaCas9 or SluCas9 and a guide RNA are provided.

Accordingly, the following non-limiting embodiments are provided.
Embodiment A1 is a composition comprising:

a. a single nucleic acid molecule comprising:
i. a nucleic acid encoding Staphylococcus aureus Cas9 (SaCas9) or Staphylococcus lugdunensis (SluCas9) and at least one, at least two, or at least three guide RNAs; or
ii. a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or
iii. a nucleic acid encoding Staphylococcus aureus Cas9 (SaCas9) or Staphylococcus lugdunensis (SluCas9) and 1, 2, or 3 guide RNAs; or

b. two nucleic acid molecules comprising:
i. a first nucleic acid encoding Staphylococcus aureus Cas9 (SaCas9) or Staphylococcus lugdunensis (SluCas9); and
   a second nucleic acid that does not encode a SaCas9 or SluCas9 and encodes any one of the following:
      1. at least one, at least two, at least three, at least four, at least five, or at least six guide RNAs; or
      2. from one to n guide RNAs, wherein n is no more than the maximum number of guide RNAs that can be expressed from said nucleic acid; or
      3. from one to six guide RNAs; or
ii. a first nucleic acid encoding Staphylococcus aureus Cas9 (SaCas9) or Staphylococcus lugdunensis (SluCas9) and
   1. at least one, at least two, or at least three guide RNAs; or
   2. from one to n guide RNAs, wherein n is no more than the maximum number of guide RNAs that can be expressed from said nucleic acid; or
   3. 1, 2, or 3 guide RNAs; and
a second nucleic acid that does not encode a SaCas9 or SluCas9, optionally wherein the second nucleic acid comprises any one of the following:
   1. at least one, at least two, at least three, at least four, at least five, or at least six guide RNAs; or
   2. from one to n guide RNAs, wherein n is no more than the maximum number of guide RNAs that can be expressed from said nucleic acid; or
   3. from one to six guide RNAs; or
iii. a first nucleic acid encoding Staphylococcus aureus Cas9 (SaCas9) or Staphylococcus lugdunensis (SluCas9) and at least one, at least two, or at least three guide RNAs; and a second nucleic acid that does not encode a SaCas9 or SluCas9 and encodes from one to six guide RNAs; or
iv. a first nucleic acid encoding Staphylococcus aureus Cas9 (SaCas9) or Staphylococcus lugdunensis (SluCas9) and at least two guide RNAs, wherein at least one guide RNA binds upstream of a target sequence and at least one guide RNA binds downstream of the target sequence; and a second nucleic acid that does not encode a SaCas9 or SluCas9 and encodes at least one additional copy of each of the guide RNAs encoded in the first nucleic acid,

wherein the guide RNA(s) target a region in the dystrophin gene.
Embodiment A2 is a composition comprising two nucleic acid molecules comprising i) a first nucleic acid encoding Staphylococcus aureus Cas9 (SaCas9) or Staphylococcus lugdunensis (SluCas9) and a first and a second guide RNA that function to excise a portion of a DMD gene; and ii) a second nucleic acid encoding at least 2 or at least 3 copies of the first guide RNA and at least 2 or at least 3 copies of the second guide RNA.
Embodiment A3 is a composition comprising one or more nucleic acid molecules encoding an endonuclease and a pair of guide RNAs, wherein each guide RNA targets a different sequence in a DMD gene, wherein the endonuclease and pair of guide RNAs are capable of excising a DNA fragment from the DMD gene; wherein the DNA fragment is between 5-250 nucleotides in length.
Embodiment A4 is the composition of claim 3, wherein the endonuclease is a class 2, type II Cas endonuclease.
Embodiment A5 is the composition of claim 3, wherein the class 2, type II Cas endonuclease is SpCas9, SaCas9, or SluCas9.
Embodiment A6 is the composition of claim 3, wherein the endonuclease is not a class 2, type V Cas endonuclease.
Embodiment A7 is the composition of claim 3, wherein the excised DNA fragment comprises a splice acceptor site or a splice donor site.
Embodiment A8 is the composition of claim 3, wherein the excised DNA fragment comprises a premature stop codon in the DMD gene.
Embodiment A9 is the composition of claim 3, wherein the excised DNA fragment does not comprise an entire exon of the DMD gene.
Embodiment A10 is the composition of any one of claims 1-9, wherein the guide RNA comprises any one of the following:
   a. when SaCas9 is used, one or more spacer sequences selected from any one of SEQ ID NOs: 1-35, 1000-1078, and 3000-3069; or
   b. when SluCas9a is used, one or more spacer sequences selected from any one of SEQ ID NOs: 100-225, 2000-2116, and 4000-4251; or
   c. when SaCas9 is used, one or more spacer sequences comprising at least 20 contiguous nucleotides of a spacer sequence selected from any one of SEQ ID NOs: 1-35, 1000-1078, and 3000-3069; or
   d. when SluCas9a is used, one or more spacer sequences comprising at least 20 contiguous nucleotides of a spacer sequence selected from any one of SEQ ID NOs: 100-225, 2000-2116, and 4000-4251; or
   e. when SaCas9 is used, one or more spacer sequences that is at least 90% identical to any one of SEQ ID NOs: 1-35, 1000-1078, and 3000-3069; or
   f. when SluCas9 is used, one or more spacer sequences that is at least 90% identical to any one of SEQ ID NOs: 100-225, 2000-2116, and 4000-4251; or
   g. when SaCas9 is used with at least two guide RNAs, a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
   h. when SaCas9 is used with at least two guide RNAs, at least 17, 18, 19, 20, or 21 contiguous nucleotides of a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
   i. when SaCas9 is used with at least two guide RNAs, at least 90% identical to a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
   j. when SluCas9 is used with at least two guide RNAs, a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
   k. when SluCas9 is used with at least two guide RNAs, at least 17, 18, 19, 20, or 21 contiguous nucleotides of a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
   l. when SluCas9 is used with at least two guide RNAs, at least 90% identical to a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
   m. when SluCas9 is used with at least two guide RNAs, at least 90% identical to a first and second spacer sequence selected from any one of the following pairs of spacer sequences:
      i. SEQ ID NOS: 148 and 134,
      ii. SEQ ID Nos: 145 and 131,
      iii. SEQ ID Nos: 144 and 149;
      iv. SEQ ID Nos: 144 and 150; and
      v. SEQ ID Nos: 146 and 148; or
   n. when a SaCas9-KKH is used with at least two guide RNAs, at least 90% identical to a first and second spacer sequence selected from any one of the following pairs of spacer sequences:
      i. SEQ ID NOs: 12 and 1013; and
      ii. SEQ ID Nos: 12 and 1016.
Embodiment A11 is a composition comprising a single nucleic acid molecule encoding one or more guide RNAs and a Cas9, wherein the single nucleic acid molecule comprises:
   a. a first nucleic acid encoding one or more spacer sequences selected from any one of SEQ ID NOs: 1-35, 1000-1078, or 3000-3069 and a second nucleic acid encoding a Staphylococcus aureus Cas9 (SaCas9); or
   b. a first nucleic acid encoding one or more spacer sequences selected from any one of SEQ ID NOs: 100-225, 2000-2116, or 4000-4251 and a second nucleic acid encoding a Staphylococcus lugdunensis (SluCas9); or
   c. a first nucleic acid encoding one or more spacer sequences comprising at least 20 contiguous nucleotides of a spacer sequence selected from any one of SEQ ID NOs: 1-35, 1000-1078, or 3000-3069 and a second nucleic acid encoding a Staphylococcus aureus Cas9 (SaCas9); or
   d. a first nucleic acid encoding one or more spacer sequences comprising at least 20 contiguous nucleotides of a spacer sequence selected from any one of SEQ ID NOs: 100-225, 2000-2116, or 4000-4251 and a second nucleic acid encoding a Staphylococcus lugdunensis (SluCas9); or
   e. a first nucleic acid encoding one or more spacer sequences that is at least 90% identical to any one of SEQ ID NOs: 1-35, 1000-1078, or 3000-3069 and a second nucleic acid encoding a Staphylococcus aureus Cas9 (SaCas9); or
   f. a first nucleic acid encoding one or more spacer sequences that is at least 90% identical to any one of SEQ ID NOs: 100-225, 2000-2116, or 4000-4251 and a second nucleic acid encoding a Staphylococcus lugdunensis (SluCas9); or
   g. a first nucleic acid encoding a pair of guide RNAs comprising a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   h. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   i. a first nucleic acid encoding a pair of guide RNAs that is at least 90% identical to a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
   j. a first nucleic acid encoding a pair of guide RNAs comprising a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   k. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   l. a first nucleic acid encoding a pair of guide RNAs that is at least 90% identical to a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   m. a first nucleic acid encoding a pair of guide RNAs that is at least 90% identical to a first and second spacer sequence selected from any one of the following pairs of spacer sequences:
      i. SEQ ID NOS: 148 and 134,
      ii. SEQ ID Nos: 145 and 131,
      iii. SEQ ID Nos: 144 and 149;
      iv. SEQ ID Nos: 144 and 150;
      v. SEQ ID Nos: 146 and 148;
   and a second nucleic acid encoding a Staphylococcus lugdunensis (SluCas9); or
   n. a first nucleic acid encoding a pair of guide RNAs that is at least 90% identical to a first and second spacer sequence selected from any one of the following pairs of spacer sequences:
      i. SEQ ID NOs: 12 and 1013; and
      ii. SEQ ID Nos: 12 and 1016;
      iii. and a second nucleic acid encoding a SaCas9-KKH.
Embodiment A12 is a composition comprising one or more nucleic acid molecules encoding a Staphylococcus lugdunensis (SluCas9) and at least two guide RNAs, wherein a first and second guide RNA target different sequences in a DMD gene, wherein the first and a second guide RNA comprise a sequence that is at least 90% identical to a first and second spacer sequence selected from any one of the following pairs of spacer sequences:
   i. SEQ ID NOS: 148 and 134,
   ii. SEQ ID Nos: 145 and 131,
   iii. SEQ ID Nos: 144 and 149;
   iv. SEQ ID Nos: 144 and 150;
   v. SEQ ID Nos: 146 and 148.
Embodiment A13 is a composition comprising one or more nucleic acid molecules encoding an endonuclease and at least two guide RNAs, wherein the guide RNAs each target a different sequence in a DMD gene, wherein the guide RNAs each comprise a sequence that is at least 90% identical to a first and second spacer sequence selected from any one of the following pairs of spacer sequences:
   i. SEQ ID NOs: 12 and 1013; and
   ii. SEQ ID Nos: 12 and 1016; and
   b. a second nucleic acid encoding a SaCas9-KKH.
Embodiment A14 is the composition of any one of the preceding claims, wherein the first and/or the second nucleic acid, if present, encodes at least two guide RNAs.
Embodiment A15 is the composition of any one of the preceding claims, wherein the first and/or the second nucleic acid, if present, encodes at least three guide RNAs.
Embodiment A16 is the composition of any one of the preceding claims, wherein the first and/or the second nucleic acid, if present, encodes at least four guide RNAs.
Embodiment A17 is the composition of any one of the preceding claims, wherein the first and/or the second nucleic acid, if present, encodes at least five guide RNAs.
Embodiment A18 is the composition of any one of the preceding claims, wherein the first and/or the second nucleic acid, if present, encodes at least six guide RNAs.
Embodiment A19 is the composition of any one of the preceding claims, wherein the first and/or the second nucleic acid, if present, encodes at least seven guide RNAs.
Embodiment A20 is the composition of any one of the preceding claims, wherein the first and/or the second nucleic acid, if present, encodes at least eight guide RNAs.
Embodiment A21 is the composition of any one of the preceding claims, wherein the first nucleic acid comprises a nucleotide sequence encoding an endonuclease and at least one, at least two, or at least three guide RNAs.
Embodiment A22 is the composition of any one of the preceding claims, wherein the first nucleic acid comprises a nucleotide sequence encoding an endonuclease and from one to n guide RNAs, wherein n is no more than the maximum number of guide RNAs that can be expressed from said nucleic acid.
Embodiment A23 is the composition of any one of the preceding claims, wherein the first nucleic acid comprises a nucleotide sequence encoding an endonuclease and from one to three guide RNAs.
Embodiment A24 is the composition of any one of the preceding claims, wherein the second nucleic acid, if present, encodes at least one, at least two, at least three, at least four, at least five, or at least six guide RNAs.
Embodiment A25 is the composition of any one of the preceding claims, wherein the second nucleic acid, if present, encodes from one to n guide RNAs, wherein n is no more than the maximum number of guide RNAs that can be expressed from said nucleic acid.
Embodiment A26 is the composition of any one of the preceding claims, wherein the second nucleic acid, if present, encodes from one to six guide RNAs.
Embodiment A27 is the composition of any one of the preceding claims, wherein the second nucleic acid, if present, encodes 2-10, 2-9, 2-8, 2-7, 2-6, 2-5, 2-4, or 2-3 guide RNAs.
Embodiment A28 is the composition of any one of the preceding claims, wherein the second nucleic acid, if present, encodes 2, 3, 4, 5, or 6 guide RNAs.
Embodiment A29 is the composition of any one of the preceding claims, comprising at least two nucleic acid molecules, wherein the guide RNA encoded by the first nucleic acid and the second nucleic acid are the same.
Embodiment A30 is the composition of any one of the preceding claims, comprising at least two nucleic acid molecules, wherein the guide RNA encoded by the first nucleic acid and the second nucleic acid are different.
Embodiment A31 is the composition of any one of the preceding claims, comprising at least two nucleic acid molecules encoding at least two guide RNAs, wherein at least one guide RNA binds to a target sequence within an exon in the DMD gene that is upstream of a premature stop codon, and wherein at least one guide RNA binds to a target sequence within an exon in the DMD gene that is downstream of a premature stop codon.
Embodiment A32 is the composition of any one of the preceding claims, comprising at least two nucleic acid molecules, wherein the first nucleic acid molecule and the second nucleic acid molecule each encode the same guide RNA.
Embodiment A33 is the composition of any one of the preceding claims, comprising at least two nucleic acid molecules each encoding at least one guide RNA, wherein the second nucleic acid molecule encodes a guide RNA that binds to the same target sequence as the guide RNA in the first nucleic acid molecule.
Embodiment A34 is the composition of any one of the preceding claims, comprising at least two nucleic acid molecules, wherein the second nucleic acid molecule encodes at least 2, at least 3, at least 4, at least 5, or at least 6 guide RNAs, wherein the guide RNAs in the second nucleic acid molecule bind to the same target sequence as the guide RNA in the first nucleic acid molecule.
Embodiment A35 is the composition of any one of the preceding claims, wherein the composition comprises at least two nucleic acid molecules, wherein the first nucleic acid molecule comprises a sequence encoding an endonuclease, wherein the second nucleic acid molecule encodes a first guide RNA and a second guide RNA, wherein the first guide RNA and the second guide RNA are not the same sequence, and wherein the second nucleic acid molecule does not encode an endonuclease.
Embodiment A36 is the composition of claim 35, wherein the first nucleic acid molecule also encodes a copy of the first guide RNA and a copy of the second guide RNA.
Embodiment A37 is the composition of claim 35 or 36, wherein the second nucleic acid molecule encodes two copies of the first guide RNA and two copies of the second guide RNA.
Embodiment A38 is the composition of any one of claims 35-37, wherein the second nucleic acid molecule encodes three copies of the first guide RNA and three copies of the second guide RNA.
Embodiment A39 is the composition of any one of claims 35-37, wherein the first nucleic acid molecule comprises from 5′ to 3′ with respect to the plus strand: the reverse complement of a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding the endonuclease, a nucleotide sequence encoding an endonuclease, a polyadenylation sequence, a promoter for expression of the second guide RNA in the same direction as the promoter for the endonuclease, the second guide RNA sequence, and a second guide RNA scaffold sequence.
Embodiment A40 is the composition of claim 39, wherein the promoter for expression of the nucleotide sequence encoding the first guide RNA sequence in the first nucleic acid molecule is a U6 promoter and the promoter for expression of the nucleotide sequence encoding the second guide RNA in the first nucleic acid molecule is a U6 promoter.
Embodiment A41 is the composition of any one of claims 35-40, wherein the first nucleic acid molecule is in a first vector, and wherein the second nucleic acid is in a separate second vector.
Embodiment A42 is the composition of any one of claims 35-41, wherein the first nucleic acid molecule encodes a Staphylococcus aureus Cas9 (SaCas9) endonuclease, and wherein the first guide RNA comprises the first sequence and the second guide RNAs comprises the second sequence from any one the following pairs of sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018.
Embodiment A43 is the composition of claim 42, wherein the first guide RNA comprises the sequence of SEQ ID NO: 12 and the second guide RNA comprises the sequence of SEQ ID NO: 1013.
Embodiment A44 is the composition of any one of claims 35-43, wherein the first nucleic acid molecule encodes a Staphylococcus lugdunensis (SluCas9) endonuclease, and wherein the first guide RNA comprises the first sequence and the second guide RNAs comprises the second sequence from any one the following pairs of sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146.
Embodiment A45 is the composition of claim 44, wherein:
   i. the first guide RNA comprises the sequence of SEQ ID NO: 148 and the second guide RNA comprises the sequence of SEQ ID NO: 134; or
   ii. the second guide RNA comprises the sequence of SEQ ID NO: 145 and the second guide RNA comprises the sequence of SEQ ID NO: 131.
Embodiment A46 is the composition of any one of claims 35-45, wherein the first nucleic acid is in a first vector, and wherein the second nucleic acid is in a separate second vector.
Embodiment A47 is the composition of claim 46, wherein the first and second vectors are viral vectors.
Embodiment A48 is the composition of claim 47, wherein the viral vectors are AAV9 vectors.
Embodiment A49 is the composition of claim 48, wherein the AAV9 vectors are each less than 5 kb, less than 4.9 kb, less than 4.85, less than 4.8, or less than 4.75 kb from ITR to ITR in size, inclusive of both ITRs.
Embodiment A50 is the composition of claim 48 or 49, wherein the AAV9 vectors are each between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs.
Embodiment A51 is the composition of any one of claims 47-50, wherein the first vector is between 4.4-4.85 kb from ITR to ITR in size, inclusive of both ITRs.
Embodiment A52 is the composition of any one of the preceding claims, wherein the guide RNA binds to one or more target sequences within a DMD gene.
Embodiment A53 is the composition of any one of the preceding claims, wherein the guide RNA binds to one or more target sequences within an exon of the DMD gene.
Embodiment A54 is the composition of any one of the preceding claims, comprising two guide RNAs, wherein i) each guide RNA targets a sequence within an exon; ii) one guide RNA targets a sequence within an exon and one targets a sequence within an intron; or iii) each guide RNA targets a sequence within an intron.
Embodiment A55 is the composition of any one of the preceding claims, comprising at least two guide RNAs, wherein i) each guide RNA targets the same genomic target sequence; ii) each guide RNA targets a different target sequence; or iii) at least one guide RNA targets one sequence and at least one guide RNA targets a different sequence.
Embodiment A56 is the composition of any one of the preceding claims, comprising a guide RNA that binds to an exon of the DMD gene, wherein the exon is selected from exon 43, 44, 45, 50, 51, and 53.
Embodiment A57 is the composition of any one of the preceding claims, comprising at least two guide RNAs that binds to an exon of the DMD gene, wherein at least one guide RNA binds to a target sequence within an exon in the DMD gene, and at least one guide RNA binds to a different target sequence within the same exon in the DMD gene.
Embodiment A58 is the composition of any one of the preceding claims, comprising at least two guide RNAs, wherein at least one guide RNA binds to a target sequence within an exon that is upstream of a premature stop codon, and at least one guide RNA binds to a target sequence within an exon that is downstream of a premature stop codon.
Embodiment A59 is the composition of any one of the preceding claims, comprising at least two guide RNAs, wherein at least one guide RNA binds to a target sequence within an exon in the DMD gene, and at least one guide RNA binds to a different target sequence within the same exon in the DMD gene, wherein when expressed in vivo or in vivo, a portion of the exon is excised.
Embodiment A60 is the composition of any one of the preceding claims, comprising at least two guide RNAs, wherein once expressed in vitro or in vivo in combination with an RNA-guided endonuclease, the guide RNAs excise a portion of the exon.
Embodiment A61 is the composition of any one of the preceding claims, comprising at least two guide RNAs, wherein once expressed in vitro or in vivo in combination with an RNA-guided endonuclease, the guide RNAs excise a portion of the exon, and wherein the portion of the exon remaining after excision are rejoined with a one nucleotide insertion.
Embodiment A62 is the composition of any one of the preceding claims, comprising at least two guide RNAs, wherein once expressed in vitro or in vivo in combination with an RNA-guided endonuclease, the guide RNAs excise a portion of the exon, wherein the portion of the exon remaining after excision is rejoined without a nucleotide insertion.
Embodiment A63 is the composition of any one of the preceding claims, comprising at least two guide RNAs, wherein once expressed in vitro or in vivo in combination with an RNA-guided endonuclease, the guide RNAs excise a portion of the exon, wherein the size of the excised portion of the exon is between 5 and 250 nucleotides in length.
Embodiment A64 is the composition of any one of the preceding claims, comprising at least two guide RNAs, wherein once expressed in vitro or in vivo in combination with an RNA-guided endonuclease, the guide RNAs excise a portion of the exon, wherein the size of the excised portion of the exon is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.
Embodiment A65 is the composition of any one of the preceding claims, comprising at least two guide RNAs, wherein once expressed in vitro or in vivo in combination with an RNA-guided endonuclease, the guide RNAs excise a portion of the exon, wherein the size of the excised portion of the exon is between 8 and 167 nucleotides.
Embodiment A66 is the composition of any one of the preceding claims, wherein the guide RNA is an sgRNA.
Embodiment A67 is the composition of any one of the preceding claims, wherein the guide RNA is modified.
Embodiment A68 is the composition of any one of the preceding claims, wherein the guide RNA is modified, and wherein the modification alters one or more 2′ positions and/or phosphodiester linkages.
Embodiment A69 is the composition of any one of the preceding claims, wherein the guide RNA is modified, and wherein the modification alters one or more, or all, of the first three nucleotides of the guide RNA and/or the last three nucleotides of the sgRNA.
Embodiment A70 is the composition of any one of claims 66-68, wherein the modification alters one or more, or all, of the last three nucleotides of the guide RNA.
Embodiment A71 is the composition of any one of the preceding claims, wherein the guide RNA is modified, and wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.
Embodiment A72 is the composition of any one of the preceding claims, wherein the composition further comprises a pharmaceutically acceptable excipient.
Embodiment A73 is the composition of any one of the preceding claims, wherein the composition is associated with a lipid nanoparticle (LNP).
Embodiment A74 is the composition of any one of the preceding claims, wherein the composition is associated with a viral vector.
Embodiment A75 is the composition of any one of the preceding claims, wherein the composition is associated with a viral vector, and wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment A76 is the composition of any one of the preceding claims, wherein the composition is associated with a viral vector, and wherein the viral vector is an adeno-associated virus (AAV) vector.
Embodiment A77 is the composition of any one of the preceding claims, wherein the composition is associated with a viral vector, wherein the viral vector is an adeno-associated virus (AAV) vector, and wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment A78 is the composition of any one of the preceding claims, wherein the composition is associated with a viral vector, wherein the viral vector is an adeno-associated virus (AAV) vector, and wherein the AAV vector is an AAV serotype 9 (AAV9) vector.
Embodiment A79 is the composition of claim 78, wherein the AAV serotype 9 vector is less than 5 kb, less than 4.9 kb, less than 4.85, less than 4.8, or less than 4.75 kb from ITR to ITR in size, inclusive of both ITRs.
Embodiment A80 is the composition of claim 78 or 79, wherein the AAV serotype 9 vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs.
Embodiment A81 is the composition of any one of claims 78-80, wherein the AAV serotype 9 vector is between 4.4-4.85 kb from ITR to ITR in size, inclusive of both ITRs.
Embodiment A82 is the composition of any one of the preceding claims, wherein the composition is associated with a viral vector, wherein the viral vector is an adeno-associated virus (AAV) vector, and wherein the AAV vector is an AAVrh10 vector.
Embodiment A83 is the composition of any one of the preceding claims, wherein the composition is associated with a viral vector, wherein the viral vector is an adeno-associated virus (AAV) vector, and wherein the AAV vector is an AAVrh74 vector.
Embodiment A84 is the composition of any one of the preceding claims, wherein the composition is associated with a viral vector, wherein the viral vector comprises a tissue-specific promoter.
Embodiment A85 is the composition of any one of the preceding claims, wherein the composition is associated with a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.
Embodiment A86 is the composition of any one of the preceding claims, wherein the composition is associated with a viral vector, wherein the viral vector comprises any one or more of the following promoters: U6, H1, and 7SK promoter.
Embodiment A87 is the composition of any one of the preceding claims, comprising a nucleic acid encoding SaCas9, wherein at least one guide RNA comprises a spacer sequence selected from any one of SEQ ID NOs: 12, 15, 16, 20, 27, 28, 32, 33, and 35.
Embodiment A88 is the composition of any one of the preceding claims, comprising a nucleic acid encoding SaCas9, wherein the SaCas9 comprises the amino acid sequence of SEQ ID NO: 711.
Embodiment A89 is the composition of any one of the preceding claims, comprising a nucleic acid encoding SaCas9, wherein the SaCas9 is a variant of the amino acid sequence of SEQ ID NO: 711.
Embodiment A90 is the composition of any one of the preceding claims, comprising a nucleic acid encoding SaCas9, wherein the SaCas9 comprises an amino acid sequence selected from any one of SEQ ID NOs: 715-717.
Embodiment A91 is the composition of any one of the preceding claims, comprising a nucleic acid encoding SluCas9, wherein at least one guide RNA comprises a spacer sequence selected from any one of SEQ ID NOs: 131, 134, 135, 136, 139, 144, 148, 149, 150, 151, 179, 184, 201, 210, 223, 224, and 225.
Embodiment A92 is the composition of any one of the preceding claims, comprising a nucleic acid encoding SluCas9, wherein the SluCas9 comprises the amino acid sequence of SEQ ID NO: 712.
Embodiment A93 is the composition of any one of the preceding claims, comprising a nucleic acid encoding SluCas9, wherein the SluCas9 is a variant of the amino acid sequence of SEQ ID NO: 712.
Embodiment A94 is the composition of any one of the preceding claims, comprising a nucleic acid encoding SluCas9, wherein the SluCas9 comprises an amino acid sequence selected from any one of SEQ ID NOs: 718-720.
Embodiment A95 is the composition of claim 1, wherein the single nucleic acid molecule or the first nucleic acid comprises from 5′ to 3′ with respect to the plus strand: the reverse complement of a nucleotide sequence encoding a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding Staphylococcus aureus Cas9 (SaCas9) or Staphylococcus lugdunensis (SluCas9), a nucleotide sequence encoding the SaCas9 or SluCas9, a polyadenylation sequence, a promoter for expression of the second guide RNA in the same direction as the promoter for the SaCas9 or SluCas9, a nucleotide sequence encoding the second guide RNA sequence, and a nucleotide sequence encoding the second guide RNA scaffold sequence.
Embodiment A96 is the composition of claim 95, wherein the promoter for expression of the nucleic acid encoding the first guide RNA sequence is a U6 promoter and the promoter for expression of the nucleic acid encoding the second guide RNA is a U6 promoter.
Embodiment A97 is the composition of claim 95 or 96, wherein the SaCas9 or SluCas9 comprise at least two nuclear localization signals (NLSs).
Embodiment A98 is the composition of claim 97, wherein the SaCas9 or SluCas9 comprise a c-Myc NLS fused to the N-terminus of the SaCas9 or SluCas9, optionally by means of a linker. Embodiment A99 is the composition of claim 97 or 98, wherein the SaCas9 or SluCas9 comprise an SV40 NLS fused to the C-terminus of the SaCas9 or SluCas9, optionally by means of a linker.
Embodiment A100 is the composition of any one of claims 97-99, wherein the SaCas9 or SluCas9 comprise a nucleoplasmin NLS fused to the C-terminus of the SaCas9 or SluCas9, optionally by means of a linker.
Embodiment A101 is the composition of any one of claims 97-100, wherein the SaCas9 or SluCas9 comprise:
   i. a c-Myc NLS fused to the N-terminus of the SaCas9 or SluCas9, optionally by means of a linker,
   ii. an SV40 NLS fused to the C-terminus of the SaCas9 or SluCas9, optionally by means of a linker, and
   iii. a nucleoplasmin NLS fused to the C-terminus of the SV40 NLS, optionally by means of a linker,
Embodiment A102 is the composition of any one of claims 95-101, wherein the scaffold sequence for the first guide RNA comprises the sequence of SEQ ID NO: 901.
Embodiment A103 is the composition of any one of claims 95-101, wherein the scaffold sequence for the second guide RNA comprises the sequence of SEQ ID NO: 901.
Embodiment A104 is the composition of any one claims 95-103, wherein the single nucleic acid molecule or the first nucleic acid is less than 5 kb, less than 4.9 kb, 4.85, 4.8, or 4.75 kb in size.
Embodiment A105 is the composition of any one of claims 95-104, wherein the single nucleic acid molecule or the first nucleic acid is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb in size.
Embodiment A106 is the composition of claim 105, wherein the single nucleic acid molecule or the first nucleic acid is between 4.4-4.85 kb from ITR to ITR in size.
Embodiment A107 is a composition comprising a guide RNA encoded by a sequence comprising any one of SEQ ID NOs: 1-35, 1000-1078, or 3000-3069 or complements thereof.
Embodiment A108 is a composition comprising a guide RNA encoded by a sequence comprising any one of SEQ ID NOs: 100-225, 2000-2116, or 4000-4251 or complements thereof.
Embodiment A109 is the composition of any one of claims 1-108 for use in treating Duchenne Muscular Dystrophy (DMD).
Embodiment A110 is the composition of any one of claims 1-108 for use in making one or more double strand breaks in any one or more of exons 43, 44, 45, 50, 51, or 53 of the dystrophin gene.
Embodiment A111 is a method of treating Duchenne Muscular Dystrophy (DMD), the method comprising delivering to a cell the composition of any one of claims 1-107.
Embodiment A112 is a method of treating Duchenne Muscular Dystrophy (DMD), the method comprising delivering to a cell a single nucleic acid molecule comprising:
   i. a nucleic acid encoding one or more guide RNAs, wherein the one or more guide RNAs comprise:
      1. a spacer sequence selected from SEQ ID NOs: 1-35, 1000-1078, or
      2. a spacer sequence comprising at least 20 contiguous nucleotides of a spacer sequence selected from SEQ ID NOs: 1-35, 1000-1078, or 3000-3069; or
      3. a spacer sequence that is at least 90% identical to any one of SEQ ID NOs: 1-35, 1000-1078, 3000-3069; and
   ii. a nucleic acid encoding a Staphylococcus aureus Cas9 (SaCas9).
Embodiment A113 is a method of treating Duchenne Muscular Dystrophy (DMD), the method comprising delivering to a cell a single nucleic acid molecule comprising:
   a. a nucleic acid molecule encoding one or more guide RNAs, wherein the one or more guide RNAs comprise:
      i. a spacer sequence selected from SEQ ID NOs: 100-225, 2000-2116, or 4000-4251;
      ii. a spacer sequence comprising at least 20 contiguous nucleotides of a spacer sequence selected from SEQ ID NOs: 100-225, 2000-2116, or 4000-4251; or
      iii. a spacer sequence that is at least 90% identical to any one of SEQ ID NOs: 100-225, 2000-2116, or 4000-4251; and
   b. a nucleic acid molecule encoding Staphylococcus lugdunensis (SluCas9).
Embodiment A114 is a method of treating Duchenne Muscular Dystrophy (DMD), the method comprising delivering to a cell a single nucleic acid molecule comprising:
   i. a nucleic acid encoding a pair of guide RNAs comprising:
      1. a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018;
      2. a first and second spacer sequence comprising at least 17, 18, 19, 20, or 21 contiguous nucleotides of any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
      3. a first and second spacer sequence that is at least 90% identical to any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and
   ii. a nucleic acid encoding a Staphylococcus aureus Cas9 (SaCas9).
Embodiment A115 is a method of treating Duchenne Muscular Dystrophy (DMD), the method comprising delivering to a cell a single nucleic acid molecule comprising:
   i. a nucleic acid encoding a pair of guide RNAs comprising:
      1. a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; and 146 and 148;
      2. a first and second spacer sequence comprising at least 17, 18, 19, 20, or 21 contiguous nucleotides of any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
      3. a first and second spacer sequence that is at least 90% identical to any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and

a nucleic acid encoding a Staphylococcus lugdunensis (SluCas9).
Embodiment A116 is the method of any one of the previous method or use claims, wherein the single nucleic acid molecule is delivered to the cell on a single vector.
Embodiment A117 is the method of any one of the previous method or use claims, comprising a nucleic acid molecule encoding SaCas9, wherein the spacer sequence is selected from any one of SEQ ID NOs: 12, 15, 16, 20, 27, 28, 32, 33, and 35.
Embodiment A118 is the method of any one of the previous method or use claims, comprising a nucleic acid molecule encoding SaCas9, wherein the SaCas9 comprises the amino acid sequence of SEQ ID NO: 711.
Embodiment A119 is the method of any one of the previous method or use claims, comprising a nucleic acid molecule encoding SaCas9, wherein the SaCas9 is a variant of the amino acid sequence of SEQ ID NO: 711.
Embodiment A120 is the method of any one of the previous method or use claims, comprising a nucleic acid molecule encoding SaCas9, wherein the SaCas9 comprises an amino acid sequence selected from any one of SEQ ID NOs: 715-717.
Embodiment A121 is the method of any one of the previous method or use claims, comprising a nucleic acid molecule encoding SluCas9, wherein the spacer sequence is selected from any one of SEQ ID NOs: 131, 134, 135, 136, 139, 144, 148, 149, 150, 151, 179, 184, 201, 210, 223, 224, and 225.
Embodiment A122 is the method of any one of the previous method or use claims, comprising a nucleic acid molecule encoding SluCas9, wherein the SluCas9 comprises the amino acid sequence of SEQ ID NO: 712.
Embodiment A123 is the method of any one of the previous method or use claims, comprising a nucleic acid molecule encoding SluCas9, wherein the SluCas9 is a variant of the amino acid sequence of SEQ ID NO: 712.
Embodiment A124 is the method of any one of the previous method or use claims, comprising a nucleic acid molecule encoding SluCas9, wherein the SluCas9 comprises an amino acid sequence selected from any one of SEQ ID NOs: 718-720.
Embodiment A125 is a method of excising a portion of an exon with a premature stop codon in a subject with Duchenne Muscular Dystrophy (DMD), the method comprising delivering to a cell a single nucleic acid molecule comprising:
   i. a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and
   ii. a nucleic acid encoding a Staphylococcus aureus Cas9 (SaCas9);

wherein the pair of guide RNAs and SaCas9 excise a portion of the exon.
Embodiment A126 is a method of excising a portion of an exon with a premature stop codon in a subject with Duchenne Muscular Dystrophy (DMD), the method comprising delivering to a cell a single nucleic acid molecule comprising:
   i. a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and
   ii. a nucleic acid encoding a Staphylococcus lugdunensis (SluCas9);

wherein the pair of guide RNAs and SaCas9 excise a portion of the exon.
Embodiment A127 is the method of any one of the previous method claims, comprising a single nucleic acid molecule, wherein the single nucleic acid molecule is delivered to the cell on a single vector.
Embodiment A128 is the method of any one of the previous method claims, wherein a portion of a DMD gene is excised, and wherein the portions of the gene remaining after excision are rejoined with a one-nucleotide insertion.
Embodiment A129 is the method of any one of the previous method claims, wherein a portion of a DMD gene is excised, and wherein the portions of the exon remaining after excision are rejoined without a nucleotide insertion.
Embodiment A130 is the method of any one of the previous method claims, wherein a portion of a DMD gene is excised, wherein the size of the excised portion of the gene is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.
Embodiment A131 is the method of any one of the previous method claims, wherein a portion of a DMD gene is excised, wherein the size of the excised portion of the exon is between 8 and 167 nucleotides.
Embodiment A132 is the method of any one of the previous method claims, wherein a portion of a DMD gene is excised, wherein the portion is within exon 43, 44, 45, 50, 51, or 53.
Embodiment A133 is the method of any one of the previous method claims, comprising a pair of guide RNAs, wherein the pair of guide RNA comprise a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018.
Embodiment A134 is the method of any one of the previous method claims, comprising a pair of guide RNAs, wherein the pair of guide RNAs comprise a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146.
Embodiment A135 is the method of any one of the previous method claims, comprising SaCas9, wherein the SaCas9 comprises the amino acid sequence of SEQ ID NO: 715.
Embodiment A136 is the composition or method of any one of the preceding claims, wherein the single nucleic acid molecule is an AAV vector, wherein the vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding a SaCas9, a polyadenylation sequence, a promoter for expression of a second sgRNA in the same direction as the promoter for SaCas9, a second sgRNA guide sequence, and a second sgRNA scaffold sequence.
Embodiment A137 is the composition or method of claim 136, wherein the promoter for expression of the first sgRNA guide sequence is an hU6 promoter.
Embodiment A138 is the composition or method of any one of claims 136-137, wherein the promoter for expression of the second sgRNA guide sequence is an hU6 promoter.
Embodiment A139 is the composition or method of any one of claims 136-137, wherein the promoter for the expression of the first sgRNA guide sequence is an hU6 promoter, and the promoter for the expression of the second sgRNA guide sequence is an hU6 promoter.
Embodiment A140 is the composition or method of any one of claims 136-137, wherein the promoter for expression of the nucleic acid encoding the first sgRNA guide sequence is an 7SK promoter.
Embodiment A141 is the composition or method of any one of claims 136-137, wherein the promoter for expression of the nucleic acid encoding the second sgRNA guide sequence is an 7SK promoter.
Embodiment A142 is the composition or method of any one of claims 136-137, wherein the promoter for expression of the nucleic acid encoding the second sgRNA guide sequence is an H1m promoter.
Embodiment A143 is the composition or method of any one of the preceding claims, wherein the nucleic acid sequence encoding SaCas9 or SluCas9 is fused to a nucleic acid sequence encoding one or more nuclear localization sequences (NLSs).
Embodiment A144 is the composition or method of any one of the preceding claims, wherein the nucleic acid sequence encoding SaCas9 or SluCas9 is fused to a nucleic acid sequence encoding a nuclear localization sequence (NLS).
Embodiment A145 is the composition or method of any one of the preceding claims, wherein the nucleic acid sequence encoding SaCas9 or SluCas9 is fused to two nucleic acid sequences each encoding a nuclear localization sequence (NLS).
Embodiment A146 is the composition or method of any one of the preceding claims, wherein the nucleic acid sequence encoding SaCas9 or SluCas9 is fused to three nucleic acid sequences each encoding a nuclear localization sequence (NLS).
Embodiment A147 is the composition or method of any one of claims 143-146, wherein the one or more NLSs comprises an SV40 NLS.
Embodiment A148 is the composition or method of any one of claims 143-147, wherein the one or more NLSs comprises an c-Myc NLS.
Embodiment A149 is the composition or method of any one of claims 143-148, wherein the one or more NLSs comprises a nucleoplasmin NLS.
Embodiment A150 is the composition or method of any one of the preceding claims, wherein the nucleic acid encoding the guide RNA or the nucleic acid encoding the pair of guide RNAs comprises a sequence selected from any one of SEQ ID NOs: 600, 601, or 900-917, and wherein the composition or method comprises a nucleic acid encoding SluCas9.
Embodiment A151 is the composition or method of any one of the preceding claims, wherein the nucleic acid encoding the guide RNA or the nucleic acid encoding the pair of guide RNAs comprises a sequence selected from any one of SEQ ID NOs: 901-917, and wherein the composition or method comprises a nucleic acid encoding SluCas9.
Embodiment A152 is the composition of any one of the preceding claims, wherein the nucleic acid molecule encodes at least a first guide RNA and a second guide RNA.
Embodiment A153 is the composition of claim 152, wherein the nucleic acid molecule encodes a spacer sequence for the first guide RNA, a scaffold sequence for the first guide RNA, a spacer sequence for the second guide RNA, and a scaffold sequence for the second guide RNA.
Embodiment A154 is the composition of claim 152, wherein the spacer sequence for the first guide RNA and the spacer sequence for the second guide RNA are the same.
Embodiment A155 is the composition of claim 152, wherein the spacer sequence for the first guide RNA and the spacer sequence for the second guide RNA are different.
Embodiment A156 is the composition of any one of claims 153-155, wherein the scaffold sequence for the first guide RNA and the scaffold sequence for the second guide RNA are the same.
Embodiment A157 is the composition of any one of claims 153-154, wherein the scaffold sequence for the first guide RNA and the scaffold sequence for the second guide RNA are different.
Embodiment A158 is the composition of claim 157, wherein the scaffold sequence for the first guide RNA comprises a sequence selected from the group consisting of SEQ ID NOs: 901-916, and wherein the scaffold sequence for the second guide RNA comprises a different sequence selected from the group consisting of SEQ ID NOs: 901-916.

### DESCRIPTION OF FIGURES

FIG. 1 provides a simplified description of several representative vector configurations. Single-line arrows indicate directionality of expression of the sgRNA(s), while a double-line arrow indicates directionality of expression of the Cas9 protein. For the promoter column, representative promoters for the expression of the sgRNAs are indicated. In a particular embodiment, the Cas9 promoter may be CK8e.

FIG. 2 shows an exemplary schematic of locus-specific indel profiling, as described in Example 2, including editing categories for insertions and deletions.

FIGS. 3A-3B show the editing frequency and indel profile of selected SluCas9 and SaCas9 sgRNAs that target exon 45 of the DMD gene in human HEK293FT cells (FIG. 3A) and mouse Neuro-2a cells (FIG. 3B). A high-performing sgRNA for SpCas9 was included as reference (E45Sp52). Each bar represents a sgRNA and the bar height depicts the average total indel frequency. Indel profiles are shown for each sgRNA as follows: solid black (1-nucleotide (nt) insertion leading to reframe, sometimes referred to as “RF.+1”); solid white (indels other than 1-nt insertion leading to reframe, sometimes referred to as “RF.Other”); checkerboard pattern (indels that disrupt the ±6=nt window of the exon/intron boundaries, referred to as “Exon skipping”); diagonal lines (other indels, sometimes referred to as “OE”). Data are the average of 3 replicates.

FIGS. 4A-4B show the editing frequency and indel profile of selected SluCas9 and SaCas9 sgRNAs that target exon 51 of the DMD gene in human HEK293FT cells (FIG. 4A) and mouse Neuro-2a cells (FIG. 4B). A high performing sgRNA for SpCas9 was included as reference (E51Sp32). Each bar represents a sgRNA and the bar height depicts the average total indel frequency. Indel profiles are shown for each sgRNA as follows: solid black (1-nucleotide (nt) insertion leading to reframe, referred to as “RF.+1”); solid white (indels other than 1-nt insertion leading to reframe, referred to as “RF.Other”); checkerboard pattern (indels that disrupt the ±6=nt window of the exon/intron boundaries, referred to as “Exon skipping”); diagonal lines (other indels, sometimes referred to as “OE” or “Other indel”). Data are the average of 3 replicates.

FIGS. 5A-5B show the editing frequency and indel profile of selected SluCas9 and SaCas9 sgRNAs within exon 53 of the DMD gene in human HEK293FT cells (FIG. 5A) and mouse Neuro-2a cells (FIG. 5B). A high performing sgRNA for SpCas9 was included as reference (E53Sp63). Each bar represents a sgRNA and the bar height depicts the average total indel frequency. Indel profiles are shown for each sgRNA using distinct colors as follows: solid black (1-nucleotide (nt) insertion leading to reframe, referred to as “RF.+1”); solid white (indels other than 1-nt insertion leading to reframe, referred to as “RF.Other”); checkerboard pattern (indels that disrupt the ±6=nt window of the exon/intron boundaries, referred to as “Exon skipping”); diagonal lines (other indels, referred to as “OE” or “Other indel”). Data are the average of 3 replicates.

FIGS. 6A-D show editing frequency and indel profile of selected SaCas9-KKH and SluCas9 sgRNA pairs within exon 45 in HEK293FT cells (FIG. 6B) and Neuro-2a cells (FIG. 6D). FIG. 6A shows a schematic of editing. A single cut sgRNA for SaCas9 (SaCas9-4) was included as reference (FIG. 6C). Each bar represents a sgRNA and the bar height depicts the average total indel frequency. Indel profiles are shown for each sgRNA using distinct patterns. Error bar=upper limit of SEM for each indel group.

FIG. 7A shows the nucleotide composition and RNA secondary structure of the stem-loop I in different SluCas9 single-guide RNA scaffolds. Key differences in the sequence and secondary structure between Slu-VCGT-4.5, Slu-VCGT-4 and Slu-VCGT-5 are depicted. Squares and triangles show the difference in the secondary structure in the upper stem. Diamond and pentagon shapes show the single nucleotide change in the bottom stem.

FIG. 7B is a histogram showing the percentage of different types of indels generated by two SluCas9 sgRNA candidates: SluCas9-23 and SluCas9-24. For each guide RNA, three scaffolds were tested, including Slu-VCGT-4.5, Slu-VCGT-4 and Slu-VCGT-5. Each sgRNA was tested at three different RNP doses. The exact amounts of SluCas9 protein and sgRNA tested were: 6.75 pmol:37.5 pmol for low dose, 12.5 pmol:75 pmol for middle dose, and 25 pmol:150 pmol for high dose. The different colors of the bars in the histogram represent different types of indels generated by sgRNAs. Black represents the percentage of +1 bp insertions. White represents the percentage of other insertions and deletions that have the potential to restore the reading frame of particular DMD patient mutations of interest. These are referred to as “RF other”, which represents the sum of 2, 5, 8, 11 bp deletions within the alignment window of −20 bp to +20 bp around the Cas9 cut site. The remaining indels shown in pattern are classified as “Other indels”.

FIG. 8 shows editing frequency and indel profile of selected SluCas9 sgRNA pairs within exon 45 in primary human skeletal muscle myoblasts (HsMMs). Two single cut sgRNAs for SpCas9 (EX-145, E45Sp52) and SluCas9 (SluCas9-24) were included as reference. Each bar represents a sgRNA or a sgRNA pair and the bar height depicts the average total indel frequency. Indel profiles are shown using distinct patterns. Error bar=upper limit of SD for each indel group.

FIGS. 9A and 9B show editing frequency and indel profile of selected SaCas9-KKH and SluCas9 sgRNA pairs within exon 51 in HEK293FT cells. Each bar represents a sgRNA or a sgRNA pair and the bar height depicts the average total indel frequency. Indel profiles are shown using distinct patterns. Error bar=upper limit of SD for each indel group.

FIGS. 10A and 10B show editing frequency and indel profile of selected AAV vectors in C2C12 myotubes. Three test guides are shown for FIG. 10A and 4 test guides for FIG. 10B. Each bar represents a different AAV configuration and the bar height depicts the average total indel frequency. Indel profiles are shown using distinct patterns. Error bar=upper limit of SD for each indel group.

FIG. 11 shows vector genome quantitation of selected AAV vectors in C2C12 myotubes. Each bar represents a different AAV configuration and the bar height depicts the average vector genome copy number per μg DNA. Error bar=upper limit of SD.

FIGS. 12A and 12B show transgene expression of selected AAV vectors in C2C12 myotubes. FIG. 12A: each bar represents a different AAV configuration and the bar height depicts the average Cas9 transgene copy per ug RNA. Cas9 nucleases are shown using distinct patterns. FIG. 12B: each bar represents a different sgRNA and the bar height depicts the average sgRNA transgene copy per ug RNA. Promoter combinations are shown using distinct patterns. Error bar=upper limit of SD.

FIG. 13 shows Cas9 localization of selected AAV vectors in C2C12 myotubes. In FIG. 13 and throughout the application, single-line arrows indicate directionality of expression of the sgRNA(s), while a double-line or “thick” arrow (with or without an embedded “X”) indicates directionality of expression of the Cas9 protein. SluCas9's location is shown in the bottom panel of each; MYOG is shown with representative arrows, DAPI is shown with representative bold, larger arrows. Image shown at 20-25 μm scale.

FIGS. 14A and 14B show editing frequency and indel profile of selected AAV vectors in heart (FIG. 14A) and quadriceps (FIG. 14B) of dEx44 mouse model. Each bar represents a different AAV configuration and the bar height depicts the average total indel frequency. Indel profiles are shown using distinct patterns. Error bar=upper limit of SD for each indel group.

FIGS. 15A and 15B show dystrophin restoration of selected AAV vectors in heart (FIG. 15A) and quadriceps (FIG. 15B) of dEx44 mouse model. Each group represents a different AAV configuration and the horizontal bar depicts the average total dystrophin protein level.

FIGS. 16A and 16B show vector genome quantitation of selected AAV vectors in heart (FIG. 16A) and quadriceps (FIG. 16B) of dEx44 mouse model. Each group represents a different AAV configuration and the horizontal bar depicts the average vector genome copy number per ug DNA.

FIGS. 17A and 17B show Cas9 and sgRNA transgene expression of selected AAV vectors in heart (FIG. 17A) and quadriceps (FIG. 17B) of dEx44 mouse model. Each group represents a different AAV configuration and the horizontal bar depicts the average transgene copy number per ug RNA. Closed circles indicate Cas9 mRNA expression and open circles indicate sgRNA expression.

FIGS. 18A-18C show editing frequency and indel profile of selected Cas12i2 guide RNAs used with Cas12i2 endonuclease in exon 45 in HEK293FT cells. FIG. 18A shows a schematic of the experiment, FIG. 18B shows indel frequency for selected pairs of guide RNAs, and FIG. 18C shows a single cut sgRNA for SaCas9 (SaCas9-4) and for SluCas9 (SluCas9-24) for reference.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 17, 18, 19, 20, 21, 22, 23, 24, or 25 contiguous nucleotides of a sequence selected from SEQ ID NOs: 1-35 (for SaCas9), and 100-225 (for SluCas9). In some embodiments, the guide sequence comprises a sequence selected from SEQ ID NOs: 1-35, 100-225, 1000-1078, 2000-2116, 3000-3069, or 4000-4251. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, 20, 21, 22, 23, 24, or 25 contiguous nucleotides of a sequence selected from SEQ ID NOs: 1-35, 100-225, 1000-1078, 2000-2116, 3000-3069, or 4000-4251. In some embodiments, the guide sequence comprises a sequence with about 75%, 80%, 85%, 90%, 95%, 96%, 97%, 98%, 99%, or 100% identity to a sequence selected from SEQ ID NOs: 1-35, 100-225, 1000-1078, 2000-2116, 3000-3069, or 4000-4251. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

In some embodiments, the guide sequence comprises a sequence selected from SEQ ID NOs: 1-35, 100-225, 1000-1078, 2000-2116, 3000-3069, or 4000-4251, wherein if the 5′ terminal nucleotide is not guanine, one or more guanine (g) is added to the sequence at its 5′ end. The 5′ g or gg is required in some instances for transcription, for example, for expression by the RNA polymerase III-dependent U6 promoter or the T7 promoter. In some embodiments, a 5′ guanine is added to any one of the guide sequences or pairs of guide sequences disclosed herein.

Target sequences for Cas9s include both the positive and negative strands of genomic DNA (i.e., the sequence given and the sequence's reverse compliment), as a nucleic acid substrate for a Cas9 is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “ribonucleoprotein” (RNP) or “RNP complex” refers to a guide RNA together with a Cas9. In some embodiments, the guide RNA guides the Cas9 such as Cas9 to a target sequence, and the guide RNA hybridizes with and the agent binds to the target sequence, which can be followed by cleaving or nicking (in the context of a modified “nickase” Cas9).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Guide sequences useful in the guide RNA compositions and methods described herein are shown, for example, in Table 1A, Table 1B, and Table 5 and throughout the specification.

As used herein, a “target sequence” refers to a sequence of nucleic acid in a target gene that has complementarity to at least a portion of the guide sequence of the guide RNA. The interaction of the target sequence and the guide sequence directs a Cas9 to bind, and potentially nick or cleave (depending on the activity of the agent), within the target sequence.

As used herein, “treatment” refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. For example, treatment of DMD may comprise alleviating symptoms of DMD.

As used herein, “ameliorating” refers to any beneficial effect on a phenotype or symptom, such as reducing its severity, slowing or delaying its development, arresting its development, or partially or completely reversing or eliminating it. In the case of quantitative phenotypes such as expression levels, ameliorating encompasses changing the expression level so that it is closer to the expression level seen in healthy or unaffected cells or individuals.

A “pharmaceutically acceptable excipient” refers to an agent that is included in a pharmaceutical formulation that is not the active ingredient. Pharmaceutically acceptable excipients may e.g., aid in drug delivery or support or enhance stability or bioavailability.

The term “about” or “approximately” means an acceptable error for a particular value as determined by one of ordinary skill in the art, which depends in part on how the value is measured or determined.

As used herein, “Staphylococcus aureus Cas9” may also be referred to as SaCas9, and includes wild type SaCas9 (e.g., SEQ ID NO: 711) and variants thereof. A variant of SaCas9 comprises one or more amino acid changes as compared to SEQ ID NO: 711, including insertion, deletion, or substitution of one or more amino acids, or a chemical modification to one or more amino acids.

As used herein, “Staphylococcus lugdunensis Cas9” may also be referred to as SluCas9, and includes wild type SluCas9 (e.g., SEQ ID NO: 712) and variants thereof. A variant of SluCas9 comprises one or more amino acid changes as compared to SEQ ID NO: 712, including insertion, deletion, or substitution of one or more amino acids, or a chemical modification to one or more amino acids.

### II. Compositions

Provided herein are compositions useful for treating Duchenne Muscular Dystrophy (DMD), e.g., using a single or multiple (e.g., at least 2) nucleic acid molecule encoding 1) one or more guide RNAs comprising one or more guide sequences of Table 1A, Table 1B, or Table 5; and 2) SaCas9 (for SEQ ID NOs: 1-35, 1000-1078 or 3000-3069) or SluCas9 (for SEQ ID NOs: 100-225, 2000-2116, or 4000-4251). Such compositions may be administered to subjects having or suspected of having DMD.

In some embodiments, a composition is provided comprising, consisting of, or consisting essentially of a single nucleic acid molecule comprising: i) a nucleic acid encoding Staphylococcus aureus Cas9 (SaCas9) or Staphylococcus lugdunensis (SluCas9) and at least one, at least two, or at least three guide RNAs; or ii) a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or iii) a nucleic acid encoding Staphylococcus aureus Cas9 (SaCas9) or Staphylococcus lugdunensis (SluCas9) and one to three guide RNAs.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9); and a second nucleic acid that does not encode a SaCas9 or SluCas9 and encodes any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and i) at least one, at least two, or at least three guide RNAs; or ii) from one to n guide RNAs, wherein n is no more than the maximum number of guide RNAs that can be expressed from said nucleic acid; or iii) one to three guide RNAs; and a second nucleic acid that does not encode a SaCas9 or SluCas9, optionally wherein the second nucleic acid comprises any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and at least one, at least two, or at least three guide RNAs; and a second nucleic acid that does not encode a SaCas9 or SluCas9 and encodes from one to six guide RNAs.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and at least two guide RNAs, wherein at least one guide RNA binds upstream of sequence to be excised and at least one guide RNA binds downstream of sequence to be excised; and a second nucleic acid that does not encode a SaCas9 or SluCas9 and encodes at least one additional copy of the guide RNAs encoded in the first nucleic acid. In some embodiments, the guide RNA excises a portion of a DMD gene, optionally an exon, intron, or exon/intron junction.

In some embodiments, a composition is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and a first and a second guide RNA that function to excise a portion of a DMD gene; and a second nucleic acid encoding at least 2 or at least 3 copies of the first guide RNA and at least 2 or at least 3 copies of the second guide RNA.

In some embodiments, a composition is provided comprising, consisting of, or consisting essentially of one or more nucleic acid molecules encoding an endonuclease and a pair of guide RNAs, wherein each guide RNA targets a different sequence in a DMD gene, wherein the endonuclease and pair of guide RNAs are capable of excising a target sequence in DNA that is between 5-250 nucleotides in length. In some embodiments, the endonuclease is a class 2, type II Cas endonuclease. In some embodiments, the class 2, type II Cas endonuclease is SpCas9, SaCas9, or SluCas9. In some embodiments, the endonuclease is not a class 2, type V Cas endonuclease. In some embodiments, the excised target sequence comprises a splice acceptor site or a splice donor site. In some embodiments, the excised target sequence comprises a premature stop codon in the DMD gene. In some embodiments, the excised target sequence does not comprise an entire exon of the DMD gene. In some embodiments, any of the methods and/or ribonucleoprotein complexes disclosed herein do not destroy/specifically alter the sequence of a splice acceptor site, splice donor site, or premature stop codon site.

In some embodiments, the guide RNA in the composition comprises any one of the following:
a. when SaCas9 is used, one or more spacer sequences selected from any one of SEQ ID NOs: 1-35, 1000-1078, and 3000-3069; or
b. when SluCas9a is used, one or more spacer sequences selected from any one of SEQ ID NOs: 100-225, 2000-2116, and 4000-4251; or
c. when SaCas9 is used, one or more spacer sequences comprising at least 17, 18, 19, or 20 contiguous nucleotides of a spacer sequence selected from any one of SEQ ID NOs: 1-35, 1000-1078, and 3000-3069; or
d. when SluCas9a is used, one or more spacer sequences comprising at least 17, 18, 19, or 20 contiguous nucleotides of a spacer sequence selected from any one of SEQ ID NOs: 100-225, 2000-2116, and 4000-4251; or
e. when SaCas9 is used, one or more spacer sequences that is at least 90% identical to any one of SEQ ID NOs: 1-35, 1000-1078, and 3000-3069; or
f. when SluCas9a is used, one or more spacer sequences that is at least 90% identical to any one of SEQ ID NOs: 100-225, 2000-2116, and 4000-4251; or
g. when SaCas9 is used with at least two guide RNAs, a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
h. when SaCas9 is used with at least two guide RNAs, at least 17, 18, 19, 20, or 21 contiguous nucleotides of a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
i. when SaCas9 is used with at least two guide RNAs, at least 90% identical to a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
j. when SluCas9 is used with at least two guide RNAs, a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
k. when SluCas9 is used with at least two guide RNAs, at least 17, 18, 19, 20, or 21 contiguous nucleotides of a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
l. when SluCas9 is used with at least two guide RNAs, at least 90% identical to a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
m. when SluCas9 is used with at least two guide RNAs, at least 90% identical to a first and second spacer sequence selected from any one of:
   i. SEQ ID NOS: 148 and 134,
   ii. SEQ ID Nos: 145 and 131,
   iii. SEQ ID Nos: 144 and 149;
   iv. SEQ ID Nos: 144 and 150; and
   v. SEQ ID Nos: 146 and 148; or
n. when SaCas9 is used with at least two guide RNAs, at least 90% identical to a first and second spacer sequence selected from any one of:
   i. SEQ ID NOs: 12 and 1013; and
   ii. SEQ ID Nos: 12 and 1016.

In some embodiments, the disclosure provides for a composition comprising: a) one or more nucleic acid molecules encoding a SluCas9 and b) a first spacer sequence and a second spacer sequence selected from SEQ ID Nos: 148 and 134. In some embodiments, the composition comprises: a) one or more nucleic acid molecules encoding a SluCas9 and b) a first spacer sequence and a second spacer sequence selected from SEQ ID Nos: 145 and 131. In some embodiments, the composition comprises: a) one or more nucleic acid molecules encoding a SluCas9 and b) a first spacer sequence and a second spacer sequence selected from SEQ ID Nos: 144 and 149. In some embodiments, the composition comprises: a) one or more nucleic acid molecules encoding a SluCas9 and b) a first spacer sequence and a second spacer sequence selected from SEQ ID Nos: 144 and 150. In some embodiments, the composition comprises: a) one or more nucleic acid molecules encoding a SluCas9 and b) a first spacer sequence and a second spacer sequence selected from SEQ ID Nos: 146 and 148. In some embodiments, the composition comprises: a) one or more nucleic acid molecules encoding a SaCas9 (e.g., an SaCas9-KKH) and b) a first spacer sequence and a second spacer sequence selected from SEQ ID Nos: 12 and 1013. In some embodiments, the composition comprises: a) one or more nucleic acid molecules encoding a SaCas9 (e.g., an SaCas9-KKH) and b) a first spacer sequence and a second spacer sequence selected from SEQ ID Nos: 12 and 1016.

In some embodiments, the one or more guide RNAs direct the Cas9 to a site in or near any one of exon 43, 44, 45, 50, 51, or 53 of dystrophin. For example, the Cas9 may be directed to cut within 10, 20, 30, 40, or 50 nucleotides of a target sequence.

The disclosure herein may reference a “first and a second spacer” or a “first and a second guide RNA, gRNA, or sgRNA” followed by one or more pairs of specific sequences. It should be noted that the order of the sequences in the pair is not intended to be restricted to the order in which they are presented. For example, the phrase “the first sgRNA and the second sgRNA comprise the sequences of SEQ ID NOs: 10 and 15” could mean that the first sgRNA comprises the sequence of SEQ ID NO: 10 and the second sgRNA sequence comprises the sequence of SEQ ID NO: 15, or this phrase could mean that the first sgRNA comprises the sequence of SEQ ID NO: 15 and the second sgRNA sequence comprises the sequence of SEQ ID NO: 10.

In some embodiments, a composition comprising a single nucleic acid molecule encoding one or more guide RNAs and a Cas9 is provided, wherein the single nucleic acid molecule comprises:
a. a first nucleic acid encoding one or more spacer sequences selected from any one of SEQ ID NOs: 1-35, 1000-1078, or 3000-3069 and a second nucleic acid encoding a Staphylococcus aureus Cas9 (SaCas9);
b. a first nucleic acid encoding one or more spacer sequences selected from any one of SEQ ID NOs: 100-225, 2000-2116, or 4000-4251 and a second nucleic acid encoding a Staphylococcus lugdunensis (SluCas9);
c. a first nucleic acid encoding one or more spacer sequences comprising at least 17, 18, 19, or 20 contiguous nucleotides of a spacer sequence selected from any one of SEQ ID NOs: 1-35, 1000-1078, or 3000-3069 and a second nucleic acid encoding a Staphylococcus aureus Cas9 (SaCas9);
d. a first nucleic acid encoding one or more spacer sequences comprising at least 17, 18, 19, or 20 contiguous nucleotides of a spacer sequence selected from any one of SEQ ID NOs: 100-225, 2000-2116, or 4000-4251 and a second nucleic acid encoding a Staphylococcus lugdunensis (SluCas9);
e. a first nucleic acid encoding one or more spacer sequences that is at least 90% identical to any one of SEQ ID NOs: 1-35, 1000-1078, or 3000-3069 and a second nucleic acid encoding a Staphylococcus aureus Cas9 (SaCas9);
f. a first nucleic acid encoding one or more spacer sequences that is at least 90% identical to any one of SEQ ID NOs: 100-225, 2000-2116, or 4000-4251 and a second nucleic acid encoding a Staphylococcus lugdunensis (SluCas9);
g. a first nucleic acid encoding a pair of guide RNAs comprising a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9);
h. a first nucleic acid encoding a pair of guide RNAs comprising at least 17, 18, 19, 20, or 21 contiguous nucleotides of a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9);
i. a first nucleic acid encoding a pair of guide RNAs that is at least 90% identical to a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9);
j. a first nucleic acid encoding a pair of guide RNAs comprising a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9);
k. a first nucleic acid encoding a pair of guide RNAs comprising at least 17, 18, 19, 20, or 21 contiguous nucleotides of a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9);
l. a first nucleic acid encoding a pair of guide RNAs that is at least 90% identical to a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9);
m. a first nucleic acid encoding a pair of guide RNAs that is at least 90% identical to a first and second spacer sequence selected from any one of:
   i. SEQ ID NOS: 148 and 134,
   ii. SEQ ID Nos: 145 and 131,
   iii. SEQ ID Nos: 144 and 149;
   iv. SEQ ID Nos: 144 and 150;
   v. SEQ ID Nos: 146 and 148; and a second nucleic acid encoding a Staphylococcus lugdunensis (SluCas9); or
n. a first nucleic acid encoding a pair of guide RNAs that is at least 90% identical to a first and second spacer sequence selected from any one of:
   i. SEQ ID NOs: 12 and 1013; and
   ii. SEQ ID Nos: 12 and 1016; and a second nucleic acid encoding a Staphylococcus aureus Cas9 (SaCas9).

In some embodiments, a composition is provided comprising, consisting of, or consisting essentially of one or more nucleic acid molecules encoding a Staphylococcus lugdunensis (SluCas9) and at least two guide RNAs, wherein a first and second guide RNA target different sequences in a DMD gene, wherein the first and a second guide RNA comprise a sequence that is at least 90% identical to a first and second spacer sequence selected from any one of:
i. SEQ ID NOS: 148 and 134,
ii. SEQ ID Nos: 145 and 131,
iii. SEQ ID Nos: 144 and 149;
iv. SEQ ID Nos: 144 and 150;
v. SEQ ID Nos: 146 and 148.

In some embodiments, a composition is provided comprising, consisting of, or consisting essentially of one or more nucleic acid molecules encoding an endonuclease and at least two guide RNAs, wherein the guide RNAs each target a different sequence in a DMD gene, wherein the guide RNAs each comprise a sequence that is at least 90% identical to a first and second spacer sequence selected from any one of:
i. SEQ ID NOs: 12 and 1013; and
ii. SEQ ID Nos: 12 and 1016; and

a second nucleic acid encoding a Staphylococcus aureus Cas9 (SaCas9).

In some embodiments, the first and/or the second nucleic acid, if present, comprises at least two guide RNAs. In some embodiments, the first and/or the second nucleic acid, if present, comprises at least three guide RNAs. In some embodiments, the first and/or the second nucleic acid, if present, comprises at least four guide RNAs. In some embodiments, the first and/or the second nucleic acid, if present, comprises at least five guide RNAs. In some embodiments, the first and/or the second nucleic acid, if present, comprises at least six guide RNAs. In some embodiments, the first nucleic acid comprises an endonuclease and at least one, at least two, or at least three guide RNAs. In some embodiments, the first nucleic acid comprises an endonuclease and from one to n guide RNAs, wherein n is no more than the maximum number of guide RNAs that can be expressed from said nucleic acid. In some embodiments, the first nucleic acid comprises an endonuclease and from one to three guide RNAs.

In some embodiments, the second nucleic acid, if present, encodes at least one, at least two, at least three, at least four, at least five, or at least six guide RNAs. In some embodiments, the second nucleic acid, if present, encodes from one to n guide RNAs, wherein n is no more than the maximum number of guide RNAs that can be expressed from said nucleic acid In some embodiments, the second nucleic acid, if present, encodes from one to six guide RNAs. In some embodiments, the second nucleic acid, if present, encodes 2-10, 2-9, 2-8, 2-7, 2-6, 2-5, 2-4, or 2-3 guide RNAs. In some embodiments, the second nucleic acid, if present, encodes 2, 3, 4, 5, or 6 guide RNAs.

In some embodiments comprising at least two nucleic acid molecules, the guide RNA encoded by the first nucleic acid and the second nucleic acid are the same. In some embodiments comprising at least two nucleic acid molecules, the guide RNA encoded by the first nucleic acid and the second nucleic acid are different. In some embodiments comprising at least two nucleic acid molecules, at least one guide RNA binds to a target sequence within an exon in the DMD gene that is upstream of a premature stop codon, and wherein at least one guide RNA binds to a target sequence within an exon in the DMD gene that is downstream of a premature stop codon. In some embodiments comprising at least two nucleic acid molecules, the same guide RNA is encoded by the nucleic acid of the first and second nucleic acid molecule. In some embodiments comprising at least two nucleic acid molecules, the second nucleic acid molecule encodes a guide RNA that binds to the same target sequence as the guide RNA in the first nucleic acid molecule. In some embodiments comprising at least two nucleic acid molecules, the second nucleic acid molecule encodes at least 2, at least 3, at least 4, at least 5, or at least 6 guide RNAs, wherein the guide RNAs in the second nucleic acid molecule bind to the same target sequence as the guide RNA in the first nucleic acid molecule.

In some embodiments, the guide RNA binds to one or more target sequences within a DMD gene. In some embodiments, the guide RNA binds to one or more target sequences within an exon of the DMD gene. In some embodiments comprising two guide RNAs, i) each guide RNA targets a sequence within an exon; ii) one guide RNA targets a sequence within an exon and one targets a sequence within an intron; or iii) each guide RNA targets a sequence within an intron.

In some embodiments comprising at least two guide RNAs, i) each guide RNA targets the same genomic target sequence; ii) each guide RNA targets a different target sequence; or iii) at least one guide RNA targets one sequence and at least one guide RNA targets a different sequence.

In some embodiments comprising a guide RNA that binds to an exon of the DMD gene, the exon is selected from exon 43, 44, 45, 50, 51, and 53.

In some embodiments comprising at least two guide RNAs that binds to an exon of the DMD gene, at least one guide RNA binds to a target sequence within an exon in the DMD gene, and at least one guide RNA binds to a different target sequence within the same exon in the DMD gene.

In some embodiments comprising at least two guide RNAs, at least one guide RNA binds to a target sequence within an exon that is upstream of a premature stop codon, and at least one guide RNA binds to a target sequence within an exon that is downstream of a premature stop codon.

In some embodiments comprising at least two guide RNAs, at least one guide RNA binds to a target sequence within an exon in the DMD gene, and at least one guide RNA binds to a different target sequence within the same exon in the DMD gene, wherein when expressed in vivo or in vivo, a portion of the exon is excised. In some embodiments comprising at least two guide RNAs, wherein once expressed in vivo or in vivo, the guide RNAs excise a portion of the exon.

In some embodiments comprising at least two guide RNAs, wherein once expressed in vivo or in vivo, the guide RNAs excise a portion of the exon, and wherein the portion of the exon remaining after excision are rejoined with a one nucleotide insertion. In some embodiments comprising at least two guide RNAs, wherein once expressed in vivo or in vivo, the guide RNAs excise a portion of the exon, wherein the portion of the exon remaining after excision is rejoined without a nucleotide insertion.

In some embodiments comprising at least two guide RNAs, wherein once expressed in vivo or in vivo, the guide RNAs excise a portion of the exon, wherein the size of excised portion of the exon is between 5, 6, 7, 8, 9, 10, 15, or 20 and 250 nucleotides in length. In some embodiments comprising at least two guide RNAs, once expressed in vivo or in vivo, the guide RNAs excise a portion of the exon, wherein the size of excised portion of the exon is between 5 and 250, 5 and 200, 5 and 150, 5 and 100, 5 and 75, 5 and 50, 5 and 25, 5 and 10, 20 and 250, 20 and 200, 20 and 150, 20 and 100, 20 and 75, 20 and 50, 20 and 25, 50 and 250, 50 and 200, 50 and 150, 50 and 100, and 50 and 75 nucleotides.

In some embodiments comprising at least two guide RNAs, wherein once expressed in vivo or in vivo, the guide RNAs excise a portion of the exon, wherein the size of excised portion of the exon is between 8 and 167 nucleotides.

In some embodiments, a guide RNA and a Cas9 are provided on a single nucleic acid molecule. In some embodiments, the single nucleic acid molecule comprises a nucleic acid encoding a guide RNA and a nucleic acid encoding a SaCas9 or SluCas9. In some embodiments, two guide RNAs and a Cas9 are provided on a single nucleic acid molecule. In some embodiments, the single nucleic acid molecule comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

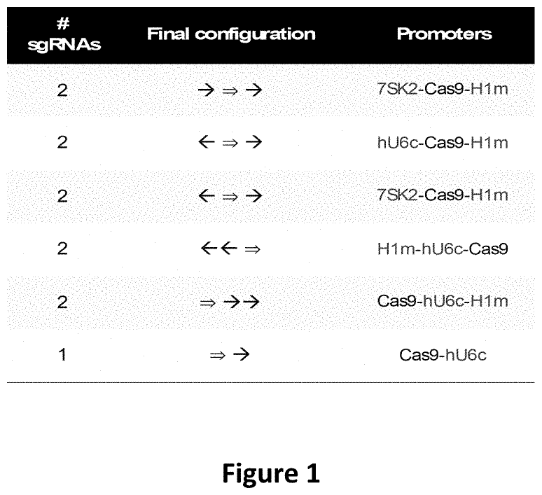

In some embodiments, the single nucleic acid molecule is a single vector. In some embodiments, the single vector expresses the one or two guide RNAs and Cas9 according to the schemes of FIG. 1. In some embodiments, a guide RNA and a Cas9 are provided on a single vector. In some embodiments, the single vector comprises a nucleic acid encoding a guide RNA and a nucleic acid encoding a SaCas9 or SluCas9. In some embodiments, two guide RNAs and a Cas9 are provided on a single vector. In some embodiments, the single vector comprises a nucleic acid encoding a first guide RNA, a nucleic acid encoding a second guide RNA, and a nucleic acid encoding a SaCas9 or SluCas9. In some embodiments, the spacer sequences of the first and second guide RNAs are identical. In some embodiments, the spacer sequences of the first and second guide RNAs are not identical.

Each of the guide sequences shown in Table 1A, Table 1B, and Table 5 may further comprise additional nucleotides to form or encode a crRNA, e.g., using any known sequence appropriate for the Cas9 being used. In some embodiments, the crRNA comprises (5′ to 3′) at least a spacer sequence and a first complementarity domain. The first complementary domain is sufficiently complementary to a second complementarity domain, which may be part of the same molecule in the case of an sgRNA or in a tracrRNA in the case of a dual or modular gRNA, to form a duplex. See, e.g., US 2017/0007679 for detailed discussion of crRNA and gRNA domains, including first and second complementarity domains.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is: GTTTTAGTACTCTGGAAACAGAATCTACTAAAACAAGGCAAAATGCCGTGTTTATCTCGT CAACTTGTTGGCGAGAT (SEQ ID NO: 501) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 910, or a sequence that differs from SEQ ID NO: 910 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 502) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 911, or a sequence that differs from SEQ ID NO: 911 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is: GTTTAAGTACTCTGGAAACAGAATCTACTTAAACAAGGCAAAATGCCGTGTTTATCTCGT CAACTTGTTGGCGAGAT (SEQ ID NO: 503) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 912, or a sequence that differs from SEQ ID NO: 912 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is: GTTTCAGTACTCTGGAAACAGAATCTACTGAAACAAGGCAAAATGCCGTGTTTATCTCGT CAACTTGTTGGCGAGAT (SEQ ID NO: 932) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 932, or a sequence that differs from SEQ ID NO: 932 by no more than 1, 2, 3, 4, 5, 10, 15, 20, or 25 nucleotides.

Two exemplary scaffold sequences suitable for use with SluCas9 to follow the guide sequence at its 3′ end is: GTTTTAGTACTCTGGAAACAGAATCTACTGAAACAAGACAATATGTCGTGTTTATCCCAT CAATTTATTGGTGGGA (SEQ ID NO: 900), and GTTTAAGTACTCTGTGCTGGAAACAGCACAGAATCTACTGAAACAAGACAATATGTCGT GTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 900 or SEQ ID NO: 601, or a sequence that differs from SEQ ID NO: 900 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown below in the 5′ to 3′ orientation:

| | | | | Streak of |
|---|---|---|---|---|
| | | | | Homology to |
| Scaffold | SEQ | | Homology | Slu v5 |
| ID | ID NO | Scaffold Sequence (5′ to 3′) | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 | | ATCCCATCAATTTATTGGTGGGA | | |
| Slu v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SluCas9 to follow the guide sequence at its 3′ end is selected from any one of SEQ ID NOs: 900 or 601, or 901-917 in 5′ to 3 orientation (see below). In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 900 or 601, or 901-917, or a sequence that differs from any one of SEQ ID NOs: 900 or 601, or 901-917 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 901-917 in 5′ to 3 orientation (see below). In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 901-917, or a sequence that differs from any one of SEQ ID NOs: 901-917 by no more than 1, 2, 3, 4, 5, 10, 15, 20, or 25 nucleotides.

In some embodiments, the nucleic acid encoding the gRNA or the nucleic acid encoding the pair of gRNAs comprises a sequence comprising SEQ ID NO: 900. In some embodiments, the nucleic acid encoding the gRNA or the nucleic acid encoding the pair of gRNAs comprises a sequence comprising SEQ ID NO: 601. In some embodiments, the nucleic acid encoding the gRNA or the nucleic acid encoding the pair of gRNAs comprises a sequence comprising SEQ ID NO: 900. In some embodiments, the nucleic acid encoding the gRNA or the nucleic acid encoding the pair of gRNAs comprises a sequence comprising SEQ ID NO: 901. In some embodiments, the nucleic acid encoding the gRNA or the nucleic acid encoding the pair of gRNAs comprises a sequence comprising SEQ ID NO: 902. In some embodiments, the nucleic acid encoding the gRNA or the nucleic acid encoding the pair of gRNAs comprises a sequence comprising SEQ ID NO: 903. In some embodiments, the nucleic acid encoding the gRNA or the nucleic acid encoding the pair of gRNAs comprises a sequence comprising SEQ ID NO: 904. In some embodiments, the nucleic acid encoding the gRNA or the nucleic acid encoding the pair of gRNAs comprises a sequence comprising SEQ ID NO: 905. In some embodiments, the nucleic acid encoding the gRNA or the nucleic acid encoding the pair of gRNAs comprises a sequence comprising SEQ ID NO: 906. In some embodiments, the nucleic acid encoding the gRNA or the nucleic acid encoding the pair of gRNAs comprises a sequence comprising SEQ ID NO: 907. In some embodiments, the nucleic acid encoding the gRNA or the nucleic acid encoding the pair of gRNAs comprises a sequence comprising SEQ ID NO: 908. In some embodiments, the nucleic acid encoding the gRNA or the nucleic acid encoding the pair of gRNAs comprises a sequence comprising SEQ ID NO: 909. In some embodiments, the nucleic acid encoding the gRNA or the nucleic acid encoding the pair of gRNAs comprises a sequence comprising SEQ ID NO: 910. In some embodiments, the nucleic acid encoding the gRNA or the nucleic acid encoding the pair of gRNAs comprises a sequence comprising SEQ ID NO: 911. In some embodiments, the nucleic acid encoding the gRNA or the nucleic acid encoding the pair of gRNAs comprises a sequence comprising SEQ ID NO: 912. In some embodiments, the nucleic acid encoding the gRNA or the nucleic acid encoding the pair of gRNAs comprises a sequence comprising SEQ ID NO: 913. In some embodiments, the nucleic acid encoding the gRNA or the nucleic acid encoding the pair of gRNAs comprises a sequence comprising SEQ ID NO: 914. In some embodiments, the nucleic acid encoding the gRNA or the nucleic acid encoding the pair of gRNAs comprises a sequence comprising SEQ ID NO: 915. In some embodiments, the nucleic acid encoding the gRNA or the nucleic acid encoding the pair of gRNAs comprises a sequence comprising SEQ ID NO: 916. In some embodiments, the nucleic acid encoding the gRNA or the nucleic acid encoding the pair of gRNAs comprises a sequence comprising SEQ ID NO: 917. In some embodiments, comprising a pair of gRNAs, one of the gRNAs comprises a sequence selected from any one of SEQ ID NOs: 900 or 601, or 901-917. In some embodiments, comprising a pair of gRNAs, both of the gRNAs comprise a sequence selected from any one of SEQ ID NOs: 900 or 601, or 901-917. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the scaffold sequence comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID Nos: 500, or 910-912 (for SaCas9), and 900 or 601, or 901-917 (for SluCas9)) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In general, in the case of a DNA nucleic acid construct encoding a guide RNA, the U residues in any of the RNA sequences described herein may be replaced with T residues, and in the case of a guide RNA construct encoded by a DNA, the T residues may be replaced with U residues.

Provided herein are compositions comprising one or more guide RNAs or one or more nucleic acids encoding one or more guide RNAs comprising a guide sequence disclosed herein in Table 1A, Table 1B, and Table 5 and throughout the specification.

In some embodiments, a composition is provided comprising a guide RNA, or nucleic acid encoding a guide RNA, wherein the guide RNA comprises 17, 18, 19, or 20 contiguous nucleotides of any one of the guide sequences disclosed herein in Table 1A, Table 1B, and Table 5 and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to at least 17, 18, 19, or 20 contiguous nucleotides of a guide sequence shown in Table 1A, Table 1B, and Table 5 and throughout the specification.

In some embodiments, a composition is provided comprising a guide RNA, or nucleic acid encoding a guide RNA, wherein the guide RNA comprises a sequence with about 75%, 80%, 85%, 90%, 95%, 96%, 97%, 98%, 99%, or 100% identity to a guide sequence shown in Table 1A, Table 1B, and Table 5 and throughout the specification.

In some embodiments, a composition is provided comprising a guide RNA, or nucleic acid encoding a guide RNA, wherein the guide RNA comprises a spacer sequence selected from any one of SEQ ID NOs: 10, 12, 15, 16, 20, 27, 28, 32, 33, 35, 1001, 1003, 1005, 1010, 1012, 1013, 1016, 1017, and 1018 (for SaCas9). In some embodiments, the spacer sequence is SEQ ID NO: 10. In some embodiments, the spacer sequence is SEQ ID NO: 12. In some embodiments, the spacer sequence is SEQ ID NO: 15. In some embodiments, the spacer sequence is SEQ ID NO: 16. In some embodiments, the spacer sequence is SEQ ID NO: 20. In some embodiments, the spacer sequence is SEQ ID NO: 27. In some embodiments, the spacer sequence is SEQ ID NO: 28. In some embodiments, the spacer sequence is SEQ ID NO: 32. In some embodiments, the spacer sequence is SEQ ID NO: 33. In some embodiments, the spacer sequence is SEQ ID NO: 35. In some embodiments, the spacer sequence is SEQ ID NO: 1001. In some embodiments, the spacer sequence is SEQ ID NO: 1003. In some embodiments, the spacer sequence is SEQ ID NO: 1005. In some embodiments, the spacer sequence is SEQ ID NO: 1010. In some embodiments, the spacer sequence is SEQ ID NO: 1012. In some embodiments, the spacer sequence is SEQ ID NO: 1013. In some embodiments, the spacer sequence is SEQ ID NO: 1016. In some embodiments, the spacer sequence is SEQ ID NO: 1017. In some embodiments, the spacer sequence is SEQ ID NO: 1018.

In some embodiments, a composition is provided comprising a guide RNA, or nucleic acid encoding a guide RNA, wherein the guide RNA comprises a spacer sequence selected from any one of SEQ ID NOs: 3022, 3023, 3028, 3029, 3030, 3031, 3038, 3039, 3052, 3053, 3054, 3055, 3062, 3063, 3064, 3065, 3068, and 3069 (for SaCas9). In some embodiments, the spacer sequence is SEQ ID NO: 3022. In some embodiments, the spacer sequence is SEQ ID NO: 3023. In some embodiments, the spacer sequence is SEQ ID NO: 3028. In some embodiments, the spacer sequence is SEQ ID NO: 3029. In some embodiments, the spacer sequence is SEQ ID NO: 3030. In some embodiments, the spacer sequence is SEQ ID NO: 3031. In some embodiments, the spacer sequence is SEQ ID NO: 3038. In some embodiments, the spacer sequence is SEQ ID NO: 3039. In some embodiments, the spacer sequence is SEQ ID NO: 3052. In some embodiments, the spacer sequence is SEQ ID NO: 3053. In some embodiments, the spacer sequence is SEQ ID NO: 3054. In some embodiments, the spacer sequence is SEQ ID NO: 3055. In some embodiments, the spacer sequence is SEQ ID NO: 3062. In some embodiments, the spacer sequence is SEQ ID NO: 3063. In some embodiments, the spacer sequence is SEQ ID NO: 3064. In some embodiments, the spacer sequence is SEQ ID NO: 3065. In some embodiments, the spacer sequence is SEQ ID NO: 3068. In some embodiments, the spacer sequence is SEQ ID NO: 3069.

In some embodiments, a composition is provided comprising a guide RNA, or nucleic acid encoding a guide RNA, wherein the guide RNA comprises a spacer sequence selected from any one of SEQ ID NOs: 131, 134, 135, 136, 139, 140, 141, 144, 145, 146, 148, 149, 150, 151, 179, 184, 201, 210, 223, 224, 225 (for SluCas9). In some embodiments, the spacer sequence is SEQ ID NO: 131. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 139. In some embodiments, the spacer sequence is SEQ ID NO: 140. In some embodiments, the spacer sequence is SEQ ID NO: 141. In some embodiments, the spacer sequence is SEQ ID NO: 144. In some embodiments, the spacer sequence is SEQ ID NO: 145. In some embodiments, the spacer sequence is SEQ ID NO: 146. In some embodiments, the spacer sequence is SEQ ID NO: 148. In some embodiments, the spacer sequence is SEQ ID NO: 149. In some embodiments, the spacer sequence is SEQ ID NO: 150. In some embodiments, the spacer sequence is SEQ ID NO: 151. In some embodiments, the spacer sequence is SEQ ID NO: 179. In some embodiments, the spacer sequence is SEQ ID NO: 184. In some embodiments, the spacer sequence is SEQ ID NO: 201. In some embodiments, the spacer sequence is SEQ ID NO: 223. In some embodiments, the spacer sequence is SEQ ID NO: 224. In some embodiments, the spacer sequence is SEQ ID NO: 225.

In some embodiments, a composition is provided comprising a guide RNA, or nucleic acid encoding a guide RNA, wherein the guide RNA comprises a spacer sequence selected from any one of SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251 (for SluCas9). In some embodiments, the spacer sequence is SEQ ID NO: 4062. In some embodiments, the spacer sequence is SEQ ID NO: 4063. In some embodiments, the spacer sequence is SEQ ID NO: 4068. In some embodiments, the spacer sequence is SEQ ID NO: 4069. In some embodiments, the spacer sequence is SEQ ID NO: 4070. In some embodiments, the spacer sequence is SEQ ID NO: 4071. In some embodiments, the spacer sequence is SEQ ID NO: 4072. In some embodiments, the spacer sequence is SEQ ID NO: 4073. In some embodiments, the spacer sequence is SEQ ID NO: 4078. In some embodiments, the spacer sequence is SEQ ID NO: 4079. In some embodiments, the spacer sequence is SEQ ID NO: 4088. In some embodiments, the spacer sequence is SEQ ID NO: 4089. In some embodiments, the spacer sequence is SEQ ID NO: 4096. In some embodiments, the spacer sequence is SEQ ID NO: 4097. In some embodiments, the spacer sequence is SEQ ID NO: 4098. In some embodiments, the spacer sequence is SEQ ID NO: 4099. In some embodiments, the spacer sequence is SEQ ID NO: 4100. In some embodiments, the spacer sequence is SEQ ID NO: 4101. In some embodiments, the spacer sequence is SEQ ID NO: 4102. In some embodiments, the spacer sequence is SEQ ID NO: 4103. In some embodiments, the spacer sequence is SEQ ID NO: 4158. In some embodiments, the spacer sequence is SEQ ID NO: 4159. In some embodiments, the spacer sequence is SEQ ID NO: 4168. In some embodiments, the spacer sequence is SEQ ID NO: 4169. In some embodiments, the spacer sequence is SEQ ID NO: 4202. In some embodiments, the spacer sequence is SEQ ID NO: 4203. In some embodiments, the spacer sequence is SEQ ID NO: 4220. In some embodiments, the spacer sequence is SEQ ID NO: 4221. In some embodiments, the spacer sequence is SEQ ID NO: 4246. In some embodiments, the spacer sequence is SEQ ID NO: 4247. In some embodiments, the spacer sequence is SEQ ID NO: 4248. In some embodiments, the spacer sequence is SEQ ID NO: 4249. In some embodiments, the spacer sequence is SEQ ID NO: 4250. In some embodiments, the spacer sequence is SEQ ID NO: 4251.

In some embodiments, a composition is provided comprising a guide RNA, or nucleic acid encoding a guide RNA, wherein the guide RNA further comprises a trRNA. In each composition and method embodiment described herein, the crRNA (comprising the spacer sequence) and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In one aspect, a composition is provided comprising a single nucleic acid molecule encoding, or two nucleic acid molecules where one molecule encodes, 1) one or more guide RNA that comprises a guide sequence selected from any one of SEQ ID NOs: 1-35, 1000-1078, or 3000-3069; and 2) a SaCas9. In one aspect, a composition is provided comprising a single nucleic acid molecule encoding, or two nucleic acid molecules where one molecule encodes, 1) one or more guide RNA that comprises a guide sequence selected from any one of SEQ ID NOs: 100-225, 2000-2116, or 4000-4251; and 2) a SluCas9.

In one aspect, a composition is provided comprising a single nucleic acid molecule encoding, or two nucleic acid molecules where one molecule encodes, 1) one or more guide RNA that comprises a guide sequence that is at least 99%, 98%, 97%, 96%, 95%, 94%, 93%, 92%, 91%, or 90% identical to any one of SEQ ID NOs: 1-35, 1000-1078, 3000-3069; and 2) a SaCas9. In one aspect, a composition is provided comprising a single nucleic acid molecule encoding 1) one or more guide RNA that comprises a guide sequence that is at least 99%, 98%, 97%, 96%, 95%, 94%, 93%, 92%, 91%, or 90% identical to any one of SEQ ID NOs: 100-225, 2000-2116, or 4000-4251; and 2) a SluCas9.

In another aspect, a composition is provided comprising a single nucleic acid molecule encoding, or two nucleic acid molecules where one molecule encodes, 1) one or more guide RNA that comprises a guide sequence comprising at least 17, 18, 19, or 20 contiguous nucleotides of a spacer sequence selected from any one of SEQ ID NOs: 1-35, 1000-1078, or 3000-3069; and 2) a SaCas9. In another aspect, a composition is provided comprising a single nucleic acid molecule encoding, or two nucleic acid molecules where one molecule encodes, 1) one or more guide RNA that comprises a guide sequence comprising at least 17, 18, 19, or 20 contiguous nucleotides of a spacer sequence selected from any one of SEQ ID NOs: 100-225, 2000-2116, or 4000-4251; and 2) a SluCas9.

In any embodiment comprising a nucleic acid molecule encoding a guide RNA and/or a Cas9, the nucleic acid molecule may be a vector. In some embodiments, a composition is provided comprising a single nucleic acid molecule encoding a guide RNA and Cas9, wherein the nucleic acid molecule is a vector.

Any type of vector, such as any of those described herein, may be used. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a muscle-specific promoter. Exemplary muscle-specific promoters include a muscle creatine kinase promoter, a desmin promoter, an MHCK7 promoter, or an SPc5-12 promoter. See US 2004/0175727 A1; Wang et al., Expert Opin Drug Deliv. (2014) 11, 345-364; Wang et al., Gene Therapy (2008) 15, 1489-1499. In some embodiments, the muscle-specific promoter is a CK8 promoter. In some embodiments, the muscle-specific promoter is a CK8e promoter. In any of the foregoing embodiments, the vector may be an adeno-associated virus vector (AAV).

In some embodiments, the muscle specific promoter is the CK8 promoter. The CK8 promoter has the following sequence (SEQ ID NO. 700):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. In some embodiments, the size of the CK8e promoter is 436 bp. The CK8e promoter has the following sequence (SEQ ID NO. 701):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

In some embodiments, the vector comprises one or more of a U6, H1, or 7SK promoter. In some embodiments, the U6 promoter is the human U6 promoter (e.g., the U6L promoter or U6S promoter). In some embodiments, the promoter is the murine U6 promoter. In some embodiments, the 7SK promoter is a human 7SK promoter. In some embodiments, the 7SK promoter is the 7SK1 promoter. In some embodiments, the 7SK promoter is the 7SK2 promoter. In some embodiments, the H1 promoter is a human H1 promoter (e.g., the H1L promoter or the H1S promoter). In some embodiments, the vector comprises multiple guide sequences, wherein each guide sequence is under the control of a separate promoter. In some embodiments, each of the multiple guide sequences comprises a different sequence. In some embodiments, each of the multiple guide sequences comprise the same sequence (e.g., each of the multiple guide sequences comprise the same spacer sequence). In some embodiments, each of the multiple guide sequences comprises the same spacer sequence and the same scaffold sequence. In some embodiments, each of the multiple guide sequences comprises different spacer sequences and different scaffold sequences. In some embodiments, each of the multiple guide sequences comprises the same spacer sequence, but comprises a different scaffold sequence. In some embodiments, each of the multiple guide sequences comprises different spacer sequences and different scaffold sequences. In some embodiments, each of the separate promoters comprises the same nucleotide sequence (e.g., the U6 promoter sequence). In some embodiments, each of the separate promoters comprises a different nucleotide sequence (e.g., the U6, H1, and/or 7SK promoter sequence).

In some embodiments, the U6 promoter comprises a nucleotide sequence that is at least 90%, 91%, 92%, 93%, 94%, 95%, 96%, 97%, 98%, 99% or 100% identical to the sequence of SEQ ID NO: 702:

| | |
|---|---|
| cgagtccaac acccgtggga atcccatggg caccatggcc cctcgctcca aaaatgcttt | 60 |
| cgcgtcgcgc agacactgct cggtagtttc ggggatcagc gtttgagtaa gagcccgcgt | 120 |
| ctgaaccctc cgcgccgccc cggccccagt ggaaagacgc gcaggcaaaa cgcaccacgt | 180 |
| gacggagcgt gaccgcgcgc cgagcgcgcg ccaaggtcgg gcaggaagag ggcctatttc | 240 |
| ccatgattcc ttcatatttg catatacgat acaaggctgt tagagagata attagaatta | 300 |
| atttgactgt aaacacaaag atattagtac aaaatacgtg acgtagaaag taataatttc | 360 |
| ttgggtagtt tgcagtttta aaattatgtt ttaaaatgga ctatcatatg cttaccgtaa | 420 |
| cttgaaagta tttcgatttc ttggctttat atatcttgtg gaaaggacga aa | 472 |

In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 703:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the 7SK promoter comprises a nucleotide sequence that is at least 90%, 91%, 92%, 93%, 94%, 95%, 96%, 97%, 98%, 99% or 100% identical to the sequence of SEQ ID NO. 704:

| | |
|---|---|
| tgacggcgcg ccctgcagta tttagcatgc cccacccatc tgcaaggcat tctggatagt | 60 |
| gtcaaaacag ccggaaatca agtccgttta tctcaaactt tagcattttg ggaataaatg | 120 |
| atatttgcta tgctggttaa attagatttt agttaaattt cctgctgaag ctctagtacg | 180 |
| ataagtaact tgacctaagt gtaaagttga gatttccttc aggtttatat agcttgtgcg | 240 |
| ccgcctgggt a | 251 |

In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 705:

GAGGGCCTATTTCCCATGATTCCTTCATATTTGCATATACGATACAAGG
CTGTTAGAGAGATAATTGGAATTAATTTGACTGTAAACACAAAGATATT
AGTACAAAATACGTGACGTAGAAAGTAATAATTTCTTGGGTAGTTTGCA
GTTTTAAAATTATGTTTTAAAATGGACTATCATATGCTTACCGTAACTT
GAAAGTATTTCGATTTCTTGGCTTTATATATCTTGTGGAAAGGACGAAA
CACC.

In some embodiments, the U6 promoter is a variant of the hU6c promoter. In some embodiments, the variant of the hU6c promoter comprises alternative nucleotides as compared to the sequence of SEQ ID NO: 705. In some embodiments, the variant of the hU6c promoter comprises fewer nucleotides as compared to the 249 nucleotides of SEQ ID NO: 705. In some embodiments, the variant of the hU6c promoter has fewer nucleotides in the nucleosome binding sequence of the hU6c promoter of SEQ ID NO: 705. In some embodiments, the variant of the hU6c promoter lacks all of or at least a portion of (e.g., at least 5, 10, 15, 20, 25, or 30 nucleotides) the nucleotides corresponding to nucleotides 96-125 of SEQ ID NO: 705. In some embodiments, the variant of the hU6c promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 705. In some embodiments, the variant of the hU6c promoter lacks all of or at least a portion of (e.g., at least 10, 20, 30, 40, 50, 60, 65, 70, 75, 80, or 85 nucleotides) the nucleotides corresponding to nucleotides 66-150 of SEQ ID NO: 705. In some embodiments, the variant of the hU6c promoter lacks all of or at least a portion of (e.g., at least 10, 20, 30, 40, 50, 60, 70, 80, 90, 100, 110, or 120 nucleotides) the nucleotides corresponding to nucleotides 51-170 of SEQ ID NO: 705. In some embodiments, the variant of the hU6c promoter lacks the nucleotides corresponding to nucleotides 96-125 of SEQ ID NO: 705. In some embodiments, the variant of the hU6c promoter comprises 129-219 nucleotides. In some embodiments, the variant of the hU6c promoter comprises 219 nucleotides. In some embodiments, the variant of the hU6c promoter comprises 189 nucleotides. In some embodiments, the variant of the hU6c promoter comprises 159 nucleotides. In some embodiments, the variant of the hU6c promoter comprises 129 nucleotides.

In some embodiments, the U6 promoter is hU6d30 and comprises a nucleotide sequence that is at least 90%, 91%, 92%, 93%, 94%, 95%, 96%, 97%, 98%, 99% or 100% identical to the sequence of SEQ ID NO: 9001:

GAGGGCCTATTTCCCATGATTCCTTCATATTTGCATATACGATACAAGG
CTGTTAGAGAGATAATTGGAATTAATTTGACTGTAAACACAAAGATATA
ATTTCTTGGGTAGTTTGCAGTTTTAAAATTATGTTTTAAAATGGACTAT
CATATGCTTACCGTAACTTGAAAGTATTTCGATTTCTTGGCTTTATATA
TCTTGTGGAAAGGACGAAACACC.

In some embodiments, the U6 promoter is hU6d60 and comprises a nucleotide sequence that is at least 90%, 91%, 92%, 93%, 94%, 95%, 96%, 97%, 98%, 99% or 100% identical to the sequence of SEQ ID NO: 9002:

GAGGGCCTATTTCCCATGATTCCTTCATATTTGCATATACGATACAAGG
CTGTTAGAGAGATAATTGGAATTAATTTGACGTTTGCAGTTTTAAAATT
ATGTTTTAAAATGGACTATCATATGCTTACCGTAACTTGAAAGTATTTC
GATTTCTTGGCTTTATATATCTTGTGGAAAGGACGAAACACC.

In some embodiments, the U6 promoter is hU6d90 and comprises a nucleotide sequence that is at least 90%, 91%, 92%, 93%, 94%, 95%, 96%, 97%, 98%, 99% or 100% identical to the sequence of SEQ ID NO: 9003:

GAGGGCCTATTTCCCATGATTCCTTCATATTTGCATATACGATACAAGG
CTGTTAGAGAGATAATATTATGTTTTAAAATGGACTATCATATGCTTAC
CGTAACTTGAAAGTATTTCGATTTCTTGGCTTTATATATCTTGTGGAAA
GGACGAAACACC.

In some embodiments, the U6 promoter is hU6d120 and comprises a nucleotide sequence that is at least 90%, 91%, 92%, 93%, 94%, 95%, 96%, 97%, 98%, 99% or 100% identical to the sequence of SEQ ID NO: 9004:

GAGGGCCTATTTCCCATGATTCCTTCATATTTGCATATACGATACAAGG
CGGACTATCATATGCTTACCGTAACTTGAAAGTATTTCGATTTCTTGGC
TTTATATATCTTGTGGAAAGGACGAAACACC.

In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 706:

CTGCAGTATTTAGCATGCCCCACCCATCTGCAAGGCATTCTGGATAGTG
TCAAAACAGCCGGAAATCAAGTCCGTTTATCTCAAACTTTAGCATTTTG
GGAATAAATGATATTTGCTATGCTGGTTAAATTAGATTTTAGTTAAATT
TCCTGCTGAAGCTCTAGTACGATAAGCAACTTGACCTAAGTGTAAAGTT
GAGACTTCCTTCAGGTTTATATAGCTTGTGCGCCGCTTGGGTACCTC.

In some embodiments, the 7SK promoter is a variant of the 7SK2 promoter. In some embodiments, the variant of the 7SK2 promoter comprises alternative nucleotides as compared to the sequence of SEQ ID NO: 706. In some embodiments, the variant of the 7SK2 promoter e.g., comprises fewer nucleotides as compared to the 243 nucleotides of SEQ ID NO: 706. In some embodiments, the variant of the 7SK2 promoter has fewer nucleotides in the nucleosome binding sequence of the 7SK2 promoter of SEQ ID NO: 706. In some embodiments, the variant of the 7SK2 promoter lacks all of or at least a portion of (e.g., at least 5, 10, 15, 20, 25, or 30 nucleotides) the nucleotides corresponding to nucleotides 95-124 of SEQ ID NO: 706. In some embodiments, the variant of the 7SK2 promoter lacks all of or at least a portion of (e.g., at least 5, 10, 15, 20, 25, 30, 35, 40, 45, 50, 55, or 60 nucleotides) the nucleotides corresponding to nucleotides 81-140 of SEQ ID NO: 706. In some embodiments, the variant of the 7SK2 promoter lacks all of or at least a portion of (e.g., at least 10, 20, 30, 40, 50, 60, 65, 70, 75, 80, 85 or 90 nucleotides) the nucleotides corresponding to nucleotides 67-156 of SEQ ID NO: 706. In some embodiments, the variant of the 7SK2 promoter lacks all of or at least a portion of (e.g., at least 10, 20, 30, 40, 50, 60, 70, 80, 90, 100, 110, or 120 nucleotides) the nucleotides corresponding to nucleotides 52-171 of SEQ ID NO: 706. In some embodiments, the variant of the 7SK2 promoter comprises 123-213 nucleotides. In some embodiments, the variant of the 7SK2 promoter comprises 213 nucleotides. In some embodiments, the variant of the 7SK2 promoter comprises 183 nucleotides. In some embodiments, the variant of the 7SK2 promoter comprises 153 nucleotides. In some embodiments, the variant of the 7SK2 promoter comprises 123 nucleotides.

In some embodiments, the 7SK promoter is 7SKd30 and comprises a nucleotide sequence that is at least 90%, 91%, 92%, 93%, 94%, 95%, 96%, 97%, 98%, 99% or 100% identical to the sequence of SEQ ID NO: 9006:

CTGCAGTATTTAGCATGCCCCACCCATCTGCAAGGCATTCTGGATAGTG
TCAAAACAGCCGGAAATCAAGTCCGTTTATCTCAAACTTTAGCATTTAA
ATTAGATTTTAGTTAAATTTCCTGCTGAAGCTCTAGTACGATAAGCAAC
TTGACCTAAGTGTAAAGTTGAGACTTCCTTCAGGTTTATATAGCTTGTG
CGCCGCTTGGGTACCTC.

In some embodiments, the 7SK promoter is 7SKd60 and comprises a nucleotide sequence that is at least 90%, 91%, 92%, 93%, 94%, 95%, 96%, 97%, 98%, 99% or 100% identical to the sequence of SEQ ID NO: 9007:

CTGCAGTATTTAGCATGCCCCACCCATCTGCAAGGCATTCTGGATAGTG
TCAAAACAGCCGGAAATCAAGTCCGTTTATCTTAAATTTCCTGCTGAAG
CTCTAGTACGATAAGCAACTTGACCTAAGTGTAAAGTTGAGACTTCCTT
CAGGTTTATATAGCTTGTGCGCCGCTTGGGTACCTC.

In some embodiments, the 7SK promoter is 7SKd90 and comprises a nucleotide sequence that is at least 90%, 91%, 92%, 93%, 94%, 95%, 96%, 97%, 98%, 99% or 100% identical to the sequence of SEQ ID NO: 9008:

CTGCAGTATTTAGCATGCCCCACCCATCTGCAAGGCATTCTGGATAGTG
TCAAAACAGCCGGAAATAGCTCTAGTACGATAAGCAACTTGACCTAAGT
GTAAAGTTGAGACTTCCTTCAGGTTTATATAGCTTGTGCGCCGCTTGGG
TACCTC.

In some embodiments, the 7SK promoter is 7SKd120 and comprises a nucleotide sequence that is at least 90%, 91%, 92%, 93%, 94%, 95%, 96%, 97%, 98%, 99% or 100% identical to the sequence of SEQ ID NO: 9009:

CTGCAGTATTTAGCATGCCCCACCCATCTGCAAGGCATTCTGGATAGTG
TCAGCAACTTGACCTAAGTGTAAAGTTGAGACTTCCTTCAGGTTTATAT
AGCTTGTGCGCCGCTTGGGTACCTC.

In some embodiments, the H1 promoter is a H1m or mH1 promoter and comprises a nucleotide sequence that is at least 90%, 91%, 92%, 93%, 94%, 95%, 96%, 97%, 98%, 99% or 100% identical to the sequence of SEQ ID NO: 707:

AATATTTGCATGTCGCTATGTGTTCTGGGAAATCACCATAAACGTGAAA
TGTCTTTGGATTTGGGAATCTTATAAGTTCTGTATGAGACCACTCTTTC
CC.

In some embodiments, the Ck8e promoter comprises a nucleotide sequence that is at least 90%, 91%, 92%, 93%, 94%, 95%, 96%, 97%, 98%, 99% or 100% identical to the sequence of SEQ ID NO: 701

TGCCCATGTAAGGAGGCAAGGCCTGGGGACACCCGAGATGCCTGGTTAT
AATTAACCCAGACATGTGGCTGCCCCCCCCCCCCCAACACCTGCTGCCT
CTAAAAATAACCCTGCATGCCATGTTCCCGGCGAAGGGCCAGCTGTCCC
CCGCCAGCTAGACTCAGCACTTAGTTTAGGAACCAGTGAGCAAGTCAGC
CCTTGGGGCAGCCCATACAAGGCCATGGGGCTGGGCAAGCTGCACGCCT
GGGTCCGGGGTGGGCACGGTGCCCGGGCAACGAGCTGAAAGCTCATCTG
CTCTCAGGGGCCCCTCCCTGGGGACAGCCCCTCCTGGCTAGTCACACCC
TGTAGGCTCCTCTATATAACCCAGGGGCACAGGGGCTGCCCTCATTCTA
CCACCACCTCCACAGCACAGACAGACACTCAGGAGCCAGCCAGC.

In some embodiments, the vector comprises multiple inverted terminal repeats (ITRs). These ITRs may be of an AAV1, AAV2, AAV3, AAV4, AAVS, AAV6, AAV7, AAV8, or AAV9 serotype. In some embodiments, the ITRs are of an AAV2 serotype. In some embodiments, the 5′ ITR comprises a sequence that is at least 80%, 85%, 90%, 91%, 92%, 93%, 94%, 95%, 96%, 97%, 98%, 99%, or 100% identical to the sequence of SEQ ID NO: 709:

GGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT.

In some embodiments, the 3′ITR comprises a sequence that is at least 80%, 85%, 90%, 91%, 92%, 93%, 94%, 95%, 96%, 97%, 98%, 99%, or 100% identical to the sequence of SEQ ID NO: 710:

AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGAGAGGGA.

In some embodiments, a vector comprising a single nucleic acid molecule encoding 1) one or more guide RNA comprising any one or more of the spacer sequences of SEQ ID Nos: 1-35, 100-225, 1000-1078, 2000-2116, 3000-3069, or 4000-4251; and 2) a SaCas9 (for SEQ ID Nos: 1-35, 1000-1078, and 3000-3069) or SluCas9 (for SEQ ID NO: 100-225, 2000-2116, and 4000-4251) is provided. In some embodiments, the vector is an AAV vector. In some embodiments, the AAV vector is administered to a subject to treat DMD. In some embodiments, only one vector is needed due to the use of a particular guide sequence that is useful in the context of SaCas9 or SluCas9.

In some embodiments, the vector comprises a nucleic acid encoding a Cas9 protein (e.g., an SaCas9 or SluCas9 protein) and further comprises a nucleic acid encoding one or more single guide RNA(s). In some embodiments, the nucleic acid encoding the Cas9 protein is under the control of a CK8e promoter. In some embodiments, the nucleic acid encoding the guide RNA sequence is under the control of a hU6c promoter. In some embodiments, the vector is AAV9.

In some embodiments, the vector comprises multiple nucleic acids encoding more than one guide RNA. In some embodiments, the vector comprises two nucleic acids encoding two guide RNA sequences.

In some embodiments, the vector comprises a nucleic acid encoding a Cas9 protein (e.g., an SaCas9 protein or SluCas9 protein), a nucleic acid encoding a first guide RNA, and a nucleic acid encoding a second guide RNA. In some embodiments, the vector does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the vector comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the vector comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9 or SluCas9 protein) is under the control of the CK8e promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA. In some embodiments, any of the vectors disclosed herein is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb from ITR to ITR in size, inclusive of both ITRs. In some embodiments, the vector is an AAV9 vector.

In some embodiments, any of the vectors disclosed herein comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA. In some embodiments, the scaffold-encoding sequence for the first guide RNA comprises a sequence selected from the group consisting of SEQ ID Nos: 901-916, and the scaffold-encoding sequence for the second guide RNA comprises a different sequence selected from the group consisting of SEQ ID Nos: 901-916. In some embodiments, the scaffold-encoding sequence for the first guide RNA comprises the sequence of SEQ ID NO: 901, and the scaffold-encoding sequence for the second guide RNA comprises the sequence of SEQ ID NO: 902. In some embodiments, the scaffold-encoding sequence for the first guide RNA comprises the sequence of SEQ ID NO: 901, and the scaffold-encoding sequence for the second guide RNA comprises the sequence of SEQ ID NO: 903. In some embodiments, the scaffold-encoding sequence for the first guide RNA comprises the sequence of SEQ ID NO: 901, and the scaffold-encoding sequence for the second guide RNA comprises the sequence of SEQ ID NO: 904. In some embodiments, the scaffold-encoding sequence for the first guide RNA comprises the sequence of SEQ ID NO: 901, and the scaffold-encoding sequence for the second guide RNA comprises the sequence of SEQ ID NO: 905. In some embodiments, the scaffold-encoding sequence for the first guide RNA comprises the sequence of SEQ ID NO: 901, and the scaffold-encoding sequence for the second guide RNA comprises the sequence of SEQ ID NO: 906. In some embodiments, the scaffold-encoding sequence for the first guide RNA comprises the sequence of SEQ ID NO: 901, and the scaffold-encoding sequence for the second guide RNA comprises the sequence of SEQ ID NO: 907. In some embodiments, the scaffold-encoding sequence for the first guide RNA comprises the sequence of SEQ ID NO: 901, and the scaffold-encoding sequence for the second guide RNA comprises the sequence of SEQ ID NO: 908. In some embodiments, the scaffold-encoding sequence for the first guide RNA comprises the sequence of SEQ ID NO: 901, and the scaffold-encoding sequence for the second guide RNA comprises the sequence of SEQ ID NO: 909. In some embodiments, the scaffold-encoding sequence for the first guide RNA comprises the sequence of SEQ ID NO: 901, and the scaffold-encoding sequence for the second guide RNA comprises the sequence of SEQ ID NO: 910. In some embodiments, the scaffold-encoding sequence for the first guide RNA comprises the sequence of SEQ ID NO: 901, and the scaffold-encoding sequence for the second guide RNA comprises the sequence of SEQ ID NO: 911. In some embodiments, the scaffold-encoding sequence for the first guide RNA comprises the sequence of SEQ ID NO: 901, and the scaffold-encoding sequence for the second guide RNA comprises the sequence of SEQ ID NO: 912. In some embodiments, the scaffold-encoding sequence for the first guide RNA comprises the sequence of SEQ ID NO: 901, and the scaffold-encoding sequence for the second guide RNA comprises the sequence of SEQ ID NO: 913. In some embodiments, the scaffold-encoding sequence for the first guide RNA comprises the sequence of SEQ ID NO: 901, and the scaffold-encoding sequence for the second guide RNA comprises the sequence of SEQ ID NO: 914. In some embodiments, the scaffold-encoding sequence for the first guide RNA comprises the sequence of SEQ ID NO: 901, and the scaffold-encoding sequence for the second guide RNA comprises the sequence of SEQ ID NO: 915. In some embodiments, the scaffold-encoding sequence for the first guide RNA comprises the sequence of SEQ ID NO: 901, and the scaffold-encoding sequence for the second guide RNA comprises the sequence of SEQ ID NO: 916. In some embodiments, the spacer encoding sequence for the first guide RNA is the same as the spacer-encoding sequence in the second guide RNA, and the scaffold-encoding sequence for the first guide RNA is different from the scaffold-encoding sequence in the nucleic acid encoding the second guide RNA.

The disclosure provides for novel AAV vector configurations. Some examples of these novel AAV vector configurations are provided herein, and the order of elements in these exemplary vectors are referenced in a 5′ to 3′ manner with respect to the plus strand. For these configurations, it should be understood that the recited elements may not be directly contiguous, and that one or more nucleotides or one or more additional elements may be present between the recited elements. However, in some embodiments, it is possible that no nucleotides or no additional elements are present between the recited elements. Also, unless otherwise stated, “a promoter for expression of element X” means that the promoter is oriented in a manner to facilitate expression of the recited element X. In addition, unless otherwise stated, references to an “sgRNA scaffold sequence” or “a guide RNA scaffold sequence” are synonymous with “a nucleotide sequence/nucleic acid encoding an sgRNA scaffold sequence” or “a nucleotide sequence/nucleic acid encoding a guide RNA scaffold sequence.” In some embodiments, the disclosure provides for a nucleic acid encoding an SaCas9 (e.g., an SaCas9-KKH) or SluCas9. In some embodiments, the nucleic acid encodes for one or more nuclear localization signals (e.g., the SV40 NLS and/or the c-Myc NLS) on the C-terminus of the encoded SaCas9 or SluCas9. In some embodiments, the nucleic acid encodes for one or more NLSs (e.g., the SV40 NLS and/or the c-Myc NLS) on the C-terminus of the encoded SaCas9 or SluCas9, and the nucleic acid does not encode for an NLS on the N-terminus of the encoded SaCas9 or SluCas9. In some embodiments, the nucleic acid encodes for one or more nuclear localization signals (e.g., the SV40 NLS and/or the c-Myc NLS) on the N-terminus of the encoded SaCas9 or SluCas9. In some embodiments, the nucleic acid encodes for one or more NLSs (e.g., the SV40 NLS and/or the c-Myc NLS) on the N-terminus of the encoded SaCas9 or SluCas9, and the nucleic acid does not encode for an NLS on the C-terminus of the encoded SaCas9 or SluCas9. In some embodiments, the nucleic acid encodes for one or more nuclear localization signals (e.g., the SV40 NLS and/or the c-Myc NLS) on the C-terminus of the encoded SaCas9 or SluCas9 and also encodes for one or more NLSs on the N-terminus of the encoded SaCas9 or SluCas9 (e.g., the SV40 NLS and/or the c-Myc NLS). In some embodiments, the nucleic acid encodes one NLS. In some embodiments, the nucleic acid encodes two NLSs. In some embodiments, the nucleic acid encodes three NLSs. The one, two, or three NLS may all be C-terminal, N-terminal, or any combination of C- and N-terminal. The NLS may be fused/attached directly to the C- or N-terminus or to another NLS, or may be fused/attached indirectly attached through a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein, with or without a linker. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of a second sgRNA, a second sgRNA guide sequence, and a second sgRNA scaffold sequence. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9001. In some embodiments the promoter for expression of the nucleic acid encoding the first sgRNA comprises SEQ ID NO: 9002. In some embodiments the promoter for expression of the nucleic acid encoding the first sgRNA comprises SEQ ID NO: 9003. In some embodiments the promoter for expression of the nucleic acid encoding the first sgRNA comprises SEQ ID NO: 9004. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9006. In some embodiments the promoter for expression of the nucleic acid encoding the first sgRNA comprises SEQ ID NO: 9007. In some embodiments the promoter for expression of the nucleic acid encoding the first sgRNA comprises SEQ ID NO: 9008. In some embodiments the promoter for expression of the nucleic acid encoding the first sgRNA comprises SEQ ID NO: 9009. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9001. In some embodiments the promoter for expression of the nucleic acid encoding the second sgRNA comprises SEQ ID NO: 9002. In some embodiments the promoter for expression of the nucleic acid encoding the second sgRNA comprises SEQ ID NO: 9003. In some embodiments the promoter for expression of the nucleic acid encoding the second sgRNA comprises SEQ ID NO: 9004. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9006. In some embodiments the promoter for expression of the nucleic acid encoding the second sgRNA comprises SEQ ID NO: 9007. In some embodiments the promoter for expression of the nucleic acid encoding the second sgRNA comprises SEQ ID NO: 9008. In some embodiments the promoter for expression of the nucleic acid encoding the second sgRNA comprises SEQ ID NO: 9009. In some embodiments the promoter for expression of the nucleic acid encoding the second sgRNA is any of the H1m promoters disclosed herein. In some embodiments, the promoter for SaCas9 is the CK8e promoter. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an hU6c promoter for expression of a second sgRNA, a second sgRNA guide sequence, and a second sgRNA scaffold sequence. In some embodiments, the first sgRNA and the second sgRNA comprise the sequences of SEQ ID NOs: 10 and 15. In some embodiments, the first sgRNA and the second sgRNA comprise the sequences of SEQ ID NOs: 10 and 16. In some embodiments, the first sgRNA and the second sgRNA comprise the sequences of SEQ ID NOs: 12 and 16. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 15 and 10. In some embodiments, the first sgRNA and the second sgRNA comprise the sequences of SEQ ID NOs: 16 and 10. In some embodiments, the first sgRNA and the second sgRNA comprise the sequences of SEQ ID NOs: 16 and 12. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an 7SK promoter for expression of a second sgRNA, a second sgRNA guide sequence, and a second sgRNA scaffold sequence. In some embodiments, the first sgRNA and the second sgRNA comprise the sequences of SEQ ID NOs: 10 and 15. In some embodiments, the first sgRNA and the second sgRNA comprise the sequences of SEQ ID NOs: 10 and 16. In some embodiments, the first sgRNA and the second sgRNA comprise the sequences of SEQ ID NOs: 12 and 16. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 15 and 10. In some embodiments, the first sgRNA and the second sgRNA comprise the sequences of SEQ ID NOs: 16 and 10. In some embodiments, the first sgRNA and the second sgRNA comprise the sequences of SEQ ID NOs: 16 and 12. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9, a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 10 and 15. In some embodiments, the first sgRNA and the second sgRNA comprise the sequences of SEQ ID NOs: 10 and 16. In some embodiments, the first sgRNA and the second sgRNA comprise the sequences of SEQ ID NOs: 12 and 16. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 15 and 10. In some embodiments, the first sgRNA and the second sgRNA comprise the sequences of SEQ ID NOs: 16 and 10. In some embodiments, the first sgRNA and the second sgRNA comprise the sequences of SEQ ID NOs: 16 and 12. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK promoter for expression of the nucleic acid encoding the first sgRNA, a promoter for expression of a nucleic acid encoding SaCas9 (e.g., CK8e), a nucleic acid encoding SaCas9, a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 10 and 15. In some embodiments, the first sgRNA and the second sgRNA comprise the sequences of SEQ ID NOs: 10 and 16. In some embodiments, the first sgRNA and the second sgRNA comprise the sequences of SEQ ID NOs: 12 and 16. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 15 and 10. In some embodiments, the first sgRNA and the second sgRNA comprise the sequences of SEQ ID NOs: 16 and 10. In some embodiments, the first sgRNA and the second sgRNA comprise the sequences of SEQ ID NOs: 16 and 12. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9-KKH (e.g., a sequence that is at least 85%, 90%, 91%, 92%, 93%, 94%, 95%, 96%, 97%, 98%, 99% or 100% identical to SEQ ID NO: 715 or functional fragments thereof), a polyadenylation sequence, an hU6c promoter for expression of a second sgRNA, a second sgRNA guide sequence, and a second sgRNA scaffold sequence. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9-KKH (e.g., a sequence that is at least 85%, 90%, 91%, 92%, 93%, 94%, 95%, 96%, 97%, 98%, 99% or 100% identical to SEQ ID NO: 715 or functional fragments thereof), a polyadenylation sequence, an 7SK promoter for expression of a second sgRNA, a second sgRNA guide sequence, and a second sgRNA scaffold sequence. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SaCas9 (e.g., CK8e), a nucleic acid encoding SaCas9-KKH (e.g., a sequence that is at least 85%, 90%, 91%, 92%, 93%, 94%, 95%, 96%, 97%, 98%, 99% or 100% identical to SEQ ID NO: 715 or functional fragments thereof), a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK promoter for expression of the nucleic acid encoding the first sgRNA, a promoter for expression of a nucleic acid encoding SaCas9 (e.g., CK8e), a nucleic acid encoding SaCas9-KKH (e.g., a sequence that is at least 85%, 90%, 91%, 92%, 93%, 94%, 95%, 96%, 97%, 98%, 99% or 100% identical to SEQ ID NO: 715 or functional fragments thereof), a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 1001 and 1005. In some embodiments, the first sgRNA and the second sgRNA comprise the sequences of SEQ ID NOs: 1001 and 15. In some embodiments, the first sgRNA and the second sgRNA comprise the sequences of SEQ ID NOs: 1001 and 16. In some embodiments, the first sgRNA and the second sgRNA comprise the sequences of SEQ ID NOs: 1003 and 1005. In some embodiments, the first sgRNA and the second sgRNA comprise the sequences of SEQ ID NOs: 16 and 1003. In some embodiments, the first sgRNA and the second sgRNA comprise the sequences of SEQ ID NOs: 12 and 1010. In some embodiments, the first sgRNA and the second sgRNA comprise the sequences of SEQ ID NOs: 12 and 1012. In some embodiments, the first sgRNA and the second sgRNA comprise the sequences of SEQ ID NOs: 12 and 1013. In some embodiments, the first sgRNA and the second sgRNA comprise the sequences of SEQ ID NOs: 10 and 1016. In some embodiments, the first sgRNA and the second sgRNA comprise the sequences of SEQ ID NOs: 1017 and 16. In some embodiments, the first sgRNA and the second sgRNA comprise the sequences of SEQ ID NOs: 1018 and 16. In some embodiments, the first sgRNA and the second sgRNA comprise the sequences of SEQ ID NOs: 1005 and 1001. In some embodiments, the first sgRNA and the second sgRNA comprise the sequences of SEQ ID NOs: 15 and 1001. In some embodiments, the first sgRNA and the second sgRNA comprise the sequences of SEQ ID NOs: 16 and 1001. In some embodiments, the first sgRNA and the second sgRNA comprise the sequences of SEQ ID NOs: 1005 and 1003. In some embodiments, the first sgRNA and the second sgRNA comprise the sequences of SEQ ID NOs: 1003 and 16. In some embodiments, the first sgRNA and the second sgRNA comprise the sequences of SEQ ID NOs: 1010 and 12. In some embodiments, the first sgRNA and the second sgRNA comprise the sequences of SEQ ID NOs: 1012 and 12. In some embodiments, the first sgRNA and the second sgRNA comprise the sequences of SEQ ID NOs: 1013 and 12. In some embodiments, the first sgRNA and the second sgRNA comprise the sequences of SEQ ID NOs: 1016 and 10. In some embodiments, the first sgRNA and the second sgRNA comprise the sequences of SEQ ID NOs: 16 and 1017. In some embodiments, the first sgRNA and the second sgRNA comprise the sequences of SEQ ID NOs: 16 and 1018. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SluCas9 (e.g., CK8e), a nucleic acid encoding SluCas9, a polyadenylation sequence, a promoter for expression of a second sgRNA, a second sgRNA guide sequence, and a second sgRNA scaffold sequence. In some embodiments the promoter for expression of the nucleic acid encoding the first sgRNA is any of the hU6c promoters disclosed herein. In some embodiments the promoter for expression of the nucleic acid encoding the first sgRNA comprises SEQ ID NO: 705. In some embodiments the promoter for expression of the nucleic acid encoding the first sgRNA comprises SEQ ID NO: 9001. In some embodiments the promoter for expression of the nucleic acid encoding the first sgRNA comprises SEQ ID NO: 9002. In some embodiments the promoter for expression of the nucleic acid encoding the first sgRNA comprises SEQ ID NO: 9003. In some embodiments the promoter for expression of the nucleic acid encoding the first sgRNA comprises SEQ ID NO: 9004. In some embodiments the promoter for expression of the nucleic acid encoding the first sgRNA is any of the 7SK2 promoters disclosed herein. In some embodiments the promoter for expression of the nucleic acid encoding the first sgRNA comprises SEQ ID NO: 7005. In some embodiments the promoter for expression of the nucleic acid encoding the first sgRNA comprises SEQ ID NO: 9006. In some embodiments the promoter for expression of the nucleic acid encoding the first sgRNA comprises SEQ ID NO: 9007. In some embodiments the promoter for expression of the nucleic acid encoding the first sgRNA comprises SEQ ID NO: 9008. In some embodiments the promoter for expression of the nucleic acid encoding the first sgRNA comprises SEQ ID NO: 9009. In some embodiments the promoter for expression of the nucleic acid encoding the second sgRNA is any of the hU6c promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9001. In some embodiments the promoter for expression of the nucleic acid encoding the second sgRNA comprises SEQ ID NO: 9002. In some embodiments the promoter for expression of the nucleic acid encoding the second sgRNA comprises SEQ ID NO: 9003. In some embodiments the promoter for expression of the nucleic acid encoding the second sgRNA comprises SEQ ID NO: 9004. In some embodiments the promoter for expression of the nucleic acid encoding the second sgRNA is any of the 7SK2 promoters disclosed herein. In some embodiments the promoter for expression of the nucleic acid encoding the second sgRNA comprises SEQ ID NO: 705. In some embodiments the promoter for expression of the nucleic acid encoding the second sgRNA comprises SEQ ID NO: 9006. In some embodiments the promoter for expression of the nucleic acid encoding the second sgRNA comprises SEQ ID NO: 9007. In some embodiments the promoter for expression of the nucleic acid encoding the second sgRNA comprises SEQ ID NO: 9008. In some embodiments the promoter for expression of the nucleic acid encoding the second sgRNA comprises SEQ ID NO: 9009. In some embodiments the promoter for expression of the nucleic acid encoding the second sgRNA is any of the H1m promoters disclosed herein. In some embodiments, the promoter for SluCas9 is the CK8e promoter. In some embodiments, the nucleic acid sequence encoding SluCas9 is fused to a nucleic acid sequence encoding a nuclear localization sequence (NLS). In some embodiments, the nucleic acid sequence encoding SluCas9 is fused to two nucleic acid sequences each encoding a nuclear localization sequence (NLS). In some embodiments, the nucleic acid sequence encoding SluCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SluCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an hU6c promoter for expression of a second sgRNA, a second sgRNA guide sequence, and a second sgRNA scaffold sequence. In some embodiments, the first sgRNA and the second sgRNA comprise the sequences of SEQ ID NOs: 148 and 134. In some embodiments, the first sgRNA and the second sgRNA comprise the sequences of SEQ ID NOs: 149 and 135. In some embodiments, the first sgRNA and the second sgRNA comprise the sequences of SEQ ID NOs: 150 and 135. In some embodiments, the first sgRNA and the second sgRNA comprise the sequences of SEQ ID NOs: 131 and 136. In some embodiments, the first sgRNA and the second sgRNA comprise the sequences of SEQ ID NOs: 151 and 136. In some embodiments, the first sgRNA and the second sgRNA comprise the sequences of SEQ ID NOs: 139 and 131. In some embodiments, the first sgRNA and the second sgRNA comprise the sequences of SEQ ID NOs: 140 and 131. In some embodiments, the first sgRNA and the second sgRNA comprise the sequences of SEQ ID NOs: 140 and 151. In some embodiments, the first sgRNA and the second sgRNA comprise the sequences of SEQ ID NOs: 141 and 148. In some embodiments, the first sgRNA and the second sgRNA comprise the sequences of SEQ ID NOs: 144 and 149. In some embodiments, the first sgRNA and the second sgRNA comprise the sequences of SEQ ID NOs: 144 and 150. In some embodiments, the first sgRNA and the second sgRNA comprise the sequences of SEQ ID NOs: 145 and 131. In some embodiments, the first sgRNA and the second sgRNA comprise the sequences of SEQ ID NOs: 145 and 151. In some embodiments, the first sgRNA and the second sgRNA comprise the sequences of SEQ ID NOs: 146 and 148. In some embodiments, the first sgRNA and the second sgRNA comprise the sequences of SEQ ID NOs: 134 and 148. In some embodiments, the first sgRNA and the second sgRNA comprise the sequences of SEQ ID NOs: 135 and 149. In some embodiments, the first sgRNA and the second sgRNA comprise the sequences of SEQ ID NOs: 135 and 150. In some embodiments, the first sgRNA and the second sgRNA comprise the sequences of SEQ ID NOs: 136 and 131. In some embodiments, the first sgRNA and the second sgRNA comprise the sequences of SEQ ID NOs: 136 and 151. In some embodiments, the first sgRNA and the second sgRNA comprise the sequences of SEQ ID NOs: 131 and 139. In some embodiments, the first sgRNA and the second sgRNA comprise the sequences of SEQ ID NOs: 131 and 140. In some embodiments, the first sgRNA and the second sgRNA comprise the sequences of SEQ ID NOs: 151 and 140. In some embodiments, the first sgRNA and the second sgRNA comprise the sequences of SEQ ID NOs: 148 and 141. In some embodiments, the first sgRNA and the second sgRNA comprise the sequences of SEQ ID NOs: 149 and 144. In some embodiments, the first sgRNA and the second sgRNA comprise the sequences of SEQ ID NOs: 150 and 144. In some embodiments, the first sgRNA and the second sgRNA comprise the sequences of SEQ ID NOs: 131 and 145. In some embodiments, the first sgRNA and the second sgRNA comprise the sequences of SEQ ID NOs: 151 and 145. In some embodiments, the first sgRNA and the second sgRNA comprise the sequences of SEQ ID NOs: 148 and 146. In some embodiments, the nucleic acid sequence encoding SluCas9 is fused to a nucleic acid sequence encoding a nuclear localization sequence (NLS). In some embodiments, the nucleic acid sequence encoding SluCas9 is fused to two nucleic acid sequences each encoding a nuclear localization sequence (NLS). In some embodiments, the nucleic acid sequence encoding SluCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SluCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an 7SK promoter for expression of a second sgRNA, a second sgRNA guide sequence, and a second sgRNA scaffold sequence. In some embodiments, the first sgRNA and the second sgRNA comprise the sequences of SEQ ID NOs: 148 and 134. In some embodiments, the first sgRNA and the second sgRNA comprise the sequences of SEQ ID NOs: 149 and 135. In some embodiments, the first sgRNA and the second sgRNA comprise the sequences of SEQ ID NOs: 150 and 135. In some embodiments, the first sgRNA and the second sgRNA comprise the sequences of SEQ ID NOs: 131 and 136. In some embodiments, the first sgRNA and the second sgRNA comprise the sequences of SEQ ID NOs: 151 and 136. In some embodiments, the first sgRNA and the second sgRNA comprise the sequences of SEQ ID NOs: 139 and 131. In some embodiments, the first sgRNA and the second sgRNA comprise the sequences of SEQ ID NOs: 140 and 131. In some embodiments, the first sgRNA and the second sgRNA comprise the sequences of SEQ ID NOs: 140 and 151. In some embodiments, the first sgRNA and the second sgRNA comprise the sequences of SEQ ID NOs: 141 and 148. In some embodiments, the first sgRNA and the second sgRNA comprise the sequences of SEQ ID NOs: 144 and 149. In some embodiments, the first sgRNA and the second sgRNA comprise the sequences of SEQ ID NOs: 144 and 150. In some embodiments, the first sgRNA and the second sgRNA comprise the sequences of SEQ ID NOs: 145 and 131. In some embodiments, the first sgRNA and the second sgRNA comprise the sequences of SEQ ID NOs: 145 and 151. In some embodiments, the first sgRNA and the second sgRNA comprise the sequences of SEQ ID NOs: 146 and 148. In some embodiments, the first sgRNA and the second sgRNA comprise the sequences of SEQ ID NOs: 134 and 148. In some embodiments, the first sgRNA and the second sgRNA comprise the sequences of SEQ ID NOs: 135 and 149. In some embodiments, the first sgRNA and the second sgRNA comprise the sequences of SEQ ID NOs: 135 and 150. In some embodiments, the first sgRNA and the second sgRNA comprise the sequences of SEQ ID NOs: 136 and 131. In some embodiments, the first sgRNA and the second sgRNA comprise the sequences of SEQ ID NOs: 136 and 151. In some embodiments, the first sgRNA and the second sgRNA comprise the sequences of SEQ ID NOs: 131 and 139. In some embodiments, the first sgRNA and the second sgRNA comprise the sequences of SEQ ID NOs: 131 and 140. In some embodiments, the first sgRNA and the second sgRNA comprise the sequences of SEQ ID NOs: 151 and 140. In some embodiments, the first sgRNA and the second sgRNA comprise the sequences of SEQ ID NOs: 148 and 141. In some embodiments, the first sgRNA and the second sgRNA comprise the sequences of SEQ ID NOs: 149 and 144. In some embodiments, the first sgRNA and the second sgRNA comprise the sequences of SEQ ID NOs: 150 and 144. In some embodiments, the first sgRNA and the second sgRNA comprise the sequences of SEQ ID NOs: 131 and 145. In some embodiments, the first sgRNA and the second sgRNA comprise the sequences of SEQ ID NOs: 151 and 145. In some embodiments, the first sgRNA and the second sgRNA comprise the sequences of SEQ ID NOs: 148 and 146. In some embodiments, the nucleic acid sequence encoding SluCas9 is fused to a nucleic acid sequence encoding a nuclear localization sequence (NLS). In some embodiments, the nucleic acid sequence encoding SluCas9 is fused to two nucleic acid sequences each encoding a nuclear localization sequence (NLS). In some embodiments, the nucleic acid sequence encoding SluCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SluCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an hU6c promoter for expression of the nucleic acid encoding the first sgRNA, a promoter for expression of a nucleic acid encoding SluCas9 (e.g., CK8e), a nucleic acid encoding SluCas9, a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 148 and 134. In some embodiments, the first sgRNA and the second sgRNA comprise the sequences of SEQ ID NOs: 149 and 135. In some embodiments, the first sgRNA and the second sgRNA comprise the sequences of SEQ ID NOs: 150 and 135. In some embodiments, the first sgRNA and the second sgRNA comprise the sequences of SEQ ID NOs: 131 and 136. In some embodiments, the first sgRNA and the second sgRNA comprise the sequences of SEQ ID NOs: 151 and 136. In some embodiments, the first sgRNA and the second sgRNA comprise the sequences of SEQ ID NOs: 139 and 131. In some embodiments, the first sgRNA and the second sgRNA comprise the sequences of SEQ ID NOs: 140 and 131. In some embodiments, the first sgRNA and the second sgRNA comprise the sequences of SEQ ID NOs: 140 and 151. In some embodiments, the first sgRNA and the second sgRNA comprise the sequences of SEQ ID NOs: 141 and 148. In some embodiments, the first sgRNA and the second sgRNA comprise the sequences of SEQ ID NOs: 144 and 149. In some embodiments, the first sgRNA and the second sgRNA comprise the sequences of SEQ ID NOs: 144 and 150. In some embodiments, the first sgRNA and the second sgRNA comprise the sequences of SEQ ID NOs: 145 and 131. In some embodiments, the first sgRNA and the second sgRNA comprise the sequences of SEQ ID NOs: 145 and 151. In some embodiments, the first sgRNA and the second sgRNA comprise the sequences of SEQ ID NOs: 146 and 148. In some embodiments, the first sgRNA and the second sgRNA comprise the sequences of SEQ ID NOs: 134 and 148. In some embodiments, the first sgRNA and the second sgRNA comprise the sequences of SEQ ID NOs: 135 and 149. In some embodiments, the first sgRNA and the second sgRNA comprise the sequences of SEQ ID NOs: 135 and 150. In some embodiments, the first sgRNA and the second sgRNA comprise the sequences of SEQ ID NOs: 136 and 131. In some embodiments, the first sgRNA and the second sgRNA comprise the sequences of SEQ ID NOs: 136 and 151. In some embodiments, the first sgRNA and the second sgRNA comprise the sequences of SEQ ID NOs: 131 and 139. In some embodiments, the first sgRNA and the second sgRNA comprise the sequences of SEQ ID NOs: 131 and 140. In some embodiments, the first sgRNA and the second sgRNA comprise the sequences of SEQ ID NOs: 151 and 140. In some embodiments, the first sgRNA and the second sgRNA comprise the sequences of SEQ ID NOs: 148 and 141. In some embodiments, the first sgRNA and the second sgRNA comprise the sequences of SEQ ID NOs: 149 and 144. In some embodiments, the first sgRNA and the second sgRNA comprise the sequences of SEQ ID NOs: 150 and 144. In some embodiments, the first sgRNA and the second sgRNA comprise the sequences of SEQ ID NOs: 131 and 145. In some embodiments, the first sgRNA and the second sgRNA comprise the sequences of SEQ ID NOs: 151 and 145. In some embodiments, the first sgRNA and the second sgRNA comprise the sequences of SEQ ID NOs: 148 and 146. In some embodiments, the nucleic acid sequence encoding SluCas9 is fused to a nucleic acid sequence encoding a nuclear localization sequence (NLS). In some embodiments, the nucleic acid sequence encoding SluCas9 is fused to two nucleic acid sequences each encoding a nuclear localization sequence (NLS). In some embodiments, the nucleic acid sequence encoding SluCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SluCas9 with a linker.

In some embodiments, the AAV vector comprises from 5′ to 3′ with respect to the plus strand: the reverse complement of a first sgRNA scaffold sequence, the reverse complement of a nucleic acid encoding a first sgRNA guide sequence, the reverse complement of an 7SK promoter for expression of the nucleic acid encoding the first sgRNA, a promoter for expression of a nucleic acid encoding SluCas9 (e.g., CK8e), a nucleic acid encoding SluCas9, a polyadenylation sequence, an H1m promoter for expression of a second sgRNA, a second sgRNA guide sequence, and a second sgRNA scaffold sequence. In some embodiments, the first sgRNA and the second sgRNA comprise the sequences of SEQ ID NOs: 148 and 134. In some embodiments, the first sgRNA and the second sgRNA comprise the sequences of SEQ ID NOs: 149 and 135. In some embodiments, the first sgRNA and the second sgRNA comprise the sequences of SEQ ID NOs: 150 and 135. In some embodiments, the first sgRNA and the second sgRNA comprise the sequences of SEQ ID NOs: 131 and 136. In some embodiments, the first sgRNA and the second sgRNA comprise the sequences of SEQ ID NOs: 151 and 136. In some embodiments, the first sgRNA and the second sgRNA comprise the sequences of SEQ ID NOs: 139 and 131. In some embodiments, the first sgRNA and the second sgRNA comprise the sequences of SEQ ID NOs: 140 and 131. In some embodiments, the first sgRNA and the second sgRNA comprise the sequences of SEQ ID NOs: 140 and 151. In some embodiments, the first sgRNA and the second sgRNA comprise the sequences of SEQ ID NOs: 141 and 148. In some embodiments, the first sgRNA and the second sgRNA comprise the sequences of SEQ ID NOs: 144 and 149. In some embodiments, the first sgRNA and the second sgRNA comprise the sequences of SEQ ID NOs: 144 and 150. In some embodiments, the first sgRNA and the second sgRNA comprise the sequences of SEQ ID NOs: 145 and 131. In some embodiments, the first sgRNA and the second sgRNA comprise the sequences of SEQ ID NOs: 145 and 151. In some embodiments, the first sgRNA and the second sgRNA comprise the sequences of SEQ ID NOs: 146 and 148. In some embodiments, the first sgRNA and the second sgRNA comprise the sequences of SEQ ID NOs: 134 and 148. In some embodiments, the first sgRNA and the second sgRNA comprise the sequences of SEQ ID NOs: 135 and 149. In some embodiments, the first sgRNA and the second sgRNA comprise the sequences of SEQ ID NOs: 135 and 150. In some embodiments, the first sgRNA and the second sgRNA comprise the sequences of SEQ ID NOs: 136 and 131. In some embodiments, the first sgRNA and the second sgRNA comprise the sequences of SEQ ID NOs: 136 and 151. In some embodiments, the first sgRNA and the second sgRNA comprise the sequences of SEQ ID NOs: 131 and 139. In some embodiments, the first sgRNA and the second sgRNA comprise the sequences of SEQ ID NOs: 131 and 140. In some embodiments, the first sgRNA and the second sgRNA comprise the sequences of SEQ ID NOs: 151 and 140. In some embodiments, the first sgRNA and the second sgRNA comprise the sequences of SEQ ID NOs: 148 and 141. In some embodiments, the first sgRNA and the second sgRNA comprise the sequences of SEQ ID NOs: 149 and 144. In some embodiments, the first sgRNA and the second sgRNA comprise the sequences of SEQ ID NOs: 150 and 144. In some embodiments, the first sgRNA and the second sgRNA comprise the sequences of SEQ ID NOs: 131 and 145. In some embodiments, the first sgRNA and the second sgRNA comprise the sequences of SEQ ID NOs: 151 and 145. In some embodiments, the first sgRNA and the second sgRNA comprise the sequences of SEQ ID NOs: 148 and 146. In some embodiments, the nucleic acid sequence encoding SluCas9 is fused to a nucleic acid sequence encoding a nuclear localization sequence (NLS). In some embodiments, the nucleic acid sequence encoding SluCas9 is fused to two nucleic acid sequences each encoding a nuclear localization sequence (NLS). In some embodiments, the nucleic acid sequence encoding SluCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SluCas9 with a linker.

In some embodiments, the disclosure provides for a composition comprising at least two nucleic acids. In some embodiments, the composition comprises at least two nucleic acid molecules, wherein the first nucleic acid molecule comprises a sequence encoding any of the endonucleases disclosed herein (e.g., a SaCas9 or SluCas9), wherein the second nucleic acid molecule encodes a first guide RNA and a second guide RNA, wherein the first guide RNA and the second guide RNA are not the same sequence, and wherein the second nucleic acid molecule does not encode an endonuclease. In some embodiments, the first nucleic acid molecule also encodes a copy of the first guide RNA and a copy of the second guide RNA. In some embodiments, the first nucleic acid molecule does not encode any guide RNAs. In some embodiments, the second nucleic acid molecule encodes two copies of the first guide RNA and two copies of the second guide RNA. In some embodiments, the second nucleic molecule encodes two copies of the first guide RNA, and one copy of the second guide RNA. In some embodiments, the second nucleic acid molecule encodes one copy of the first guide RNA, and two copies of the second guide RNA. In some embodiments, the second nucleic acid molecule comprises two copies of the first guide RNA, and three copies of the second guide RNA. In some embodiments, the second nucleic acid molecule comprises three copies of the first guide RNA, and two copies of the second guide RNA. In some embodiments, the second nucleic acid molecule encodes three copies of the first guide RNA and three copies of the second guide RNA. In some embodiments, the first nucleic acid molecule comprises from 5′ to 3′ with respect to the plus strand: the reverse complement of a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding the endonuclease, a nucleotide sequence encoding an endonuclease, a polyadenylation sequence, a promoter for expression of the second guide RNA in the same direction as the promoter for the endonuclease, the second guide RNA sequence, and a second guide RNA scaffold sequence. In some embodiments, the promoter for expression of the nucleotide sequence encoding the first guide RNA sequence in the first nucleic acid molecule is a U6 promoter and the promoter for expression of the nucleotide sequence encoding the second guide RNA in the first nucleic acid molecule is a U6 promoter. In some embodiments, the first nucleic acid molecule encodes a Staphylococcus aureus Cas9 (SaCas9) endonuclease, and the first guide RNA comprises the first sequence and the second guide RNA comprises the second sequence from any one the following pairs of sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018. In some embodiments, the first guide RNA comprises the sequence of SEQ ID NO: 12 and the second guide RNA comprises the sequence of SEQ ID NO: 1013. In some embodiments, the first guide RNA comprises the sequence of SEQ ID NO: 1013 and the second guide RNA comprises the sequence of SEQ ID NO: 12. In some embodiments, the first nucleic acid molecule encodes a Staphylococcus lugdunensis (SluCas9) endonuclease, and wherein the first guide RNA comprises the first sequence and the second guide RNAs comprises the second sequence from any one the following pairs of sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146. In some embodiments, a) the first guide RNA comprises the sequence of SEQ ID NO: 148 and the second guide RNA comprises the sequence of SEQ ID NO: 134; b) the second guide RNA comprises the sequence of SEQ ID NO: 145 and the second guide RNA comprises the sequence of SEQ ID NO: 131, c) the first guide RNA comprises the sequence of SEQ ID NO: 134 and the second guide RNA comprises the sequence of SEQ ID NO: 148; d) the second guide RNA comprises the sequence of SEQ ID NO: 131 and the second guide RNA comprises the sequence of SEQ ID NO: 145.

In some embodiments, the first nucleic acid molecule is in a first vector (e.g., AAV9), and the second nucleic acid is in a separate second vector. In some embodiments, the first vector is AAV9. In some embodiments, the second vector is AAV9. In preferred embodiments, the AAV9 vector is less than 5 kb from ITR to ITR in size, inclusive of both ITRs. In particular embodiments, the AAV9 vector is less than 4.9 kb from ITR to ITR in size, inclusive of both ITRs. In further embodiments, the AAV9 vector is less than 4.85 kb from ITR to ITR in size, inclusive of both ITRs. In further embodiments, the AAV9 vector is less than 4.8 kb from ITR to ITR in size, inclusive of both ITRs. In further embodiments, the AAV9 vector is less than 4.75 kb from ITR to ITR in size, inclusive of both ITRs. In further embodiments, the AAV9 vector is less than 4.7 kb from ITR to ITR in size, inclusive of both ITRs. In some embodiments, the second vector comprises from 5′ to 3′ with respect to the plus strand: a promoter for expression of a first copy of a first guide RNA (e.g., a U6 promoter), a first copy of a nucleotide sequence encoding a first guide RNA, a first copy of a nucleotide sequence encoding a first guide RNA scaffold, a promoter for expression of a second copy of the first guide RNA (e.g., a H1 promoter), a second copy of the nucleotide sequence encoding the first guide RNA, a second copy of the nucleotide sequence encoding the first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises from 5′ to 3′ with respect to the plus strand: a promoter for expression of a first guide RNA (e.g., a U6 promoter), a nucleotide sequence encoding a first guide RNA, a nucleotide sequence encoding a first guide RNA scaffold, a promoter for expression of a second guide RNA (e.g., a 7SK promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the nucleotide sequence encoding the first guide scaffold sequence and the promoter for expression of the second guide sequence. In some embodiments, the second vector comprises from 5′ to 3′ with respect to the plus strand: the reverse complement of a nucleotide sequence encoding a scaffold for a first guide RNA, the reverse complement of a nucleotide sequence encoding a first guide RNA, the reverse complement of a promoter for expression of the first guide RNA (e.g., a U6c promoter), a promoter for expression of a second guide RNA (e.g., a U6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In some embodiments, the second vector comprises from 5′ to 3′ with respect to the plus strand: the reverse complement of a nucleotide sequence encoding a first copy of a first guide RNA scaffold, the reverse complement of a nucleotide sequence encoding a first copy of the first guide RNA, the reverse complement of a promoter for expression of the first copy of the first guide RNA (e.g., a 7SK2 promoter), the reverse complement of a second copy of the nucleotide sequence encoding the first guide RNA scaffold, the reverse complement of a second copy of the nucleotide sequence encoding the first guide RNA, the reverse complement of a promoter for expression of the second copy of the nucleotide sequence encoding the first guide RNA (e.g., a hU6c promoter), a promoter for expression of a first copy of a second guide RNA (e.g., a hU6c promoter), a first copy of a nucleotide sequence encoding a second guide RNA, a first copy of a nucleotide sequence encoding a second guide RNA scaffold, a promoter for expression of a second copy of the second guide RNA (e.g., a 7Sk2 promoter), a second copy of the nucleotide sequence encoding the second guide RNA, and a second copy of the nucleotide sequence encoding the second guide RNA scaffold. In some embodiments, the second vector comprises a stuffer sequence (e.g., a 3′UTR desmin sequence) between the reverse complement of the promoter for expression of the second copy of the first guide RNA and the promoter for expression of the first copy of the second guide RNA. In some embodiments, the second vector comprises from 5′ to 3′ with respect to the plus strand: the reverse complement of a nucleotide sequence encoding a first copy of a first guide RNA scaffold, the reverse complement of a first copy of a nucleotide sequence encoding the first guide RNA, the reverse complement of a promoter for expression of the first copy of the first guide RNA (e.g., a 7SK2 promoter), the reverse complement of a first copy of a nucleotide sequence encoding a second guide RNA scaffold, the reverse complement of a nucleotide sequence encoding the first copy of the second guide RNA, the reverse complement of a promoter for expression of the first copy of the second guide RNA (e.g., a hU6c promoter), a promoter for expression of a second copy of the second guide RNA (e.g., a hU6c promoter), a second copy of the nucleotide sequence encoding the second guide RNA, a second copy of the nucleotide sequence encoding the second guide RNA scaffold, a promoter for expression of a second copy of the first guide RNA (e.g., a 7SK2 promoter), a second copy of the nucleotide sequence encoding the first guide RNA, and a second copy of the nucleotide sequence encoding the first guide RNA scaffold. In some embodiments, the second vector comprises a stuffer sequence (e.g., a 3′UTR desmin sequence) between the reverse complement of the promoter for expression of the first copy of the second guide RNA and the promoter for expression of the second copy of the first guide RNA. In some embodiments, the second vector comprises from 5′ to 3′ with respect to the plus strand: the reverse complement of a nucleotide sequence encoding a first guide RNA scaffold, the reverse complement of a nucleotide sequence encoding a first guide RNA, the reverse complement of a promoter for expression of a first guide RNA (e.g., a hU6c promoter), a promoter for expression of a second guide RNA (e.g., a hU6c promoter), a nucleotide sequence encoding a second guide RNA, and a nucleotide sequence encoding a second guide RNA scaffold. In some embodiments, the second vector comprises a stuffer sequence (e.g., a 3′UTR desmin sequence) between the reverse complement of the promoter for expression of the first guide RNA and the promoter for expression of the second guide RNA. In particular embodiments, the first guide RNA is different from the second guide RNA. In some embodiments, the first guide RNA comprises the sequence of SEQ ID NO: 12 and the second guide RNA comprises the sequence of SEQ ID NO: 1013. In some embodiments, the first guide RNA comprises the sequence of SEQ ID NO: 1013 and the second guide RNA comprises the sequence of SEQ ID NO: 12. In some embodiments, a) the first guide RNA comprises the sequence of SEQ ID NO: 148 and the second guide RNA comprises the sequence of SEQ ID NO: 134; b) the second guide RNA comprises the sequence of SEQ ID NO: 145 and the second guide RNA comprises the sequence of SEQ ID NO: 131; c) the first guide RNA comprises the sequence of SEQ ID NO: 134 and the second guide RNA comprises the sequence of SEQ ID NO: 148; or d) the second guide RNA comprises the sequence of SEQ ID NO: 131 and the second guide RNA comprises the sequence of SEQ ID NO: 145. In some embodiments, the scaffold for the first guide RNA comprises the sequence of SEQ ID NO: 901. In some embodiments, the scaffold for the second guide RNA comprises the sequence of SEQ ID NO: 901. In some embodiments, any of the second vectors comprises a stuffer sequence. In some embodiments, the stuffer sequence is a 3′UTR sequence. In some embodiments, the 3′UTR desmin sequence comprises a sequence that is at least 80%, 85%, 90%, 91%, 92%, 93%, 94%, 95%, 96%, 97%, 98%, 99%, or 100% identical to the sequence of SEQ ID NO: 552 (taattaacagatttgt taatgaagaggaaatatacaaatattaatgaacaaaatagtgtgtttagaacaagactcacatacaggagacatacacatgttaaaggaggcattaga atagtggggaaaacttacaaattcagtatagggtaggagcactagatagctaagaggtggggtagggggcagagtgaaggctgtctctcttttact ccttctaaaaatattccagtggatcaaagatgtaggaaacgatggatccatgtaggcctcagccttcaagtactcctctgagatttcagggattatcctt taaaggagtcaggaagagggtagagatcacaaagataataagctcagacagtagtatgttaaataaacctcaggaggttttagtcttaaggtccttaa tctgaccttccaaactgacttttccagaaacttccaaaagcctctc) or fragments thereof.

In some embodiments, if the composition comprises one or more nucleic acids encoding an RNA-targeted endonuclease and one or more guide RNAs, the one or more nucleic acids are designed such that they express the one or more guide RNAs at an equivalent or higher level (e.g., a greater number of expressed transgene copies) as compared to the expression level of the RNA-targeted endonuclease. In some embodiments, the one or more nucleic acids are designed such that they express (e.g., on average in 100 cells) the one or more guide RNAs at least a 1.1, 1.2, 1.3, 1.4, or 1.5 times higher level (e.g., a greater number of expressed transgene copies) as compared to the expression level of the RNA-targeted endonuclease. In some embodiments, the one or more nucleic acids are designed such that they express the one or more guide RNAs at 1.01-1.5, 1.01-1.4, 1.01-1.3, 1.01-1.2, 1.01-1.1, 1.1-2.0, 1.1-1.8, 1.1-1.6, 1.1-1.4, 1.1-1.3, 1.2-2.0, 1.2-1.8, 1.2-1.6, 1.2-1.4, 1.4-2.0, 1.4-1.8, 1.4-1.6, 1.6-2.0, 1.6-1.8, or 1.8-2.0 times higher level (e.g., a greater number of expressed transgene copies) as compared to the expression level of the RNA-targeted endonuclease. In some embodiments, the one or more guide RNAs are designed to express a higher level than the RNA-targeted endonuclease by: a) utilizing one or more regulatory elements (e.g., promoters or enhancers) that express the one or more guide RNAs at a higher level as compared to the regulatory elements (e.g., promoters or enhancers) for expression of the RNA-targeted endonuclease; and/or b) expressing more copies of one or more of the guide RNAs as compared to the number of copies of the RNA-targeted endonuclease (e.g., 2× or 3× as many copies of the nucleotide sequences encoding the one or more guide RNAs as compared to the number of copies of the nucleotide sequences encoding the RNA-targeted endonuclease). For example, in some embodiments, the composition comprises multiple nucleic acid molecules (e.g., in multiple vectors), wherein for every nucleotide sequence encoding an RNA-targeted endonuclease in the nucleic acid molecules in the composition, there are two or three copies of the nucleotide sequence encoding the guide RNA in the nucleic acid molecules in the composition. In some embodiments, the composition comprises a first guide RNA and a second guide RNA, wherein the first guide RNA and the second guide RNA are not the same (e.g., any of the guide RNA pairs disclosed herein), and for every nucleotide sequence encoding an RNA-targeted endonuclease in the nucleic acid molecules in the composition, there are two or three copies of the nucleotide sequence encoding the first guide RNA and/or the second guide RNA.

In some embodiments, any of the nucleic acids disclosed herein encodes an RNA-targeted endonuclease. In some embodiments, the RNA-targeted endonuclease has cleavase activity, which can also be referred to as double-strand endonuclease activity. In some embodiments, the RNA-targeted endonuclease comprises a Cas nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. In particular embodiments, the RNA-targeted endonuclease is a type II CRISPR Cas enzyme. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaficum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromafium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 711:

KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPF
QYLSSSDSKISYETFKKHILNLAKGKGRISKTKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRELIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYNNDLIKINGELYRV
IGVNNDLLNRIEVNMIDITYREYLENMNDKRPPRIIKTIASKTQSIKKY
STDILGNLYEVKSKKHPQIIKKG.

In some embodiments, the nucleic acid encoding SaCas9 comprises the nucleic acid of SEQ ID NO: 9014:

AAGCGCAATTACATCCTGGGCCTGGATATCGGCATCACCTCCGTGGGCTACG
GCATCATCGACTATGAGACACGGGATGTGATCGACGCCGGCGTGAGACTGTTCAAGGAG
GCCAACGTGGAGAACAATGAGGGCCGGCGGAGCAAGAGGGGAGCAAGGCGCCTGAAGC
GGAGAAGGCGCCACAGAATCCAGAGAGTGAAGAAGCTGCTGTTCGATTACAACCTGCTG
ACCGACCACTCCGAGCTGTCTGGCATCAATCCTTATGAGGCCCGGGTGAAGGGCCTGTCC
CAGAAGCTGTCTGAGGAGGAGTTTTCTGCCGCCCTGCTGCACCTGGCAAAGAGGAGAGG
CGTGCACAACGTGAATGAGGTGGAGGAGGACACCGGCAACGAGCTGAGCACAAAGGAG
CAGATCAGCCGCAATTCCAAGGCCCTGGAGGAGAAGTATGTGGCCGAGCTGCAGCTGGA
GCGGCTGAAGAAGGATGGCGAGGTGAGGGGCTCCATCAATCGCTTCAAGACCTCTGACT
ACGTGAAGGAGGCCAAGCAGCTGCTGAAGGTGCAGAAGGCCTACCACCAGCTGGATCAG
AGCTTTATCGATACATATATCGACCTGCTGGAGACCAGGCGCACATACTATGAGGGACC
AGGAGAGGGCTCCCCCTTCGGCTGGAAGGACATCAAGGAGTGGTACGAGATGCTGATGG
GCCACTGCACCTATTTTCCAGAGGAGCTGAGATCCGTGAAGTACGCCTATAACGCCGATC
TGTACAACGCCCTGAATGACCTGAACAACCTGGTCATCACCAGGGATGAGAACGAGAAG
CTGGAGTACTATGAGAAGTTCCAGATCATCGAGAACGTGTTCAAGCAGAAGAAGAAGCC
TACACTGAAGCAGATCGCCAAGGAGATCCTGGTGAACGAGGAGGACATCAAGGGCTACC
GCGTGACCAGCACAGGCAAGCCAGAGTTCACCAATCTGAAGGTGTATCACGATATCAAG
GACATCACAGCCCGGAAGGAGATCATCGAGAACGCCGAGCTGCTGGATCAGATCGCCAA
GATCCTGACCATCTATCAGAGCTCCGAGGACATCCAGGAGGAGCTGACCAACCTGAATA
GCGAGCTGACACAGGAGGAGATCGAGCAGATCAGCAATCTGAAGGGCTACACCGGCAC
ACACAACCTGTCCCTGAAGGCCATCAATCTGATCCTGGATGAGCTGTGGCACACAAACG
ACAATCAGATCGCCATCTTTAACAGGCTGAAGCTGGTGCCAAAGAAGGTGGACCTGAGC
CAGCAGAAGGAGATCCCAACCACACTGGTGGACGATTTCATCCTGTCCCCCGTGGTGAA
GCGGAGCTTCATCCAGAGCATCAAAGTGATCAACGCCATCATCAAGAAGTACGGCCTGC
CCAATGATATCATCATCGAGCTGGCCAGGGAGAAGAACTCTAAGGACGCCCAGAAGATG
ATCAATGAGATGCAGAAGAGGAACCGCCAGACCAATGAGCGGATCGAGGAGATCATCA
GAACCACAGGCAAGGAGAACGCCAAGTACCTGATCGAGAAGATCAAGCTGCACGATAT
GCAGGAGGGCAAGTGTCTGTATAGCCTGGAGGCCATCCCTCTGGAGGACCTGCTGAACA
ATCCATTCAACTACGAGGTGGATCACATCATCCCCCGGAGCGTGAGCTTCGACAATTCCT
TTAACAATAAGGTGCTGGTGAAGCAGGAGGAGAACTCTAAGAAGGGCAATAGGACCCCT
TTCCAGTACCTGTCTAGCTCCGATTCTAAGATCAGCTACGAGACCTTCAAGAAGCACATC
CTGAATCTGGCCAAGGGCAAGGGCCGCATCTCTAAGACCAAGAAGGAGTACCTGCTGGA
GGAGCGGGACATCAACAGATTCAGCGTGCAGAAGGACTTCATCAACCGGAATCTGGTGG
ACACCAGATACGCCACACGCGGCCTGATGAATCTGCTGCGGTCCTATTTCAGAGTGAACA
ATCTGGATGTGAAGGTGAAGAGCATCAACGGCGGCTTCACCTCCTTTCTGCGGAGAAAG
TGGAAGTTTAAGAAGGAGAGAAACAAGGGCTATAAGCACCACGCCGAGGATGCCCTGAT
CATCGCCAATGCCGACTTCATCTTTAAGGAGTGGAAGAAGCTGGACAAGGCCAAGAAAG
TGATGGAGAACCAGATGTTCGAGGAGAAGCAGGCCGAGAGCATGCCCGAGATCGAGAC
CGAGCAGGAGTACAAGGAGATTTTCATCACACCTCACCAGATCAAGCACATCAAGGACT
TCAAGGACTACAAGTATTCCCACAGGGTGGATAAGAAGCCCAACCGCGAGCTGATCAAT
GACACCCTGTATTCTACAAGGAAGGACGATAAGGGCAATACCCTGATCGTGAACAATCT
GAACGGCCTGTACGACAAGGATAATGACAAGCTGAAGAAGCTGATCAACAAGAGCCCC
GAGAAGCTGCTGATGTACCACCACGATCCTCAGACATATCAGAAGCTGAAGCTGATCAT
GGAGCAGTACGGCGACGAGAAGAACCCACTGTATAAGTACTATGAGGAGACCGGCAACT
ACCTGACAAAGTATTCCAAGAAGGATAATGGCCCCGTGATCAAGAAGATCAAGTACTAT
GGCAACAAGCTGAATGCCCACCTGGACATCACCGACGATTACCCCAACAGCCGGAATAA
GGTGGTGAAGCTGAGCCTGAAGCCATACAGGTTCGACGTGTACCTGGACAACGGCGTGT
ATAAGTTTGTGACAGTGAAGAATCTGGATGTGATCAAGAAGGAGAACTACTATGAAGTG
AATAGCAAGTGCTACGAGGAGGCCAAGAAGCTGAAGAAGATCAGCAACCAGGCCGAGT
TCATCGCCTCTTTTTACAACAATGACCTGATCAAGATCAATGGCGAGCTGTATAGAGTGA
TCGGCGTGAACAATGATCTGCTGAACCGCATCGAAGTGAATATGATCGACATCACCTACC
GGGAGTATCTGGAGAACATGAATGATAAGAGGCCCCCTCGCATCATCAAGACCATCGCC
TCTAAGACACAGAGCATCAAGAAGTACTCTACAGACATCCTGGGCAACCTGTATGAGGT
GAAGAGCAAGAAGCACCCTCAGATCATCAAGAAGGGC.

In some embodiments comprising a nucleic acid encoding SaCas9, the SaCas9 comprises an amino acid sequence of SEQ ID NO: 711.

In some embodiments, the SaCas9 is a variant of the amino acid sequence of SEQ ID NO: 711. In some embodiments, the SaCas9 comprises an amino acid other than an E at the position corresponding to position 781 of SEQ ID NO: 711. In some embodiments, the SaCas9 comprises an amino acid other than an N at the position corresponding to position 967 of SEQ ID NO: 711. In some embodiments, the SaCas9 comprises an amino acid other than an R at the position corresponding to position 1014 of SEQ ID NO: 711. In some embodiments, the SaCas9 comprises a K at the position corresponding to position 781 of SEQ ID NO: 711. In some embodiments, the SaCas9 comprises a K at the position corresponding to position 967 of SEQ ID NO: 711. In some embodiments, the SaCas9 comprises an H at the position corresponding to position 1014 of SEQ ID NO: 711. In some embodiments, the SaCas9 comprises an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid other than an R at the position corresponding to position 244 of SEQ ID NO: 711. In some embodiments, the SaCas9 comprises an amino acid other than an N at the position corresponding to position 412 of SEQ ID NO: 711. In some embodiments, the SaCas9 comprises an amino acid other than an N at the position corresponding to position 418 of SEQ ID NO: 711. In some embodiments, the SaCas9 comprises an amino acid other than an R at the position corresponding to position 653 of SEQ ID NO: 711. In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; and an amino acid other than an R at the position corresponding to position 653 of SEQ ID NO: 711. In some embodiments, the SaCas9 comprises an A at the position corresponding to position 244 of SEQ ID NO: 711. In some embodiments, the SaCas9 comprises an A at the position corresponding to position 412 of SEQ ID NO: 711. In some embodiments, the SaCas9 comprises an A at the position corresponding to position 418 of SEQ ID NO: 711. In some embodiments, the SaCas9 comprises an A at the position corresponding to position 653 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; and an A at the position corresponding to position 653 of SEQ ID NO: 711.

In some embodiments, the SaCas9 comprises an amino acid other than an R at the position corresponding to position 244 of SEQ ID NO: 711; an amino acid other than an N at the position corresponding to position 412 of SEQ ID NO: 711; an amino acid other than an N at the position corresponding to position 418 of SEQ ID NO: 711; an amino acid other than an R at the position corresponding to position 653 of SEQ ID NO: 711; an amino acid other than an E at the position corresponding to position 781 of SEQ ID NO: 711; an amino acid other than an N at the position corresponding to position 967 of SEQ ID NO: 711; and an amino acid other than an R at the position corresponding to position 1014 of SEQ ID NO: 711. In some embodiments, the SaCas9 comprises an A at the position corresponding to position 244 of SEQ ID NO: 711; an A at the position corresponding to position 412 of SEQ ID NO: 711; an A at the position corresponding to position 418 of SEQ ID NO: 711; an A at the position corresponding to position 653 of SEQ ID NO: 711; a K at the position corresponding to position 781 of SEQ ID NO: 711; a K at the position corresponding to position 967 of SEQ ID NO: 711; and an H at the position corresponding to position 1014 of SEQ ID NO: 711.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 715 (designated herein as SaCas9-KKH or SACAS9KKH):

KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPF
QYLSSSDSKISYETFKKHILNLAKGKGRISKTKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRKLIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYKNDLIKINGELYRV
IGVNNDLLNRIEVNMIDITYREYLENMNDKRPPHIIKTIASKTQSIKKY
STDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 716 (designated herein as SaCas9-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 717 (designated herein as SaCas9-KKH-HF):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELASVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDAQIAIFARLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATAGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 712:

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 is a variant of the amino acid sequence of SEQ ID NO: 712. In some embodiments, the SluCas9 comprises an amino acid other than an Q at the position corresponding to position 781 of SEQ ID NO: 712. In some embodiments, the SluCas9 comprises an amino acid other than an R at the position corresponding to position 1013 of SEQ ID NO: 712. In some embodiments, the SluCas9 comprises a K at the position corresponding to position 781 of SEQ ID NO: 712. In some embodiments, the SluCas9 comprises a K at the position corresponding to position 966 of SEQ ID NO: 712. In some embodiments, the SluCas9 comprises an H at the position corresponding to position 1013 of SEQ ID NO: 712. In some embodiments, the SluCas9 comprises an amino acid other than an Q at the position corresponding to position 781 of SEQ ID NO: 712; and an amino acid other than an R at the position corresponding to position 1013 of SEQ ID NO: 712. In some embodiments, the SluCas9 comprises a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid other than an R at the position corresponding to position 246 of SEQ ID NO: 712. In some embodiments, the SluCas9 comprises an amino acid other than an N at the position corresponding to position 414 of SEQ ID NO: 712. In some embodiments, the SluCas9 comprises an amino acid other than a T at the position corresponding to position 420 of SEQ ID NO: 712. In some embodiments, the SluCas9 comprises an amino acid other than an R at the position corresponding to position 655 of SEQ ID NO: 712. In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; and an amino acid other than an R at the position corresponding to position 655 of SEQ ID NO: 712. In some embodiments, the SluCas9 comprises an A at the position corresponding to position 246 of SEQ ID NO: 712. In some embodiments, the SluCas9 comprises an A at the position corresponding to position 414 of SEQ ID NO: 712. In some embodiments, the SluCas9 comprises an A at the position corresponding to position 420 of SEQ ID NO: 712. In some embodiments, the SluCas9 comprises an A at the position corresponding to position 655 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; and an A at the position corresponding to position 655 of SEQ ID NO: 712.

In some embodiments, the SluCas9 comprises an amino acid other than an R at the position corresponding to position 246 of SEQ ID NO: 712; an amino acid other than an N at the position corresponding to position 414 of SEQ ID NO: 712; an amino acid other than a T at the position corresponding to position 420 of SEQ ID NO: 712; an amino acid other than an R at the position corresponding to position 655 of SEQ ID NO: 712; an amino acid other than an Q at the position corresponding to position 781 of SEQ ID NO: 712; a K at the position corresponding to position 966 of SEQ ID NO: 712; and an amino acid other than an R at the position corresponding to position 1013 of SEQ ID NO: 712. In some embodiments, the SluCas9 comprises an A at the position corresponding to position 246 of SEQ ID NO: 712; an A at the position corresponding to position 414 of SEQ ID NO: 712; an A at the position corresponding to position 420 of SEQ ID NO: 712; an A at the position corresponding to position 655 of SEQ ID NO: 712; a K at the position corresponding to position 781 of SEQ ID NO: 712; a K at the position corresponding to position 966 of SEQ ID NO: 712; and an H at the position corresponding to position 1013 of SEQ ID NO: 712.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 718 (designated herein as SluCas9-KH or SLUCAS9KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 719 (designated herein as SluCas9-HF):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the SluCas9 comprises an amino acid sequence that is at least 80%, 85%, 90%, 91%, 92%, 93%, 94%, 95%, 96%, 97%, 98%, 99% or 100% identical to the sequence of SEQ ID NO: 720 (designated herein as SluCas9-HF-KH):

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELASVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRAQMEIFAHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATAELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRKLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
HIKKTIGKKVNSIEKLTTDVLGNVFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7021 (designated herein as sRGN1):

MNQKFILGLDIGITSVGYGLIDYETKNIIDAGVRLFPEANVENNEGRRSKRGSRRLKRRRIHRL
DRVKHLLAEYDLLDLTNIPKSTNPYQTRVKGLNEKLSKDELVIALLHIAKRRGIHNVDVAAD
KEETASDSLSTKDQINKNAKFLESRYVCELQKERLENEGHVRGVENRFLTKDIVREAKKIIDT
QMQYYPEIDETFKEKYISLVETRREYFEGPGKGSPFGWEGNIKKWFEQMMGHCTYFPEELRS
VKYSYSAELFNALNDLNNLVITRDEDAKLNYGEKFQIIENVFKQKKTPNLKQIAIEIGVHETEI
KGYRVNKSGTPEFTEFKLYHDLKSIVFDKSILENEAILDQIAEILTIYQDEQSIKEELNKLPEILN
EQDKAEIAKLIGYNGTHRLSLKCIHLINEELWQTSRNQMEIFNYLNIKPNKVDLSEQNKIPKD
MVNDFILSPVVKRTFIQSINVINKVIEKYGIPEDIIIELARENNSDDRKKFINNLQKKNEATRKRI
NEIIGQTGNQNAKRIVEKIRLHDQQEGKCLYSLKDIPLEDLLRNPNNYDIDHIIPRSVSFDDSM
HNKVLVRREQNAKKNNQTPYQYLTSGYADIKYSVFKQHVLNLAENKDRMTKKKREYLLEE
RDINKFEVQKEFINRNLVDTRYATRELTNYLKAYFSANNMNVKVKTINGSFTDYLRKVWKF
KKERNHGYKHHAEDALIIANADFLFKENKKLKAVNSVLEKPEIETKQLDIQVDSEDNYSEMFI
IPKQVQDIKDFRNFKYSHRVDKKPNRQLINDTLYSTRKKDNSTYIVQTIKDIYAKDNTTLKKQ
FDKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7022 (designated herein as sRGN2):

MNQKFILGLDIGITSVGYGLIDYETKNIIDAGVRLFPEANVENNEGRRSKRGSRRLKRRRIHRL
ERVKSLLSEYKIISGLAPTNNQPYNIRVKGLTEQLTKDELAVALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPEFTEFKLYHDLKSVLFDQSILENEDVLDQIAEILTIYQDKDSIKSKLTELDILLNEEDK
ENIAQLTGYNGTHRLSLKCIRLVLEEQWYSSRNQMEIFTHLNIKPKKINLTAANKIPKAMIDEF
ILSPVVKRTFIQSINVINKVIEKYGIPEDIIIELARENNSDDRKKFINNLQKKNEATRKRINEIIGQ
TGNQNAKRIVEKIRLHDQQEGKCLYSLESIALMDLLNNPQNYEVDHIIPRSVAFDNSIHNKVL
VKQIENSKKGNRTPYQYLNSSDAKLSYNQFKQHILNLSKSKDRISKKKKDYLLEERDINKFEV
QKEFINRNLVDTRYATRELTSYLKAYFSANNMDVKVKTINGSFTNHLRKVWRFDKYRNHGY
KHHAEDALIIANADFLFKENKKLKAVNSVLEKPEIETKQLDIQVDSEDNYSEMFIIPKQVQDIK
DFRNFKYSHRVDKKPNRQLINDTLYSTRKKDNSTYIVQTIKDIYAKDNTTLKKQFDKSPEKFL
MYQHDPRTFEKLEVIMKQYANEKNPLAKYHEETGEYLTKYSKKNNGPIVKSLKYIGNKLGS
HLDVTHQFKSSTKKLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYKEYCELNNIKGEPRIK
KTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7023 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7024 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7025 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7026 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKF SHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7027 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7028 (designated herein as Staphylococcus hyicus Cas9 or ShyCas9):

MNNYILGLDIGITSVGYGIVDSDTREIKDAGVRLFPEANVDNNEGRRSKRGARRLKR
RRIHRLDRVKHLLAEYDLLDLTNIPKSTNPYQTRVKGLNEKLSKDELVIALLHIAKRRGIHNV
NVMMDDNDSGNELSTKDQLKKNAKALSDKYVCELQLERFEQDYKVRGEKNRFKTEDFVRE
ARKLLETQSKFFEIDQTFIMRYIELIETRREYFEGPGKGSPFGWEGNIKKWFEQMMGHCTYFP
EELRSVKYSYSAELFNALNDLNNLVITRDEDAKLNYGEKFQIIENVFKQKKTPNLKQIAIEIGV
HETEIKGYRVNKSGKPEFTQFKLYHDLKNIFKDPKYLNDIQLMDNIAEIITIYQDAESIIKELNQ
LPELLSEREKEKISALSGYSGTHRLSLKCINLLLDDLWESSLNQMELFTKLNLKPKKIDLSQQH
KIPSKLVDDFILSPVVKRAFIQSIQVVNAIIDKYGLPEDIIIELARENNSDDRRKFLNQLQKQNE
ETRKQVEKVLREYGNDNAKRIVQKIKLHNMQEGKCLYSLKDIPLEDLLRNPHHYEVDHIIPRS
VAFDNSMHNKVLVRADENSKKGNRTPYQYLNSSESSLSYNEFKQHILNLSKTKDRITKKKRE
YLLEERDINKFDVQKEFINRNLVDTRYATRELTSLLKAYFSANNLDVKVKTINGSFTNYLRKV
WKFDKDRNKGYKHHAEDALIIANADFLFKHNKKLRNINKVLDAPSKEVDKKRVTVQSEDEY
NQIFEDTQKAQAIKKFEIRKFSHRVDKKPNRQLINDTLYSTRNIDGIEYVVESIKDIYSVNNDK
VKTKFKKDPHRLLMYRNDPQTFEKFEKVFKQYESEKNPFAKYYEETGEKIRKFSKTGQGPYI
NKIKYLRERLGRHCDVTNKYINSRNKIVQLKIYSYRFDIYQYGNNYKMITISYIDLEQKSNYY
YISREKYEQKKKDKQIDDSYKFIGSFYKNDIINYNGEMYRVIGVNDSEKNKIQLDMIDISIKDY
MELNNIKKTGVIYKTIGKSTTHIEKYTTDILGNLYKAAPPKKPQLIFK.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7029 (designated herein as Staphylococcus microti Cas9 or Smi Cas9):

MEKDYILGLDIGIGSVGYGLIDYDTKSIIDAGVRLFPEANADNNLGRRAKRGARRLKRRRIHR
LERVKSLLSEYKIISGLAPTNNQPYNIRVKGLTEQLTKDELAVALLHIAKRRGIHNVDVAADK
EETASDSLSTKDQINKNAKFLESRYVCELQKERLENEGHVRGVENRFLTKDIVREAKKIIDTQ
MQYYPEIDETFKEKYISLVETRREYYEGPGKGSPYGWDADVKKWYQLMMGHCTYFPVEFRS
VKYAYTADLYNALNDLNNLTIARDDNPKLEYHEKYHIIENVFKQKRNPTLKQIAKEIGVNDI
NISGYRVTKSGKPQFTSFKLFHDLKKVVKDHAILDDIDLLNQIAEILTIYQDKDSIVAELGQLE
YLMSEADKQSISELTGYTGTHSLSLKCMNMIIDELWHSSMNQMEVFTYLNMRPKKYELKGY
QRIPTDMIDDAILSPVVKRSFKQAIGVVNAIIKKYGLPKDIIIELARESNSAEKSRYLRAIQKKN
EKTRERIEAIIKEYGNENAKGLVQKIKLHDAQEGKCLYSLKDIPLEDLLRNPNNYDIDHIIPRS
VSFDDSMHNKVLVRREQNAKKNNQTPYQYLTSGYADIKYSVFKQHVLNLAENKDRMTKKK
REYLLEERNINKYDVQKEFINRNLVDTRYTTRELTTLLKTYFTINNLDVKVKTINGSFTDFLR
KRWGFKKNRDEGYKHHAEDALIIANADYLFKEHKLLKEIKDVSDLAGDERNSNVKDEDQYE
EVFGGYFKIEDIKKYKIKKFSHRVDKKPNRQLINDTIYSTRVKDDKRYLINTLKNLYDKSNGD
LKERMQKDPESLLMYHHDPQTFEKLKIVMSQYENEKNPLAKYFEETGQYLTKYAKHDNGPA
IHKIKYYGNKLVEHLDITKNYHNPQNKVVQLSQKSFRFDVYQTDKGYKFISIAYLTLKNEKN
YYAISQEKYDQLKSEKKISNNAVFIGSFYTSDIIEINNEKFRVIGVNSDKNNLIEVDRIDIRQKEF
IELEEEKKNNRIKVTIGRKTTNIEKFHTDILGNMYKSKRPKAPQLVFKKG.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 7030 (designated herein as Staphylococcus pasteuri Cas9 or Spa Cas9):

MKEKYILGLDLGITSVGYGIINFETKKIIDAGVRLFPEANVDNNEGRRSKRGSRRLKRRRIHRL
ERVKLLLTEYDLINKEQIPTSNNPYQIRVKGLSEILSKDELAIALLHLAKRRGIHNINVSSEDED
ASNELSTKEQINRNNKLLKDKYVCEVQLQRLKEGQIRGEKNRFKTTDILKEIDQLLKVQKDY
HNLDIDFINQYKEIVETRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPQFTEFKLYHDLKSIVFDKSILENEAILDQIAEILTIYQDEQSIKEELNKLPEILNEQDK
AEIAKLIGYNGTHRLSLKCIHLINEELWQTSRNQMEIFNYLNIKPNKVDLSEQNKIPKDMVND
FILSPVVKRTFIQSINVINKVIEKYGIPEDIIIELARENNSDDRKKFINNLQKKNEATRKRINEIIG
QTGNQNAKRIVEKIRLHDQQEGKCLYSLESIALMDLLNNPQNYEVDHIIPRSVAFDNSIHNKV
LVKQIENSKKGNRTPYQYLNSSDAKLSYNQFKQHILNLSKSKDRISKKKKDYLLEERDINKFE
VQKEFINRNLVDTRYATRELTSYLKAYFSANNMDVKVKTINGSFTNHLRKVWRFDKYRNHG
YKHHAEDALIIANADFLFKENKKLQNTNKILEKPTIENNTKKVTVEKEEDYNNVFETPKLVED
IKQYRDYKFSHRVDKKPNRQLINDTLYSTRMKDEHDYIVQTITDIYGKDNTNLKKQFNKNPE
KFLMYQNDPKTFEKLSIIMKQYSDEKNPLAKYYEETGEYLTKYSKKNNGPIVKKIKLLGNKV
GNHLDVTNKYENSTKKLVKLSIKNYRFDVYLTEKGYKFVTIAYLNVFKKDNYYYIPKDKYQ
ELKEKKKIKDTDQFIASFYKNDLIKLNGDLYKIIGVNSDDRNIIELDYYDIKYKDYCEINNIKG
EPRIKKTIGKKTESIEKFTTDVLGNLYLHSTEKAPQLIFKRGL.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7031 (designated herein as Cas12i1):

MSNKEKNASETRKAYTTKMIPRSHDRMKLLGNFMDYLMDGTPIFFELWNQFGGGIDRDIISG
TANKDKISDDLLLAVNWFKVMPINSKPQGVSPSNLANLFQQYSGSEPDIQAQEYFASNFDTE
KHQWKDMRVEYERLLAELQLSRSDMHHDLKLMYKEKCIGLSLSTAHYITSVMFGTGAKNN
RQTKHQFYSKVIQLLEESTQINSVEQLASIILKAGDCDSYRKLRIRCSRKGATPSILKIVQDYEL
GTNHDDEVNVPSLIANLKEKLGRFEYECEWKCMEKIKAFLASKVGPYYLGSYSAMLENALS
PIKGMTTKNCKFVLKQIDAKNDIKYENEPFGKIVEGFFDSPYFESDTNVKWVLHPHHIGESNI
KTLWEDLNAIHSKYEEDIASLSEDKKEKRIKVYQGDVCQTINTYCEEVGKEAKTPLVQLLRY
LYSRKDDIAVDKIIDGITFLSKKHKVEKQKINPVIQKYPSFNFGNNSKLLGKIISPKDKLKHNL
KCNRNQVDNYIWIEIKVLNTKTMRWEKHHYALSSTRFLEEVYYPATSENPPDALAARFRTKT
NGYEGKPALSAEQIEQIRSAPVGLRKVKKRQMRLEAARQQNLLPRYTWGKDFNINICKRGN
NFEVTLATKVKKKKEKNYKVVLGYDANIVRKNTYAAIEAHANGDGVIDYNDLPVKPIESGF
VTVESQVRDKSYDQLSYNGVKLLYCKPHVESRRSFLEKYRNGTMKDNRGNNIQIDFMKDFE
AIADDETSLYYFNMKYCKLLQSSIRNHSSQAKEYREEIFELLRDGKLSVLKLSSLSNLSFVMF
KVAKSLIGTYFGHLLKKPKNSKSDVKAPPITDEDKQKADPEMFALRLALEEKRLNKVKSKKE
VIANKIVAKALELRDKYGPVLIKGENISDTTKKGKKSSTNSFLMDWLARGVANKVKEMVM
MHQGLEFVEVNPNFTSHQDPFVHKNPENTFRARYSRCTPSELTEKNRKEILSFLSDKPSKRPT
NAYYNEGAMAFLATYGLKKNDVLGVSLEKFKQIMANILHQRSEDQLLFPSRGGMFYLATYK
LDADATSVNWNGKQFWVCNADLVAAYNVGLVDIQKDFKKK.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7032 (designated herein as Cas12i2):

MSSAIKSYKSVLRPNERKNQLLKSTIQCLEDGSAFFFKMLQGLFGGITPEIVRFSTEQEKQQQD
IALWCAVNWFRPVSQDSLTHTIASDNLVEKFEEYYGGTASDAIKQYFSASIGESYYWNDCRQ
QYYDLCRELGVEVSDLTHDLEILCREKCLAVATESNQNNSIISVLFGTGEKEDRSVKLRITKKI
LEAISNLKEIPKNVAPIQEIILNVAKATKETFRQVYAGNLGAPSTLEKFIAKDGQKEFDLKKLQ
TDLKKVIRGKSKERDWCCQEELRSYVEQNTIQYDLWAWGEMFNKAHTALKIKSTRNYNFA
KQRLEQFKEIQSLNNLLVVKKLNDFFDSEFFSGEETYTICVHHLGGKDLSKLYKAWEDDPAD
PENAIVVLCDDLKNNFKKEPIRNILRYIFTIRQECSAQDILAAAKYNQQLDRYKSQKANPSVL
GNQGFTWTNAVILPEKAQRNDRPNSLDLRIWLYLKLRHPDGRWKKHHIPFYDTRFFQEIYAA
GNSPVDTCQFRTPRFGYHLPKLTDQTAIRVNKKHVKAAKTEARIRLAIQQGTLPVSNLKITEIS
ATINSKGQVRIPVKFDVGRQKGTLQIGDRFCGYDQNQTASHAYSLWEVVKEGQYHKELGCF
VRFISSGDIVSITENRGNQFDQLSYEGLAYPQYADWRKKASKFVSLWQITKKNKKKEIVTVE
AKEKFDAICKYQPRLYKFNKEYAYLLRDIVRGKSLVELQQIRQEIFRFIEQDCGVTRLGSLSLS
TLETVKAVKGITYSYFSTALNASKNNPISDEQRKEFDPELFALLEKLELIRTRKKKQKVERIAN
SLIQTCLENNIKFIRGEGDLSTTNNATKKKANSRSMDWLARGVFNKIRQLAPMHNITLFGCGS
LYTSHQDPLVHRNPDKAMKCRWAAIPVKDIGDWVLRKLSQNLRAKNIGTGEYYHQGVKEF
LSHYELQDLEEELLKWRSDRKSNIPCWVLQNRLAEKLGNKEAVVYIPVRGGRIYFATHKVAT
GAVSIVFDQKQVWVCNADHVAAANIALTVKGIGEQSSDEENPDGSRIKLQLTS.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 7033 (designated herein as SpCas9):

MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD.

### Modified Guide RNAs

In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. In some embodiments, a modified guide RNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified guide RNAs comprising nucleosides and nucleotides (collectively “residues”) that can have two, three, four, or more modifications. For example, a modified residue can have a modified sugar and a modified nucleobase, or a modified sugar and a modified phosphodiester. In some embodiments, every base of a guide RNA is modified, e.g., all bases have a modified phosphate group, such as a phosphorothioate group. In certain embodiments, all, or substantially all, of the phosphate groups of an guide RNA molecule are replaced with phosphorothioate groups. In some embodiments, modified guide RNAs comprise at least one modified residue at or near the 5′ end of the RNA. In some embodiments, modified guide RNAs comprise at least one modified residue at or near the 3′ end of the RNA.

In some embodiments, the guide RNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified guide RNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the guide RNAs described herein can contain one or more modified nucleosides or nucleotides, e.g., to introduce stability toward intracellular or serum-based nucleases. In some embodiments, the modified guide RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino) In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification.

Modifications of 2′-O-methyl are encompassed.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Modifications of 2′-fluoro (2′-F) are encompassed.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

Abasic nucleotides refer to those which lack nitrogenous bases.

Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide.

### Ribonucleoprotein Complex

In some embodiments, a composition is encompassed comprising: a) one or more guide RNAs comprising one or more guide sequences from Table 1A, Table 1B, or Table 5 and b) saCas9 (when combined with a gRNA comprising any one of or combination of SEQ ID Nos: 1-35, 1000-1078, and 3000-3069) or sluCas9 (when combined with a gRNA comprising any one of or combination of SEQ ID Nos: 100-225, 2000-2116, and 4000-4251), or any of the mutant Cas9 proteins disclosed herein. In some embodiments, the guide RNA together with a Cas9 is called a ribonucleoprotein complex (RNP).

In some embodiments, the disclosure provides for an RNP complex, wherein the guide RNA (e.g., any of the guide RNAs disclosed herein) binds to or is capable of binding to a target sequence in the dystrophin gene (e.g., a splice acceptor site or a splice donor site for exons 45, 51, or 53, including e.g., the Exon 51 intron-exon junction having the sequence ccagagtaacagtctgagtaggagctaaaatattagggtattgcaa (SEQ ID NO: 721), or a target sequence bound by any of the sequences disclosed in Table 1A, Table 1B, and 5), wherein the dystrophin gene comprises a PAM recognition sequence position upstream of the target sequence, and wherein the RNP cuts at a position that is 3 nucleotides upstream (−3) of the PAM in the dystrophin gene. In some embodiments, the RNP also cuts at a position that is 2 nucleotides upstream (−2), 4 nucleotides upstream (−4), 5 nucleotides upstream (−5), or 6 nucleotides upstream (−6) of the PAM in the dystrophin gene. In some embodiments, the RNP cuts at a position that is 3 nucleotides upstream (−3) and 4 nucleotides upstream (−4) of the PAM in the dystrophin gene.

In some embodiments, chimeric Cas9 (SaCas9 or SluCas9) nucleases are used, where one domain or region of the protein is replaced by a portion of a different protein. In some embodiments, a Cas9 nuclease domain may be replaced with a domain from a different nuclease such as Fok1. In some embodiments, a Cas9 nuclease may be a modified nuclease.

In some embodiments, the Cas9 is modified to contain only one functional nuclease domain. For example, the agent protein may be modified such that one of the nuclease domains is mutated or fully or partially deleted to reduce its nucleic acid cleavage activity.

In some embodiments, a conserved amino acid within a Cas9 protein nuclease domain is substituted to reduce or alter nuclease activity. In some embodiments, a Cas9 nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas9 nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)). Further exemplary amino acid substitutions include D10A and N580A (based on the S. aureus Cas9 protein). See, e.g., Friedland et al., 2015, Genome Biol., 16:257.

In some embodiments, the Cas9 lacks cleavase activity. In some embodiments, the Cas9 comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the Cas9 lacking cleavase activity or the dCas DNA-binding polypeptide is a version of a Cas nuclease (e.g., a Cas9 nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the Cas9 comprises one or more heterologous functional domains (e.g., is or comprises a fusion polypeptide).

In some embodiments, the heterologous functional domain may facilitate transport of the Cas9 into the nucleus of a cell. For example, the heterologous functional domain may be a nuclear localization signal (NLS). In some embodiments, the Cas9 may be fused with 1-10 NLS(s). In some embodiments, the Cas9 may be fused with 1-5 NLS(s). In some embodiments, the Cas9 may be fused with 1-3 NLS(s). In some embodiments, the Cas9 may be fused with one NLS. Where one NLS is used, the NLS may be attached at the N-terminus or the C-terminus of the Cas9 sequence, and may be directly fused/attached or fused/attached via a linker. It may also be inserted within the Cas9 sequence. In other embodiments, the Cas9 may be fused with more than one NLS. In some embodiments, the Cas9 may be fused with 2, 3, 4, or 5 NLSs. In some embodiments, the Cas9 may be fused with two NLSs. In certain circumstances, the two NLSs may be the same (e.g., two SV40 NLSs) or different. In some embodiments, the Cas9 protein is fused with one or more SV40 NLSs. In some embodiments, the SV40 NLS comprises the amino acid sequence of SEQ ID NO: 713 (PKKKRKV). In some embodiments, the Cas9 protein (e.g., the SaCas9 or SluCas9 protein) is fused to one or more nucleoplasmin NLSs. In some embodiments, the Cas protein is fused to one or more c-myc NLSs. In some embodiments, the Cas protein is fused to one or more E1A NLSs. In some embodiments, the Cas protein is fused to one or more BP (bipartite) NLSs. In some embodiments, the nucleoplasmin NLS comprises the amino acid sequence of SEQ ID NO: 714 (KRPAATKKAGQAKKKK). In some embodiments, the Cas9 protein is fused with a c-Myc NLS. In some embodiments, the c-Myc NLS is encoded by the nucleic acid sequence of SEQ ID NO: 722 (CCGGCAGCTAAGAAAAAGAAACTGGAT). In some embodiments, the Cas9 is fused to two SV40 NLS sequences linked at the carboxy terminus. In some embodiments, the Cas9 may be fused with two NLSs, one linked at the N-terminus and one at the C-terminus. In some embodiments, the Cas9 may be fused with 3 NLSs. In some embodiments, the Cas9 may be fused with 3 NLSs, two linked at the N-terminus and one linked at the C-terminus. In some embodiments, the Cas9 may be fused with 3 NLSs, one linked at the N-terminus and two linked at the C-terminus. In some embodiments, the Cas9 may be fused with no NLS. In some embodiments, the Cas9 may be fused with one NLS. In some embodiments, the Cas9 may be fused with an NLS on the C-terminus and does not comprise an NLS fused on the N-terminus. In some embodiments, the Cas9 may be fused with an NLS on the N-terminus and does not comprise an NLS fused on the C-terminus. In some embodiments, the Cas9 protein is fused to an SV40 NLS and to a nucleoplasmin NLS. In some embodiments, the Cas9 protein is fused to an SV40 NLS and to a c-Myc NLS. In some embodiments, the SV40 NLS is fused to the C-terminus of the Cas9, while the nucleoplasmin NLS is fused to the N-terminus of the Cas9 protein. In some embodiments, the SV40 NLS is fused to the C-terminus of the Cas9, while the c-Myc NLS is fused to the N-terminus of the Cas9 protein. In some embodiments, the SV40 NLS is fused to the N-terminus of the Cas9, while the nucleoplasmin NLS is fused to the C-terminus of the Cas9 protein. In some embodiments, the SV40 NLS is fused to the N-terminus of the Cas9, while the c-Myc NLS is fused to the C-terminus of the Cas9 protein. In some embodiments, the SV40 NLS is fused to the Cas9 protein by means of a linker. In some embodiments, the SV40 NLS and linker is encoded by the nucleic acid sequence of SEQ ID NO: 723 (ATGATGGCCCCAAAGAAGAAGCGGAAGGTCGGTATCCACGGAGTCCCAGCAGCC). In some embodiments, the nucleoplasmin NLS is fused to the Cas9 protein by means of a linker. In some embodiments, the c-Myc NLS is fused to the Cas9 protein by means of a linker. In some embodiments, an additional domain may be: a) fused to the N- or C-terminus of the Cas protein (e.g., a Cas9 protein), b) fused to the N-terminus of an NLS fused to the N-terminus of a Cas protein, or c) fused to the C-terminus of an NLS fused to the C-terminus of a Cas protein. In some embodiments, an NLS is fused to the N- and/or C-terminus of the Cas protein by means of a linker. In some embodiments, an NLS is fused to the N-terminus of an N-terminally-fused NLS on a Cas protein by means of a linker, and/or an NLS is fused to the C-terminus of a C-terminally fused NLS on a Cas protein by means of a linker. In some embodiments, the linker is GSVD (SEQ ID NO: 550) or GSGS (SEQ ID NO: 551). In some embodiments, the Cas protein comprises a c-Myc NLS fused to the N-terminus of the Cas protein (or to an N-terminally-fused NLS on the Cas protein), optionally by means of a linker. In some embodiments, the Cas protein comprises an SV40 NLS fused to the C-terminus of the Cas protein (or to a C-terminally-fused NLS on the Cas protein), optionally by means of a linker. In some embodiments, the Cas protein comprises a nucleoplasmin NLS fused to the C-terminus of the Cas protein (or to a C-terminally-fused NLS on the Cas protein), optionally by means of a linker. In some embodiments, the Cas protein comprises: a) a c-Myc NLS fused to the N-terminus of the Cas protein, optionally by means of a linker, b) an SV40 NLS fused to the C-terminus of the Cas protein, optionally by means of a linker, and c) a nucleoplasmin NLS fused to the C-terminus of the SV40 NLS, optionally by means of a linker. In some embodiments, the Cas protein comprises: a) a c-Myc NLS fused to the N-terminus of the Cas protein, optionally by means of a linker, b) a nucleoplasmin NLS fused to the C-terminus of the Cas protein, optionally by means of a linker, and c) an SV40 NLS fused to the C-terminus of the nucleoplasmin NLS, optionally by means of a linker.

In some embodiments, the heterologous functional domain may be capable of modifying the intracellular half-life of the Cas9. In some embodiments, the half-life of the Cas9 may be increased. In some embodiments, the half-life of the Cas9 may be reduced. In some embodiments, the heterologous functional domain may be capable of increasing the stability of the Cas9. In some embodiments, the heterologous functional domain may be capable of reducing the stability of the Cas9. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the Cas9 may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the Cas9 to a specific organelle, cell type, tissue, or organ. In some embodiments, the heterologous functional domain may target the Cas9 to muscle.

In further embodiments, the heterologous functional domain may be an effector domain. When the Cas9 is directed to its target sequence, e.g., when a Cas9 is directed to a target sequence by a guide RNA, the effector domain may modify or affect the target sequence. In some embodiments, the effector domain may be chosen from a nucleic acid binding domain or a nuclease domain (e.g., a non-Cas nuclease domain). In some embodiments, the heterologous functional domain is a nuclease, such as a FokI nuclease. See, e.g., U.S. Pat. No. 9,023,649.

In some embodiments, any of the compositions disclosed herein comprising any of the guides and/or endonucleases disclosed herein is sterile and/or substantially pyrogen-free. In particular embodiments, any of the compositions disclosed herein comprise a pharmaceutically acceptable carrier. The phrase “pharmaceutically or pharmacologically acceptable” refers to molecular entities and compositions that do not produce an adverse, allergic, or other untoward reaction when administered to an animal or human. As used herein “pharmaceutically acceptable carrier” includes any and all solvents (e.g., water), dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. In some embodiments, the composition comprises a preservative to prevent the growth of microorganisms.

### Determination of Efficacy of Guide RNAs

In some embodiments, the efficacy of a guide RNA is determined when delivered or expressed together with other components forming an RNP. In some embodiments, the guide RNA is expressed together with a SaCas9 or SluCas9. In some embodiments, the guide RNA is delivered to or expressed in a cell line that already stably expresses an SaCas9 or SluCas9. In some embodiments the guide RNA is delivered to a cell as part of an RNP. In some embodiments, the guide RNA is delivered to a cell along with a nucleic acid (e.g., mRNA) encoding SaCas9 or SluCas9.

In some embodiments, the efficacy of particular guide RNAs is determined based on in vitro models. In some embodiments, the in vitro model is a cell line.

In some embodiments, the efficacy of particular guide RNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of particular guide RNAs is determined based on in vivo models. In some embodiments, the in vivo model is a rodent model. In some embodiments, the rodent model is a mouse which expresses a mutated dystrophin gene, e.g., a mdx mouse. In some embodiments, the in vivo model is a non-human primate, for example cynomolgus monkey.

### III. Methods of Gene Editing and Treating DMD

This disclosure provides methods for gene editing and treating Duchenne Muscular Dystrophy (DMD). In some embodiments, any of the compositions described herein may be administered to a subject in need thereof for use in making a double or single strand break in any one or more of exons 43, 44, 45, 50, 51, or 53 of the dystrophin (DMD) gene. In some embodiments, pairs of guide RNAs described herein, in any of the vector configurations described herein, may be administered to a subject in need thereof to excise a portion of a DMD, thereby treating DMD. In some embodiments, any of the compositions described herein may be administered to a subject in need thereof for use in treating DMD. In some embodiments, a nucleic acid molecule comprising a first nucleic acid encoding one or more guide RNAs of Table 1A, Table 1B, or Table 5 and a second nucleic acid encoding either SaCas9 or SluCas9 (depending on the guide) is administered to a subject to treat DMD. In some embodiments, a single nucleic acid molecule (which may be a vector, including an AAV vector) comprising a first nucleic acid encoding one or more guide RNAs of Table 1A, Table 1B, or Table 5 and a second nucleic acid encoding either SaCas9 or SluCas9 (depending on the guide) is administered to a subject to treat DMD.

In some embodiments, any of the compositions described herein is administered to a subject in need thereof to treat Duchenne Muscular Dystrophy (DMD).

In some embodiments, any of the compositions described herein is administered to a subject in need thereof to induce a double strand break in any one or more of exons 43, 44, 45, 50, 51, or 53 of the dystrophin gene.

In some embodiments, a method of treating Duchenne Muscular Dystrophy (DMD) is provided, the method comprising delivering to a cell any one of the compositions described herein, wherein the cell comprises a mutation in the dystrophin gene that is known to be associated with DMD.

In particular, in some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 1-35, 1000-1078, and 3000-3069; a nucleic acid encoding one or more spacer sequences comprising at least 17, 18, 19, or 20 contiguous nucleotides of a spacer sequence selected from SEQ ID NOs: 1-35, 1000-1078, and 3000-3069; or a nucleic acid encoding one or more spacer sequences that is at least 90% identical to any one of SEQ ID NOs: 1-35, 1000-1078, 3000-3069; and 2) a Staphylococcus aureus Cas9 (SaCas9) or a nucleic acid encoding (SaCas9).

In some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 1-35, 1000-1078, and 3000-3069; a nucleic acid encoding one or more spacer sequences comprising at least 17, 18, 19, or 20 contiguous nucleotides of a spacer sequence selected from SEQ ID NOs: 1-35, 1000-1078, and 3000-3069; or a nucleic acid encoding one or more spacer sequences that is at least 90% identical to any one of SEQ ID NOs: 1-35, 1000-1078, 3000-3069; and 2) a Staphylococcus aureus Cas9 (SaCas9) or a nucleic acid encoding (SaCas9); wherein the cell comprises a mutation in the dystrophin gene that is known to be associated with DMD.

In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 1-35, 1000-1078, and 3000-3069; a nucleic acid encoding one or more spacer sequences comprising at least 17, 18, 19, or 20 contiguous nucleotides of a spacer sequence selected from SEQ ID NOs: 1-35, 1000-1078, and 3000-3069; or a nucleic acid encoding one or more spacer sequences that is at least 90% identical to any one of SEQ ID NOs: 1-35, 1000-1078, and 3000-3069; and 2) a nucleic acid encoding SaCas9.

In some embodiments, a method of treating Duchenne Muscular Dystrophy (DMD) is provided, the method comprising delivering to a cell a single nucleic acid molecule comprising: 1) a nucleic acid encoding one or more spacer sequences selected from SEQ ID NOs: 10, 12, 15, 16, 20, 27, 28, 32, 33, 35, 1001, 1003, 1005, 1010, 1012, 1013, 1016, 1017, and 1018; a nucleic acid encoding one or more spacer sequence comprising at least 17, 18, 19, or 20 contiguous nucleotides of a spacer sequence selected from SEQ ID NOs: 10, 12, 15, 16, 20, 27, 28, 32, 33, 35, 1001, 1003, 1005, 1010, 1012, 1013, 1016, 1017, and 1018; or a nucleic acid encoding one or more spacer sequence that is at least 90% identical to any one of SEQ ID NOs: 10, 12, 15, 16, 20, 27, 28, 32, 33, 35, 1001, 1003, 1005, 1010, 1012, 1013, 1016, 1017, and 1018; and 2) a nucleic acid encoding a Staphylococcus aureus Cas9 (SaCas9). In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 10, 12, 15, 16, 20, 27, 28, 32, 33, 35, 1001, 1003, 1005, 1010, 1012, 1013, 1016, 1017, and 1018; a nucleic acid encoding one or more spacer sequences comprising at least 17, 18, 19, or 20 contiguous nucleotides of one or more spacer sequences selected from SEQ ID NOs: 10, 12, 15, 16, 20, 27, 28, 32, 33, 35, 1001, 1003, 1005, 1010, 1012, 1013, 1016, 1017, and 1018; or a nucleic acid encoding one or more spacer sequences that is at least 90% identical to any one of SEQ ID NOs: 10, 12, 15, 16, 20, 27, 28, 32, 33, 35, 1001, 1003, 1005, 1010, 1012, 1013, 1016, 1017, and 1018; and 2) a nucleic acid encoding SaCas9. In some embodiments, the spacer sequence is SEQ ID NO: 10. In some embodiments, the spacer sequence is SEQ ID NO: 12. In some embodiments, the spacer sequence is SEQ ID NO: 15. In some embodiments, the spacer sequence is SEQ ID NO: 16. In some embodiments, the spacer sequence is SEQ ID NO: 20. In some embodiments, the spacer sequence is SEQ ID NO: 27. In some embodiments, the spacer sequence is SEQ ID NO: 28. In some embodiments, the spacer sequence is SEQ ID NO: 32. In some embodiments, the spacer sequence is SEQ ID NO: 33. In some embodiments, the spacer sequence is SEQ ID NO: 35. In some embodiments, the spacer sequence is SEQ ID NO: 1001. In some embodiments, the spacer sequence is SEQ ID NO: 1003. In some embodiments, the spacer sequence is SEQ ID NO: 1005. In some embodiments, the spacer sequence is SEQ ID NO: 1010. In some embodiments, the spacer sequence is SEQ ID NO: 1012. In some embodiments, the spacer sequence is SEQ ID NO: 1013. In some embodiments, the spacer sequence is SEQ ID NO: 1016. In some embodiments, the spacer sequence is SEQ ID NO: 1017. In some embodiments, the spacer sequence is SEQ ID NO: 1018.

In some embodiments, a method of treating Duchenne Muscular Dystrophy (DMD) is provided, the method comprising delivering to a cell a single nucleic acid molecule comprising: 1) a nucleic acid encoding one or more spacer sequences selected from SEQ ID NOs: 3022, 3023, 3028, 3029, 3030, 3031, 3038, 3039, 3052, 3053, 3054, 3055, 3062, 3063, 3064, 3065, 3068, and 3069; a nucleic acid encoding one or more spacer sequence comprising at least 17, 18, 19, or 20 contiguous nucleotides of a spacer sequence selected from SEQ ID NOs: 3022, 3023, 3028, 3029, 3030, 3031, 3038, 3039, 3052, 3053, 3054, 3055, 3062, 3063, 3064, 3065, 3068, and 3069; or a nucleic acid encoding one or more spacer sequence that is at least 90% identical to any one of SEQ ID NOs: 3022, 3023, 3028, 3029, 3030, 3031, 3038, 3039, 3052, 3053, 3054, 3055, 3062, 3063, 3064, 3065, 3068, and 3069; and 2) a nucleic acid encoding a Staphylococcus aureus Cas9 (SaCas9). In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 3022, 3023, 3028, 3029, 3030, 3031, 3038, 3039, 3052, 3053, 3054, 3055, 3062, 3063, 3064, 3065, 3068, and 3069; a nucleic acid encoding one or more spacer sequences comprising at least 17, 18, 19, or 20 contiguous nucleotides of one or more spacer sequences selected from SEQ ID NOs: 3022, 3023, 3028, 3029, 3030, 3031, 3038, 3039, 3052, 3053, 3054, 3055, 3062, 3063, 3064, 3065, 3068, and 3069; or a nucleic acid encoding one or more spacer sequences that is at least 90% identical to any one of SEQ ID NOs: 3022, 3023, 3028, 3029, 3030, 3031, 3038, 3039, 3052, 3053, 3054, 3055, 3062, 3063, 3064, 3065, 3068, and 3069; and 2) a nucleic acid encoding SaCas9. In some embodiments, the spacer sequence is SEQ ID NO: 3022. In some embodiments, the spacer sequence is SEQ ID NO: 3023. In some embodiments, the spacer sequence is SEQ ID NO: 3028. In some embodiments, the spacer sequence is SEQ ID NO: 3029. In some embodiments, the spacer sequence is SEQ ID NO: 3030. In some embodiments, the spacer sequence is SEQ ID NO: 3031. In some embodiments, the spacer sequence is SEQ ID NO: 3038. In some embodiments, the spacer sequence is SEQ ID NO: 3039. In some embodiments, the spacer sequence is SEQ ID NO: 3052. In some embodiments, the spacer sequence is SEQ ID NO: 3053. In some embodiments, the spacer sequence is SEQ ID NO: 3054. In some embodiments, the spacer sequence is SEQ ID NO: 3055. In some embodiments, the spacer sequence is SEQ ID NO: 3062. In some embodiments, the spacer sequence is SEQ ID NO: 3063. In some embodiments, the spacer sequence is SEQ ID NO: 3064. In some embodiments, the spacer sequence is SEQ ID NO: 3065. In some embodiments, the spacer sequence is SEQ ID NO: 3068. In some embodiments, the spacer sequence is SEQ ID NO: 3069.

In particular, in some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 100-225, 2000-2116, and 4000-4251; a nucleic acid encoding one or more spacer sequences comprising at least 17, 18, 19, or 20 contiguous nucleotides of a spacer sequence selected from SEQ ID NOs: 100-225, 2000-2116, and 4000-4251; or a nucleic acid encoding one or more spacer sequences that is at least 90% identical to any one of SEQ ID NOs: 100-225, 2000-2116, and 4000-4251; and 2) a Staphylococcus lugdunensis (SluCas9) or a nucleic acid molecule encoding SluCas9.

In particular, in some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 100-225, 2000-2116, and 4000-4251; a nucleic acid encoding one or more spacer sequences comprising at least 17, 18, 19, or 20 contiguous nucleotides of a spacer sequence selected from SEQ ID NOs: 100-225, 2000-2116, and 4000-4251; or a nucleic acid encoding one or more spacer sequences that is at least 90% identical to any one of SEQ ID NOs: 100-225, 2000-2116, and 4000-4251; and 2) a Staphylococcus lugdunensis (SluCas9) or a nucleic acid molecule encoding SluCas9; wherein the cell comprises a mutation in the dystrophin gene that is known to be associated with DMD.

In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 100-225, 2000-2116, and 4000-4251; a nucleic acid encoding one or more spacer sequences comprising at least 17, 18, 19, or 20 contiguous nucleotides of a spacer sequence selected from SEQ ID NOs: 100-225, 2000-2116, and 4000-4251; or a nucleic acid encoding one or more spacer sequences that is at least 90% identical to any one of SEQ ID NOs: 100-225, 2000-2116, and 4000-4251; and 2) a nucleic acid encoding Staphylococcus lugdunensis (SluCas9).

In some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 131, 134, 135, 136, 139, 140, 141, 144, 145, 146, 148, 149, 150, 151, 179, 184, 201, 210, 223, 224, 225; a nucleic acid encoding a spacer sequence comprising at least 17, 18, 19, or 20 contiguous nucleotides of one or more spacer sequences selected from SEQ ID NOs: 131, 134, 135, 136, 139, 140, 141, 144, 145, 146, 148, 149, 150, 151, 179, 184, 201, 210, 223, 224, 225; or a nucleic acid encoding one or more spacer sequences that is at least 90% identical to any one of SEQ ID NOs: 131, 134, 135, 136, 139, 140, 141, 144, 145, 146, 148, 149, 150, 151, 179, 184, 201, 210, 223, 224, 225; and 2) a Staphylococcus lugdunensis (SluCas9) or a nucleic acid molecule encoding SluCas9. In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 131, 134, 135, 136, 139, 140, 141, 144, 145, 146, 148, 149, 150, 151, 179, 184, 201, 210, 223, 224, 225; a nucleic acid encoding one or more spacer sequences comprising at least 17, 18, 19, or 20 contiguous nucleotides of one or more spacer sequences selected from SEQ ID NOs: 131, 134, 135, 136, 139, 140, 141, 144, 145, 146, 148, 149, 150, 151, 179, 184, 201, 210, 223, 224, 225; or a nucleic acid encoding one or more spacer sequences that is at least 90% identical to any one of SEQ ID NOs: 131, 134, 135, 136, 139, 144, 148, 149, 150, 151, 179, 184, 201, 210, 223, 224, 225; and 2) a nucleic acid encoding Staphylococcus lugdunensis (SluCas9). In some embodiments, the spacer sequence is SEQ ID NO: 131. In some embodiments, the spacer sequence is SEQ ID NO: 134. In some embodiments, the spacer sequence is SEQ ID NO: 135. In some embodiments, the spacer sequence is SEQ ID NO: 136. In some embodiments, the spacer sequence is SEQ ID NO: 139. In some embodiments, the spacer sequence is SEQ ID NO: 140. In some embodiments, the spacer sequence is SEQ ID NO: 141. In some embodiments, the spacer sequence is SEQ ID NO: 144. In some embodiments, the spacer sequence is SEQ ID NO: 145. In some embodiments, the spacer sequence is SEQ ID NO: 146. In some embodiments, the spacer sequence is SEQ ID NO: 148. In some embodiments, the spacer sequence is SEQ ID NO: 149. In some embodiments, the spacer sequence is SEQ ID NO: 150. In some embodiments, the spacer sequence is SEQ ID NO: 151. In some embodiments, the spacer sequence is SEQ ID NO: 179. In some embodiments, the spacer sequence is SEQ ID NO: 184. In some embodiments, the spacer sequence is SEQ ID NO: 201. In some embodiments, the spacer sequence is SEQ ID NO: 223. In some embodiments, the spacer sequence is SEQ ID NO: 224. In some embodiments, the spacer sequence is SEQ ID NO: 225.

In some embodiments, a method of treating Duchenne Muscular Dystrophy (DMD) is provided, the method comprising delivering to a cell: 1) a nucleic acid molecule comprising: a nucleic acid encoding one or more spacer sequences selected from SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; a nucleic acid encoding a spacer sequence comprising at least 17, 18, 19, or 20 contiguous nucleotides of one or more spacer sequences selected from SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; or a nucleic acid encoding one or more spacer sequences that is at least 90% identical to any one of SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; and 2) a Staphylococcus lugdunensis (SluCas9) or a nucleic acid molecule encoding SluCas9. In some embodiments, a method of treating Duchenne Muscular Dystrophy (DMD) is provided, the method comprising delivering to a cell: a single nucleic acid molecule comprising: 1) a nucleic acid encoding one or more spacer sequences selected from SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; a nucleic acid encoding one or more spacer sequences comprising at least 17, 18, 19, or 20 contiguous nucleotides of one or more spacer sequences selected from SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; or a nucleic acid encoding one or more spacer sequences that is at least 90% identical to any one of SEQ ID NOs: 4062, 4063, 4068, 4069, 4070, 4071, 4072, 4073, 4078, 4079, 4088, 4089, 4096, 4097, 4098, 4099, 4100, 4101, 4102, 4103, 4158, 4159, 4168, 4169, 4202, 4203, 4220, 4221, 4246, 4247, 4248, 4249, 4250, 4251; and 2) a nucleic acid encoding Staphylococcus lugdunensis (SluCas9). In some embodiments, the spacer sequence is SEQ ID NO: 4062. In some embodiments, the spacer sequence is SEQ ID NO: 4063. In some embodiments, the spacer sequence is SEQ ID NO: 4068. In some embodiments, the spacer sequence is SEQ ID NO: 4069. In some embodiments, the spacer sequence is SEQ ID NO: 4070. In some embodiments, the spacer sequence is SEQ ID NO: 4071. In some embodiments, the spacer sequence is SEQ ID NO: 4072. In some embodiments, the spacer sequence is SEQ ID NO: 4073. In some embodiments, the spacer sequence is SEQ ID NO: 4078. In some embodiments, the spacer sequence is SEQ ID NO: 4079. In some embodiments, the spacer sequence is SEQ ID NO: 4088. In some embodiments, the spacer sequence is SEQ ID NO: 4089. In some embodiments, the spacer sequence is SEQ ID NO: 4096. In some embodiments, the spacer sequence is SEQ ID NO: 4097. In some embodiments, the spacer sequence is SEQ ID NO: 4098. In some embodiments, the spacer sequence is SEQ ID NO: 4099. In some embodiments, the spacer sequence is SEQ ID NO: 4100. In some embodiments, the spacer sequence is SEQ ID NO: 4101. In some embodiments, the spacer sequence is SEQ ID NO: 4102. In some embodiments, the spacer sequence is SEQ ID NO: 4103. In some embodiments, the spacer sequence is SEQ ID NO: 4158. In some embodiments, the spacer sequence is SEQ ID NO: 4159. In some embodiments, the spacer sequence is SEQ ID NO: 4168. In some embodiments, the spacer sequence is SEQ ID NO: 4169. In some embodiments, the spacer sequence is SEQ ID NO: 4202. In some embodiments, the spacer sequence is SEQ ID NO: 4203. In some embodiments, the spacer sequence is SEQ ID NO: 4220. In some embodiments, the spacer sequence is SEQ ID NO: 4221. In some embodiments, the spacer sequence is SEQ ID NO: 4246. In some embodiments, the spacer sequence is SEQ ID NO: 4247. In some embodiments, the spacer sequence is SEQ ID NO: 4248. In some embodiments, the spacer sequence is SEQ ID NO: 4249. In some embodiments, the spacer sequence is SEQ ID NO: 4250. In some embodiments, the spacer sequence is SEQ ID NO: 4251.

In some embodiments, the methods comprise delivering to a cell a nucleic acid molecule encoding SaCas9, wherein the SaCas9 comprises the amino acid sequence of SEQ ID NO: 711. In some embodiments, the methods comprise delivering to a cell a nucleic acid molecule encoding SaCas9, wherein the SaCas9 is a variant of the amino acid sequence of SEQ ID NO: 711. In some embodiments, the methods comprise delivering to a cell a nucleic acid molecule encoding SaCas9, wherein the SaCas9 comprises an amino acid sequence selected from any one of SEQ ID NOs: 715-717. In some embodiments, the methods comprise delivering to a cell a nucleic acid molecule encoding SaCas9, wherein the SaCas9 comprises the amino acid of SEQ ID NO: 715.

In some embodiments, the methods comprise delivering to a cell a nucleic acid molecule encoding SluCas9, wherein the SluCas9 comprises the amino acid sequence of SEQ ID NO: 712. In some embodiments, the methods comprise delivering to a cell a nucleic acid molecule encoding SluCas9, wherein the SaCas9 is a variant of the amino acid sequence of SEQ ID NO: 712. In some embodiments, the methods comprise delivering to a cell a nucleic acid molecule encoding SluCas9, wherein the SluCas9 comprises an amino acid sequence selected from any one of SEQ ID NOs: 718-720.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9). In some embodiments, the first and second spacer sequence are selected from SEQ ID NOs: 10 and 15. In some embodiments, the first and second spacer sequence are selected from SEQ ID NOs: 10 and 16. In some embodiments, the first and second spacer sequence are selected from SEQ ID NOs: 12 and 16. In some embodiments, the first and second spacer sequence are selected from SEQ ID NOs: 1001 and 1005. In some embodiments, the first and second spacer sequence are selected from SEQ ID NOs: 1001 and 15. In some embodiments, the first and second spacer sequence are selected from SEQ ID NOs: 1001 and 16. In some embodiments, the first and second spacer sequence are selected from SEQ ID NOs: 1003 and 1005. In some embodiments, the first and second spacer sequence are selected from SEQ ID NOs: 16 and 1003. In some embodiments, the first and second spacer sequence are selected from SEQ ID NOs: 12 and 1010. In some embodiments, the first and second spacer sequence are selected from SEQ ID NOs: 12 and 1012. In some embodiments, the first and second spacer sequence are selected from SEQ ID NOs: 12 and 1013. In some embodiments, the first and second spacer sequence are selected from SEQ ID NOs: 10 and 1016. In some embodiments, the first and second spacer sequence are selected from SEQ ID NOs: 1017 and 16. In some embodiments, the first and second spacer sequence are selected from SEQ ID NOs: 1018 and 16.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence comprising at least 17, 18, 19, 20, or 21 contiguous nucleotides of any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9).

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence that are selected from spacer sequences that are at least 90% identical to any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9).

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence selected from any one of SEQ ID NOs: 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9), wherein the nucleic acid encoding the SaCas9 comprises the amino acid sequence of SEQ ID NO: 715. In some embodiments, the first and second spacer sequence are selected from SEQ ID NOs: 1001 and 1005. In some embodiments, the first and second spacer sequence are selected from SEQ ID NOs: 1001 and 15. In some embodiments, the first and second spacer sequence are selected from SEQ ID NOs: 1001 and 16. In some embodiments, the first and second spacer sequence are selected from SEQ ID NOs: 1003 and 1005. In some embodiments, the first and second spacer sequence are selected from SEQ ID NOs: 16 and 1003. In some embodiments, the first and second spacer sequence are selected from SEQ ID NOs: 12 and 1010. In some embodiments, the first and second spacer sequence are selected from SEQ ID NOs: 12 and 1012. In some embodiments, the first and second spacer sequence are selected from SEQ ID NOs: 12 and 1013. In some embodiments, the first and second spacer sequence are selected from SEQ ID NOs: 10 and 1016. In some embodiments, the first and second spacer sequence are selected from SEQ ID NOs: 1017 and 16. In some embodiments, the first and second spacer sequence are selected from SEQ ID NOs: 1018 and 16.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence comprising at least 17, 18, 19, 20, or 21 contiguous nucleotides of any one of SEQ ID NOs: 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9), wherein the nucleic acid encoding the SaCas9 comprises the amino acid sequence of SEQ ID NO: 715.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence that is at least 90% identical to any one of SEQ ID NOs: 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; and 1018 and 16; and a nucleic acid encoding a Staphylococcus aureus Cas9 (SaCas9), wherein the nucleic acid encoding the SaCas9 comprises the amino acid sequence of SEQ ID NO: 715.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; and 146 and 148; and a nucleic acid encoding a Staphylococcus lugdunensis (SluCas9). In some embodiments, the first and second spacer sequence are selected from SEQ ID NOs: 148 and 134. In some embodiments, the first and second spacer sequence are selected from SEQ ID NOs: 149 and 135. In some embodiments, the first and second spacer sequence are selected from SEQ ID NOs: 150 and 135. In some embodiments, the first and second spacer sequence are selected from SEQ ID NOs: 131 and 136. In some embodiments, the first and second spacer sequence are selected from SEQ ID NOs: 151 and 136. In some embodiments, the first and second spacer sequence are selected from SEQ ID NOs: 139 and 131. In some embodiments, the first and second spacer sequence are selected from SEQ ID NOs: 139 and 151. In some embodiments, the first and second spacer sequence are selected from SEQ ID NOs: 140 and 131. In some embodiments, the first and second spacer sequence are selected from SEQ ID NOs: 140 and 151. In some embodiments, the first and second spacer sequence are selected from SEQ ID NOs: 141 and 148. In some embodiments, the first and second spacer sequence are selected from SEQ ID NOs: 144 and 149. In some embodiments, the first and second spacer sequence are selected from SEQ ID NOs: 144 and 150. In some embodiments, the first and second spacer sequence are selected from SEQ ID NOs: 145 and 131. In some embodiments, the first and second spacer sequence are selected from SEQ ID NOs: 145 and 151. In some embodiments, the first and second spacer sequence are selected from SEQ ID NOs: 146 and 148.

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence comprising at least 17, 18, 19, 20, or 21 contiguous nucleotides of any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; and 146 and 148; and a nucleic acid encoding a Staphylococcus lugdunensis (SluCas9).

In some embodiments, methods are provided for treating Duchenne Muscular Dystrophy (DMD), the method comprising delivering to a cell a single nucleic acid molecule comprising: a nucleic acid encoding a pair of guide RNAs comprising: a first and second spacer sequence that are selected from spacer sequences that are at least 90% identical to any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; and 146 and 148; and a nucleic acid encoding a Staphylococcus lugdunensis (SluCas9).

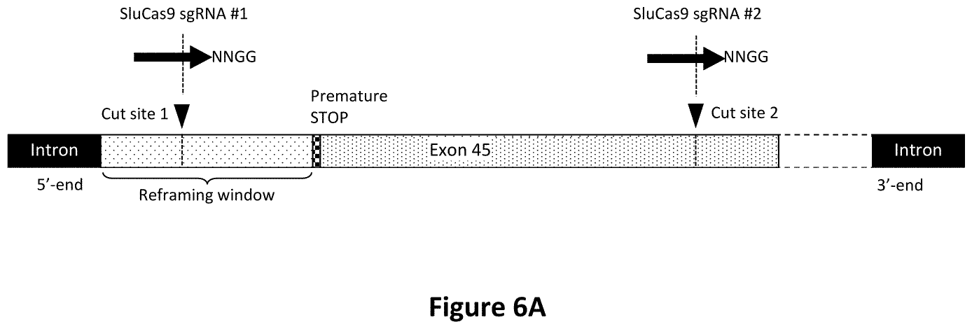

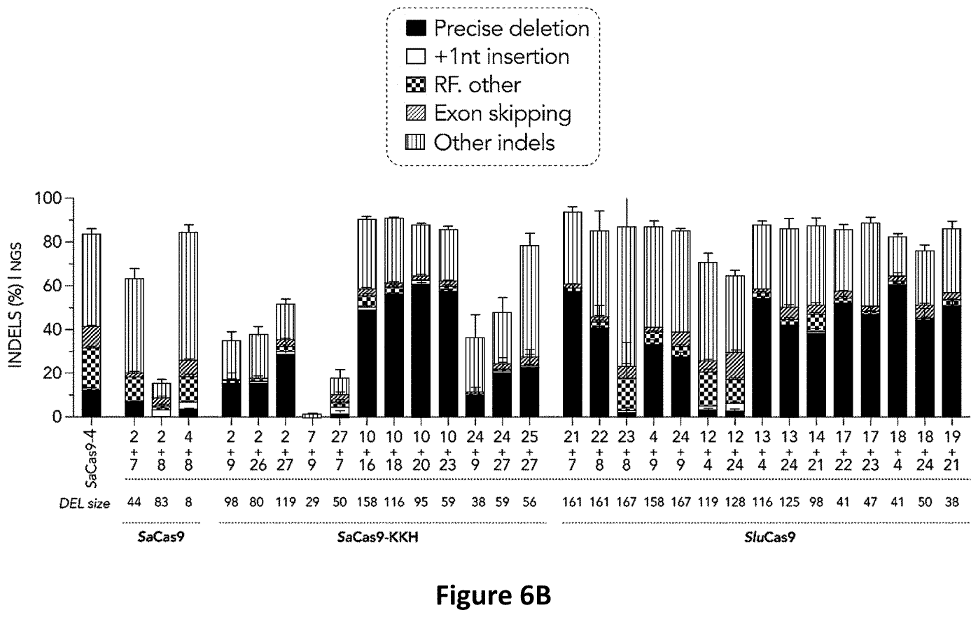

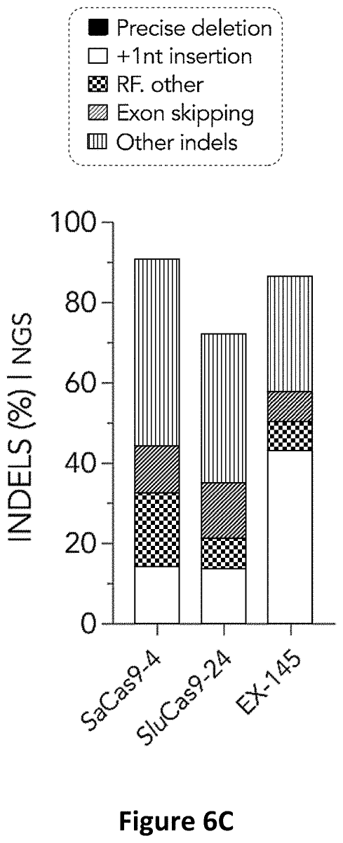

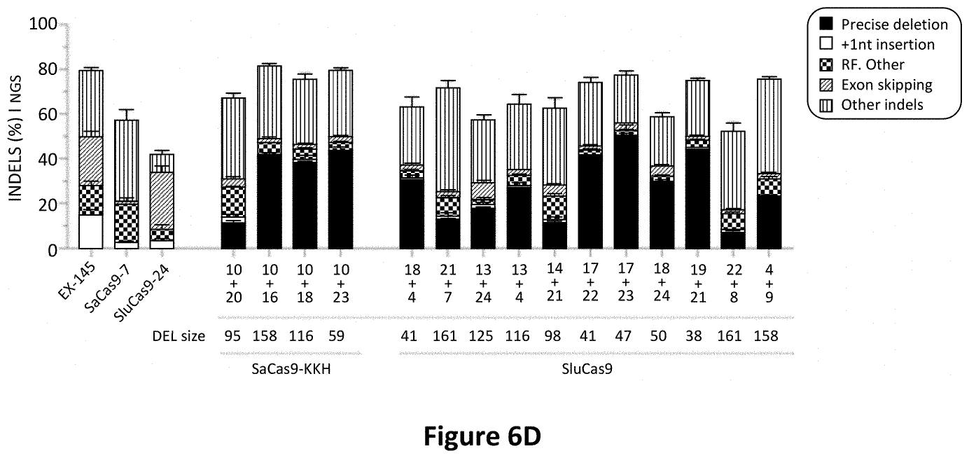

In some embodiments, methods are provided for excising a portion of an exon with a premature stop codon in a subject with Duchenne Muscular Dystrophy (DMD), the method comprising delivering to a cell a single nucleic acid molecule comprising: (i) a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and (ii) a nucleic acid encoding a Staphylococcus aureus Cas9 (SaCas9); wherein the pair of guide RNAs and SaCas9 excise a portion of the exon. In some embodiments, the single nucleic acid molecule is delivered to the cell on a single vector. In some embodiments, the portions of the exon remaining after excision are rejoined with a one nucleotide insertion. In some embodiments, the portions of the exon remaining after excision are rejoined without a nucleotide insertion. Precise segmental deletion means that the dual cut process takes out a specific deletion. This precise deletion can lead to exon reframing (see e.g., FIG. 6). In some embodiments, the size of excised portion is between 5 and 250, 5 and 225, 5 and 200, 5 and 190, 5 and 180, 5 and 170, 5 and 160, 5 and 150, 5 and 125, 5 and 120, 5 and 115, 5 and 110, 5 and 100, 5 and 95, 5 and 90, 5 and 85, 5 and 80, 5 and 75, 5 and 70, 5 and 65, 5 and 60, 5 and 55, 5 and 50, 5 and 45, 5 and 40, 5 and 35, 5 and 30, 5 and 25, 5 and 20, 5 and 15, and 5-10 nucleotides. In some embodiments, the size of excised portion is between 20 and 250, 20 and 225, 20 and 200, 20 and 190, 20 and 180, 20 and 170, 20 and 160, 20 and 150, 20 and 125, 20 and 120, 20 and 115, 20 and 110, 20 and 100, 20 and 95, 20 and 90, 20 and 85, 20 and 80, 20 and 75, 20 and 70, 20 and 65, 20 and 60, 20 and 55, 20 and 50, 20 and 45, 20 and 40, 20 and 35, 20 and 30, and 20 and 25 nucleotides. In some embodiments, the size of excised portion is between 50 and 250, 50 and 225, 50 and 200, 50 and 190, 50 and 180, 50 and 170, 50 and 160, 50 and 150, 50 and 125, 50 and 120, 50 and 115, 50 and 110, and 50 and 100 nucleotides. In some embodiments, the size of excised portion of the exon is between 8 and 167 nucleotides. In some embodiments, the exon is exon 45 of the dystrophin gene. In some embodiments, the pair of guide RNAs comprise a first and second spacer sequence of SEQ ID NOs: 10 and 15 (or SEQ ID Nos: 15 and 10). In some embodiments, the pair of guide RNAs comprise a first and second spacer sequence of SEQ ID NOs: 10 and 16 (or SEQ ID Nos: 16 and 10). In some embodiments, the pair of guide RNAs comprise a first and second spacer sequence of SEQ ID NOs: 12 and 16 (or SEQ ID Nos: 16 and 12). In some embodiments, the pair of guide RNAs comprise a first and second spacer sequence of SEQ ID NOs: 1001 and 1005 (or SEQ ID Nos: 1005 and 1001). In some embodiments, the pair of guide RNAs comprise a first and second spacer sequence of SEQ ID NOs: 1001 and 15 (or SEQ ID Nos: 15 and 1001). In some embodiments, the pair of guide RNAs comprise a first and second spacer sequence of SEQ ID NOs: 1001 and 16 (or SEQ ID Nos: 16 and 1001). In some embodiments, the pair of guide RNAs comprise a first and second spacer sequence of SEQ ID NOs: 1003 and 1005 (or SEQ ID Nos: 1005 and 1003). In some embodiments, the pair of guide RNAs comprise a first and second spacer sequence of SEQ ID NOs: 16 and 1003 (or SEQ ID Nos: 1003 and 16). In some embodiments, the pair of guide RNAs comprise a first and second spacer sequence of SEQ ID NOs: 12 and 1010 (or SEQ ID Nos: 1010 and 12). In some embodiments, the pair of guide RNAs comprise a first and second spacer sequence of SEQ ID NOs: 12 and 1012 (or SEQ ID Nos: 1012 and 12). In some embodiments, the pair of guide RNAs comprise a first and second spacer sequence of SEQ ID NOs: 12 and 1013 (or SEQ ID Nos: 1013 and 12). In some embodiments, the pair of guide RNAs comprise a first and second spacer sequence of SEQ ID NOs: 10 and 1016 (or SEQ ID Nos: 1016 and 10). In some embodiments, the pair of guide RNAs comprise a first and second spacer sequence of SEQ ID NOs: 1017 and 16 (or SEQ ID Nos: 16 and 1017). In some embodiments, the pair of guide RNAs comprise a first and second spacer sequence of SEQ ID NOs: 1018 and 16 (or SEQ ID Nos: 16 and 1018). In some embodiments, the SaCas9 comprises the amino acid sequence of SEQ ID NO: 715.

In some embodiments, methods are provided for excising a portion of an exon with a premature stop codon in a subject with Duchenne Muscular Dystrophy (DMD), the method comprising delivering to a cell a single nucleic acid molecule comprising: (i) a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and (ii) a nucleic acid encoding a Staphylococcus lugdunensis (SluCas9); wherein the pair of guide RNAs and SluCas9 excise a portion of the exon. In some embodiments, the single nucleic acid molecule is delivered to the cell on a single vector. In some embodiments, the portions of the exon remaining after excision are rejoined with a one nucleotide insertion. In some embodiments, the portions of the exon remaining after excision are rejoined without a nucleotide insertion. In some embodiments, the size of excised portion of the exon is between 8 and 167 nucleotides. In some embodiments, the exon is exon 45 of the dystrophin gene. In some embodiments, the pair of guide RNAs comprise a first and second spacer sequence of SEQ ID NOs: 148 and 134 (or SEQ ID Nos: 134 and 148). In some embodiments, the pair of guide RNAs comprise a first and second spacer sequence of SEQ ID NOs: 149 and 135 (or SEQ ID Nos: 135 and 149). In some embodiments, the pair of guide RNAs comprise a first and second spacer sequence of SEQ ID NOs: 150 and 135 (or SEQ ID Nos: 135 and 150). In some embodiments, the pair of guide RNAs comprise a first and second spacer sequence of SEQ ID NOs: 131 and 136 (or SEQ ID Nos: 136 and 131). In some embodiments, the pair of guide RNAs comprise a first and second spacer sequence of SEQ ID NOs: 151 and 136 (or SEQ ID Nos: 136 and 151). In some embodiments, the pair of guide RNAs comprise a first and second spacer sequence of SEQ ID NOs: 139 and 131 (or SEQ ID Nos: 131 and 139). In some embodiments, the pair of guide RNAs comprise a first and second spacer sequence of SEQ ID NOs: 139 and 151 (or SEQ ID Nos: 151 and 139). In some embodiments, the pair of guide RNAs comprise a first and second spacer sequence of SEQ ID NOs: 140 and 131 (or SEQ ID Nos: 131 and 140). In some embodiments, the pair of guide RNAs comprise a first and second spacer sequence of SEQ ID NOs: 140 and 151 (or SEQ ID Nos: 151 and 140). In some embodiments, the pair of guide RNAs comprise a first and second spacer sequence of SEQ ID NOs: 141 and 148 (or SEQ ID Nos: 148 and 141). In some embodiments, the pair of guide RNAs comprise a first and second spacer sequence of SEQ ID NOs: 144 and 149 (or SEQ ID Nos: 149 and 144). In some embodiments, the pair of guide RNAs comprise a first and second spacer sequence of SEQ ID NOs: 144 and 150 (or SEQ ID Nos: 150 and 144). In some embodiments, the pair of guide RNAs comprise a first and second spacer sequence of SEQ ID NOs: 145 and 131 (or SEQ ID Nos: 131 and 145). In some embodiments, the pair of guide RNAs comprise a first and second spacer sequence of SEQ ID NOs: 145 and 151 (or SEQ ID Nos: 151 and 145). In some embodiments, the pair of guide RNAs comprise a first and second spacer sequence of SEQ ID NOs: 146 and 148 (or SEQ ID Nos: 148 and 146).

In some embodiments, the subject is a mammal. In some embodiments, the subject is human

For treatment of a subject (e.g., a human), any of the compositions disclosed herein may be administered in a manner compatible with the dosage formulation and in such amount as is therapeutically effective. The compositions may be readily administered in a variety of dosage forms, such as injectable solutions. For parenteral administration in an aqueous solution, for example, the solution will generally be suitably buffered and the liquid diluent first rendered isotonic with, for example, sufficient saline or glucose. Such aqueous solutions may be used, for example, for intravenous, intramuscular, subcutaneous, and/or intraperitoneal administration.

### Combination Therapy

In some embodiments, the invention comprises combination therapies comprising any of the methods or uses described herein together with an additional therapy suitable for ameliorating DMD.

### Delivery of Guide RNA Compositions

The methods and uses disclosed herein may use any suitable approach for delivering the guide RNAs and compositions described herein. Exemplary delivery approaches include vectors, such as viral vectors; lipid nanoparticles; transfection; and electroporation. In some embodiments, vectors or LNPs associated with the single-vector guide RNAs/Cas9's disclosed herein are for use in preparing a medicament for treating DMD.

Where a vector is used, it may be a viral vector, such as a non-integrating viral vector. In some embodiments, viral vector is an adeno-associated virus vector, a lentiviral vector, an integrase-deficient lentiviral vector, an adenoviral vector, a vaccinia viral vector, an alphaviral vector, or a herpes simplex viral vector. In some embodiments, the viral vector is an adeno-associated virus (AAV) vector. In some embodiments, the AAV vector is an AAV1, AAV2, AAV3, AAV4, AAVS, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), or AAV9 vector, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. In some embodiments, the AAV vector size is measured in length of nucleotides from ITR to ITR, inclusive of both ITRs. In some embodiments, the AAV vector is less than 5 kb in size from ITR to ITR, inclusive of both ITRs. In particular embodiments, the AAV vector is less than 4.9 kb from ITR to ITR in size, inclusive of both ITRs. In further embodiments, the AAV vector is less than 4.85 kb in size from ITR to ITR, inclusive of both ITRs. In further embodiments, the AAV vector is less than 4.8 kb in size from ITR to ITR, inclusive of both ITRs. In further embodiments, the AAV vector is less than 4.75 kb in size from ITR to ITR, inclusive of both ITRs. In further embodiments, the AAV vector is less than 4.7 kb in size from ITR to ITR, inclusive of both ITRs. In some embodiments, the vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb from ITR to ITR in size, inclusive of both ITRs. In some embodiments, the vector is between 4.4-4.85 kb in size from ITR to ITR, inclusive of both ITRs. In some embodiments, the vector is an AAV9 vector.

In some embodiments, the vector (e.g., viral vector, such as an adeno-associated viral vector) comprises a tissue-specific (e.g., muscle-specific) promoter, e.g., which is operatively linked to a sequence encoding the guide RNA. In some embodiments, the muscle-specific promoter is a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods Clin Dev. 2016; 3:16081, and references cited therein for detailed discussion of tissue-specific promoters including neuron-specific promoters.

In some embodiments, in addition to guide RNA and Cas9 sequences, the vectors further comprise nucleic acids that do not encode guide RNAs. Nucleic acids that do not encode guide RNA and Cas9 include, but are not limited to, promoters, enhancers, and regulatory sequences. In some embodiments, the vector comprises one or more nucleotide sequence(s) encoding a crRNA, a trRNA, or a crRNA and trRNA.

Lipid nanoparticles (LNPs) are a known means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

Electroporation is a well-known means for delivery of cargo, and any electroporation methodology may be used for delivering the single vectors disclosed herein.

In some embodiments, the invention comprises a method for delivering any one of the single vectors disclosed herein to an ex vivo cell, wherein the guide RNA is encoded by a vector, associated with an LNP, or in aqueous solution. In some embodiments, the guide RNA/LNP or guide RNA is also associated with a Cas9 or sequence encoding Cas9 (e.g., in the same vector, LNP, or solution).

In some embodiments, the disclosure provides for methods of using any of the guides, endonucleases, cells, or compositions disclosed herein in research methods. For example, any of the guides or endonucleases disclosed herein may be used alone or in combination in experiments under various parameters (e.g., temperatures, pH, types of cells) or combined with other reagents to evaluate the activity of the guides and/or endonucleases.

Further embodiments encompassed by the disclosure are as follows.
Embodiment B 1 is a composition comprising a single nucleic acid molecule encoding one or more guide RNAs and a Cas9, wherein the single nucleic acid molecule comprises:
   a. a first nucleic acid encoding one or more spacer sequences selected from any one of SEQ ID NOs: 1-35, 1000-1078, or 3000-3069 and a second nucleic acid encoding a Staphylococcus aureus Cas9 (SaCas9);
   b. a first nucleic acid encoding one or more spacer sequences selected from any one of SEQ ID NOs: 100-225, 2000-2116, or 4000-4251 and a second nucleic acid encoding a Staphylococcus lugdunensis (SluCas9);
   c. a first nucleic acid encoding one or more spacer sequences comprising at least 17, 18, 19, or 20 contiguous nucleotides of a spacer sequence selected from any one of SEQ ID NOs: 1-35, 1000-1078, or 3000-3069 and a second nucleic acid encoding a Staphylococcus aureus Cas9 (SaCas9);
   d. a first nucleic acid encoding one or more spacer sequences comprising at least 17, 18, 19, or 20 contiguous nucleotides of a spacer sequence selected from any one of SEQ ID NOs: 100-225, 2000-2116, or 4000-4251 and a second nucleic acid encoding a Staphylococcus lugdunensis (SluCas9);
   e. a first nucleic acid encoding one or more spacer sequences that is at least 90% identical to any one of SEQ ID NOs: 1-35, 1000-1078, or 3000-3069 and a second nucleic acid encoding a Staphylococcus aureus Cas9 (SaCas9);
   f. a first nucleic acid encoding one or more spacer sequences that is at least 90% identical to any one of SEQ ID NOs: 100-225, 2000-2116, or 4000-4251 and a second nucleic acid encoding a Staphylococcus lugdunensis (SluCas9);
   g. a first nucleic acid encoding a pair of guide RNAs comprising a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).
   h. a first nucleic acid encoding a pair of guide RNAs comprising at least 17, 18, 19, 20, or 21 contiguous nucleotides of a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9);
   i. a first nucleic acid encoding a pair of guide RNAs that is at least 90% identical to a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9);
   j. a first nucleic acid encoding a pair of guide RNAs comprising a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9);
   k. a first nucleic acid encoding a pair of guide RNAs comprising at least 17, 18, 19, 20, or 21 contiguous nucleotides of a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   l. a first nucleic acid encoding a pair of guide RNAs that is at least 90% identical to a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9).
Embodiment B 2 is the composition of claim 1, wherein the guide RNA is an sgRNA.
Embodiment B 3 is the composition of claim 1, wherein the guide RNA is modified.
Embodiment B 4 is the composition of claim 3, wherein the modification alters one or more 2′ positions and/or phosphodiester linkages.
Embodiment B 5 is the composition of any one of claims 3-4, wherein the modification alters one or more, or all, of the first three nucleotides of the guide RNA.
Embodiment B 6 is the composition of any one of claims 3-5, wherein the modification alters one or more, or all, of the last three nucleotides of the guide RNA.
Embodiment B 7 is the composition of any one of claims 3-6, wherein the modification includes one or more of a phosphorothioate modification, a 2′-OMe modification, a 2′-O-MOE modification, a 2′-F modification, a 2′-O-methine-4′ bridge modification, a 3′-thiophosphonoacetate modification, or a 2′-deoxy modification.
Embodiment B 8 is the composition of any one of the preceding claims, wherein the composition further comprises a pharmaceutically acceptable excipient.
Embodiment B 9 is the composition of any one of the preceding claims, wherein the single nucleic acid molecule is associated with a lipid nanoparticle (LNP).
Embodiment B 10 is the composition of any one of claims 1-8, wherein the single nucleic acid molecule is a viral vector.
Embodiment B 11 is the composition of claim 10, wherein the viral vector is an adeno-associated virus vector, a lentiviral vector, an integrase-deficient lentiviral vector, an adenoviral vector, a vaccinia viral vector, an alphaviral vector, or a herpes simplex viral vector.
Embodiment B 12 is the composition of claim 10, wherein the viral vector is an adeno-associated virus (AAV) vector.
Embodiment B 13 is the composition of claim 12, wherein the AAV vector is an AAV1, AAV2, AAV3, AAV4, AAV5, AAV6, AAV7, AAV8, AAVrh10, AAVrh74, or AAV9 vector, wherein the number following AAV indicates the AAV serotype.
Embodiment B 14 is the composition of claim 13, wherein the AAV vector is an AAV serotype 9 vector.
Embodiment B 15 is the composition of claim 13, wherein the AAV vector is an AAVrh10 vector.
Embodiment B 16 is the composition of claim 13, wherein the AAV vector is an AAVrh74 vector.
Embodiment B 17 is the composition of any one of claims 10-16, comprising a viral vector, wherein the viral vector comprises a tissue-specific promoter.
Embodiment B 18 is the composition of any one of claims 10-16, comprising a viral vector, wherein the viral vector comprises a muscle-specific promoter, optionally wherein the muscle-specific promoter is a muscle creatine kinase promoter, a desmin promoter, an MHCK7 promoter, an SPc5-12 promoter, or a CK8e promoter.
Embodiment B 19 is the composition of any one of claims 10-16, comprising a viral vector, wherein the viral vector comprises a U6, H1, or 7SK promoter.
Embodiment B 20 is the composition of any one of claims 1-19, comprising a nucleic acid encoding SaCas9, wherein the spacer sequence is selected from any one of SEQ ID NOs: 12, 15, 16, 20, 27, 28, 32, 33, and 35.
Embodiment B 21 is the composition of any one of claims 1-20, comprising a nucleic acid encoding SaCas9, wherein the SaCas9 comprises the amino acid sequence of SEQ ID NO: 711.
Embodiment B 22 is the composition of any one of claims 1-20, comprising a nucleic acid encoding SaCas9, wherein the SaCas9 is a variant of the amino acid sequence of SEQ ID NO: 711.
Embodiment B 23 is the composition of any one of claims 1-20, comprising a nucleic acid encoding SaCas9, wherein the SaCas9 comprises an amino acid sequence selected from any one of SEQ ID NOs: 715-717.
Embodiment B 24 is the composition of any one of claims 1-19, comprising a nucleic acid encoding SluCas9, wherein the spacer sequence is selected from any one of SEQ ID NOs: 131, 134, 135, 136, 139, 144, 148, 149, 150, 151, 179, 184, 201, 210, 223, 224, 225.
Embodiment B 25 is the composition of any one of claim 1-19, or 24, comprising a nucleic acid encoding SluCas9, wherein the SluCas9 comprises the amino acid sequence of SEQ ID NO: 712.
Embodiment B 26 is the composition of any one of claim 1-19, or 24, comprising a nucleic acid encoding SluCas9, wherein the SluCas9 is a variant of the amino acid sequence of SEQ ID NO: 712.
Embodiment B 27 is the composition of any one of claim 1-19, or 24, comprising a nucleic acid encoding SluCas9, wherein the SluCas9 comprises an amino acid sequence selected from any one of SEQ ID NOs: 718-720.
Embodiment B 28 is the composition of any one of claims 1-27 and a pharmaceutically acceptable excipient.
Embodiment B 29 is a composition comprising a guide RNA comprising any one of SEQ ID

NOs: 1-35, 1000-1078, or 3000-3069.
Embodiment B 30 is a composition comprising a guide RNA comprising any one of SEQ ID

NOs: 100-225, 2000-2116, or 4000-4251.
Embodiment B 31 is the composition of any one of claims 1-30 for use in treating Duchenne

Muscular Dystrophy (DMD).
Embodiment B 32 is the composition of any one of claims 1-30 for use in making a double strand break in any one or more of exons 43, 44, 45, 50, 51, or 53 of the dystrophin gene.
Embodiment B 33 is a method of treating Duchenne Muscular Dystrophy (DMD), the method comprising delivering to a cell the composition of any one of claims 1-30.
Embodiment B 34 is a method of treating Duchenne Muscular Dystrophy (DMD), the method comprising delivering to a cell a single nucleic acid molecule comprising:
   i) a nucleic acid encoding one or more guide RNAs, wherein the one or more guide RNAs comprise:
      a. a spacer sequence selected from SEQ ID NOs: 1-35, 1000-1078, or 3000-3069;
      b. a spacer sequence comprising at least 17, 18, 19, or 20 contiguous nucleotides of a spacer sequence selected from SEQ ID NOs: 1-35, 1000-1078, or 3000-3069; or
      c. a spacer sequence that is at least 90% identical to any one of SEQ ID NOs: 1-35, 1000-1078, 3000-3069; and
   ii) a nucleic acid encoding a Staphylococcus aureus Cas9 (SaCas9).
Embodiment B 35 is a method of treating Duchenne Muscular Dystrophy (DMD), the method comprising delivering to a cell a single nucleic acid molecule comprising:
   i) a nucleic acid molecule encoding one or more guide RNAs, wherein the one or more guide RNAs comprise:
      a. a spacer sequence selected from SEQ ID NOs: 100-225, 2000-2116, or 4000-4251;
      b. a spacer sequence comprising at least 17, 18, 19, or 20 contiguous nucleotides of a spacer sequence selected from SEQ ID NOs: 100-225, 2000-2116, or 4000-4251; or
      c. a spacer sequence that is at least 90% identical to any one of SEQ ID NOs: 100-225, 2000-2116, or 4000-4251; and
   ii) a nucleic acid molecule encoding Staphylococcus lugdunensis (SluCas9).
Embodiment B 36 is a method of treating Duchenne Muscular Dystrophy (DMD), the method comprising delivering to a cell a single nucleic acid molecule comprising:
   i) a nucleic acid encoding a pair of guide RNAs comprising:
      m. a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018;
      n. a first and second spacer sequence comprising at least 17, 18, 19, 20, or 21 contiguous nucleotides of any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; or
      o. a first and second spacer sequence that is at least 90% identical to any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and
   ii) a nucleic acid encoding a Staphylococcus aureus Cas9 (SaCas9).
Embodiment B 37 is a method of treating Duchenne Muscular Dystrophy (DMD), the method comprising delivering to a cell a single nucleic acid molecule comprising:
   i) a nucleic acid encoding a pair of guide RNAs comprising:
      p. a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; and 146 and 148;
      q. a first and second spacer sequence comprising at least 17, 18, 19, 20, or 21 contiguous nucleotides of any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; or
      r. a first and second spacer sequence that is at least 90% identical to any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and
   ii) a nucleic acid encoding a Staphylococcus lugdunensis (SluCas9).
Embodiment B 38 is the method of any one of claims 34-37, wherein the single nucleic acid molecule is delivered to the cell on a single vector.
Embodiment B 39 is the method of any one of claims 34-38, comprising a nucleic acid molecule encoding SaCas9, wherein the spacer sequence is selected from any one of SEQ ID NOs: 12, 15, 16, 20, 27, 28, 32, 33, and 35.
Embodiment B 40 is the method of any one of claims 34-39, comprising a nucleic acid molecule encoding SaCas9, wherein the SaCas9 comprises the amino acid sequence of SEQ ID NO: 711.
Embodiment B 41 is the method of any one of claims 34-39, comprising a nucleic acid molecule encoding SaCas9, wherein the SaCas9 is a variant of the amino acid sequence of SEQ ID NO: 711.
Embodiment B 42 is the method of any one of claims 34-39, comprising a nucleic acid molecule encoding SaCas9, wherein the SaCas9 comprises an amino acid sequence selected from any one of SEQ ID NOs: 715-717.
Embodiment B 43 is the method of any one of claims 34-38, comprising a nucleic acid molecule encoding SluCas9, wherein the spacer sequence is selected from any one of SEQ ID NOs: 131, 134, 135, 136, 139, 144, 148, 149, 150, 151, 179, 184, 201, 210, 223, 224, 225.
Embodiment B 44 is the method of any one of claim 34-38, or 43, comprising a nucleic acid molecule encoding SluCas9, wherein the SluCas9 comprises the amino acid sequence of SEQ ID NO: 712.
Embodiment B 45 is the method of any one of claim 34-38, or 43, comprising a nucleic acid molecule encoding SluCas9, wherein the SluCas9 is a variant of the amino acid sequence of SEQ ID NO: 712.
Embodiment B 46 is the method of any one of claim 34-38, or 43, comprising a nucleic acid molecule encoding SluCas9, wherein the SluCas9 comprises an amino acid sequence selected from any one of SEQ ID NOs: 718-720.
Embodiment B 47 is a method of excising a portion of an exon with a premature stop codon in a subject with Duchenne Muscular Dystrophy (DMD), the method comprising delivering to a cell a single nucleic acid molecule comprising:
   i) a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and
   ii) a nucleic acid encoding a Staphylococcus aureus Cas9 (SaCas9);
      wherein the pair of guide RNAs and SaCas9 excise a portion of the exon.
Embodiment B 48 is a method of excising a portion of an exon with a premature stop codon in a subject with Duchenne Muscular Dystrophy (DMD), the method comprising delivering to a cell a single nucleic acid molecule comprising:
   i) a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and
   ii) a nucleic acid encoding a Staphylococcus lugdunensis (SluCas9);
      wherein the pair of guide RNAs and SaCas9 excise a portion of the exon.
Embodiment B 49 is the method of any one of claims 47-48, wherein the single nucleic acid molecule is delivered to the cell on a single vector.
Embodiment B 50 is the method of any one of claims 47-49, wherein the portions of the exon remaining after excision are rejoined with a one nucleotide insertion.
Embodiment B 51 is the method of any one of claims 47-49, wherein the portions of the exon remaining after excision are rejoined without a nucleotide insertion.
Embodiment B 52 is the method of claim 51, wherein the size of excised portion of the exon is between 8 and 167 nucleotides.
Embodiment B 53 is the method of any one of claims 47-52, wherein the exon is exon 45.
Embodiment B 54 is the method of any one of claims 47, and 49-53, wherein the pair of guide RNA comprise a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018.
Embodiment B 55 is the method of any one of claims 48-53, wherein the pair of guide RNA comprise a first and second spacer sequence selected from any one of SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146.
Embodiment B 56 is the method of any one of claims 34, 36, 38-39, 47, and 49-54, wherein the SaCas9 comprises the amino acid sequence of SEQ ID NO: 715.
Embodiment B 57 is the composition or method of any one of the preceding claims, wherein the single nucleic acid molecule is an AAV vector, wherein the vector comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding a SaCas9, a polyadenylation sequence, a promoter for expression of a second sgRNA in the same direction as the promoter for SaCas9, a second sgRNA guide sequence, and a second sgRNA scaffold sequence.
Embodiment B 58 is the composition or method of claim 57, wherein the promoter for expression of the nucleic acid encoding the first sgRNA guide sequence is an hU6 promoter.
Embodiment B 59 is the composition or method of any one of claims 57-58, wherein the promoter for expression of the nucleic acid encoding the second sgRNA guide sequence is an hU6 promoter.
Embodiment B 60 is the composition or method of claim 57, wherein the promoter for expression of the nucleic acid encoding the first sgRNA guide sequence is an 7SK promoter.
Embodiment B 61 is the composition or method of any one of claim 57-58, or 60, wherein the promoter for expression of the nucleic acid encoding the second sgRNA guide sequence is an 7SK promoter.
Embodiment B 62 is the composition or method of any one of claim 57-58, or 60, wherein the promoter for expression of the nucleic acid encoding the second sgRNA guide sequence is an H1m promoter.
Embodiment B 63 is the composition or method of any one of the preceding claims wherein the nucleic acid sequence encoding SaCas9 or SluCas9 is fused to a nucleic acid sequence encoding one or more nuclear localization sequences (NLSs).
Embodiment B 64 is the composition or method of any one of the preceding claims wherein the nucleic acid sequence encoding SaCas9 or SluCas9 is fused to a nucleic acid sequence encoding a nuclear localization sequence (NLS).
Embodiment B 65 is the composition or method of any one of the preceding claims wherein the nucleic acid sequence encoding SaCas9 or SluCas9 is fused to two nucleic acid sequences each encoding a nuclear localization sequence (NLS).
Embodiment B 66 is the composition or method of any one of the preceding claims wherein the nucleic acid sequence encoding SaCas9 or SluCas9 is fused to three nucleic acid sequences each encoding a nuclear localization sequence (NLS).
Embodiment B 67 is the composition or method of any one of claims 64-66, wherein the one or more NLSs is an SV40 NLS.
Embodiment B 68 is the composition or method of any one of claims 64-66, wherein the one or more NLSs is an c-Myc NLS.

### EXAMPLES

The following examples are provided to illustrate certain disclosed embodiments and are not to be construed as limiting the scope of this disclosure in any way.

### Example 1: Exemplary DMD sgRNAs

Guide RNA comprising the guide sequences shown in Table 1A and Table 1B below are prepared according to standard methods in a single guide (sgRNA) format. A single AAV vector is prepared that expresses one or more of the guide RNAs and a SaCas9 (for guide sequences having SEQ ID NOs: 1-35, or SEQ ID NOs: 3000-3069) or SluCas9 (for guide sequences having SEQ ID NOs: 100-225, or SEQ ID NOs: 4000-4251). See, Table 1A and Table 1B. The AAV vector is administered to cells in vitro and to mice (e.g., mdx mice) in vivo to assess the ability of the AAV to express the guide RNA and Cas9, edit the targeted exon (see Table 1A and Table 1B), and thereby treat DMD.

In particular, the ability of in vivo single AAV-mediated delivery of gene-editing components to successfully remove the mutant genomic sequence by exon skipping in the cardiac and skeletal muscle cells of mdx mice is tested.

TABLE 1A
Exemplary DMD guide sequences (human-hg38.p12)

| Sequence | | | | | |
|---|---|---|---|---|---|
| ID No. | | | | | |
| of Guide | | | | | |
| Sequence | EXON | CAS9 | strand | Guide sequence | pam |
| 1 | EXON43 | SACAS9 | + | GCAATGCTGCTGTCTTCTTGCT | ATGAAT |
| 2 | EXON43 | SACAS9 | - | AACAAAATGTACAAGGACCGAC | AAGGGT |
| 3 | EXON43 | SACAS9 | - | TGCAAAGTGCAACGCCTGTGGA | AAGGGT |
| 4 | EXON43 | SACAS9 | - | ATAGTCTACAACAAAGCTCAGG | TCGGAT |
| 5 | EXON43 | SACAS9 | - | CTGTTTTAAAATTTTTATATTA | CAGAAT |
| 6 | EXON44 | SACAS9 | + | ATTTAGCATGTTCCCAATTCTC | AGGAAT |
| 7 | EXON44 | SACAS9 | + | AATCGCCTGCAGGTAAAAGCAT | ATGGAT |
| 8 | EXON44 | SACAS9 | - | TCTCAGAAAGACACAAATTCCT | GAGAAT |
| 9 | EXON45 | SACAS9 | + | TCAGGCTTCCCAATTTTTCCTG | TAGAAT |
| 10 | EXON45 | SACAS9 | + | TAGAATACTGGCATCTGTTTTT | GAGGAT |
| 11 | EXON45 | SACAS9 | + | TGGCATCTGTTTTTGAGGATTG | CTGAAT |
| 12 | EXON45 | SACAS9 | + | TTGCCGCTGCCCAATGCCATCC | TGGAGT |
| 13 | EXON45 | SACAS9 | - | GAGGTAGGGCGACAGATCTAAT | AGGAAT |
| 14 | EXON45 | SACAS9 | - | TCTACAGGAAAAATTGGGAAGC | CTGAAT |
| 15 | EXON45 | SACAS9 | - | GCGGCAAACTGTTGTCAGAACA | TTGAAT |
| 16 | EXON45 | SACAS9 | - | TTTTGGTATCTTACAGGAACTC | CAGGAT |
| 17 | EXON50 | SACAS9 | - | ACTATTGGAGCCTGTAAGTATA | CTGGAT |
| 18 | EXON50 | SACAS9 | - | AGGAAGTTAGAAGATCTGAGCT | CTGAGT |
| 19 | EXON51 | SACAS9 | + | TAGTAACCACAGGTTGTGTCAC | CAGAGT |
| 20 | EXON51 | SACAS9 | + | GTTGTGTCACCAGAGTAACAGT | CTGAGT |
| 21 | EXON51 | SACAS9 | + | TCTGAGTAGGAGCTAAAATATT | TTGGGT |
| 22 | EXON51 | SACAS9 | - | GAGGGTGATGGTGGGTGACCTT | GAGGAT |
| 23 | EXON51 | SACAS9 | - | TATAAAATCACAGAGGGTGATG | GTGGGT |
| 24 | EXON51 | SACAS9 | - | TTGATCAAGTTATAAAATCACA | GAGGGT |
| 25 | EXON53 | SACAS9 | + | CCTTGGTTTCTGTGATTTTCTT | TTGGAT |
| 26 | EXON53 | SACAS9 | + | TCCTTAGCTTCCAGCCATTGTG | TTGAAT |
| 27 | EXON53 | SACAS9 | + | CTTGTACTTCATCCCACTGATT | CTGAAT |
| 28 | EXON53 | SACAS9 | + | ACTGATTCTGAATTCTTTCAAC | TAGAAT |
| 29 | EXON53 | SACAS9 | - | AGCCAAGCTTGAGTCATGGAAG | GAGGGT |
| 30 | EXON53 | SACAS9 | - | TTAGGACAGGCCAGAGCCAAGC | TTGAGT |
| 31 | EXON53 | SACAS9 | - | GCAACAGTTGAATGAAATGTTA | AAGGAT |
| 32 | EXON53 | SACAS9 | - | CCTTCAGAACCGGAGGCAACAG | TTGAAT |
| 33 | EXON53 | SACAS9 | - | AGTTGAAAGAATTCAGAATCAG | TGGGAT |
| 34 | EXON53 | SACAS9 | - | TTTTATTCTAGTTGAAAGAATT | CAGAAT |
| 35 | EXON53 | SACAS9 | - | TTTTTCCTTTTATTCTAGTTGA | AAGAAT |
| 100 | EXON43 | SLUCAS9 | + | ATATATGTGTTACCTACCCTTG | TCGG |
| 101 | EXON43 | SLUCAS9 | + | ACATTTTGTTAACTTTTTCCCA | TTGG |
| 102 | EXON43 | SLUCAS9 | + | CTTTTTCCCATTGGAAATCAAG | CTGG |
| 103 | EXON43 | SLUCAS9 | + | TTTTTCCCATTGGAAATCAAGC | TGGG |
| 104 | EXON43 | SLUCAS9 | + | TCCTGTAGCTTCACCCTTTCCA | CAGG |
| 105 | EXON43 | SLUCAS9 | - | AATGTACAAGGACCGACAAGGG | TAGG |
| 106 | EXON43 | SLUCAS9 | - | ACAAAATGTACAAGGACCGACA | AGGG |
| 107 | EXON43 | SLUCAS9 | - | AACAAAATGTACAAGGACCGAC | AAGG |
| 108 | EXON43 | SLUCAS9 | - | GGAAAAAGTTAACAAAATGTAC | AAGG |
| 109 | EXON43 | SLUCAS9 | - | TCTCTCCCAGCTTGATTTCCAA | TGGG |
| 110 | EXON43 | SLUCAS9 | - | CTCTCTCCCAGCTTGATTTCCA | ATGG |
| 111 | EXON43 | SLUCAS9 | - | GCCTGTGGAAAGGGTGAAGCTA | CAGG |
| 112 | EXON43 | SLUCAS9 | - | GCAAAGTGCAACGCCTGTGGAA | AGGG |
| 113 | EXON43 | SLUCAS9 | - | TGCAAAGTGCAACGCCTGTGGA | AAGG |
| 114 | EXON43 | SLUCAS9 | - | AGCATTGCAAAGTGCAACGCCT | GTGG |
| 115 | EXON43 | SLUCAS9 | - | ATAGTCTACAACAAAGCTCAGG | TCGG |
| 116 | EXON43 | SLUCAS9 | - | AAAGATAGTCTACAACAAAGCT | CAGG |
| 117 | EXON44 | SLUCAS9 | + | TATTTAGCATGTTCCCAATTCT | CAGG |
| 118 | EXON44 | SLUCAS9 | + | AACAGATCTGTCAAATCGCCTG | CAGG |
| 119 | EXON44 | SLUCAS9 | + | AATCGCCTGCAGGTAAAAGCAT | ATGG |
| 120 | EXON44 | SLUCAS9 | - | TGCTAAATACAAATGGTATCTT | AAGG |
| 121 | EXON44 | SLUCAS9 | - | ATTGGGAACATGCTAAATACAA | ATGG |
| 122 | EXON44 | SLUCAS9 | - | AAAGACACAAATTCCTGAGAAT | TGGG |
| 123 | EXON44 | SLUCAS9 | - | GAAAGACACAAATTCCTGAGAA | TTGG |
| 124 | EXON44 | SLUCAS9 | - | ATGATATAAAGATATTTAATCA | GTGG |
| 125 | EXON44 | SLUCAS9 | - | TTGACAGATCTGTTGAGAAATG | GCGG |
| 126 | EXON44 | SLUCAS9 | - | GATTTGACAGATCTGTTGAGAA | ATGG |
| 127 | EXON44 | SLUCAS9 | - | TTGATCCATATGCTTTTACCTG | CAGG |
| 128 | EXON45 | SLUCAS9 | + | AGACCTCCTGCCACCGCAGATT | CAGG |
| 129 | EXON45 | SLUCAS9 | + | TCCCAATTTTTCCTGTAGAATA | CTGG |
| 130 | EXON45 | SLUCAS9 | + | TAGAATACTGGCATCTGTTTTT | GAGG |
| 131 | EXON45 | SLUCAS9 | + | TTTGCCGCTGCCCAATGCCATC | CTGG |
| 132 | EXON45 | SLUCAS9 | + | GGAGTTCCTGTAAGATACCAAA | AAGG |
| 133 | EXON45 | SLUCAS9 | - | AGAGGTAGGGCGACAGATCTAA | TAGG |
| 134 | EXON45 | SLUCAS9 | - | CTGTCAGACAGAAAAAAGAGGT | AGGG |
| 135 | EXON45 | SLUCAS9 | - | GCTGTCAGACAGAAAAAAGAGG | TAGG |
| 136 | EXON45 | SLUCAS9 | - | AACAGCTGTCAGACAGAAAAAA | GAGG |
| 137 | EXON45 | SLUCAS9 | - | AAGCCTGAATCTGCGGTGGCAG | GAGG |
| 138 | EXON45 | SLUCAS9 | - | GGGAAGCCTGAATCTGCGGTGG | CAGG |
| 139 | EXON45 | SLUCAS9 | - | AATTGGGAAGCCTGAATCTGCG | GTGG |
| 140 | EXON45 | SLUCAS9 | - | AAAAATTGGGAAGCCTGAATCT | GCGG |
| 141 | EXON45 | SLUCAS9 | - | GCCAGTATTCTACAGGAAAAAT | TGGG |
| 142 | EXON45 | SLUCAS9 | - | TGCCAGTATTCTACAGGAAAAA | TTGG |
| 143 | EXON45 | SLUCAS9 | - | AAAAACAGATGCCAGTATTCTA | CAGG |
| 144 | EXON45 | SLUCAS9 | - | TGTCAGAACATTGAATGCAACT | GGGG |
| 145 | EXON45 | SLUCAS9 | - | TTGTCAGAACATTGAATGCAAC | TGGG |
| 146 | EXON45 | SLUCAS9 | - | GTTGTCAGAACATTGAATGCAA | CTGG |
| 147 | EXON45 | SLUCAS9 | - | AACTCCAGGATGGCATTGGGCA | GCGG |
| 148 | EXON45 | SLUCAS9 | - | TACAGGAACTCCAGGATGGCAT | TGGG |
| 149 | EXON45 | SLUCAS9 | - | TTACAGGAACTCCAGGATGGCA | TTGG |
| 150 | EXON45 | SLUCAS9 | - | GGTATCTTACAGGAACTCCAGG | ATGG |
| 151 | EXON45 | SLUCAS9 | - | TTTTGGTATCTTACAGGAACTC | CAGG |
| 152 | EXON45 | SLUCAS9 | - | GTTTTGCCTTTTTGGTATCTTA | CAGG |
| 153 | EXON45 | SLUCAS9 | - | TCTTTTCTCAAATAAAAAGACA | TGGG |
| 154 | EXON50 | SLUCAS9 | + | AGAATGGGATCCAGTATACTTA | CAGG |
| 155 | EXON50 | SLUCAS9 | + | AGTATACTTACAGGCTCCAATA | GTGG |
| 156 | EXON50 | SLUCAS9 | + | CAGGCTCCAATAGTGGTCAGTC | CAGG |
| 157 | EXON50 | SLUCAS9 | + | CAATAGTGGTCAGTCCAGGAGC | TAGG |
| 158 | EXON50 | SLUCAS9 | + | GTGGTCAGTCCAGGAGCTAGGT | CAGG |
| 159 | EXON50 | SLUCAS9 | + | TTGCCCTCAGCTCTTGAAGTAA | ACGG |
| 160 | EXON50 | SLUCAS9 | - | AGTATACTGGATCCCATTCTCT | TTGG |
| 161 | EXON50 | SLUCAS9 | - | ACTATTGGAGCCTGTAAGTATA | CTGG |
| 162 | EXON50 | SLUCAS9 | - | CTAGCTCCTGGACTGACCACTA | TTGG |
| 163 | EXON50 | SLUCAS9 | - | GCAAAGCAGCCTGACCTAGCTC | CTGG |
| 164 | EXON50 | SLUCAS9 | - | AAACCGTTTACTTCAAGAGCTG | AGGG |
| 165 | EXON50 | SLUCAS9 | - | TAAACCGTTTACTTCAAGAGCT | GAGG |
| 166 | EXON50 | SLUCAS9 | - | AGATCTGAGCTCTGAGTGGAAG | GCGG |
| 167 | EXON50 | SLUCAS9 | - | AGAAGATCTGAGCTCTGAGTGG | AAGG |
| 168 | EXON50 | SLUCAS9 | - | AGTTAGAAGATCTGAGCTCTGA | GTGG |
| 169 | EXON50 | SLUCAS9 | - | ATGTGTATGCTTTTCTGTTAAA | GAGG |
| 170 | EXON51 | SLUCAS9 | + | TGATCATCTCGTTGATATCCTC | AAGG |
| 171 | EXON51 | SLUCAS9 | + | TTGATCAAGCAGAGAAAGCCAG | TCGG |
| 172 | EXON51 | SLUCAS9 | + | AGTCGGTAAGTTCTGTCCAAGC | CCGG |
| 173 | EXON51 | SLUCAS9 | + | GCCCGGTTGAAATCTGCCAGAG | CAGG |
| 174 | EXON51 | SLUCAS9 | + | CAGAGCAGGTACCTCCAACATC | AAGG |
| 175 | EXON51 | SLUCAS9 | + | GGTACCTCCAACATCAAGGAAG | ATGG |
| 176 | EXON51 | SLUCAS9 | + | CAAGGAAGATGGCATTTCTAGT | TTGG |
| 177 | EXON51 | SLUCAS9 | + | AGATGGCATTTCTAGTTTGGAG | ATGG |
| 178 | EXON51 | SLUCAS9 | + | ATGGCAGTTTCCTTAGTAACCA | CAGG |
| 179 | EXON51 | SLUCAS9 | + | GTCACCAGAGTAACAGTCTGAG | TAGG |
| 180 | EXON51 | SLUCAS9 | + | TCTGAGTAGGAGCTAAAATATT | TTGG |
| 181 | EXON51 | SLUCAS9 | + | CTGAGTAGGAGCTAAAATATTT | TGGG |
| 182 | EXON51 | SLUCAS9 | + | AAATATTTTGGGTTTTTGCAAA | AAGG |
| 183 | EXON51 | SLUCAS9 | - | GTATGAGAAAAAATGATAAAAG | TTGG |
| 184 | EXON51 | SLUCAS9 | - | CAACGAGATGATCATCAAGCAG | AAGG |
| 185 | EXON51 | SLUCAS9 | - | GAGGGTGATGGTGGGTGACCTT | GAGG |
| 186 | EXON51 | SLUCAS9 | - | ATAAAATCACAGAGGGTGATGG | TGGG |
| 187 | EXON51 | SLUCAS9 | - | TATAAAATCACAGAGGGTGATG | GTGG |
| 188 | EXON51 | SLUCAS9 | - | AGTTATAAAATCACAGAGGGTG | ATGG |
| 189 | EXON51 | SLUCAS9 | - | TGATCAAGTTATAAAATCACAG | AGGG |
| 190 | EXON51 | SLUCAS9 | - | TTGATCAAGTTATAAAATCACA | GAGG |
| 191 | EXON51 | SLUCAS9 | - | GGGCTTGGACAGAACTTACCGA | CTGG |
| 192 | EXON51 | SLUCAS9 | - | CTCTGGCAGATTTCAACCGGGC | TTGG |
| 193 | EXON51 | SLUCAS9 | - | ACCTGCTCTGGCAGATTTCAAC | CGGG |
| 194 | EXON51 | SLUCAS9 | - | TACCTGCTCTGGCAGATTTCAA | CCGG |
| 195 | EXON51 | SLUCAS9 | - | CTTGATGTTGGAGGTACCTGCT | CTGG |
| 196 | EXON51 | SLUCAS9 | - | AATGCCATCTTCCTTGATGTTG | GAGG |
| 197 | EXON51 | SLUCAS9 | - | AGAAATGCCATCTTCCTTGATG | TTGG |
| 198 | EXON51 | SLUCAS9 | - | GGTGACACAACCTGTGGTTACT | AAGG |
| 199 | EXON51 | SLUCAS9 | - | TGTTACTCTGGTGACACAACCT | GTGG |
| 200 | EXON51 | SLUCAS9 | - | AGCTCCTACTCAGACTGTTACT | CTGG |
| 201 | EXON53 | SLUCAS9 | + | AAAGGTATCTTTGATACTAACC | TTGG |
| 202 | EXON53 | SLUCAS9 | + | CCTTGGTTTCTGTGATTTTCTT | TTGG |
| 203 | EXON53 | SLUCAS9 | + | CTTTTGGATTGCATCTACTGTA | TAGG |
| 204 | EXON53 | SLUCAS9 | + | TTTTGGATTGCATCTACTGTAT | AGGG |
| 205 | EXON53 | SLUCAS9 | + | ACCCTCCTTCCATGACTCAAGC | TTGG |
| 206 | EXON53 | SLUCAS9 | + | CTTCCATGACTCAAGCTTGGCT | CTGG |
| 207 | EXON53 | SLUCAS9 | + | ACATTTCATTCAACTGTTGCCT | CCGG |
| 208 | EXON53 | SLUCAS9 | + | TCAACTGTTGCCTCCGGTTCTG | AAGG |
| 209 | EXON53 | SLUCAS9 | + | TGAATTCTTTCAACTAGAATAA | AAGG |
| 210 | EXON53 | SLUCAS9 | - | CCAAAAGAAAATCACAGAAACC | AAGG |
| 211 | EXON53 | SLUCAS9 | - | GCCAAGCTTGAGTCATGGAAGG | AGGG |
| 212 | EXON53 | SLUCAS9 | - | AGCCAAGCTTGAGTCATGGAAG | GAGG |
| 213 | EXON53 | SLUCAS9 | - | CAGAGCCAAGCTTGAGTCATGG | AAGG |
| 214 | EXON53 | SLUCAS9 | - | AGGCCAGAGCCAAGCTTGAGTC | ATGG |
| 215 | EXON53 | SLUCAS9 | - | AGAAGCTGAGCAGGTCTTAGGA | CAGG |
| 216 | EXON53 | SLUCAS9 | - | AAGGAAGAAGCTGAGCAGGTCT | TAGG |
| 217 | EXON53 | SLUCAS9 | - | GGAAGCTAAGGAAGAAGCTGAG | CAGG |
| 218 | EXON53 | SLUCAS9 | - | TTCAACACAATGGCTGGAAGCT | AAGG |
| 219 | EXON53 | SLUCAS9 | - | GTTAAAGGATTCAACACAATGG | CTGG |
| 220 | EXON53 | SLUCAS9 | - | AAATGTTAAAGGATTCAACACA | ATGG |
| 221 | EXON53 | SLUCAS9 | - | GCAACAGTTGAATGAAATGTTA | AAGG |
| 222 | EXON53 | SLUCAS9 | - | TACAAGAACACCTTCAGAACCG | GAGG |
| 223 | EXON53 | SLUCAS9 | - | AAGTACAAGAACACCTTCAGAA | CCGG |
| 224 | EXON53 | SLUCAS9 | - | AGTTGAAAGAATTCAGAATCAG | TGGG |
| 225 | EXON53 | SLUCAS9 | - | TAGTTGAAAGAATTCAGAATCA | GTGG |

TABLE 1B
Exemplary DMD guide sequences
(20-nucleotides and 21-nucleotides)

| Se- | | | | |
|---|---|---|---|---|
| quence | | | | |
| ID No. | | | | |
| of | | | | |
| Guide | | | | |
| Se- | | | | |
| quence | EXON | CAS9 | Strand | Guide sequence |
| 3000 | EXON43 | SACAS9 | + | AATGCTGCTGTCTTCTTGCT |
| 3001 | EXON43 | SACAS9 | + | CAATGCTGCTGTCTTCTTGCT |
| 1 | EXON43 | SACAS9 | + | GCAATGCTGCTGTCTTCTTGCT |
| 3002 | EXON43 | SACAS9 | − | AACAAAATGTACAAGGACCG |
| 3003 | EXON43 | SACAS9 | − | AACAAAATGTACAAGGACCGA |
| 2 | EXON43 | SACAS9 | − | AACAAAATGTACAAGGACCGAC |
| 3004 | EXON43 | SACAS9 | − | TGCAAAGTGCAACGCCTGTG |
| 3005 | EXON43 | SACAS9 | − | TGCAAAGTGCAACGCCTGTGG |
| 3 | EXON43 | SACAS9 | − | TGCAAAGTGCAACGCCTGTGGA |
| 3006 | EXON43 | SACAS9 | − | ATAGTCTACAACAAAGCTCA |
| 3007 | EXON43 | SACAS9 | − | ATAGTCTACAACAAAGCTCAG |
| 4 | EXON43 | SACAS9 | − | ATAGTCTACAACAAAGCTCAGG |
| 3008 | EXON43 | SACAS9 | − | CTGTTTTAAAATTTTTATAT |
| 3009 | EXON43 | SACAS9 | − | CTGTTTTAAAATTTTTATATT |
| 5 | EXON43 | SACAS9 | − | CTGTTTTAAAATTTTTATATTA |
| 3010 | EXON44 | SACAS9 | + | TTAGCATGTTCCCAATTCTC |
| 3011 | EXON44 | SACAS9 | + | TTTAGCATGTTCCCAATTCTC |
| 6 | EXON44 | SACAS9 | + | ATTTAGCATGTTCCCAATTCTC |
| 3012 | EXON44 | SACAS9 | + | TCGCCTGCAGGTAAAAGCAT |
| 3013 | EXON44 | SACAS9 | + | ATCGCCTGCAGGTAAAAGCAT |
| 7 | EXON44 | SACAS9 | + | AATCGCCTGCAGGTAAAAGCAT |
| 3014 | EXON44 | SACAS9 | − | TCTCAGAAAGACACAAATTC |
| 3015 | EXON44 | SACAS9 | − | TCTCAGAAAGACACAAATTCC |
| 8 | EXON44 | SACAS9 | − | TCTCAGAAAGACACAAATTCCT |
| 3016 | EXON45 | SACAS9 | + | AGGCTTCCCAATTTTTCCTG |
| 3017 | EXON45 | SACAS9 | + | CAGGCTTCCCAATTTTTCCTG |
| 9 | EXON45 | SACAS9 | + | TCAGGCTTCCCAATTTTTCCTG |
| 3018 | EXON45 | SACAS9 | + | GAATACTGGCATCTGTTTTT |
| 3019 | EXON45 | SACAS9 | + | AGAATACTGGCATCTGTTTTT |
| 10 | EXON45 | SACAS9 | + | TAGAATACTGGCATCTGTTTTT |
| 3020 | EXON45 | SACAS9 | + | GCATCTGTTTTTGAGGATTG |
| 3021 | EXON45 | SACAS9 | + | GGCATCTGTTTTTGAGGATTG |
| 11 | EXON45 | SACAS9 | + | TGGCATCTGTTTTTGAGGATTG |
| 3022 | EXON45 | SACAS9 | + | GCCGCTGCCCAATGCCATCC |
| 3023 | EXON45 | SACAS9 | + | TGCCGCTGCCCAATGCCATCC |
| 12 | EXON45 | SACAS9 | + | TTGCCGCTGCCCAATGCCATCC |
| 3024 | EXON45 | SACAS9 | − | GAGGTAGGGCGACAGATCTA |
| 3025 | EXON45 | SACAS9 | − | GAGGTAGGGCGACAGATCTAA |
| 13 | EXON45 | SACAS9 | − | GAGGTAGGGCGACAGATCTAAT |
| 3026 | EXON45 | SACAS9 | − | TCTACAGGAAAAATTGGGAA |
| 3027 | EXON45 | SACAS9 | − | TCTACAGGAAAAATTGGGAAG |
| 14 | EXON45 | SACAS9 | − | TCTACAGGAAAAATTGGGAAGC |
| 3028 | EXON45 | SACAS9 | − | GCGGCAAACTGTTGTCAGAA |
| 3029 | EXON45 | SACAS9 | − | GCGGCAAACTGTTGTCAGAAC |
| 15 | EXON45 | SACAS9 | − | GCGGCAAACTGTTGTCAGAACA |
| 3030 | EXON45 | SACAS9 | − | TTTTGGTATCTTACAGGAAC |
| 3031 | EXON45 | SACAS9 | − | TTTTGGTATCTTACAGGAACT |
| 16 | EXON45 | SACAS9 | − | TTTTGGTATCTTACAGGAACTC |
| 3032 | EXON50 | SACAS9 | − | ACTATTGGAGCCTGTAAGTA |
| 3033 | EXON50 | SACAS9 | − | ACTATTGGAGCCTGTAAGTAT |
| 17 | EXON50 | SACAS9 | − | ACTATTGGAGCCTGTAAGTATA |
| 3034 | EXON50 | SACAS9 | − | AGGAAGTTAGAAGATCTGAG |
| 3035 | EXON50 | SACAS9 | − | AGGAAGTTAGAAGATCTGAGC |
| 18 | EXON50 | SACAS9 | − | AGGAAGTTAGAAGATCTGAGCT |
| 3036 | EXON51 | SACAS9 | + | GTAACCACAGGTTGTGTCAC |
| 3037 | EXON51 | SACAS9 | + | AGTAACCACAGGTTGTGTCAC |
| 19 | EXON51 | SACAS9 | + | TAGTAACCACAGGTTGTGTCAC |
| 3038 | EXON51 | SACAS9 | + | TGTGTCACCAGAGTAACAGT |
| 3039 | EXON51 | SACAS9 | + | TTGTGTCACCAGAGTAACAGT |
| 20 | EXON51 | SACAS9 | + | GTTGTGTCACCAGAGTAACAGT |
| 3040 | EXON51 | SACAS9 | + | TGAGTAGGAGCTAAAATATT |
| 3041 | EXON51 | SACAS9 | + | CTGAGTAGGAGCTAAAATATT |
| 21 | EXON51 | SACAS9 | + | TCTGAGTAGGAGCTAAAATATT |
| 3042 | EXON51 | SACAS9 | − | GAGGGTGATGGTGGGTGACC |
| 3043 | EXON51 | SACAS9 | − | GAGGGTGATGGTGGGTGACCT |
| 22 | EXON51 | SACAS9 | − | GAGGGTGATGGTGGGTGACCTT |
| 3044 | EXON51 | SACAS9 | − | TATAAAATCACAGAGGGTGA |
| 3045 | EXON51 | SACAS9 | − | TATAAAATCACAGAGGGTGAT |
| 23 | EXON51 | SACAS9 | − | TATAAAATCACAGAGGGTGATG |
| 3046 | EXON51 | SACAS9 | − | TTGATCAAGTTATAAAATCA |
| 3047 | EXON51 | SACAS9 | − | TTGATCAAGTTATAAAATCAC |
| 24 | EXON51 | SACAS9 | − | TTGATCAAGTTATAAAATCACA |
| 3048 | EXON53 | SACAS9 | + | TTGGTTTCTGTGATTTTCTT |
| 3049 | EXON53 | SACAS9 | + | CTTGGTTTCTGTGATTTTCTT |
| 25 | EXON53 | SACAS9 | + | CCTTGGTTTCTGTGATTTTCTT |
| 3050 | EXON53 | SACAS9 | + | CTTAGCTTCCAGCCATTGTG |
| 3051 | EXON53 | SACAS9 | + | CCTTAGCTTCCAGCCATTGTG |
| 26 | EXON53 | SACAS9 | + | TCCTTAGCTTCCAGCCATTGTG |
| 3052 | EXON53 | SACAS9 | + | TGTACTTCATCCCACTGATT |
| 3053 | EXON53 | SACAS9 | + | TTGTACTTCATCCCACTGATT |
| 27 | EXON53 | SACAS9 | + | CTTGTACTTCATCCCACTGATT |
| 3054 | EXON53 | SACAS9 | + | TGATTCTGAATTCTTTCAAC |
| 3055 | EXON53 | SACAS9 | + | CTGATTCTGAATTCTTTCAAC |
| 28 | EXON53 | SACAS9 | + | ACTGATTCTGAATTCTTTCAAC |
| 3056 | EXON53 | SACAS9 | − | AGCCAAGCTTGAGTCATGGA |
| 3057 | EXON53 | SACAS9 | − | AGCCAAGCTTGAGTCATGGAA |
| 29 | EXON53 | SACAS9 | − | AGCCAAGCTTGAGTCATGGAAG |
| 3058 | EXON53 | SACAS9 | − | TTAGGACAGGCCAGAGCCAA |
| 3059 | EXON53 | SACAS9 | − | TTAGGACAGGCCAGAGCCAAG |
| 30 | EXON53 | SACAS9 | − | TTAGGACAGGCCAGAGCCAAGC |
| 3060 | EXON53 | SACAS9 | − | GCAACAGTTGAATGAAATGT |
| 3061 | EXON53 | SACAS9 | − | GCAACAGTTGAATGAAATGTT |
| 31 | EXON53 | SACAS9 | − | GCAACAGTTGAATGAAATGTTA |
| 3062 | EXON53 | SACAS9 | − | CCTTCAGAACCGGAGGCAAC |
| 3063 | EXON53 | SACAS9 | − | CCTTCAGAACCGGAGGCAACA |
| 32 | EXON53 | SACAS9 | − | CCTTCAGAACCGGAGGCAACAG |
| 3064 | EXON53 | SACAS9 | − | AGTTGAAAGAATTCAGAATC |
| 3065 | EXON53 | SACAS9 | − | AGTTGAAAGAATTCAGAATCA |
| 33 | EXON53 | SACAS9 | − | AGTTGAAAGAATTCAGAATCAG |
| 3066 | EXON53 | SACAS9 | − | TTTTATTCTAGTTGAAAGAA |
| 3067 | EXON53 | SACAS9 | − | TTTTATTCTAGTTGAAAGAAT |
| 34 | EXON53 | SACAS9 | − | TTTTATTCTAGTTGAAAGAATT |
| 3068 | EXON53 | SACAS9 | − | TTTTTCCTTTTATTCTAGTT |
| 3069 | EXON53 | SACAS9 | − | TTTTTCCTTTTATTCTAGTTG |
| 35 | EXON53 | SACAS9 | − | TTTTTCCTTTTATTCTAGTTGA |
| 4000 | EXON43 | SLUCAS9 | + | ATATGTGTTACCTACCCTTG |
| 4001 | EXON43 | SLUCAS9 | + | TATATGTGTTACCTACCCTTG |
| 100 | EXON43 | SLUCAS9 | + | ATATATGTGTTACCTACCCTTG |
| 4002 | EXON43 | SLUCAS9 | + | ATTTTGTTAACTTTTTCCCA |
| 4003 | EXON43 | SLUCAS9 | + | CATTTTGTTAACTTTTTCCCA |
| 101 | EXON43 | SLUCAS9 | + | ACATTTTGTTAACTTTTTCCCA |
| 4004 | EXON43 | SLUCAS9 | + | TTTTCCCATTGGAAATCAAG |
| 4005 | EXON43 | SLUCAS9 | + | TTTTTCCCATTGGAAATCAAG |
| 102 | EXON43 | SLUCAS9 | + | CTTTTTCCCATTGGAAATCAAG |
| 4006 | EXON43 | SLUCAS9 | + | TTTCCCATTGGAAATCAAGC |
| 4007 | EXON43 | SLUCAS9 | + | TTTTCCCATTGGAAATCAAGC |
| 103 | EXON43 | SLUCAS9 | + | TTTTTCCCATTGGAAATCAAGC |
| 4008 | EXON43 | SLUCAS9 | + | CTGTAGCTTCACCCTTTCCA |
| 4009 | EXON43 | SLUCAS9 | + | CCTGTAGCTTCACCCTTTCCA |
| 104 | EXON43 | SLUCAS9 | + | TCCTGTAGCTTCACCCTTTCCA |
| 4010 | EXON43 | SLUCAS9 | − | AATGTACAAGGACCGACAAG |
| 4011 | EXON43 | SLUCAS9 | − | AATGTACAAGGACCGACAAGG |
| 105 | EXON43 | SLUCAS9 | − | AATGTACAAGGACCGACAAGGG |
| 4012 | EXON43 | SLUCAS9 | − | ACAAAATGTACAAGGACCGA |
| 4013 | EXON43 | SLUCAS9 | − | ACAAAATGTACAAGGACCGAC |
| 106 | EXON43 | SLUCAS9 | − | ACAAAATGTACAAGGACCGACA |
| 4014 | EXON43 | SLUCAS9 | − | AACAAAATGTACAAGGACCG |
| 4015 | EXON43 | SLUCAS9 | − | AACAAAATGTACAAGGACCGA |
| 107 | EXON43 | SLUCAS9 | − | AACAAAATGTACAAGGACCGAC |
| 4016 | EXON43 | SLUCAS9 | − | GGAAAAAGTTAACAAAATGT |
| 4017 | EXON43 | SLUCAS9 | − | GGAAAAAGTTAACAAAATGTA |
| 108 | EXON43 | SLUCAS9 | − | GGAAAAAGTTAACAAAATGTAC |
| 4018 | EXON43 | SLUCAS9 | − | TCTCTCCCAGCTTGATTTCC |
| 4019 | EXON43 | SLUCAS9 | − | TCTCTCCCAGCTTGATTTCCA |
| 109 | EXON43 | SLUCAS9 | − | TCTCTCCCAGCTTGATTTCCAA |
| 4020 | EXON43 | SLUCAS9 | − | CTCTCTCCCAGCTTGATTTC |
| 4021 | EXON43 | SLUCAS9 | − | CTCTCTCCCAGCTTGATTTCC |
| 110 | EXON43 | SLUCAS9 | − | CTCTCTCCCAGCTTGATTTCCA |
| 4022 | EXON43 | SLUCAS9 | − | GCCTGTGGAAAGGGTGAAGC |
| 4023 | EXON43 | SLUCAS9 | − | GCCTGTGGAAAGGGTGAAGCT |
| 111 | EXON43 | SLUCAS9 | − | GCCTGTGGAAAGGGTGAAGCTA |
| 4024 | EXON43 | SLUCAS9 | − | GCAAAGTGCAACGCCTGTGG |
| 4025 | EXON43 | SLUCAS9 | − | GCAAAGTGCAACGCCTGTGGA |
| 112 | EXON43 | SLUCAS9 | − | GCAAAGTGCAACGCCTGTGGAA |
| 4026 | EXON43 | SLUCAS9 | − | TGCAAAGTGCAACGCCTGTG |
| 4027 | EXON43 | SLUCAS9 | − | TGCAAAGTGCAACGCCTGTGG |
| 113 | EXON43 | SLUCAS9 | − | TGCAAAGTGCAACGCCTGTGGA |
| 4028 | EXON43 | SLUCAS9 | − | AGCATTGCAAAGTGCAACGC |
| 4029 | EXON43 | SLUCAS9 | − | AGCATTGCAAAGTGCAACGCC |
| 114 | EXON43 | SLUCAS9 | − | AGCATTGCAAAGTGCAACGCCT |
| 4030 | EXON43 | SLUCAS9 | − | ATAGTCTACAACAAAGCTCA |
| 4031 | EXON43 | SLUCAS9 | − | ATAGTCTACAACAAAGCTCAG |
| 115 | EXON43 | SLUCAS9 | − | ATAGTCTACAACAAAGCTCAGG |
| 4032 | EXON43 | SLUCAS9 | − | AAAGATAGTCTACAACAAAG |
| 4033 | EXON43 | SLUCAS9 | − | AAAGATAGTCTACAACAAAGC |
| 116 | EXON43 | SLUCAS9 | − | AAAGATAGTCTACAACAAAGCT |
| 4034 | EXON44 | SLUCAS9 | + | TTTAGCATGTTCCCAATTCT |
| 4035 | EXON44 | SLUCAS9 | + | ATTTAGCATGTTCCCAATTCT |
| 117 | EXON44 | SLUCAS9 | + | TATTTAGCATGTTCCCAATTCT |
| 4036 | EXON44 | SLUCAS9 | + | CAGATCTGTCAAATCGCCTG |
| 4037 | EXON44 | SLUCAS9 | + | ACAGATCTGTCAAATCGCCTG |
| 118 | EXON44 | SLUCAS9 | + | AACAGATCTGTCAAATCGCCTG |
| 4038 | EXON44 | SLUCAS9 | + | TCGCCTGCAGGTAAAAGCAT |
| 4039 | EXON44 | SLUCAS9 | + | ATCGCCTGCAGGTAAAAGCAT |
| 119 | EXON44 | SLUCAS9 | + | AATCGCCTGCAGGTAAAAGCAT |
| 4040 | EXON44 | SLUCAS9 | − | TGCTAAATACAAATGGTATC |
| 4041 | EXON44 | SLUCAS9 | − | TGCTAAATACAAATGGTATCT |
| 120 | EXON44 | SLUCAS9 | − | TGCTAAATACAAATGGTATCTT |
| 4042 | EXON44 | SLUCAS9 | − | ATTGGGAACATGCTAAATAC |
| 4043 | EXON44 | SLUCAS9 | − | ATTGGGAACATGCTAAATACA |
| 121 | EXON44 | SLUCAS9 | − | ATTGGGAACATGCTAAATACAA |
| 4044 | EXON44 | SLUCAS9 | − | AAAGACACAAATTCCTGAGA |
| 4045 | EXON44 | SLUCAS9 | − | AAAGACACAAATTCCTGAGAA |
| 122 | EXON44 | SLUCAS9 | − | AAAGACACAAATTCCTGAGAAT |
| 4046 | EXON44 | SLUCAS9 | − | GAAAGACACAAATTCCTGAG |
| 4047 | EXON44 | SLUCAS9 | − | GAAAGACACAAATTCCTGAGA |
| 123 | EXON44 | SLUCAS9 | − | GAAAGACACAAATTCCTGAGAA |
| 4048 | EXON44 | SLUCAS9 | − | ATGATATAAAGATATTTAAT |
| 4049 | EXON44 | SLUCAS9 | − | ATGATATAAAGATATTTAATC |
| 124 | EXON44 | SLUCAS9 | − | ATGATATAAAGATATTTAATCA |
| 4050 | EXON44 | SLUCAS9 | − | TTGACAGATCTGTTGAGAAA |
| 4051 | EXON44 | SLUCAS9 | − | TTGACAGATCTGTTGAGAAAT |
| 125 | EXON44 | SLUCAS9 | − | TTGACAGATCTGTTGAGAAATG |
| 4052 | EXON44 | SLUCAS9 | − | GATTTGACAGATCTGTTGAG |
| 4053 | EXON44 | SLUCAS9 | − | GATTTGACAGATCTGTTGAGA |
| 126 | EXON44 | SLUCAS9 | − | GATTTGACAGATCTGTTGAGAA |
| 4054 | EXON44 | SLUCAS9 | − | TTGATCCATATGCTTTTACC |
| 4055 | EXON44 | SLUCAS9 | − | TTGATCCATATGCTTTTACCT |
| 127 | EXON44 | SLUCAS9 | − | TTGATCCATATGCTTTTACCTG |
| 4056 | EXON45 | SLUCAS9 | + | ACCTCCTGCCACCGCAGATT |
| 4057 | EXON45 | SLUCAS9 | + | GACCTCCTGCCACCGCAGATT |
| 128 | EXON45 | SLUCAS9 | + | AGACCTCCTGCCACCGCAGATT |
| 4058 | EXON45 | SLUCAS9 | + | CCAATTTTTCCTGTAGAATA |
| 4059 | EXON45 | SLUCAS9 | + | CCCAATTTTTCCTGTAGAATA |
| 129 | EXON45 | SLUCAS9 | + | TCCCAATTTTTCCTGTAGAATA |
| 4060 | EXON45 | SLUCAS9 | + | GAATACTGGCATCTGTTTTT |
| 4061 | EXON45 | SLUCAS9 | + | AGAATACTGGCATCTGTTTTT |
| 130 | EXON45 | SLUCAS9 | + | TAGAATACTGGCATCTGTTTTT |
| 4062 | EXON45 | SLUCAS9 | + | TGCCGCTGCCCAATGCCATC |
| 4063 | EXON45 | SLUCAS9 | + | TTGCCGCTGCCCAATGCCATC |
| 131 | EXON45 | SLUCAS9 | + | TTTGCCGCTGCCCAATGCCATC |
| 4064 | EXON45 | SLUCAS9 | + | AGTTCCTGTAAGATACCAAA |
| 4065 | EXON45 | SLUCAS9 | + | GAGTTCCTGTAAGATACCAAA |
| 132 | EXON45 | SLUCAS9 | + | GGAGTTCCTGTAAGATACCAAA |
| 4066 | EXON45 | SLUCAS9 | − | AGAGGTAGGGCGACAGATCT |
| 4067 | EXON45 | SLUCAS9 | − | AGAGGTAGGGCGACAGATCTA |
| 133 | EXON45 | SLUCAS9 | − | AGAGGTAGGGCGACAGATCTAA |
| 4068 | EXON45 | SLUCAS9 | − | CTGTCAGACAGAAAAAAGAG |
| 4069 | EXON45 | SLUCAS9 | − | CTGTCAGACAGAAAAAAGAGG |
| 134 | EXON45 | SLUCAS9 | − | CTGTCAGACAGAAAAAAGAGGT |
| 4070 | EXON45 | SLUCAS9 | − | GCTGTCAGACAGAAAAAAGA |
| 4071 | EXON45 | SLUCAS9 | − | GCTGTCAGACAGAAAAAAGAG |
| 135 | EXON45 | SLUCAS9 | − | GCTGTCAGACAGAAAAAAGAGG |
| 4072 | EXON45 | SLUCAS9 | − | AACAGCTGTCAGACAGAAAA |
| 4073 | EXON45 | SLUCAS9 | − | AACAGCTGTCAGACAGAAAAA |
| 136 | EXON45 | SLUCAS9 | − | AACAGCTGTCAGACAGAAAAAA |
| 4074 | EXON45 | SLUCAS9 | − | AAGCCTGAATCTGCGGTGGC |
| 4075 | EXON45 | SLUCAS9 | − | AAGCCTGAATCTGCGGTGGCA |
| 137 | EXON45 | SLUCAS9 | − | AAGCCTGAATCTGCGGTGGCAG |
| 4076 | EXON45 | SLUCAS9 | − | GGGAAGCCTGAATCTGCGGT |
| 4077 | EXON45 | SLUCAS9 | − | GGGAAGCCTGAATCTGCGGTG |
| 138 | EXON45 | SLUCAS9 | − | GGGAAGCCTGAATCTGCGGTGG |
| 4078 | EXON45 | SLUCAS9 | − | AATTGGGAAGCCTGAATCTG |
| 4079 | EXON45 | SLUCAS9 | − | AATTGGGAAGCCTGAATCTGC |
| 139 | EXON45 | SLUCAS9 | − | AATTGGGAAGCCTGAATCTGCG |
| 4080 | EXON45 | SLUCAS9 | − | AAAAATTGGGAAGCCTGAAT |
| 4081 | EXON45 | SLUCAS9 | − | AAAAATTGGGAAGCCTGAATC |
| 140 | EXON45 | SLUCAS9 | − | AAAAATTGGGAAGCCTGAATCT |
| 4082 | EXON45 | SLUCAS9 | − | GCCAGTATTCTACAGGAAAA |
| 4083 | EXON45 | SLUCAS9 | − | GCCAGTATTCTACAGGAAAAA |
| 141 | EXON45 | SLUCAS9 | − | GCCAGTATTCTACAGGAAAAAT |
| 4084 | EXON45 | SLUCAS9 | − | TGCCAGTATTCTACAGGAAA |
| 4085 | EXON45 | SLUCAS9 | − | TGCCAGTATTCTACAGGAAAA |
| 142 | EXON45 | SLUCAS9 | − | TGCCAGTATTCTACAGGAAAAA |
| 4086 | EXON45 | SLUCAS9 | − | AAAAACAGATGCCAGTATTC |
| 4087 | EXON45 | SLUCAS9 | − | AAAAACAGATGCCAGTATTCT |
| 143 | EXON45 | SLUCAS9 | − | AAAAACAGATGCCAGTATTCTA |
| 4088 | EXON45 | SLUCAS9 | − | TGTCAGAACATTGAATGCAA |
| 4089 | EXON45 | SLUCAS9 | − | TGTCAGAACATTGAATGCAAC |
| 144 | EXON45 | SLUCAS9 | − | TGTCAGAACATTGAATGCAACT |
| 4090 | EXON45 | SLUCAS9 | − | TTGTCAGAACATTGAATGCA |
| 4091 | EXON45 | SLUCAS9 | − | TTGTCAGAACATTGAATGCAA |
| 145 | EXON45 | SLUCAS9 | − | TTGTCAGAACATTGAATGCAAC |
| 4092 | EXON45 | SLUCAS9 | − | GTTGTCAGAACATTGAATGC |
| 4093 | EXON45 | SLUCAS9 | − | GTTGTCAGAACATTGAATGCA |
| 146 | EXON45 | SLUCAS9 | − | GTTGTCAGAACATTGAATGCAA |
| 4094 | EXON45 | SLUCAS9 | − | AACTCCAGGATGGCATTGGG |
| 4095 | EXON45 | SLUCAS9 | − | AACTCCAGGATGGCATTGGGC |
| 147 | EXON45 | SLUCAS9 | − | AACTCCAGGATGGCATTGGGCA |
| 4096 | EXON45 | SLUCAS9 | − | TACAGGAACTCCAGGATGGC |
| 4097 | EXON45 | SLUCAS9 | − | TACAGGAACTCCAGGATGGCA |
| 148 | EXON45 | SLUCAS9 | − | TACAGGAACTCCAGGATGGCAT |
| 4098 | EXON45 | SLUCAS9 | − | TTACAGGAACTCCAGGATGG |
| 4099 | EXON45 | SLUCAS9 | − | TTACAGGAACTCCAGGATGGC |
| 149 | EXON45 | SLUCAS9 | − | TTACAGGAACTCCAGGATGGCA |
| 4100 | EXON45 | SLUCAS9 | − | GGTATCTTACAGGAACTCCA |
| 4101 | EXON45 | SLUCAS9 | − | GGTATCTTACAGGAACTCCAG |
| 150 | EXON45 | SLUCAS9 | − | GGTATCTTACAGGAACTCCAGG |
| 4102 | EXON45 | SLUCAS9 | − | TTTTGGTATCTTACAGGAAC |
| 4103 | EXON45 | SLUCAS9 | − | TTTTGGTATCTTACAGGAACT |
| 151 | EXON45 | SLUCAS9 | − | TTTTGGTATCTTACAGGAACTC |
| 4104 | EXON45 | SLUCAS9 | − | GTTTTGCCTTTTTGGTATCT |
| 4105 | EXON45 | SLUCAS9 | − | GTTTTGCCTTTTTGGTATCTT |
| 152 | EXON45 | SLUCAS9 | − | GTTTTGCCTTTTTGGTATCTTA |
| 4106 | EXON45 | SLUCAS9 | − | TCTTTTCTCAAATAAAAAGA |
| 4107 | EXON45 | SLUCAS9 | − | TCTTTTCTCAAATAAAAAGAC |
| 153 | EXON45 | SLUCAS9 | − | TCTTTTCTCAAATAAAAAGACA |
| 4108 | EXON50 | SLUCAS9 | + | AATGGGATCCAGTATACTTA |
| 4109 | EXON50 | SLUCAS9 | + | GAATGGGATCCAGTATACTTA |
| 154 | EXON50 | SLUCAS9 | + | AGAATGGGATCCAGTATACTTA |
| 4110 | EXON50 | SLUCAS9 | + | TATACTTACAGGCTCCAATA |
| 4111 | EXON50 | SLUCAS9 | + | GTATACTTACAGGCTCCAATA |
| 155 | EXON50 | SLUCAS9 | + | AGTATACTTACAGGCTCCAATA |
| 4112 | EXON50 | SLUCAS9 | + | GGCTCCAATAGTGGTCAGTC |
| 4113 | EXON50 | SLUCAS9 | + | AGGCTCCAATAGTGGTCAGTC |
| 156 | EXON50 | SLUCAS9 | + | CAGGCTCCAATAGTGGTCAGTC |
| 4114 | EXON50 | SLUCAS9 | + | ATAGTGGTCAGTCCAGGAGC |
| 4115 | EXON50 | SLUCAS9 | + | AATAGTGGTCAGTCCAGGAGC |
| 157 | EXON50 | SLUCAS9 | + | CAATAGTGGTCAGTCCAGGAGC |
| 4116 | EXON50 | SLUCAS9 | + | GGTCAGTCCAGGAGCTAGGT |
| 4117 | EXON50 | SLUCAS9 | + | TGGTCAGTCCAGGAGCTAGGT |
| 158 | EXON50 | SLUCAS9 | + | GTGGTCAGTCCAGGAGCTAGGT |
| 4118 | EXON50 | SLUCAS9 | + | GCCCTCAGCTCTTGAAGTAA |
| 4119 | EXON50 | SLUCAS9 | + | TGCCCTCAGCTCTTGAAGTAA |
| 159 | EXON50 | SLUCAS9 | + | TTGCCCTCAGCTCTTGAAGTAA |
| 4120 | EXON50 | SLUCAS9 | − | AGTATACTGGATCCCATTCT |
| 4121 | EXON50 | SLUCAS9 | − | AGTATACTGGATCCCATTCTC |
| 160 | EXON50 | SLUCAS9 | − | AGTATACTGGATCCCATTCTCT |
| 4122 | EXON50 | SLUCAS9 | − | ACTATTGGAGCCTGTAAGTA |
| 4123 | EXON50 | SLUCAS9 | − | ACTATTGGAGCCTGTAAGTAT |
| 161 | EXON50 | SLUCAS9 | − | ACTATTGGAGCCTGTAAGTATA |
| 4124 | EXON50 | SLUCAS9 | − | CTAGCTCCTGGACTGACCAC |
| 4125 | EXON50 | SLUCAS9 | − | CTAGCTCCTGGACTGACCACT |
| 162 | EXON50 | SLUCAS9 | − | CTAGCTCCTGGACTGACCACTA |
| 4126 | EXON50 | SLUCAS9 | − | GCAAAGCAGCCTGACCTAGC |
| 4127 | EXON50 | SLUCAS9 | − | GCAAAGCAGCCTGACCTAGCT |
| 163 | EXON50 | SLUCAS9 | − | GCAAAGCAGCCTGACCTAGCTC |
| 4128 | EXON50 | SLUCAS9 | − | AAACCGTTTACTTCAAGAGC |
| 4129 | EXON50 | SLUCAS9 | − | AAACCGTTTACTTCAAGAGCT |
| 164 | EXON50 | SLUCAS9 | − | AAACCGTTTACTTCAAGAGCTG |
| 4130 | EXON50 | SLUCAS9 | − | TAAACCGTTTACTTCAAGAG |
| 4131 | EXON50 | SLUCAS9 | − | TAAACCGTTTACTTCAAGAGC |
| 165 | EXON50 | SLUCAS9 | − | TAAACCGTTTACTTCAAGAGCT |
| 4132 | EXON50 | SLUCAS9 | − | AGATCTGAGCTCTGAGTGGA |
| 4133 | EXON50 | SLUCAS9 | − | AGATCTGAGCTCTGAGTGGAA |
| 166 | EXON50 | SLUCAS9 | − | AGATCTGAGCTCTGAGTGGAAG |
| 4134 | EXON50 | SLUCAS9 | − | AGAAGATCTGAGCTCTGAGT |
| 4135 | EXON50 | SLUCAS9 | − | AGAAGATCTGAGCTCTGAGTG |
| 167 | EXON50 | SLUCAS9 | − | AGAAGATCTGAGCTCTGAGTGG |
| 4135 | EXON50 | SLUCAS9 | − | AGTTAGAAGATCTGAGCTCT |
| 4137 | EXON50 | SLUCAS9 | − | AGTTAGAAGATCTGAGCTCTG |
| 168 | EXON50 | SLUCAS9 | − | AGTTAGAAGATCTGAGCTCTGA |
| 4138 | EXON50 | SLUCAS9 | − | ATGTGTATGCTTTTCTGTTA |
| 4139 | EXON50 | SLUCAS9 | − | ATGTGTATGCTTTTCTGTTAA |
| 169 | EXON50 | SLUCAS9 | − | ATGTGTATGCTTTTCTGTTAAA |
| 4140 | EXON51 | SLUCAS9 | + | ATCATCTCGTTGATATCCTC |
| 4141 | EXON51 | SLUCAS9 | + | GATCATCTCGTTGATATCCTC |
| 170 | EXON51 | SLUCAS9 | + | TGATCATCTCGTTGATATCCTC |
| 4142 | EXON51 | SLUCAS9 | + | GATCAAGCAGAGAAAGCCAG |
| 4143 | EXON51 | SLUCAS9 | + | TGATCAAGCAGAGAAAGCCAG |
| 171 | EXON51 | SLUCAS9 | + | TTGATCAAGCAGAGAAAGCCAG |
| 4144 | EXON51 | SLUCAS9 | + | TCGGTAAGTTCTGTCCAAGC |
| 4145 | EXON51 | SLUCAS9 | + | GTCGGTAAGTTCTGTCCAAGC |
| 172 | EXON51 | SLUCAS9 | + | AGTCGGTAAGTTCTGTCCAAGC |
| 4146 | EXON51 | SLUCAS9 | + | CCGGTTGAAATCTGCCAGAG |
| 4147 | EXON51 | SLUCAS9 | + | CCCGGTTGAAATCTGCCAGAG |
| 173 | EXON51 | SLUCAS9 | + | GCCCGGTTGAAATCTGCCAGAG |
| 4148 | EXON51 | SLUCAS9 | + | GAGCAGGTACCTCCAACATC |
| 4149 | EXON51 | SLUCAS9 | + | AGAGCAGGTACCTCCAACATC |
| 174 | EXON51 | SLUCAS9 | + | CAGAGCAGGTACCTCCAACATC |
| 4150 | EXON51 | SLUCAS9 | + | TACCTCCAACATCAAGGAAG |
| 4151 | EXON51 | SLUCAS9 | + | GTACCTCCAACATCAAGGAAG |
| 175 | EXON51 | SLUCAS9 | + | GGTACCTCCAACATCAAGGAAG |
| 4152 | EXON51 | SLUCAS9 | + | AGGAAGATGGCATTTCTAGT |
| 4153 | EXON51 | SLUCAS9 | + | AAGGAAGATGGCATTTCTAGT |
| 176 | EXON51 | SLUCAS9 | + | CAAGGAAGATGGCATTTCTAGT |
| 4154 | EXON51 | SLUCAS9 | + | ATGGCATTTCTAGTTTGGAG |
| 4155 | EXON51 | SLUCAS9 | + | GATGGCATTTCTAGTTTGGAG |
| 177 | EXON51 | SLUCAS9 | + | AGATGGCATTTCTAGTTTGGAG |
| 4156 | EXON51 | SLUCAS9 | + | GGCAGTTTCCTTAGTAACCA |
| 4157 | EXON51 | SLUCAS9 | + | TGGCAGTTTCCTTAGTAACCA |
| 178 | EXON51 | SLUCAS9 | + | ATGGCAGTTTCCTTAGTAACCA |
| 4158 | EXON51 | SLUCAS9 | + | CACCAGAGTAACAGTCTGAG |
| 4159 | EXON51 | SLUCAS9 | + | TCACCAGAGTAACAGTCTGAG |
| 179 | EXON51 | SLUCAS9 | + | GTCACCAGAGTAACAGTCTGAG |
| 4160 | EXON51 | SLUCAS9 | + | TGAGTAGGAGCTAAAATATT |
| 4161 | EXON51 | SLUCAS9 | + | CTGAGTAGGAGCTAAAATATT |
| 180 | EXON51 | SLUCAS9 | + | TCTGAGTAGGAGCTAAAATATT |
| 4162 | EXON51 | SLUCAS9 | + | GAGTAGGAGCTAAAATATTT |
| 4163 | EXON51 | SLUCAS9 | + | TGAGTAGGAGCTAAAATATTT |
| 181 | EXON51 | SLUCAS9 | + | CTGAGTAGGAGCTAAAATATTT |
| 4164 | EXON51 | SLUCAS9 | + | ATATTTTGGGTTTTTGCAAA |
| 4165 | EXON51 | SLUCAS9 | + | AATATTTTGGGTTTTTGCAAA |
| 182 | EXON51 | SLUCAS9 | + | AAATATTTTGGGTTTTTGCAAA |
| 4166 | EXON51 | SLUCAS9 | − | GTATGAGAAAAAATGATAAA |
| 4167 | EXON51 | SLUCAS9 | − | GTATGAGAAAAAATGATAAAA |
| 183 | EXON51 | SLUCAS9 | − | GTATGAGAAAAAATGATAAAAG |
| 4168 | EXON51 | SLUCAS9 | − | CAACGAGATGATCATCAAGC |
| 4169 | EXON51 | SLUCAS9 | − | CAACGAGATGATCATCAAGCA |
| 184 | EXON51 | SLUCAS9 | − | CAACGAGATGATCATCAAGCAG |
| 4170 | EXON51 | SLUCAS9 | − | GAGGGTGATGGTGGGTGACC |
| 4171 | EXON51 | SLUCAS9 | − | GAGGGTGATGGTGGGTGACCT |
| 185 | EXON51 | SLUCAS9 | − | GAGGGTGATGGTGGGTGACCTT |
| 4172 | EXON51 | SLUCAS9 | − | ATAAAATCACAGAGGGTGAT |
| 4173 | EXON51 | SLUCAS9 | − | ATAAAATCACAGAGGGTGATG |
| 186 | EXON51 | SLUCAS9 | − | ATAAAATCACAGAGGGTGATGG |
| 4174 | EXON51 | SLUCAS9 | − | TATAAAATCACAGAGGGTGA |
| 4175 | EXON51 | SLUCAS9 | − | TATAAAATCACAGAGGGTGAT |
| 187 | EXON51 | SLUCAS9 | − | TATAAAATCACAGAGGGTGATG |
| 4176 | EXON51 | SLUCAS9 | − | AGTTATAAAATCACAGAGGG |
| 4177 | EXON51 | SLUCAS9 | − | AGTTATAAAATCACAGAGGGT |
| 188 | EXON51 | SLUCAS9 | − | AGTTATAAAATCACAGAGGGTG |
| 4178 | EXON51 | SLUCAS9 | − | TGATCAAGTTATAAAATCAC |
| 4179 | EXON51 | SLUCAS9 | − | TGATCAAGTTATAAAATCACA |
| 189 | EXON51 | SLUCAS9 | − | TGATCAAGTTATAAAATCACAG |
| 4180 | EXON51 | SLUCAS9 | − | TTGATCAAGTTATAAAATCA |
| 4181 | EXON51 | SLUCAS9 | − | TTGATCAAGTTATAAAATCAC |
| 190 | EXON51 | SLUCAS9 | − | TTGATCAAGTTATAAAATCACA |
| 4182 | EXON51 | SLUCAS9 | − | GGGCTTGGACAGAACTTACC |
| 4183 | EXON51 | SLUCAS9 | − | GGGCTTGGACAGAACTTACCG |
| 191 | EXON51 | SLUCAS9 | − | GGGCTTGGACAGAACTTACCGA |
| 4184 | EXON51 | SLUCAS9 | − | CTCTGGCAGATTTCAACCGG |
| 4185 | EXON51 | SLUCAS9 | − | CTCTGGCAGATTTCAACCGGG |
| 192 | EXON51 | SLUCAS9 | − | CTCTGGCAGATTTCAACCGGGC |
| 4186 | EXON51 | SLUCAS9 | − | ACCTGCTCTGGCAGATTTCA |
| 4187 | EXON51 | SLUCAS9 | − | ACCTGCTCTGGCAGATTTCAA |
| 193 | EXON51 | SLUCAS9 | − | ACCTGCTCTGGCAGATTTCAAC |
| 4188 | EXON51 | SLUCAS9 | − | TACCTGCTCTGGCAGATTTC |
| 4189 | EXON51 | SLUCAS9 | − | TACCTGCTCTGGCAGATTTCA |
| 194 | EXON51 | SLUCAS9 | − | TACCTGCTCTGGCAGATTTCAA |
| 4190 | EXON51 | SLUCAS9 | − | CTTGATGTTGGAGGTACCTG |
| 4191 | EXON51 | SLUCAS9 | − | CTTGATGTTGGAGGTACCTGC |
| 195 | EXON51 | SLUCAS9 | − | CTTGATGTTGGAGGTACCTGCT |
| 4192 | EXON51 | SLUCAS9 | − | AATGCCATCTTCCTTGATGT |
| 4193 | EXON51 | SLUCAS9 | − | AATGCCATCTTCCTTGATGTT |
| 196 | EXON51 | SLUCAS9 | − | AATGCCATCTTCCTTGATGTTG |
| 4194 | EXON51 | SLUCAS9 | − | AGAAATGCCATCTTCCTTGA |
| 4195 | EXON51 | SLUCAS9 | − | AGAAATGCCATCTTCCTTGAT |
| 197 | EXON51 | SLUCAS9 | − | AGAAATGCCATCTTCCTTGATG |
| 4196 | EXON51 | SLUCAS9 | − | GGTGACACAACCTGTGGTTA |
| 4197 | EXON51 | SLUCAS9 | − | GGTGACACAACCTGTGGTTAC |
| 198 | EXON51 | SLUCAS9 | − | GGTGACACAACCTGTGGTTACT |
| 4198 | EXON51 | SLUCAS9 | − | TGTTACTCTGGTGACACAAC |
| 4199 | EXON51 | SLUCAS9 | − | TGTTACTCTGGTGACACAACC |
| 199 | EXON51 | SLUCAS9 | − | TGTTACTCTGGTGACACAACCT |
| 4200 | EXON51 | SLUCAS9 | − | AGCTCCTACTCAGACTGTTA |
| 4201 | EXON51 | SLUCAS9 | − | AGCTCCTACTCAGACTGTTAC |
| 200 | EXON51 | SLUCAS9 | − | AGCTCCTACTCAGACTGTTACT |
| 4202 | EXON53 | SLUCAS9 | + | AGGTATCTTTGATACTAACC |
| 4203 | EXON53 | SLUCAS9 | + | AAGGTATCTTTGATACTAACC |
| 201 | EXON53 | SLUCAS9 | + | AAAGGTATCTTTGATACTAACC |
| 4204 | EXON53 | SLUCAS9 | + | TTGGTTTCTGTGATTTTCTT |
| 4205 | EXON53 | SLUCAS9 | + | CTTGGTTTCTGTGATTTTCTT |
| 202 | EXON53 | SLUCAS9 | + | CCTTGGTTTCTGTGATTTTCTT |
| 4206 | EXON53 | SLUCAS9 | + | TTTGGATTGCATCTACTGTA |
| 4207 | EXON53 | SLUCAS9 | + | TTTTGGATTGCATCTACTGTA |
| 203 | EXON53 | SLUCAS9 | + | CTTTTGGATTGCATCTACTGTA |
| 4208 | EXON53 | SLUCAS9 | + | TTGGATTGCATCTACTGTAT |
| 4209 | EXON53 | SLUCAS9 | + | TTTGGATTGCATCTACTGTAT |
| 204 | EXON53 | SLUCAS9 | + | TTTTGGATTGCATCTACTGTAT |
| 4210 | EXON53 | SLUCAS9 | + | CCTCCTTCCATGACTCAAGC |
| 4211 | EXON53 | SLUCAS9 | + | CCCTCCTTCCATGACTCAAGC |
| 205 | EXON53 | SLUCAS9 | + | ACCCTCCTTCCATGACTCAAGC |
| 4212 | EXON53 | SLUCAS9 | + | TCCATGACTCAAGCTTGGCT |
| 4213 | EXON53 | SLUCAS9 | + | TTCCATGACTCAAGCTTGGCT |
| 206 | EXON53 | SLUCAS9 | + | CTTCCATGACTCAAGCTTGGCT |
| 4214 | EXON53 | SLUCAS9 | + | ATTTCATTCAACTGTTGCCT |
| 4215 | EXON53 | SLUCAS9 | + | CATTTCATTCAACTGTTGCCT |
| 207 | EXON53 | SLUCAS9 | + | ACATTTCATTCAACTGTTGCCT |
| 4216 | EXON53 | SLUCAS9 | + | AACTGTTGCCTCCGGTTCTG |
| 4217 | EXON53 | SLUCAS9 | + | CAACTGTTGCCTCCGGTTCTG |
| 208 | EXON53 | SLUCAS9 | + | TCAACTGTTGCCTCCGGTTCTG |
| 4218 | EXON53 | SLUCAS9 | + | AATTCTTTCAACTAGAATAA |
| 4219 | EXON53 | SLUCAS9 | + | GAATTCTTTCAACTAGAATAA |
| 209 | EXON53 | SLUCAS9 | + | TGAATTCTTTCAACTAGAATAA |
| 4220 | EXON53 | SLUCAS9 | − | CCAAAAGAAAATCACAGAAA |
| 4221 | EXON53 | SLUCAS9 | − | CCAAAAGAAAATCACAGAAAC |
| 210 | EXON53 | SLUCAS9 | − | CCAAAAGAAAATCACAGAAACC |
| 4222 | EXON53 | SLUCAS9 | − | GCCAAGCTTGAGTCATGGAA |
| 4223 | EXON53 | SLUCAS9 | − | GCCAAGCTTGAGTCATGGAAG |
| 211 | EXON53 | SLUCAS9 | − | GCCAAGCTTGAGTCATGGAAGG |
| 4224 | EXON53 | SLUCAS9 | − | AGCCAAGCTTGAGTCATGGA |
| 4225 | EXON53 | SLUCAS9 | − | AGCCAAGCTTGAGTCATGGAA |
| 212 | EXON53 | SLUCAS9 | − | AGCCAAGCTTGAGTCATGGAAG |
| 4226 | EXON53 | SLUCAS9 | − | CAGAGCCAAGCTTGAGTCAT |
| 4227 | EXON53 | SLUCAS9 | − | CAGAGCCAAGCTTGAGTCATG |
| 213 | EXON53 | SLUCAS9 | − | CAGAGCCAAGCTTGAGTCATGG |
| 4228 | EXON53 | SLUCAS9 | − | AGGCCAGAGCCAAGCTTGAG |
| 4229 | EXON53 | SLUCAS9 | − | AGGCCAGAGCCAAGCTTGAGT |
| 214 | EXON53 | SLUCAS9 | − | AGGCCAGAGCCAAGCTTGAGTC |
| 4230 | EXON53 | SLUCAS9 | − | AGAAGCTGAGCAGGTCTTAG |
| 4231 | EXON53 | SLUCAS9 | − | AGAAGCTGAGCAGGTCTTAGG |
| 215 | EXON53 | SLUCAS9 | − | AGAAGCTGAGCAGGTCTTAGGA |
| 4232 | EXON53 | SLUCAS9 | − | AAGGAAGAAGCTGAGCAGGT |
| 4233 | EXON53 | SLUCAS9 | − | AAGGAAGAAGCTGAGCAGGTC |
| 216 | EXON53 | SLUCAS9 | − | AAGGAAGAAGCTGAGCAGGTCT |
| 4234 | EXON53 | SLUCAS9 | − | GGAAGCTAAGGAAGAAGCTG |
| 4235 | EXON53 | SLUCAS9 | − | GGAAGCTAAGGAAGAAGCTGA |
| 217 | EXON53 | SLUCAS9 | − | GGAAGCTAAGGAAGAAGCTGAG |
| 4236 | EXON53 | SLUCAS9 | − | TTCAACACAATGGCTGGAAG |
| 4237 | EXON53 | SLUCAS9 | − | TTCAACACAATGGCTGGAAGC |
| 218 | EXON53 | SLUCAS9 | − | TTCAACACAATGGCTGGAAGCT |
| 4238 | EXON53 | SLUCAS9 | − | GTTAAAGGATTCAACACAAT |
| 4239 | EXON53 | SLUCAS9 | − | GTTAAAGGATTCAACACAATG |
| 219 | EXON53 | SLUCAS9 | − | GTTAAAGGATTCAACACAATGG |
| 4240 | EXON53 | SLUCAS9 | − | AAATGTTAAAGGATTCAACA |
| 4241 | EXON53 | SLUCAS9 | − | AAATGTTAAAGGATTCAACAC |
| 220 | EXON53 | SLUCAS9 | − | AAATGTTAAAGGATTCAACACA |
| 4242 | EXON53 | SLUCAS9 | − | GCAACAGTTGAATGAAATGT |
| 4243 | EXON53 | SLUCAS9 | − | GCAACAGTTGAATGAAATGTT |
| 221 | EXON53 | SLUCAS9 | − | GCAACAGTTGAATGAAATGTTA |
| 4244 | EXON53 | SLUCAS9 | − | TACAAGAACACCTTCAGAAC |
| 4245 | EXON53 | SLUCAS9 | − | TACAAGAACACCTTCAGAACC |
| 222 | EXON53 | SLUCAS9 | − | TACAAGAACACCTTCAGAACCG |
| 4246 | EXON53 | SLUCAS9 | − | AAGTACAAGAACACCTTCAG |
| 4247 | EXON53 | SLUCAS9 | − | AAGTACAAGAACACCTTCAGA |
| 223 | EXON53 | SLUCAS9 | − | AAGTACAAGAACACCTTCAGAA |
| 4248 | EXON53 | SLUCAS9 | − | AGTTGAAAGAATTCAGAATC |
| 4249 | EXON53 | SLUCAS9 | − | AGTTGAAAGAATTCAGAATCA |
| 224 | EXON53 | SLUCAS9 | − | AGTTGAAAGAATTCAGAATCAG |
| 4250 | EXON53 | SLUCAS9 | − | TAGTTGAAAGAATTCAGAAT |
| 4251 | EXON53 | SLUCAS9 | − | TAGTTGAAAGAATTCAGAATC |
| 225 | EXON53 | SLUCAS9 | − | TAGTTGAAAGAATTCAGAATCA |

### Example 2: Evaluation of DMD sgRNAs

A. Materials and Methods

1. sgRNA Selection

A subset of sgRNAs targeting the DMD gene were selected for indel frequency and profile evaluation. The selected sgRNAs are shown in Table 2 and were prepared according to standard methods. The criteria used to select these sgRNAs included their potential to induce exon reframing and or skipping, in addition to the existence of a mouse, dog and a non-human primate (NHP) homologue counterpart. This selection included 13 sgRNAs located within exon 45, three sgRNAs located within exon 51 and ten sgRNAs located within exon 53. The number of predicted off target sites was determined for each sgRNA.

TABLE 2
Exemplary sgRNAs

| | | | | | SEQ ID | | | | SEQ ID | | | |
|---|---|---|---|---|---|---|---|---|---|---|---|---|
| | | | | | NO of | Human | | | NO of | Mouse | | |
| | | | | Human | Human | Guide | | Mouse | Mouse | Guide | | |
| | | Guide | | Guide | Guide | Se- | Human | Guide | Guide | Se- | Mouse | Mis- |
| Exon | Cas9 | type | Strand | ID | Sequence | quence | PAM | ID | Sequence | quence | PAM | matches |
| EXON45 | SluCas9 | Reframe | + | E45SL4 | 131 | TTTGCC | CTGG | mE45SL4 | 300 | cTTGaC | CTGG | 2 |
| | | | | | | GCTGCC | | | | GCTGCC | | |
| | | | | | | CAATGC | | | | CAATGC | | |
| | | | | | | CATC | | | | CATC | | |
| EXON45 | SluCas9 | Splice | − | E45SL7 | 134 | CTGTCA | AGGG | mE45SL7 | 301 | CTGgCA | AGGG | 4 |
| | | | | | | GACAGA | | | | GAaAGg | | |
| | | | | | | AAAAAG | | | | AgAAAG | | |
| | | | | | | AGGT | | | | AGGT | | |
| EXON45 | SluCas9 | Splice | − | E45SL8 | 135 | GCTGTC | TAGG | mE45SL8 | 302 | GCTGgC | TAGG | 4 |
| | | | | | | AGACAG | | | | AGAaAG | | |
| | | | | | | AAAAAA | | | | gAgAAA | | |
| | | | | | | GAGG | | | | GAGG | | |
| EXON45 | SluCas9 | Splice | − | E45SL9 | 136 | AACAGC | GAGG | mE45SL9 | 303 | AAgAGC | GAGG | 5 |
| | | | | | | TGTCAG | | | | TGgCAG | | |
| | | | | | | ACAGAA | | | | AaAGgA | | |
| | | | | | | AAAA | | | | gAAA | | |
| EXON45 | SluCas9 | Reframe | − | E45SL12 | 139 | AATTGG | GTGG | mE45SL12 | 304 | AATTaG | GTGG | 3 |
| | | Alt | | | | GAAGCC | | | | GAAGCt | | |
| | | | | | | TGAATC | | | | TGAgTC | | |
| | | | | | | TGCG | | | | TGCG | | |
| EXON45 | SluCas9 | Reframe | − | E45SL17 | 144 | TGTCAG | GGGG | mE45SL17 | 305 | TGTCAG | GGGG | 1 |
| | | Alt | | | | AACATT | | | | AACAcT | | |
| | | | | | | GAATGC | | | | GAATGC | | |
| | | | | | | AACT | | | | AACT | | |
| EXON45 | SluCas9 | Reframe | − | E45SL21 | 148 | TACAGG | TGGG | mE45SL21 | 306 | TACAGG | TGGG | 0 |
| | | | | | | AACTCC | | | | AACTCC | | |
| | | | | | | AGGATG | | | | AGGATG | | |
| | | | | | | GCAT | | | | GCAT | | |
| EXON45 | SluCas9 | Reframe | − | E45SL22 | 149 | TTACAG | TTGG | mE45SL22 | 307 | TTACAG | TTGG | 0 |
| | | | | | | GAACTC | | | | GAACTC | | |
| | | | | | | CAGGAT | | | | CAGGAT | | |
| | | | | | | GGCA | | | | GGCA | | |
| EXON45 | SluCas9 | Reframe; | − | E45SL23 | 150 | GGTATC | ATGG | mE45SL23 | 308 | GaTggC | ATGG | 3 |
| | | Splice | | | | TTACAG | | | | TTACAG | | |
| | | | | | | GAACTC | | | | GAACTC | | |
| | | | | | | CAGG | | | | CAGG | | |
| EXON45 | SluCas9 | Reframe; | − | E45SL24 | 151 | TTTTGG | CAGG | mE45SL24 | 309 | TTcTGa | CAGG | 4 |
| | | Splice | | | | TATCTT | | | | TggCTT | | |
| | | | | | | ACAGGA | | | | ACAGGA | | |
| | | | | | | ACTC | | | | ACTC | | |
| EXON51 | SluCas9 | Reframe | + | E51SL10 | 179 | GTCACC | TAGG | mE51SL10 | 310 | NA | NA | NA |
| | | | | | | AGAGTA | | | | | | |
| | | | | | | ACAGTC | | | | | | |
| | | | | | | TGAG | | | | | | |
| EXON51 | SluCas9 | Splice | − | E515L15 | 184 | CAACGA | AAGG | mE51SL15 | 311 | CAAtGA | AAGG | 3 |
| | | | | | | GATGAT | | | | aATGAT | | |
| | | | | | | CATCAA | | | | CATCAA | | |
| | | | | | | GCAG | | | | aCAG | | |
| EXON53 | SluCas9 | Splice | + | E53SL1 | 201 | AAAGGT | TTGG | mE53SL1 | 312 | AAAGaT | TTGG | 4 |
| | | | | | | ATCTTT | | | | ATgcTT | | |
| | | | | | | GATACT | | | | GAcACT | | |
| | | | | | | AACC | | | | AACC | | |
| EXON53 | SluCas9 | Splice | − | E53SL10 | 210 | CCAAAA | AAGG | mE53SL10 | 313 | CCAAAA | AAGG | 1 |
| | | | | | | GAAAAT | | | | GAAgAT | | |
| | | | | | | CACAGA | | | | CACAGA | | |
| | | | | | | AACC | | | | AACC | | |
| EXON53 | SluCas9 | Reframe | − | E53SL23 | 223 | AAGTAC | CCGG | mE53SL23 | 314 | AgGTtC | CAGG | 4 |
| | | | | | | AAGAAC | | | | AAGAAC | | |
| | | | | | | ACCTTC | | | | AgCTgC | | |
| | | | | | | AGAA | | | | AGAA | | |
| EXON53 | SluCas9 | Reframe | − | E53SL24 | 224 | AGTTGA | TGGG | mE53SL24 | 315 | AGTTGA | TGGG | 1 |
| | | | | | | AAGAAT | | | | AAGAAT | | |
| | | | | | | TCAGAA | | | | TCAGAt | | |
| | | | | | | TCAG | | | | TCAG | | |
| EXON53 | SluCas9 | Reframe | − | E53SL25 | 225 | TAGTTG | GTGG | mE53SL25 | 316 | cAGTTG | GTGG | 2 |
| | | | | | | AAAGAA | | | | AAAGAA | | |
| | | | | | | TTCAGA | | | | TTCAGA | | |
| | | | | | | ATCA | | | | tTCA | | |
| EXON45 | SaCas9 | Reframe | + | E45Sa4 | 12 | TTGCCG | TTG | mE45Sa4 | 317 | TTGaCG | TGG | 1 |
| | | | | | | CTGCCC | GAG | | | CTGCCC | AGT | |
| | | | | | | AATGCC | | | | AATGCC | | |
| | | | | | | ATCC | | | | ATCC | | |
| EXON45 | SaCas9 | Reframe | − | E45Sa7 | 15 | GCGGCA | TTG | mE45Sa7 | 318 | GCGtCA | CTG | 2 |
| | | | | | | AACTGT | AAT | | | AgCTGT | AAT | |
| | | | | | | TGTCAG | | | | TGTCAG | | |
| | | | | | | AACA | | | | AACA | | |
| EXON45 | SaCas9 | Reframe; | − | E45Sa8 | 16 | TTTTGG | CAG | mE45Sa8 | 319 | TTcTGa | CAG | 4 |
| | | Splice | | | | TATCTT | GAT | | | TggCTT | GAT | |
| | | | | | | ACAGGA | | | | ACAGGA | | |
| | | | | | | ACTC | | | | ACTC | | |
| EXON51 | SaCas9 | Reframe | + | E51Sa2 | 20 | GTTGTG | CTG | mE51Sa2 | 320 | NA | NA | NA |
| | | | | | | TCACCA | AGT | | | | | |
| | | | | | | GAGTAA | | | | | | |
| | | | | | | CAGT | | | | | | |
| EXON53 | SaCas9 | Reframe | + | E53Sa3 | 27 | CTTGTA | CTG | mE53Sa3 | 321 | CTTGaA | CTG | 3 |
| | | | | | | CTTCAT | AAT | | | CcTCAT | AAT | |
| | | | | | | CCCACT | | | | CCCACT | | |
| | | | | | | GATT | | | | GAaT | | |
| EXON53 | SaCas9 | Reframe; | + | E53Sa4 | 28 | ACTGAT | TAG | mE53Sa4 | 322 | ACTGAa | TGG | 1 |
| | | Splice | | | | TCTGAA | AAT | | | TCTGAA | AAT | |
| | | | | | | TTCTTT | | | | TTCTTT | | |
| | | | | | | CAAC | | | | CAAC | | |
| EXON53 | SaCas9 | Reframe | − | E53Sa8 | 32 | CCTTCA | TTG | mE53Sa8 | 323 | gCTgCA | TTG | 4 |
| | | | | | | GAACCG | AAT | | | GAACaG | AAT | |
| | | | | | | GAGGCA | | | | GAGaCA | | |
| | | | | | | ACAG | | | | ACAG | | |
| EXON53 | SaCas9 | Reframe | − | E53Sa9 | 33 | AGTTGA | TGG | mE53Sa9 | 324 | AGTTGA | TGG | 1 |
| | | | | | | AAGAAT | GAT | | | AAGAAT | GAT | |
| | | | | | | TCAGAA | | | | TCAGAt | | |
| | | | | | | TCAG | | | | TCAG | | |
| EXON53 | SaCas9 | Reframe; | − | E53Sa11 | 35 | TTTTTC | AAG | mE53Sa11 | 325 | TTcTTa | AAG | 4 |
| | | Splice | | | | CTTTTA | AAT | | | tTTTTA | AAT | |
| | | | | | | TTCTAG | | | | TTCcAG | | |
| | | | | | | TTGA | | | | TTGA | | |
| EXON45 | SpCas9 | Reframe | − | E45Sp52 | 400 | ATCTTA | TGG | mE45Sp52 | 403 | ggCTTA | TGG | 2 |
| | | | | | | CAGGAA | | | | CAGGAA | | |
| | | | | | | CTCCAG | | | | CTCCAG | | |
| | | | | | | GA | | | | GA | | |
| EXON51 | SpCas9 | Reframe | + | E51Sp32 | 401 | CACCAG | TAG | mE51Sp32 | 404 | CACtAG | TGG | 2 |
| | | | | | | AGTAAC | | | | AGTAAC | | |
| | | | | | | AGTCTG | | | | AGTCTG | | |
| | | | | | | AG | | | | Ac | | |
| EXON53 | SpCas9 | Reframe | − | E53Sp63 | 402 | TTGAAA | TGG | mE53Sp63 | 405 | TTGAAA | TGG | 1 |
| | | | | | | GAATTC | | | | GAATTC | | |
| | | | | | | AGAATC | | | | AGAtTC | | |
| | | | | | | AG | | | | AG | | |

2. Transfection of HECK293FT and Neuro-2a Cells

To evaluate indel frequency and profile, human HEK293FT and mouse Neuro-2a cell lines were used. HEK293FT and Neuro-2a cells were transfected in 12-well plates with 750 ng plasmid+2.25 μL of Lipofectamine 2000. Three days after transfection, cells were trypsinized and sorted for green fluorescent protein (GFP). GFP-positive cells were sorted directly into lysis buffer, and DNA extraction was performed using the GeneJet Genomic DNA Purification Kit. PCR was then performed on the DNA using exon-specific primers that targeted the relevant cut site.

3. Amplicon Deep Sequencing, Library Preparation, and Data Analysis

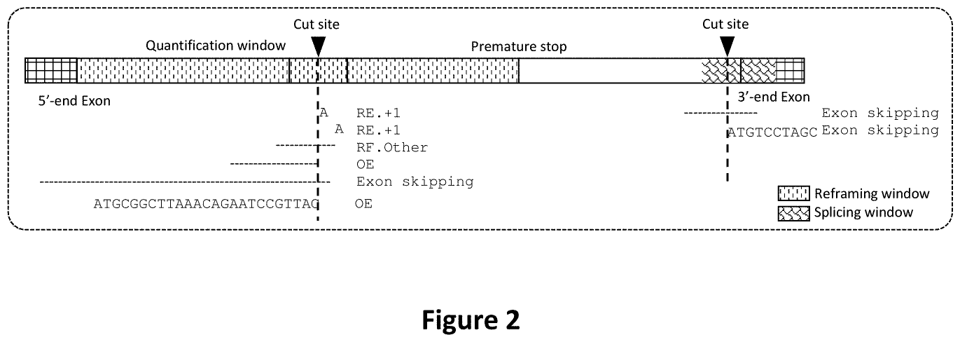

The relevant loci for each exon were amplified by PCR and the products were used to prepare sequencing libraries using MiSeq reagent kit V3. Indel analysis was performed using CRISPResso2±10-nt quantification window. (See, e.g., Clement et al., Nat Biotechnol. 2019 March; 37(3):224-226). Indel profiling consisted of 5 mutually exclusive indel categories, depicted in FIG. 2, and provided below:
a) NE: non-edited;
b) RF.+1: 1-nucleotide (nt) insertion leading to reframe;
c) RF.Other: indels other than 1-nt insertion leading to reframe:
   Deletion: not extending outside of the reframing window
   Insertion: <17-nt (i.e., <6 amino acids);
d) Exon skipping: indels that disrupt the ±6-nt window of the exon/intron boundaries leading to potential exon skipping (outcome requiring validation):
   The indel has >=9-nt overlap with the splicing window (to disrupt the GT/AG splicing sites); or
3) OE: Other indels.

The following sequences of selected primers were used for amplification of the specific human locus containing the sgRNA targeting sites (Tables 3A-3B) and the specific mouse locus containing the sgRNA targeting sites (Tables 4A-4B).

TABLE 3A
Primers for Amplification (Human)

| Tar- | | |
|---|---|---|
| geting | | |
| exon | Primer ID | Sequence |
| 45 | hE45-T7E1-F2 | GTCTTTCTGTCTTGTATCCTTTGG |
| | | (SEQ ID NO: 800) |
| | hE45-T7E1-R2 | AATGTTAGTGCCTTTCACCC |
| | | (SEQ ID NO: 801) |
| 51 | Ex51_MiSeq_R | CATGAATAAGAGTTTGGCTCAAATTG |
| | | (SEQ ID NO: 802) |
| | Ex51_MiSeq_F | GAGAGTAAAGTGATTGGTGGAAAATC |
| | | (SEQ ID NO: 803) |
| 53 | hEX53_F1 | AAATGTGAGATAACGTTTGGAAG |
| | | (SEQ ID NO: 804) |
| | hEX53_R1 | TTTCAGCTTTAACGTGATTTTCTG |
| | | (SEQ ID NO: 805) |

TABLE 3B
Primer + MiSeq Adapter Sequence (Human)

| Exon | ID | Sequence |
|---|---|---|
| 45 | MiSeq_hE45-T7E1- | TCGTCGGCAGCGTCAGATGTGTATAAGAGACAGGTCTTTCTGTCTTGTATCCTTTG |
| | F2 | G (SEQ ID NO: 806) |
| | MiSeq_hE45-T7E1- | GTCTCGTGGGCTCGGAGATGTGTATAAGAGACAGAATGTTAGTGCCTTTCACCC |
| | R2 | (SEQ ID NO: 807) |
| 51 | MiSeq_Ex51_MiSeq_ | TCGTCGGCAGCGTCAGATGTGTATAAGAGACAGCATGAATAAGAGTTTGGCTCAAA |
| | R | TTG (SEQ ID NO: 808) |
| | MiSeq_Ex51_MiSeq_ | GTCTCGTGGGCTCGGAGATGTGTATAAGAGACAGGAGAGTAAAGTGATTGGTGGAA |
| | F | AATC (SEQ ID NO: 809) |
| 53 | MiSeq_hEX53_F1 | TCGTCGGCAGCGTCAGATGTGTATAAGAGACAGAAATGTGAGATAACGTTTGGAAG |
| | | (SEQ ID NO: 810) |
| | MiSeq_hEX53_R1 | GTCTCGTGGGCTCGGAGATGTGTATAAGAGACAGTTTCAGCTTTAACGTGATTTTC |
| | | TG (SEQ ID NO: 811) |

TABLE 4A
Primers for Amplification (Mouse)

| Targeting | | |
|---|---|---|
| exon | Primer ID | Sequence |
| 45 | mEx45_F3 | TTTTCAGTGTAACTGCACATAAGAG |
| | | (SEQ ID NO: 812) |
| | mEx45_R3 | GCAAAAGTTGTCATTGTTGCT |
| | | (SEQ ID NO: 813) |
| 51 | mEx51_F3 | AAAATTGGCTCTTTTGCTTG |
| | | (SEQ ID NO: 814) |
| | mEx51_R3 | CACAGAGAAAAGGTAGCCTAAAAA |
| | | (SEQ ID NO: 815) |
| 53 | mEx53_F3 | GCACCTTGGATATATTTAATGAGAA |
| | | (SEQ ID NO: 816) |
| | mEx53_R3 | CAAGAATTCCACTTTTCACTTCC |
| | | (SEQ ID NO: 817) |

TABLE 4B
Primer + MiSeq Adapter Sequence (Mouse)

| Exon | ID | Sequence |
|---|---|---|
| 45 | mEx45_F3 | TCGTCGGCAGCGTCAGATGTGTATAAGAG |
| | | ACAGTTTTCAGTGTAACTGCACATAAGAG |
| | | (SEQ ID NO: 818) |
| | mEx45_R3 | GTCTCGTGGGCTCGGAGATGTGTATAAGA |
| | | GACAGGCAAAAGTTGTCATTGTTGCT |
| | | (SEQ ID NO: 819) |
| 51 | mEx51_F3 | TCGTCGGCAGCGTCAGATGTGTATAAGAG |
| | | ACAGAAAATTGGCTCTTTTGCTTG |
| | | (SEQ ID NO: 820) |
| | mEx51_R3 | GTCTCGTGGGCTCGGAGATGTGTATAAGA |
| | | GACAGCACAGAGAAAAGGTAGCCTAAAAA |
| | | (SEQ ID NO: 821) |
| 53 | mEx53_F3 | TCGTCGGCAGCGTCAGATGTGTATAAGAG |
| | | ACAGGCACCTTGGATATATTTAATGAGAA |
| | | (SEQ ID NO: 822) |
| | mEx53_R3 | GTCTCGTGGGCTCGGAGATGTGTATAAGA |
| | | GACAGCAAGAATTCCACTTTTCACTTCC |
| | | (SEQ ID NO: 823) |

B. Results

A set of exemplary DMD sgRNAs were evaluated for indel frequency and editing profile with either SaCas9 or SluCas9 (as indicated in Table 2). Among this selection, 13 sgRNAs were located within exon 45, 3 sgRNAs were located within exon 51 and 10 sgRNAs were located within exon 53. To evaluate indel frequency and profile, plasmid transfection was performed in HEK293FT and Neuro-2a cell lines.

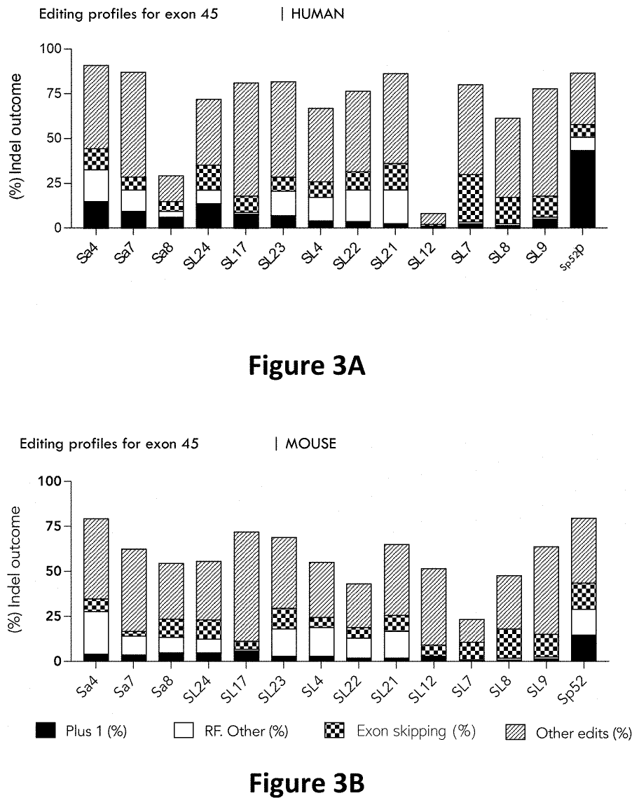

The average indel frequency of sgRNAs targeting exon 45 was determined in HEK293FT cells (FIG. 3A) and in Neuro-2a cells (FIG. 3B), with a high-performing SpCas9 sgRNA (E45Sp52) included as a reference. Of the exon 45-targeting sgRNAs evaluated, 11 sgRNAs showed an average total indel frequency higher than 50% in HEK293FT cells and a similar result was found for 10 sgRNAs tested in the Neuro-2a cell line. With regard to indel profile, E45Sa4 and E45SL24 were found to have the highest percent of combined indels that could lead to potential exon reframing/skipping in HEK293FT cells. In Neuro-2a cells, mE45Sa4 and mE45SL23 showed the highest ranking with respect to these potential outcomes. The sgRNAs E45SL17 and E45SL12 were not applicable to Δ44 mutation.

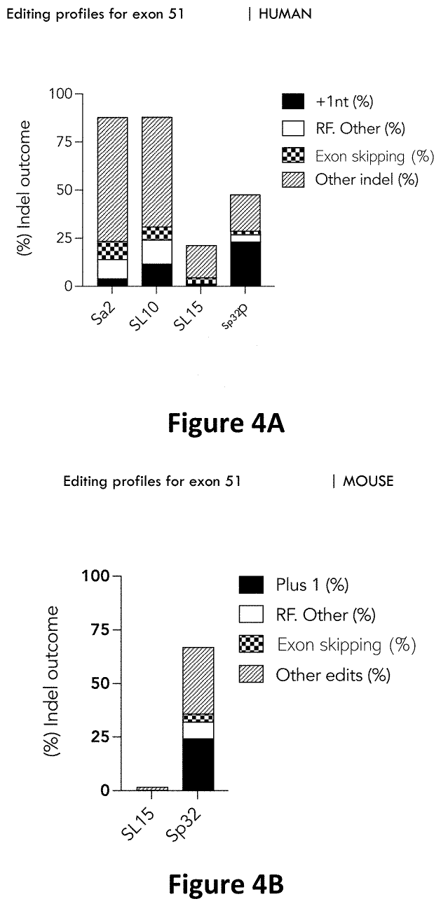

The average indel frequency of sgRNAs targeting exon 51 was determined in HEK293FT cells (FIG. 4A) and in Neuro-2a cells (FIG. 4B), with a high-performing SpCas9 sgRNA (E51Sp32) included as a reference. FIG. 4A shows an indel frequency higher than 50% and a combined indel profile that could lead to potential reframing/skipping above 25% for E51Sa2 and E51SL10 in HEK293FT cells. Due to the reduced level of sequence homology for these sgRNAs in the mouse locus, these sgRNAs could not be evaluated in the Neuro-2a cell line (e.g., sgRNAs E51Sa2 and E51SL10 do not have a mouse homolog and see FIG. 4B).

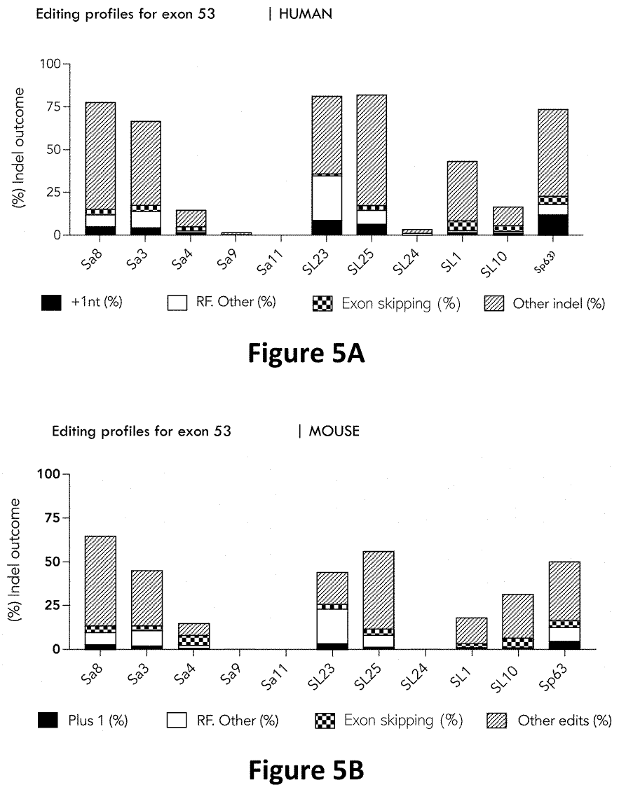

The average indel frequency of sgRNAs targeting exon 53 was determined in HEK293FT cells (FIG. 5A) and in Neuro-2a cells (FIG. 5B), with a high-performing SpCas9 sgRNA (E55Sp63) included as a reference. FIG. 5A shows an indel frequency higher than 50% for 4 sgRNAs within exon 53 in HEK293FT cells, and FIG. 5B shows a comparable result for 2 sgRNAs in Neuro-2a cells. Of the exon 53-targeting sgRNAs evaluated, E53Sa3 and mE53SL23 show the highest percent of combined indels with potential for reframing/skipping in both cell lines.

### Example 3: Exemplary DMD Guide RNAs for SaCas9 and SluCas9 Variants

Additional exemplary DMD guide RNAs were designed that may be used with SaCas9 and variants of SaCas9 (e.g., guide sequences having SEQ ID NOs: 1000-1078) and SluCas9 and variants of SluCas9 (e.g., guide sequences having SEQ ID NOs: 2000-2116) in Table 5 below. In particular, guide RNAs were designed based on SaCas9-KKH (for guide sequences having SEQ ID NOs: 1000-1078) with a PAM sequence NNNRRT (N is any nucleotide, R is purine) and SluCas9-KH (for guide sequences having SEQ ID NOs: 2000-2116) with a PAM sequence NNRG.

Guide RNAs were designed focusing on genomic coordinate regions within exons 45, 51, and 53. For exon 45, the design region was genomic coordinates chrX: 31968307-31968546. For exon 51, the design region was genomic coordinates chrX: 31773928-31774224. For exon 53, the design region was genomic coordinates chrX: 31679343-31679618.

Off-target site prediction was computationally performed for two sets of mismatches: (1) 3 mismatches+0 bulge; and (2) 2 mismatches+1 bulge. Results are shown in Table 5.

Guide RNA comprising the guide sequences shown in Table 5 below are prepared according to standard methods in a single guide (sgRNA) format. A single AAV vector is prepared that expresses the guide RNA and a variant SaCas9 (for guide sequences having SEQ ID NOs: 1000-1078) or the guide RNA and a variant SluCas9 (for guide sequences having SEQ ID NOs: 2000-2116). See, Table 5. The AAV vector is administered to cells in vitro and to mice (e.g., mdx mice) in vivo to assess the ability of the AAV to express the guide RNA and Cas9, edit the targeted exon (see Table 5), and thereby treat DMD.

In particular, the ability of in vivo single AAV-mediated delivery of gene-editing components to successfully remove the mutant genomic sequence by exon skipping in the cardiac and skeletal muscle cells of mdx mice is tested.

TABLE 5
Additional DMD Guide Sequences (human-hg38.p12)

| | | | | Genomic | | | | | |
|---|---|---|---|---|---|---|---|---|---|
| | | | | coordinate | | | | | |
| | | | | chrX_stop | | SEQ ID | | | |
| | | | Genomic | (includes | | NO | | | |
| | | | coordinate | PAM | | Guide | | | Offtargets_ |
| EXON | CAS9 | ID | chrX_start | coordinates) | Strand | sequence | Guide sequence | PAM | grouped |
| 45 | SACAS9 | E45SaCas | 31968359 | 31968387 | + | 1000 | TGTTTGCAGACCTCCTGCCACC | GCAGAT | 20 |
| | KKH | 9KKH1 | | | | | | | |
| 45 | SACAS9 | E45SaCas | 31968373 | 31968401 | + | 1001 | CTGCCACCGCAGATTCAGGCTT | CCCAAT | 9 |
| | KKH | 9KKH2 | | | | | | | |
| 45 | SACAS9 | E45SaCas | 31968387 | 31968415 | + | 9 | TCAGGCTTCCCAATTTTTCCTG | TAGAAT | 37 |
| | KKH | 9KKH3 | | | | | | | |
| 45 | SACAS9 | E45SaCas | 31968409 | 31968437 | + | 10 | TAGAATACTGGCATCTGTTTTT | GAGGAT | 63 |
| | KKH | 9KKH4 | | | | | | | |
| 45 | SACAS9 | E45SaCas | 31968417 | 31968445 | + | 11 | TGGCATCTGTTTTTGAGGATTG | CTGAAT | 35 |
| | KKH | 9KKH5 | | | | | | | |
| 45 | SACAS9 | E45SaCas | 31968432 | 31968460 | + | 1002 | AGGATTGCTGAATTATTTCTTC | CCCAGT | 42 |
| | KKH | 9KKH6 | | | | | | | |
| 45 | SACAS9 | E45SaCas | 31968442 | 31968470 | + | 1003 | AATTATTTCTTCCCCAGTTGCA | TTCAAT | 68 |
| | KKH | 9KKH7 | | | | | | | |
| 45 | SACAS9 | E45SaCas | 31968456 | 31968484 | + | 1004 | CAGTTGCATTCAATGTTCTGAC | AACAGT | 17 |
| | KKH | 9KKH8 | | | | | | | |
| 45 | SACAS9 | E45SaCas | 31968471 | 31968499 | + | 1005 | TTCTGACAACAGTTTGCCGCTG | CCCAAT | 6 |
| | KKH | 9KKH9 | | | | | | | |
| 45 | SACAS9 | E45SaCas | 31968484 | 31968512 | + | 12 | TTGCCGCTGCCCAATGCCATCC | TGGAGT | 5 |
| | KKH | 9KKH10 | | | | | | | |
| 45 | SACAS9 | E45SaCas | 31968495 | 31968523 | + | 1006 | CAATGCCATCCTGGAGTTCCTG | TAAGAT | 27 |
| | KKH | 9KKH11 | | | | | | | |
| 45 | SACAS9 | E45SaCas | 31968517 | 31968545 | + | 1007 | TAAGATACCAAAAAGGCAAAAC | AAAAAT | 96 |
| | KKH | 9KKH12 | | | | | | | |
| 45 | SACAS9 | E45SaCas | 31968313 | 31968341 | - | 13 | GAGGTAGGGCGACAGATCTAAT | AGGAAT | 10 |
| | KKH | 9KKH13 | | | | | | | |
| 45 | SACAS9 | E45SaCas | 31968319 | 31968347 | - | 1008 | AAAAAAGAGGTAGGGCGACAGA | TCTAAT | 75 |
| | KKH | 9KKH14 | | | | | | | |
| 45 | SACAS9 | E45SaCas | 31968324 | 31968352 | - | 1009 | GACAGAAAAAAGAGGTAGGGCG | ACAGAT | 51 |
| | KKH | 9KKH15 | | | | | | | |
| 45 | SACAS9 | E45SaCas | 31968336 | 31968364 | - | 1010 | AAACAGCTGTCAGACAGAAAAA | AGAGGT | 123 |
| | KKH | 9KKH16 | | | | | | | |
| 45 | SACAS9 | E45SaCas | 31968368 | 31968396 | - | 1011 | GAAGCCTGAATCTGCGGTGGCA | GGAGGT | 19 |
| | KKH | 9KKH17 | | | | | | | |
| 45 | SACAS9 | E45SaCas | 31968378 | 31968406 | - | 1012 | GAAAAATTGGGAAGCCTGAATC | TGCGGT | 29 |
| | KKH | 9KKH18 | | | | | | | |
| 45 | SACAS9 | E45SaCas | 31968385 | 31968413 | - | 14 | TCTACAGGAAAAATTGGGAAGC | CTGAAT | 64 |
| | KKH | 9KKH19 | | | | | | | |
| 45 | SACAS9 | E45SaCas | 31968399 | 31968427 | - | 1013 | ACAGATGCCAGTATTCTACAGG | AAAAAT | 22 |
| | KKH | 9KKH20 | | | | | | | |
| 45 | SACAS9 | E45SaCas | 31968415 | 31968443 | - | 1014 | TCAGCAATCCTCAAAAACAGAT | GCCAGT | 50 |
| | KKH | 9KKH21 | | | | | | | |
| 45 | SACAS9 | E45SaCas | 31968421 | 31968449 | - | 1015 | AATAATTCAGCAATCCTCAAAA | ACAGAT | 99 |
| | KKH | 9KKH22 | | | | | | | |
| 45 | SACAS9 | E45SaCas | 31968435 | 31968463 | - | 1016 | GCAACTGGGGAAGAAATAATTC | AGCAAT | 43 |
| | KKH | 9KKH23 | | | | | | | |
| 45 | SACAS9 | E45SaCas | 31968443 | 31968471 | - | 1017 | CATTGAATGCAACTGGGGAAGA | AATAAT | 54 |
| | KKH | 9KKH24 | | | | | | | |
| 45 | SACAS9 | E45SaCas | 31968446 | 31968474 | - | 1018 | GAACATTGAATGCAACTGGGGA | AGAAAT | 23 |
| | KKH | 9KKH25 | | | | | | | |
| 45 | SACAS9 | E45SaCas | 31968463 | 31968491 | - | 15 | GCGGCAAACTGTTGTCAGAACA | TTGAAT | 5 |
| | KKH | 9KKH26 | | | | | | | |
| 45 | SACAS9 | E45SaCas | 31968502 | 31968530 | - | 16 | TTTTGGTATCTTACAGGAACTC | CAGGAT | 52 |
| | KKH | 9KKH27 | | | | | | | |
| 45 | SLUCAS | E45SLCas | 31968332 | 31968358 | + | 2000 | CCCTACCTCTTTTTTCTGTCTG | ACAG | 23 |
| | 9KH | 9KH1 | | | | | | | |
| 45 | SLUCAS | E45SLCas | 31968342 | 31968368 | + | 2001 | TTTTTCTGTCTGACAGCTGTTT | GCAG | 22 |
| | 9KH | 9KH2 | | | | | | | |
| 45 | SLUCAS | E45SLCas | 31968359 | 31968385 | + | 2002 | TGTTTGCAGACCTCCTGCCACC | GCAG | 9 |
| | 9KH | 9KH3 | | | | | | | |
| 45 | SLUCAS | E45SLCas | 31968365 | 31968391 | + | 2003 | CAGACCTCCTGCCACCGCAGAT | TCAG | 9 |
| | 9KH | 9KH4 | | | | | | | |
| 45 | SLUCAS | E45SLCas | 31968366 | 31968392 | + | 128 | AGACCTCCTGCCACCGCAGATT | CAGG | 10 |
| | 9KH | 9KH5 | | | | | | | |
| 45 | SLUCAS | E45SLCas | 31968386 | 31968412 | + | 2004 | TTCAGGCTTCCCAATTTTTCCT | GTAG | 11 |
| | 9KH | 9KH6 | | | | | | | |
| 45 | SLUCAS | E45SLCas | 31968394 | 31968420 | + | 129 | TCCCAATTTTTCCTGTAGAATA | CTGG | 19 |
| | 9KH | 9KH7 | | | | | | | |
| 45 | SLUCAS | E45SLCas | 31968408 | 31968434 | + | 2005 | GTAGAATACTGGCATCTGTTTT | TGAG | 16 |
| | 9KH | 9KH8 | | | | | | | |
| 45 | SLUCAS | E45SLCas | 31968409 | 31968435 | + | 130 | TAGAATACTGGCATCTGTTTTT | GAGG | 19 |
| | 9KH | 9KH9 | | | | | | | |
| 45 | SLUCAS | E45SLCas | 31968433 | 31968459 | + | 2006 | GGATTGCTGAATTATTTCTTCC | CCAG | 18 |
| | 9KH | 9KH10 | | | | | | | |
| 45 | SLUCAS | E45SLCas | 31968457 | 31968483 | + | 2007 | AGTTGCATTCAATGTTCTGACA | ACAG | 10 |
| | 9KH | 9KH11 | | | | | | | |
| 45 | SLUCAS | E45SLCas | 31968483 | 31968509 | + | 131 | TTTGCCGCTGCCCAATGCCATC | CTGG | 2 |
| | 9KH | 9KH12 | | | | | | | |
| 45 | SLUCAS | E45SLCas | 31968485 | 31968511 | + | 2008 | TGCCGCTGCCCAATGCCATCCT | GGAG | 7 |
| | 9KH | 9KH13 | | | | | | | |
| 45 | SLUCAS | E45SLCas | 31968495 | 31968521 | + | 2009 | CAATGCCATCCTGGAGTTCCTG | TAAG | 14 |
| | 9KH | 9KH14 | | | | | | | |
| 45 | SLUCAS | E45SLCas | 31968506 | 31968532 | + | 2010 | TGGAGTTCCTGTAAGATACCAA | AAAG | 10 |
| | 9KH | 9KH15 | | | | | | | |
| 45 | SLUCAS | E45SLCas | 31968507 | 31968533 | + | 132 | GGAGTTCCTGTAAGATACCAAA | AAGG | 9 |
| | 9KH | 9KH16 | | | | | | | |
| 45 | SLUCAS | E45SLCas | 31968316 | 31968342 | - | 133 | AGAGGTAGGGCGACAGATCTAA | TAGG | 6 |
| | 9KH | 9KH17 | | | | | | | |
| 45 | SLUCAS | E45SLCas | 31968317 | 31968343 | - | 2011 | AAGAGGTAGGGCGACAGATCTA | ATAG | 5 |
| | 9KH | 9KH18 | | | | | | | |
| 45 | SLUCAS | E45SLCas | 31968326 | 31968352 | - | 2012 | GACAGAAAAAAGAGGTAGGGCG | ACAG | 31 |
| | 9KH | 9KH19 | | | | | | | |
| 45 | SLUCAS | E45SLCas | 31968332 | 31968358 | - | 134 | CTGTCAGACAGAAAAAAGAGGT | AGGG | 28 |
| | 9KH | 9KH20 | | | | | | | |
| 45 | SLUCAS | E45SLCas | 31968333 | 31968359 | - | 135 | GCTGTCAGACAGAAAAAAGAGG | TAGG | 43 |
| | 9KH | 9KH21 | | | | | | | |
| 45 | SLUCAS | E45SLCas | 31968334 | 31968360 | - | 2013 | AGCTGTCAGACAGAAAAAAGAG | GTAG | 67 |
| | 9KH | 9KH22 | | | | | | | |
| 45 | SLUCAS | E45SLCas | 31968337 | 31968363 | - | 136 | AACAGCTGTCAGACAGAAAAAA | GAGG | 55 |
| | 9KH | 9KH23 | | | | | | | |
| 45 | SLUCAS | E45SLCas | 31968338 | 31968364 | - | 2014 | AAACAGCTGTCAGACAGAAAAA | AGAG | 37 |
| | 9KH | 9KH24 | | | | | | | |
| 45 | SLUCAS | E45SLCas | 31968340 | 31968366 | - | 2015 | GCAAACAGCTGTCAGACAGAAA | AAAG | 27 |
| | 9KH | 9KH25 | | | | | | | |
| 45 | SLUCAS | E45SLCas | 31968347 | 31968373 | - | 2016 | GAGGTCTGCAAACAGCTGTCAG | ACAG | 23 |
| | 9KH | 9KH26 | | | | | | | |
| 45 | SLUCAS | E45SLCas | 31968351 | 31968377 | - | 2017 | GCAGGAGGTCTGCAAACAGCTG | TCAG | 16 |
| | 9KH | 9KH27 | | | | | | | |
| 45 | SLUCAS | E45SLCas | 31968358 | 31968384 | - | 2018 | TGCGGTGGCAGGAGGTCTGCAA | ACAG | 14 |
| | 9KH | 9KH28 | | | | | | | |
| 45 | SLUCAS | E45SLCas | 31968369 | 31968395 | - | 137 | AAGCCTGAATCTGCGGTGGCAG | GAGG | 11 |
| | 9KH | 9KH29 | | | | | | | |
| 45 | SLUCAS | E45SLCas | 31968370 | 31968396 | - | 2019 | GAAGCCTGAATCTGCGGTGGCA | GGAG | 15 |
| | 9KH | 9KH30 | | | | | | | |
| 45 | SLUCAS | E45SLCas | 31968372 | 31968398 | - | 138 | GGGAAGCCTGAATCTGCGGTGG | CAGG | 8 |
| | 9KH | 9KH31 | | | | | | | |
| 45 | SLUCAS | E45SLCas | 31968373 | 31968399 | - | 2020 | TGGGAAGCCTGAATCTGCGGTG | GCAG | 6 |
| | 9KH | 9KH32 | | | | | | | |
| 45 | SLUCAS | E45SLCas | 31968376 | 31968402 | - | 139 | AATTGGGAAGCCTGAATCTGCG | GTGG | 7 |
| | 9KH | 9KH33 | | | | | | | |
| 45 | SLUCAS | E45SLCas | 31968379 | 31968405 | - | 140 | AAAAATTGGGAAGCCTGAATCT | GCGG | 26 |
| | 9KH | 9KH34 | | | | | | | |
| 45 | SLUCAS | E45SLCas | 31968392 | 31968418 | - | 2021 | AGTATTCTACAGGAAAAATTGG | GAAG | 28 |
| | 9KH | 9KH35 | | | | | | | |
| 45 | SLUCAS | E45SLCas | 31968395 | 31968421 | - | 141 | GCCAGTATTCTACAGGAAAAAT | TGGG | 16 |
| | 9KH | 9KH36 | | | | | | | |
| 45 | SLUCAS | E45SLCas | 31968396 | 31968422 | - | 142 | TGCCAGTATTCTACAGGAAAAA | TTGG | 17 |
| | 9KH | 9KH37 | | | | | | | |
| 45 | SLUCAS | E45SLCas | 31968405 | 31968431 | - | 143 | AAAAACAGATGCCAGTATTCTA | CAGG | 21 |
| | 9KH | 9KH38 | | | | | | | |
| 45 | SLUCAS | E45SLCas | 31968406 | 31968432 | - | 2022 | CAAAAACAGATGCCAGTATTCT | ACAG | 28 |
| | 9KH | 9KH39 | | | | | | | |
| 45 | SLUCAS | E45SLCas | 31968416 | 31968442 | - | 2023 | CAGCAATCCTCAAAAACAGATG | CCAG | 23 |
| | 9KH | 9KH40 | | | | | | | |
| 45 | SLUCAS | E45SLCas | 31968423 | 31968449 | - | 2024 | AATAATTCAGCAATCCTCAAAA | ACAG | 31 |
| | 9KH | 9KH41 | | | | | | | |
| 45 | SLUCAS | E45SLCas | 31968439 | 31968465 | - | 2025 | ATGCAACTGGGGAAGAAATAAT | TCAG | 33 |
| | 9KH | 9KH42 | | | | | | | |
| 45 | SLUCAS | E45SLCas | 31968450 | 31968476 | - | 2026 | CAGAACATTGAATGCAACTGGG | GAAG | 11 |
| | 9KH | 9KH43 | | | | | | | |
| 45 | SLUCAS | E45SLCas | 31968453 | 31968479 | - | 144 | TGTCAGAACATTGAATGCAACT | GGGG | 12 |
| | 9KH | 9KH44 | | | | | | | |
| 45 | SLUCAS | E45SLCas | 31968454 | 31968480 | - | 145 | TTGTCAGAACATTGAATGCAAC | TGGG | 30 |
| | 9KH | 9KH45 | | | | | | | |
| 45 | SLUCAS | E45SLCas | 31968455 | 31968481 | - | 146 | GTTGTCAGAACATTGAATGCAA | CTGG | 12 |
| | 9KH | 9KH46 | | | | | | | |
| 45 | SLUCAS | E45SLCas | 31968473 | 31968499 | - | 2027 | ATTGGGCAGCGGCAAACTGTTG | TCAG | 3 |
| | 9KH | 9KH47 | | | | | | | |
| 45 | SLUCAS | E45SLCas | 31968487 | 31968513 | - | 147 | AACTCCAGGATGGCATTGGGCA | GCGG | 9 |
| | 9KH | 9KH48 | | | | | | | |
| 45 | SLUCAS | E45SLCas | 31968490 | 31968516 | - | 2028 | AGGAACTCCAGGATGGCATTGG | GCAG | 16 |
| | 9KH | 9KH49 | | | | | | | |
| 45 | SLUCAS | E45SLCas | 31968493 | 31968519 | - | 148 | TACAGGAACTCCAGGATGGCAT | TGGG | 10 |
| | 9KH | 9KH50 | | | | | | | |
| 45 | SLUCAS | E45SLCas | 31968494 | 31968520 | - | 149 | TTACAGGAACTCCAGGATGGCA | TTGG | 13 |
| | 9KH | 9KH51 | | | | | | | |
| 45 | SLUCAS | E45SLCas | 31968500 | 31968526 | - | 150 | GGTATCTTACAGGAACTCCAGG | ATGG | 7 |
| | 9KH | 9KH52 | | | | | | | |
| 45 | SLUCAS | E45SLCas | 31968504 | 31968530 | - | 151 | TTTTGGTATCTTACAGGAACTC | CAGG | 35 |
| | 9KH | 9KH53 | | | | | | | |
| 45 | SLUCAS | E45SLCas | 31968505 | 31968531 | - | 2029 | TTTTTGGTATCTTACAGGAACT | CCAG | 21 |
| | 9KH | 9KH54 | | | | | | | |
| 45 | SLUCAS | E45SLCas | 31968513 | 31968539 | - | 152 | GTTTTGCCTTTTTGGTATCTTA | CAGG | 32 |
| | 9KH | 9KH55 | | | | | | | |
| 45 | SLUCAS | E45SLCas | 31968514 | 31968540 | - | 2030 | TGTTTTGCCTTTTTGGTATCTT | ACAG | 58 |
| | 9KH | 9KH56 | | | | | | | |
| 51 | SACAS9 | E51SaCas | 31773943 | 31773971 | + | 1019 | TCATTTTTTCTCATACCTTCTG | CTTGAT | 91 |
| | KKH | 9KKH1 | | | | | | | |
| 51 | SACAS9 | E51SaCas | 31773946 | 31773974 | + | 1020 | TTTTTTCTCATACCTTCTGCTT | GATGAT | 132 |
| | KKH | 9KKH2 | | | | | | | |
| 51 | SACAS9 | E51SaCas | 31773958 | 31773986 | + | 1021 | CCTTCTGCTTGATGATCATCTC | GTTGAT | 23 |
| | KKH | 9KKH3 | | | | | | | |
| 51 | SACAS9 | E51SaCas | 31773969 | 31773997 | + | 1022 | ATGATCATCTCGTTGATATCCT | CAAGGT | 15 |
| | KKH | 9KKH4 | | | | | | | |
| 51 | SACAS9 | E51SaCas | 31773992 | 31774020 | + | 1023 | AAGGTCACCCACCATCACCCTC | TGTGAT | 33 |
| | KKH | 9KKH5 | | | | | | | |
| 51 | SACAS9 | E51SaCas | 31774005 | 31774033 | + | 1024 | ATCACCCTCTGTGATTTTATAA | CTTGAT | 45 |
| | KKH | 9KKH6 | | | | | | | |
| 51 | SACAS9 | E51SaCas | 31774023 | 31774051 | + | 1025 | ATAACTTGATCAAGCAGAGAAA | GCCAGT | 101 |
| | KKH | 9KKH7 | | | | | | | |
| 51 | SACAS9 | E51SaCas | 31774027 | 31774055 | + | 1026 | CTTGATCAAGCAGAGAAAGCCA | GTCGGT | 55 |
| | KKH | 9KKH8 | | | | | | | |
| 51 | SACAS9 | E51SaCas | 31774031 | 31774059 | + | 1027 | ATCAAGCAGAGAAAGCCAGTCG | GTAAGT | 16 |
| | KKH | 9KKH9 | | | | | | | |
| 51 | SACAS9 | E51SaCas | 31774047 | 31774075 | + | 1028 | CAGTCGGTAAGTTCTGTCCAAG | CCCGGT | 7 |
| | KKH | 9KKH10 | | | | | | | |
| 51 | SACAS9 | E51SaCas | 31774053 | 31774081 | + | 1029 | GTAAGTTCTGTCCAAGCCCGGT | TGAAAT | 4 |
| | KKH | 9KKH11 | | | | | | | |
| 51 | SACAS9 | E51SaCas | 31774067 | 31774095 | + | 1030 | AGCCCGGTTGAAATCTGCCAGA | GCAGGT | 7 |
| | KKH | 9KKH12 | | | | | | | |
| 51 | SACAS9 | E51SaCas | 31774088 | 31774116 | + | 1031 | AGCAGGTACCTCCAACATCAAG | GAAGAT | 23 |
| | KKH | 9KKH13 | | | | | | | |
| 51 | SACAS9 | E51SaCas | 31774100 | 31774128 | + | 1032 | CAACATCAAGGAAGATGGCATT | TCTACT | 54 |
| | KKH | 9KKH14 | | | | | | | |
| 51 | SACAS9 | E51SaCas | 31774108 | 31774136 | + | 1033 | AGGAAGATGGCATTTCTAGTTT | GGAGAT | 62 |
| | KKH | 9KKH15 | | | | | | | |
| 51 | SACAS9 | E51SaCas | 31774114 | 31774142 | + | 1034 | ATGGCATTTCTAGTTTGGAGAT | GGCAGT | 56 |
| | KKH | 9KKH16 | | | | | | | |
| 51 | SACAS9 | E51SaCas | 31774123 | 31774151 | + | 1035 | CTAGTTTGGAGATGGCAGTTTC | CTTAGT | 32 |
| | KKH | 9KKH17 | | | | | | | |
| 51 | SACAS9 | E51SaCas | 31774133 | 31774161 | + | 1036 | GATGGCAGTTTCCTTAGTAACC | ACAGGT | 24 |
| | KKH | 9KKH18 | | | | | | | |
| 51 | SACAS9 | E51SaCas | 31774147 | 31774175 | + | 19 | TAGTAACCACAGGTTGTGTCAC | CAGAGT | 18 |
| | KKH | 9KKH19 | | | | | | | |
| 51 | SACAS9 | E51SaCas | 31774153 | 31774181 | + | 1037 | CCACAGGTTGTGTCACCAGAGT | AACAGT | 20 |
| | KKH | 9KKH20 | | | | | | | |
| 51 | SACAS9 | E51SaCas | 31774159 | 31774187 | + | 20 | GTTGTGTCACCAGAGTAACAGT | CTGAGT | 21 |
| | KKH | 9KKH21 | | | | | | | |
| 51 | SACAS9 | E51SaCas | 31774171 | 31774199 | + | 1038 | GAGTAACAGTCTGAGTAGGAGC | TAAAAT | 18 |
| | KKH | 9KKH22 | | | | | | | |
| 51 | SACAS9 | E51SaCas | 31774180 | 31774208 | + | 21 | TCTGAGTAGGAGCTAAAATATT | TTGGGT | 38 |
| | KKH | 9KKH23 | | | | | | | |
| 51 | SACAS9 | E51SaCas | 31773936 | 31773964 | - | 1039 | AGAAGGTATGAGAAAAAATGAT | AAAAGT | 236 |
| | KKH | 9KKH24 | | | | | | | |
| 51 | SACAS9 | E51SaCas | 31773942 | 31773970 | - | 1040 | TCAAGCAGAAGGTATGAGAAAA | AATGAT | 99 |
| | KKH | 9KKH25 | | | | | | | |
| 51 | SACAS9 | E51SaCas | 31773945 | 31773973 | - | 1041 | TCATCAAGCAGAAGGTATGAGA | AAAAAT | 43 |
| | KKH | 9KKH26 | | | | | | | |
| 51 | SACAS9 | E51SaCas | 31773957 | 31773985 | - | 1042 | TCAACGAGATGATCATCAAGCA | GAAGGT | 10 |
| | KKH | 9KKH27 | | | | | | | |
| 51 | SACAS9 | E51SaCas | 31773972 | 31774000 | - | 1043 | GTGACCTTGAGGATATCAACGA | GATGAT | 7 |
| | KKH | 9KKH28 | | | | | | | |
| 51 | SACAS9 | E51SaCas | 31773975 | 31774003 | - | 1044 | TGGGTGACCTTGAGGATATCAA | CGAGAT | 68 |
| | KKH | 9KKH29 | | | | | | | |
| 51 | SACAS9 | E51SaCas | 31773986 | 31774014 | - | 22 | GAGGGTGATGGTGGGTGACCTT | GAGGAT | 46 |
| | KKH | 9KKH30 | | | | | | | |
| 51 | SACAS9 | E51SaCas | 31773998 | 31774026 | - | 23 | TATAAAATCACAGAGGGTGATG | GTGGGT | 42 |
| | KKH | 9KKH31 | | | | | | | |
| 51 | SACAS9 | E51SaCas | 31774002 | 31774030 | - | 1045 | AAGTTATAAAATCACAGAGGGT | GATGGT | 77 |
| | KKH | 9KKH32 | | | | | | | |
| 51 | SACAS9 | E51SaCas | 31774005 | 31774033 | - | 1046 | ATCAAGTTATAAAATCACAGAG | GGTGAT | 90 |
| | KKH | 9KKH33 | | | | | | | |
| 51 | SACAS9 | E51SaCas | 31774008 | 31774036 | - | 24 | TTGATCAAGTTATAAAATCACA | GAGGGT | 86 |
| | KKH | 9KKH34 | | | | | | | |
| 51 | SACAS9 | E51SaCas | 31774018 | 31774046 | - | 1047 | CTTTCTCTGCTTGATCAAGTTA | TAAAAT | 45 |
| | KKH | 9KKH35 | | | | | | | |
| 51 | SACAS9 | E51SaCas | 31774026 | 31774054 | - | 1048 | CCGACTGGCTTTCTCTGCTTGA | TCAAGT | 18 |
| | KKH | 9KKH36 | | | | | | | |
| 51 | SACAS9 | E51SaCas | 31774031 | 31774059 | - | 1049 | ACTTACCGACTGGCTTTCTCTG | CTTGAT | 11 |
| | KKH | 9KKH37 | | | | | | | |
| 51 | SACAS9 | E51SaCas | 31774079 | 31774107 | - | 1050 | GATGTTGGAGGTACCTGCTCTG | GCAGAT | 22 |
| | KKH | 9KKH38 | | | | | | | |
| 51 | SACAS9 | E51SaCas | 31774095 | 31774123 | - | 1051 | AAATGCCATCTTCCTTGATGTT | GGAGGT | 57 |
| | KKH | 9KKH39 | | | | | | | |
| 51 | SACAS9 | E51SaCas | 31774104 | 31774132 | - | 1052 | CCAAACTAGAAATGCCATCTTC | CTTGAT | 46 |
| | KKH | 9KKH40 | | | | | | | |
| 51 | SACAS9 | E51SaCas | 31774119 | 31774147 | - | 1053 | AGGAAACTGCCATCTCCAAACT | AGAAAT | 63 |
| | KKH | 9KKH41 | | | | | | | |
| 51 | SACAS9 | E51SaCas | 31774152 | 31774180 | - | 1054 | CTGTTACTCTGGTGACACAACC | TGTGGT | 27 |
| | KKH | 9KKH42 | | | | | | | |
| 51 | SACAS9 | E51SaCas | 31774167 | 31774195 | - | 1055 | TAGCTCCTACTCAGACTGTTAC | TCTGGT | 13 |
| | KKH | 9KKH43 | | | | | | | |
| 51 | SLUCAS | E51SLCas | 31773969 | 31773995 | + | 2031 | ATGATCATCTCGTTGATATCCT | CAAG | 6 |
| | 9KH | 9KH1 | | | | | | | |
| 51 | SLUCAS | E51SLCas | 31773970 | 31773996 | + | 170 | TGATCATCTCGTTGATATCCTC | AAGG | 6 |
| | 9KH | 9KH2 | | | | | | | |
| 51 | SLUCAS | E51SLCas | 31774011 | 31774037 | + | 2032 | CTCTGTGATTTTATAACTTGAT | CAAG | 18 |
| | 9KH | 9KH3 | | | | | | | |
| 51 | SLUCAS | E51SLCas | 31774014 | 31774040 | + | 2033 | TGTGATTTTATAACTTGATCAA | GCAG | 29 |
| | 9KH | 9KH4 | | | | | | | |
| 51 | SLUCAS | E51SLCas | 31774016 | 31774042 | + | 2034 | TGATTTTATAACTTGATCAAGC | AGAG | 15 |
| | 9KH | 9KH5 | | | | | | | |
| 51 | SLUCAS | E51SLCas | 31774020 | 31774046 | + | 2035 | TTTATAACTTGATCAAGCAGAG | AAAG | 7 |
| | 9KH | 9KH6 | | | | | | | |
| 51 | SLUCAS | E51SLCas | 31774024 | 31774050 | + | 2036 | TAACTTGATCAAGCAGAGAAAG | CCAG | 24 |
| | 9KH | 9KH7 | | | | | | | |
| 51 | SLUCAS | E51SLCas | 31774028 | 31774054 | + | 171 | TTGATCAAGCAGAGAAAGCCAG | TCGG | 23 |
| | 9KH | 9KH8 | | | | | | | |
| 51 | SLUCAS | E51SLCas | 31774032 | 31774058 | + | 2037 | TCAAGCAGAGAAAGCCAGTCGG | TAAG | 7 |
| | 9KH | 9KH9 | | | | | | | |
| 51 | SLUCAS | E51SLCas | 31774043 | 31774069 | + | 2038 | AAGCCAGTCGGTAAGTTCTGTC | CAAG | 7 |
| | 9KH | 9KH10 | | | | | | | |
| 51 | SLUCAS | E51SLCas | 31774048 | 31774074 | + | 172 | AGTCGGTAAGTTCTGTCCAAGC | CCGG | 2 |
| | 9KH | 9KH11 | | | | | | | |
| 51 | SLUCAS | E51SLCas | 31774062 | 31774088 | + | 2039 | GTCCAAGCCCGGTTGAAATCTG | CCAG | 1 |
| | 9KH | 9KH12 | | | | | | | |
| 51 | SLUCAS | E51SLCas | 31774064 | 31774090 | + | 2040 | CCAAGCCCGGTTGAAATCTGCC | AGAG | 2 |
| | 9KH | 9KH13 | | | | | | | |
| 51 | SLUCAS | E51SLCas | 31774067 | 31774093 | + | 2041 | AGCCCGGTTGAAATCTGCCAGA | GCAG | 4 |
| | 9KH | 9KH14 | | | | | | | |
| 51 | SLUCAS | E51SLCas | 31774068 | 31774094 | + | 173 | GCCCGGTTGAAATCTGCCAGAG | CAGG | 5 |
| | 9KH | 9KH15 | | | | | | | |
| 51 | SLUCAS | E51SLCas | 31774084 | 31774110 | + | 2042 | CCAGAGCAGGTACCTCCAACAT | CAAG | 14 |
| | 9KH | 9KH16 | | | | | | | |
| 51 | SLUCAS | E51SLCas | 31774085 | 31774111 | + | 174 | CAGAGCAGGTACCTCCAACATC | AAGG | 13 |
| | 9KH | 9KH17 | | | | | | | |
| 51 | SLUCAS | E51SLCas | 31774088 | 31774114 | + | 2043 | AGCAGGTACCTCCAACATCAAG | GAAG | 12 |
| | 9KH | 9KH18 | | | | | | | |
| 51 | SLUCAS | E51SLCas | 31774092 | 31774118 | + | 175 | GGTACCTCCAACATCAAGGAAG | ATGG | 6 |
| | 9KH | 9KH19 | | | | | | | |
| 51 | SLUCAS | E51SLCas | 31774101 | 31774127 | + | 2044 | AACATCAAGGAAGATGGCATTT | CTAG | 32 |
| | 9KH | 9KH20 | | | | | | | |
| 51 | SLUCAS | E51SLCas | 31774106 | 31774132 | + | 176 | CAAGGAAGATGGCATTTCTAGT | TTGG | 21 |
| | 9KH | 9KH21 | | | | | | | |
| 51 | SLUCAS | E51SLCas | 31774108 | 31774134 | + | 2045 | AGGAAGATGGCATTTCTAGTTT | GGAG | 50 |
| | 9KH | 9KH22 | | | | | | | |
| 51 | SLUCAS | E51SLCas | 31774112 | 31774138 | + | 177 | AGATGGCATTTCTAGTTTGGAG | ATGG | 10 |
| | 9KH | 9KH23 | | | | | | | |
| 51 | SLUCAS | E51SLCas | 31774115 | 31774141 | + | 2046 | TGGCATTTCTAGTTTGGAGATG | GCAG | 22 |
| | 9KH | 9KH24 | | | | | | | |
| 51 | SLUCAS | E51SLCas | 31774124 | 31774150 | + | 2047 | TAGTTTGGAGATGGCAGTTTCC | TTAG | 20 |
| | 9KH | 9KH25 | | | | | | | |
| 51 | SLUCAS | E51SLCas | 31774133 | 31774159 | + | 2048 | GATGGCAGTTTCCTTAGTAACC | ACAG | 11 |
| | 9KH | 9KH26 | | | | | | | |
| 51 | SLUCAS | E51SLCas | 31774134 | 31774160 | + | 178 | ATGGCAGTTTCCTTAGTAACCA | CAGG | 14 |
| | 9KH | 9KH27 | | | | | | | |
| 51 | SLUCAS | E51SLCas | 31774146 | 31774172 | + | 2049 | TTAGTAACCACAGGTTGTGTCA | CCAG | 7 |
| | 9KH | 9KH28 | | | | | | | |
| 51 | SLUCAS | E51SLCas | 31774148 | 31774174 | + | 2050 | AGTAACCACAGGTTGTGTCACC | AGAG | 7 |
| | 9KH | 9KH29 | | | | | | | |
| 51 | SLUCAS | E51SLCas | 31774154 | 31774180 | + | 2051 | CACAGGTTGTGTCACCAGAGTA | ACAG | 14 |
| | 9KH | 9KH30 | | | | | | | |
| 51 | SLUCAS | E51SLCas | 31774160 | 31774186 | + | 2052 | TTGTGTCACCAGAGTAACAGTC | TGAG | 9 |
| | 9KH | 9KH31 | | | | | | | |
| 51 | SLUCAS | E51SLCas | 31774163 | 31774189 | + | 2053 | TGTCACCAGAGTAACAGTCTGA | GTAG | 9 |
| | 9KH | 9KH32 | | | | | | | |
| 51 | SLUCAS | E51SLCas | 31774164 | 31774190 | + | 179 | GTCACCAGAGTAACAGTCTGAG | TAGG | 14 |
| | 9KH | 9KH33 | | | | | | | |
| 51 | SLUCAS | E51SLCas | 31774166 | 31774192 | + | 2054 | CACCAGAGTAACAGTCTGAGTA | GGAG | 11 |
| | 9KH | 9KH34 | | | | | | | |
| 51 | SLUCAS | E51SLCas | 31774180 | 31774206 | + | 180 | TCTGAGTAGGAGCTAAAATATT | TTGG | 11 |
| | 9KH | 9KH35 | | | | | | | |
| 51 | SLUCAS | E51SLCas | 31774181 | 31774207 | + | 181 | CTGAGTAGGAGCTAAAATATTT | TGGG | 21 |
| | 9KH | 9KH36 | | | | | | | |
| 51 | SLUCAS | E51SLCas | 31774194 | 31774220 | + | 2055 | AAAATATTTTGGGTTTTTGCAA | AAAG | 56 |
| | 9KH | 9KH37 | | | | | | | |
| 51 | SLUCAS | E51SLCas | 31774195 | 31774221 | + | 182 | AAATATTTTGGGTTTTTGCAAA | AAGG | 61 |
| | 9KH | 9KH38 | | | | | | | |
| 51 | SLUCAS | E51SLCas | 31773927 | 31773953 | - | 2056 | GAAAAAATGATAAAAGTTGGCA | GAAG | 77 |
| | 9KH | 9KH39 | | | | | | | |
| 51 | SLUCAS | E51SLCas | 31773930 | 31773956 | - | 2057 | TGAGAAAAAATGATAAAAGTTG | GCAG | 124 |
| | 9KH | 9KH40 | | | | | | | |
| 51 | SLUCAS | E51SLCas | 31773933 | 31773959 | - | 183 | GTATGAGAAAAAATGATAAAAG | TTGG | 99 |
| | 9KH | 9KH41 | | | | | | | |
| 51 | SLUCAS | E51SLCas | 31773937 | 31773963 | - | 2058 | GAAGGTATGAGAAAAAATGATA | AAAG | 64 |
| | 9KH | 9KH42 | | | | | | | |
| 51 | SLUCAS | E51SLCas | 31773952 | 31773978 | - | 2059 | GATGATCATCAAGCAGAAGGTA | TGAG | 11 |
| | 9KH | 9KH43 | | | | | | | |
| 51 | SLUCAS | E51SLCas | 31773958 | 31773984 | - | 184 | CAACGAGATGATCATCAAGCAG | AAGG | 7 |
| | 9KH | 9KH44 | | | | | | | |
| 51 | SLUCAS | E51SLCas | 31773959 | 31773985 | - | 2060 | TCAACGAGATGATCATCAAGCA | GAAG | 3 |
| | 9KH | 9KH45 | | | | | | | |
| 51 | SLUCAS | E51SLCas | 31773962 | 31773988 | - | 2061 | ATATCAACGAGATGATCATCAA | GCAG | 7 |
| | 9KH | 9KH46 | | | | | | | |
| 51 | SLUCAS | E51SLCas | 31773965 | 31773991 | - | 2062 | AGGATATCAACGAGATGATCAT | CAAG | 7 |
| | 9KH | 9KH47 | | | | | | | |
| 51 | SLUCAS | E51SLCas | 31773977 | 31774003 | - | 2063 | TGGGTGACCTTGAGGATATCAA | CGAG | 17 |
| | 9KH | 9KH48 | | | | | | | |
| 51 | SLUCAS | E51SLCas | 31773988 | 31774014 | - | 185 | GAGGGTGATGGTGGGTGACCTT | GAGG | 22 |
| | 9KH | 9KH49 | | | | | | | |
| 51 | SLUCAS | E51SLCas | 31773989 | 31774015 | - | 2064 | AGAGGGTGATGGTGGGTGACCT | TGAG | 48 |
| | 9KH | 9KH50 | | | | | | | |
| 51 | SLUCAS | E51SLCas | 31773999 | 31774025 | - | 186 | ATAAAATCACAGAGGGTGATGG | TGGG | 27 |
| | 9KH | 9KH51 | | | | | | | |
| 51 | SLUCAS | E51SLCas | 31774000 | 31774026 | - | 187 | TATAAAATCACAGAGGGTGATG | GTGG | 23 |
| | 9KH | 9KH52 | | | | | | | |
| 51 | SLUCAS | E51SLCas | 31774003 | 31774029 | - | 188 | AGTTATAAAATCACAGAGGGTG | ATGG | 20 |
| | 9KH | 9KH53 | | | | | | | |
| 51 | SLUCAS | E51SLCas | 31774009 | 31774035 | - | 189 | TGATCAAGTTATAAAATCACAG | AGGG | 29 |
| | 9KH | 9KH54 | | | | | | | |
| 51 | SLUCAS | E51SLCas | 31774010 | 31774036 | - | 190 | TTGATCAAGTTATAAAATCACA | GAGG | 30 |
| | 9KH | 9KH55 | | | | | | | |
| 51 | SLUCAS | E51SLCas | 31774011 | 31774037 | - | 2065 | CTTGATCAAGTTATAAAATCAC | AGAG | 14 |
| | 9KH | 9KH56 | | | | | | | |
| 51 | SLUCAS | E51SLCas | 31774013 | 31774039 | - | 2066 | TGCTTGATCAAGTTATAAAATC | ACAG | 11 |
| | 9KH | 9KH57 | | | | | | | |
| 51 | SLUCAS | E51SLCas | 31774027 | 31774053 | - | 2067 | CGACTGGCTTTCTCTGCTTGAT | CAAG | 8 |
| | 9KH | 9KH58 | | | | | | | |
| 51 | SLUCAS | E51SLCas | 31774046 | 31774072 | - | 191 | GGGCTTGGACAGAACTTACCGA | CTGG | 2 |
| | 9KH | 9KH59 | | | | | | | |
| 51 | SLUCAS | E51SLCas | 31774060 | 31774086 | - | 2068 | GGCAGATTTCAACCGGGCTTGG | ACAG | 5 |
| | 9KH | 9KH60 | | | | | | | |
| 51 | SLUCAS | E51SLCas | 31774064 | 31774090 | - | 192 | CTCTGGCAGATTTCAACCGGGC | TTGG | 1 |
| | 9KH | 9KH61 | | | | | | | |
| 51 | SLUCAS | E51SLCas | 31774069 | 31774095 | - | 193 | ACCTGCTCTGGCAGATTTCAAC | CGGG | 10 |
| | 9KH | 9KH62 | | | | | | | |
| 51 | SLUCAS | E51SLCas | 31774070 | 31774096 | - | 194 | TACCTGCTCTGGCAGATTTCAA | CCGG | 10 |
| | 9KH | 9KH63 | | | | | | | |
| 51 | SLUCAS | E51SLCas | 31774081 | 31774107 | - | 2069 | GATGTTGGAGGTACCTGCTCTG | GCAG | 7 |
| | 9KH | 9KH64 | | | | | | | |
| 51 | SLUCAS | E51SLCas | 31774084 | 31774110 | - | 195 | CTTGATGTTGGAGGTACCTGCT | CTGG | 7 |
| | 9KH | 9KH65 | | | | | | | |
| 51 | SLUCAS | E51SLCas | 31774096 | 31774122 | - | 196 | AATGCCATCTTCCTTGATGTTG | GAGG | 22 |
| | 9KH | 9KH66 | | | | | | | |
| 51 | SLUCAS | E51SLCas | 31774097 | 31774123 | - | 2070 | AAATGCCATCTTCCTTGATGTT | GGAG | 19 |
| | 9KH | 9KH67 | | | | | | | |
| 51 | SLUCAS | E51SLCas | 31774099 | 31774125 | - | 197 | AGAAATGCCATCTTCCTTGATG | TTGG | 26 |
| | 9KH | 9KH68 | | | | | | | |
| 51 | SLUCAS | E51SLCas | 31774123 | 31774149 | - | 2071 | TAAGGAAACTGCCATCTCCAAA | CTAG | 41 |
| | 9KH | 9KH69 | | | | | | | |
| 51 | SLUCAS | E51SLCas | 31774144 | 31774170 | - | 198 | GGTGACACAACCTGTGGTTACT | AAGG | 6 |
| | 9KH | 9KH70 | | | | | | | |
| 51 | SLUCAS | E51SLCas | 31774145 | 31774171 | - | 2072 | TGGTGACACAACCTGTGGTTAC | TAAG | 10 |
| | 9KH | 9KH71 | | | | | | | |
| 51 | SLUCAS | E51SLCas | 31774153 | 31774179 | - | 199 | TGTTACTCTGGTGACACAACCT | GTGG | 22 |
| | 9KH | 9KH72 | | | | | | | |
| 51 | SLUCAS | E51SLCas | 31774168 | 31774194 | - | 200 | AGCTCCTACTCAGACTGTTACT | CTGG | 2 |
| | 9KH | 9KH73 | | | | | | | |
| 51 | SLUCAS | E51SLCas | 31774181 | 31774207 | - | 2073 | CCCAAAATATTTTAGCTCCTAC | TCAG | 10 |
| | 9KH | 9KH74 | | | | | | | |
| 51 | SLUCAS | E51SLCas | 31774192 | 31774218 | - | 2074 | TTTTGCAAAAACCCAAAATATT | TTAG | 43 |
| | 9KH | 9KH75 | | | | | | | |
| 53 | SACAS9 | E53SaCas | 31679352 | 31679380 | + | 1056 | AAAAGGTATCTTTGATACTAAC | CTTGGT | 45 |
| | KKH | 9KKH1 | | | | | | | |
| 53 | SACAS9 | E53SaCas | 31679361 | 31679389 | + | 1057 | CTTTGATACTAACCTTGGTTTC | TGTGAT | 20 |
| | KKH | 9KKH2 | | | | | | | |
| 53 | SACAS9 | E53SaCas | 31679373 | 31679401 | + | 25 | CCTTGGTTTCTGTGATTTTCTT | TTGGAT | 122 |
| | KKH | 9KKH3 | | | | | | | |
| 53 | SACAS9 | E53SaCas | 31679477 | 31679505 | + | 26 | TCCTTAGCTTCCAGCCATTGTG | TTGAAT | 42 |
| | KKH | 9KKH4 | | | | | | | |
| 53 | SACAS9 | E53SaCas | 31679510 | 31679538 | + | 1058 | AACATTTCATTCAACTGTTGCC | TCCGGT | 46 |
| | KKH | 9KKH5 | | | | | | | |
| 53 | SACAS9 | E53SaCas | 31679519 | 31679547 | + | 1059 | TTCAACTGTTGCCTCCGGTTCT | GAAGGT | 9 |
| | KKH | 9KKH6 | | | | | | | |
| 53 | SACAS9 | E53SaCas | 31679543 | 31679571 | + | 1060 | AGGTGTTCTTGTACTTCATCCC | ACTGAT | 16 |
| | KKH | 9KKH7 | | | | | | | |
| 53 | SACAS9 | E53SaCas | 31679550 | 31679578 | + | 27 | CTTGTACTTCATCCCACTGATT | CTGAAT | 58 |
| | KKH | 9KKH8 | | | | | | | |
| 53 | SACAS9 | E53SaCas | 31679565 | 31679593 | + | 28 | ACTGATTCTGAATTCTTTCAAC | TAGAAT | 78 |
| | KKH | 9KKH9 | | | | | | | |
| 53 | SACAS9 | E53SaCas | 31679577 | 31679605 | + | 1061 | TTCTTTCAACTAGAATAAAAGG | AAAAAT | 86 |
| | KKH | 9KKH10 | | | | | | | |
| 53 | SACAS9 | E53SaCas | 31679581 | 31679609 | + | 1062 | TTCAACTAGAATAAAAGGAAAA | ATAAAT | 420 |
| | KKH | 9KKH11 | | | | | | | |
| 53 | SACAS9 | E53SaCas | 31679588 | 31679616 | + | 1063 | AGAATAAAAGGAAAAATAAATA | TATAGT | 5266 |
| | KKH | 9KKH12 | | | | | | | |
| 53 | SACAS9 | E53SaCas | 31679346 | 31679374 | - | 1064 | GTTAGTATCAAAGATACCTTTT | TAAAAT | 31 |
| | KKH | 9KKH13 | | | | | | | |
| 53 | SACAS9 | E53SaCas | 31679359 | 31679387 | - | 1065 | CACAGAAACCAAGGTTAGTATC | AAAGAT | 33 |
| | KKH | 9KKH14 | | | | | | | |
| 53 | SACAS9 | E53SaCas | 31679368 | 31679396 | - | 1066 | AAAGAAAATCACAGAAACCAAG | GTTACT | 456 |
| | KKH | 9KKH15 | | | | | | | |
| 53 | SACAS9 | E53SaCas | 31679372 | 31679400 | - | 1067 | TCCAGAAAATCACAGAAAC | CAAGGT | 246 |
| | KKH | 9KKH16 | | | | | | | |
| 53 | SACAS9 | E53SaCas | 31679387 | 31679415 | - | 1068 | ATACAGTAGATGCAATCCAAAA | GAAAAT | 49 |
| | KKH | 9KKH17 | | | | | | | |
| 53 | SACAS9 | E53SaCas | 31679399 | 31679427 | - | 1069 | AGGAGGGTCCCTATACAGTAGA | TGCAAT | 13 |
| | KKH | 9KKH18 | | | | | | | |
| 53 | SACAS9 | E53SaCas | 31679404 | 31679432 | - | 1070 | ATGGAAGGAGGGTCCCTATACA | GTAGAT | 20 |
| | KKH | 9KKH19 | | | | | | | |
| 53 | SACAS9 | E53SaCas | 31679408 | 31679436 | - | 1071 | AGTCATGGAAGGAGGGTCCCTA | TACAGT | 27 |
| | KKH | 9KKH20 | | | | | | | |
| 53 | SACAS9 | E53SaCas | 31679419 | 31679447 | - | 29 | AGCCAAGCTTGAGTCATGGAAG | GAGGGT | 36 |
| | KKH | 9KKH21 | | | | | | | |
| 53 | SACAS9 | E53SaCas | 31679433 | 31679461 | - | 30 | TTAGGACAGGCCAGAGCCAAGC | TTGAGT | 30 |
| | KKH | 9KKH22 | | | | | | | |
| 53 | SACAS9 | E53SaCas | 31679462 | 31679490 | - | 1072 | TGGAAGCTAAGGAAGAAGCTGA | GCAGGT | 103 |
| | KKH | 9KKH23 | | | | | | | |
| 53 | SACAS9 | E53SaCas | 31679493 | 31679521 | - | 1073 | AATGAAATGTTAAAGGATTCAA | CACAAT | 200 |
| | KKH | 9KKH24 | | | | | | | |
| 53 | SACAS9 | E53SaCas | 31679503 | 31679531 | - | 31 | GCAACAGTTGAATGAAATGTTA | AAGGAT | 155 |
| | KKH | 9KKH25 | | | | | | | |
| 53 | SACAS9 | E53SaCas | 31679513 | 31679541 | - | 1074 | AGAACCGGAGGCAACAGTTGAA | TGAAAT | 11 |
| | KKH | 9KKH26 | | | | | | | |
| 53 | SACAS9 | E53SaCas | 31679518 | 31679546 | - | 32 | CCTTCAGAACCGGAGGCAACAG | TTGAAT | 8 |
| | KKH | 9KKH27 | | | | | | | |
| 53 | SACAS9 | E53SaCas | 31679523 | 31679551 | - | 1075 | GAACACCTTCAGAACCGGAGGC | AACAGT | 10 |
| | KKH | 9KKH28 | | | | | | | |
| 53 | SACAS9 | E53SaCas | 31679555 | 31679583 | - | 1076 | AAAGAATTCAGAATCAGTGGGA | TGAAGT | 106 |
| | KKH | 9KKH29 | | | | | | | |
| 53 | SACAS9 | E53SaCas | 31679560 | 31679588 | - | 33 | ACTTGAAAGAATTCAGAATCAG | TGGGAT | 110 |
| | KKH | 9KKH30 | | | | | | | |
| 53 | SACAS9 | E53SaCas | 31679565 | 31679593 | - | 1077 | ATTCTAGTTGAAAGAATTCAGA | ATCAGT | 134 |
| | KKH | 9KKH31 | | | | | | | |
| 53 | SACAS9 | E53SaCas | 31679569 | 31679597 | - | 34 | TTTTATTCTAGTTGAAAGAATT | CAGAAT | 399 |
| | KKH | 9KKH32 | | | | | | | |
| 53 | SACAS9 | E53SaCas | 31679576 | 31679604 | - | 35 | TTTTTCCTTTTATTCTAGTTGA | AAGAAT | 475 |
| | KKH | 9KKH33 | | | | | | | |
| 53 | SACAS9 | E53SaCas | 31679585 | 31679613 | - | 1078 | ATATATTTATTTTTCCTTTTAT | TCTAGT | 2133 |
| | KKH | 9KKH34 | | | | | | | |
| 53 | SLUCAS | E53SLCas | 31679353 | 31679379 | + | 201 | AAAGGTATCTTTGATACTAACC | TTGG | 13 |
| | 9KH | 9KH1 | | | | | | | |
| 53 | SLUCAS | E53SLCas | 31679373 | 31679399 | + | 202 | CCTTGGTTTCTGTGATTTTCTT | TTGG | 51 |
| | 9KH | 9KH2 | | | | | | | |
| 53 | SLUCAS | E53SLCas | 31679391 | 31679417 | + | 2075 | TCTTTTGGATTGCATCTACTGT | ATAG | 18 |
| | 9KH | 9KH3 | | | | | | | |
| 53 | SLUCAS | E53SLCas | 31679392 | 31679418 | + | 203 | CTTTTGGATTGCATCTACTGTA | TAGG | 12 |
| | 9KH | 9KH4 | | | | | | | |
| 53 | SLUCAS | E53SLCas | 31679393 | 31679419 | + | 204 | TTTTGGATTGCATCTACTGTAT | AGGG | 15 |
| | 9KH | 9KH5 | | | | | | | |
| 53 | SLUCAS | E53SLCas | 31679414 | 31679440 | + | 2076 | TAGGGACCCTCCTTCCATGACT | CAAG | 11 |
| | 9KH | 9KH6 | | | | | | | |
| 53 | SLUCAS | E53SLCas | 31679419 | 31679445 | + | 205 | ACCCTCCTTCCATGACTCAAGC | TTGG | 11 |
| | 9KH | 9KH7 | | | | | | | |
| 53 | SLUCAS | E53SLCas | 31679425 | 31679451 | + | 206 | CTTCCATGACTCAAGCTTGGCT | CTGG | 10 |
| | 9KH | 9KH8 | | | | | | | |
| 53 | SLUCAS | E53SLCas | 31679436 | 31679462 | + | 2077 | CAAGCTTGGCTCTGGCCTGTCC | TAAG | 16 |
| | 9KH | 9KH9 | | | | | | | |
| 53 | SLUCAS | E53SLCas | 31679446 | 31679472 | + | 2078 | TCTGGCCTGTCCTAAGACCTGC | TCAG | 18 |
| | 9KH | 9KH10 | | | | | | | |
| 53 | SLUCAS | E53SLCas | 31679458 | 31679484 | + | 2079 | TAAGACCTGCTCAGCTTCTTCC | TTAG | 16 |
| | 9KH | 9KH11 | | | | | | | |
| 53 | SLUCAS | E53SLCas | 31679465 | 31679491 | + | 2080 | TGCTCAGCTTCTTCCTTAGCTT | CCAG | 24 |
| | 9KH | 9KH12 | | | | | | | |
| 53 | SLUCAS | E53SLCas | 31679511 | 31679537 | + | 207 | ACATTTCATTCAACTGTTGCCT | CCGG | 15 |
| | 9KH | 9KH13 | | | | | | | |
| 53 | SLUCAS | E53SLCas | 31679519 | 31679545 | + | 2081 | TTCAACTGTTGCCTCCGGTTCT | GAAG | 6 |
| | 9KH | 9KH14 | | | | | | | |
| 53 | SLUCAS | E53SLCas | 31679520 | 31679546 | + | 208 | TCAACTGTTGCCTCCGGTTCTG | AAGG | 4 |
| | 9KH | 9KH15 | | | | | | | |
| 53 | SLUCAS | E53SLCas | 31679564 | 31679590 | + | 2082 | CACTGATTCTGAATTCTTTCAA | CTAG | 32 |
| | 9KH | 9KH16 | | | | | | | |
| 53 | SLUCAS | E53SLCas | 31679572 | 31679598 | + | 2083 | CTGAATTCTTTCAACTAGAATA | AAAG | 71 |
| | 9KH | 9KH17 | | | | | | | |
| 53 | SLUCAS | E53SLCas | 31679573 | 31679599 | + | 209 | TGAATTCTTTCAACTAGAATAA | AAGG | 48 |
| | 9KH | 9KH18 | | | | | | | |
| 53 | SLUCAS | E53SLCas | 31679589 | 31679615 | + | 2084 | GAATAAAAGGAAAAATAAATAT | ATAG | 574 |
| | 9KH | 9KH19 | | | | | | | |
| 53 | SLUCAS | E53SLCas | 31679592 | 31679618 | + | 2085 | TAAAAGGAAAAATAAATATATA | GTAG | 765 |
| | 9KH | 9KH20 | | | | | | | |
| 53 | SLUCAS | E53SLCas | 31679361 | 31679387 | - | 2086 | CACAGAAACCAAGGTTAGTATC | AAAG | 6 |
| | 9KH | 9KH21 | | | | | | | |
| 53 | SLUCAS | E53SLCas | 31679369 | 31679395 | - | 2087 | AAGAAAATCACAGAAACCAAGG | TTAG | 88 |
| | 9KH | 9KH22 | | | | | | | |
| 53 | SLUCAS | E53SLCas | 31679373 | 31679399 | - | 210 | CCAAAAGAAAATCACAGAAACC | AAGG | 52 |
| | 9KH | 9KH23 | | | | | | | |
| 53 | SLUCAS | E53SLCas | 31679374 | 31679400 | - | 2088 | TCCAAAAGAAAATCACAGAAAC | CAAG | 75 |
| | 9KH | 9KH24 | | | | | | | |
| 53 | SLUCAS | E53SLCas | 31679382 | 31679408 | - | 2089 | AGATGCAATCCAAAAGAAAATC | ACAG | 89 |
| | 9KH | 9KH25 | | | | | | | |
| 53 | SLUCAS | E53SLCas | 31679392 | 31679418 | - | 2090 | CCTATACAGTAGATGCAATCCA | AAAG | 4 |
| | 9KH | 9KH26 | | | | | | | |
| 53 | SLUCAS | E53SLCas | 31679406 | 31679432 | - | 2091 | ATGGAAGGAGGGTCCCTATACA | GTAG | 8 |
| | 9KH | 9KH27 | | | | | | | |
| 53 | SLUCAS | E53SLCas | 31679409 | 31679435 | - | 2092 | GTCATGGAAGGAGGGTCCCTAT | ACAG | 12 |
| | 9KH | 9KH28 | | | | | | | |
| 53 | SLUCAS | E53SLCas | 31679420 | 31679446 | - | 211 | GCCAAGCTTGAGTCATGGAAGG | AGGG | 10 |
| | 9KH | 9KH29 | | | | | | | |
| 53 | SLUCAS | E53SLCas | 31679421 | 31679447 | - | 212 | AGCCAAGCTTGAGTCATGGAAG | GAGG | 21 |
| | 9KH | 9KH30 | | | | | | | |
| 53 | SLUCAS | E53SLCas | 31679422 | 31679448 | - | 2093 | GAGCCAAGCTTGAGTCATGGAA | GGAG | 14 |
| | 9KH | 9KH31 | | | | | | | |
| 53 | SLUCAS | E53SLCas | 31679424 | 31679450 | - | 213 | CAGAGCCAAGCTTGAGTCATGG | AAGG | 18 |
| | 9KH | 9KH32 | | | | | | | |
| 53 | SLUCAS | E53SLCas | 31679425 | 31679451 | - | 2094 | CCAGAGCCAAGCTTGAGTCATG | GAAG | 18 |
| | 9KH | 9KH33 | | | | | | | |
| 53 | SLUCAS | E53SLCas | 31679428 | 31679454 | - | 214 | AGGCCAGAGCCAAGCTTGAGTC | ATGG | 23 |
| | 9KH | 9KH34 | | | | | | | |
| 53 | SLUCAS | E53SLCas | 31679434 | 31679460 | - | 2095 | TAGGACAGGCCAGAGCCAAGCT | TGAG | 17 |
| | 9KH | 9KH35 | | | | | | | |
| 53 | SLUCAS | E53SLCas | 31679440 | 31679466 | - | 2096 | AGGTCTTAGGACAGGCCAGAGC | CAAG | 23 |
| | 9KH | 9KH36 | | | | | | | |
| 53 | SLUCAS | E53SLCas | 31679445 | 31679471 | - | 2097 | TGAGCAGGTCTTAGGACAGGCC | AGAG | 27 |
| | 9KH | 9KH37 | | | | | | | |
| 53 | SLUCAS | E53SLCas | 31679447 | 31679473 | - | 2098 | GCTGAGCAGGTCTTAGGACAGG | CCAG | 35 |
| | 9KH | 9KH38 | | | | | | | |
| 53 | SLUCAS | E53SLCas | 31679451 | 31679477 | - | 215 | AGAAGCTGAGCAGGTCTTAGGA | CAGG | 44 |
| | 9KH | 9KH39 | | | | | | | |
| 53 | SLUCAS | E53SLCas | 31679452 | 31679478 | - | 2099 | AAGAAGCTGAGCAGGTCTTAGG | ACAG | 16 |
| | 9KH | 9KH40 | | | | | | | |
| 53 | SLUCAS | E53SLCas | 31679456 | 31679482 | - | 216 | AAGGAAGAAGCTGAGCAGGTCT | TAGG | 53 |
| | 9KH | 9KH41 | | | | | | | |
| 53 | SLUCAS | E53SLCas | 31679457 | 31679483 | - | 2100 | TAAGGAAGAAGCTGAGCAGGTC | TTAG | 25 |
| | 9KH | 9KH42 | | | | | | | |
| 53 | SLUCAS | E53SLCas | 31679463 | 31679489 | - | 217 | GGAAGCTAAGGAAGAAGCTGAG | CAGG | 70 |
| | 9KH | 9KH43 | | | | | | | |
| 53 | SLUCAS | E53SLCas | 31679464 | 31679490 | - | 2101 | TGGAAGCTAAGGAAGAAGCTGA | GCAG | 52 |
| | 9KH | 9KH44 | | | | | | | |
| 53 | SLUCAS | E53SLCas | 31679467 | 31679493 | - | 2102 | GGCTGGAAGCTAAGGAAGAAGC | TGAG | 41 |
| | 9KH | 9KH45 | | | | | | | |
| 53 | SLUCAS | E53SLCas | 31679472 | 31679498 | - | 2103 | ACAATGGCTGGAAGCTAAGGAA | GAAG | 27 |
| | 9KH | 9KH46 | | | | | | | |
| 53 | SLUCAS | E53SLCas | 31679475 | 31679501 | - | 2104 | AACACAATGGCTGGAAGCTAAG | GAAG | 18 |
| | 9KH | 9KH47 | | | | | | | |
| 53 | SLUCAS | E53SLCas | 31679478 | 31679504 | - | 218 | TTCAACACAATGGCTGGAAGCT | AAGG | 87 |
| | 9KH | 9KH48 | | | | | | | |
| 53 | SLUCAS | E53SLCas | 31679479 | 31679505 | - | 2015 | ATTCAACACAATGGCTGGAAGC | TAAG | 24 |
| | 9KH | 9KH49 | | | | | | | |
| 53 | SLUCAS | E53SLCas | 31679484 | 31679510 | - | 2016 | AAAGGATTCAACACAATGGCTG | GAAG | 12 |
| | 9KH | 9KH50 | | | | | | | |
| 53 | SLUCAS | E53SLCas | 31679487 | 31679513 | - | 219 | GTTAAAGGATTCAACACAATGG | CTGG | 12 |
| | 9KH | 9KH51 | | | | | | | |
| 53 | SLUCAS | E53SLCas | 31679491 | 31679517 | - | 220 | AAATGTTAAAGGATTCAACACA | ATGG | 37 |
| | 9KH | 9KH52 | | | | | | | |
| 53 | SLUCAS | E53SLCas | 31679505 | 31679531 | - | 221 | GCAACAGTTGAATGAAATGTTA | AAGG | 28 |
| | 9KH | 9KH53 | | | | | | | |
| 53 | SLUCAS | E53SLCas | 31679506 | 31679532 | - | 2107 | GGCAACAGTTGAATGAAATGTT | AAAG | 25 |
| | 9KH | 9KH54 | | | | | | | |
| 53 | SLUCAS | E53SLCas | 31679524 | 31679550 | - | 2108 | AACACCTTCAGAACCGGAGGCA | ACAG | 7 |
| | 9KH | 9KH55 | | | | | | | |
| 53 | SLUCAS | E53SLCas | 31679530 | 31679556 | - | 222 | TACAAGAACACCTTCAGAACCG | GAGG | 10 |
| | 9KH | 9KH56 | | | | | | | |
| 53 | SLUCAS | E53SLCas | 31679531 | 31679557 | - | 2109 | GTACAAGAACACCTTCAGAACC | GGAG | 10 |
| | 9KH | 9KH57 | | | | | | | |
| 53 | SLUCAS | E53SLCas | 31679533 | 31679559 | - | 223 | AAGTACAAGAACACCTTCAGAA | CCGG | 17 |
| | 9KH | 9KH58 | | | | | | | |
| 53 | SLUCAS | E53SLCas | 31679539 | 31679565 | - | 2110 | GGGATGAAGTACAAGAACACCT | TCAG | 18 |
| | 9KH | 9KH59 | | | | | | | |
| 53 | SLUCAS | E53SLCas | 31679550 | 31679576 | - | 2111 | TCAGAATCAGTGGGATGAAGTA | CAAG | 27 |
| | 9KH | 9KH60 | | | | | | | |
| 53 | SLUCAS | E53SLCas | 31679556 | 31679582 | - | 2112 | AAGAATTCAGAATCAGTGGGAT | GAAG | 27 |
| | 9KH | 9KH61 | | | | | | | |
| 53 | SLUCAS | E53SLCas | 31679562 | 31679588 | - | 224 | AGTTGAAAGAATTCAGAATCAG | TGGG | 95 |
| | 9KH | 9KH62 | | | | | | | |
| 53 | SLUCAS | E53SLCas | 31679563 | 31679589 | - | 225 | TAGTTGAAAGAATTCAGAATCA | GTGG | 33 |
| | 9KH | 9KH63 | | | | | | | |
| 53 | SLUCAS | E53SLCas | 31679566 | 31679592 | - | 2113 | TTCTACTTGAAAGAATTCAGAA | TCAG | 43 |
| | 9KH | 9KH64 | | | | | | | |
| 53 | SLUCAS | E53SLCas | 31679572 | 31679598 | - | 2114 | CTTTTATTCTAGTTGAAAGAAT | TCAG | 61 |
| | 9KH | 9KH65 | | | | | | | |
| 53 | SLUCAS | E53SLCas | 31679579 | 31679605 | - | 2115 | ATTTTTCCTTTTATTCTAGTTG | AAAG | 122 |
| | 9KH | 9KH66 | | | | | | | |
| 53 | SLUCAS | E53SLCas | 31679586 | 31679612 | - | 2116 | TATATTTATTTTTCCTTTTATT | CTAG | 978 |
| | 9KH | 9KH67 | | | | | | | |

### Example 4: Evaluation of sgRNA Pairs

A. Materials and Methods

1. sgRNA Selection

A subset of SaCas9-KKH or SluCas9 sgRNAs found within the DMD gene was selected for indel frequency and profile evaluation. The criteria used to select these sgRNAs included their potential to induce exon reframing and or skipping as a pair, in addition to the existence of a mouse, dog and NHP homologue counterpart. This selection included 27 sgRNAs located within exon 45, 39 sgRNAs located within exon 51 and 29 sgRNAs located within exon 53. The number of predicted off target sites was determined for each sgRNA.

2. Transfection of HEK293FT Cells

293FT cells were transfected in 12-well plates with 750 ng plasmid+2.25 uL of Lipofectamine 2000. Three days after transfection, cells were trypsinized and sorted for GFP. GFP-positive cells were sorted directly into lysis buffer, and DNA extraction was performed using the Promega Maxwell RSC Blood DNA Kit. PCR was then performed on the DNA using exon-specific primers that targeted the relevant cut site.

3. Amplicon Deep Sequencing Library Preparation and Data Analysis

The relevant loci for each exon were amplified by PCR and the products were used to prepare sequencing libraries using MiSeq reagent kit V3 600 cycle. Indel analysis was performed using CRISPResso2±10-nt quantification window. Indel profiling consisted of 8 indel categories listed in Table 6.

TABLE 6

| | | |
|---|---|---|
| Indel group | Ranking order | Definition |

Wild-type amplicon

| | | |
|---|---|---|
| NE | 1 | insertion = 0 & deletion = 0 |
| RF + 1 | 2 | 1-nt insertion at a reframe guide |
| RF Other | 3 | 3n + 1 deletion within the exon |
| | | boundary at the only reframe guide \| no |
| | | edits at the exon guide |
| | | aggregated 3n + 1 deletion within the |
| | | exon boundary at the two reframe guides |
| | | <17-nt insertion at the only reframe |
| | | guide \| no edits at the exon guide |
| | | <17-nt aggregated 3n + 1 insertion at the |
| | | two reframe guides |
| Exon | 4 | indels overlap with the splice acceptor |
| skipping | | (AG) (5′-end of the target exon) |
| | | deletion with ≥ 9-nt overlap with the |
| | | splicing window at one of the two guides |
| | | insertion at the exact GT/AG splicing sites |
| | | and with length ≥ 9-nt at one of the two |
| | | guides |
| OE | 5 | Other indels |

Deletion amplicon

| | | |
|---|---|---|
| Precise | 1 | segmental deletion between the two cut |
| deletion | | sites |
| (Rest) | 2 | SingleCut indel profiling with 3n phase |

Inversion amplicon

| | | |
|---|---|---|
| OE | 1 | all indels |

4. AAV Configurations for the Dual Cut Single Vector Candidates

A combination of promoter orientations, promoter configurations, NLSs and scaffolds were selected for generating AAV plasmids and evaluation on sgRNA transgene expression, AAV manufacturability, and editing efficiency in vitro and in vivo. AAV plasmid configurations listed in Table 7. Promoter, NLS and scaffold sequences listed in Table 8.

TABLE 7

| Pol III | | | | | | | |
|---|---|---|---|---|---|---|---|
| Promoter | Orientation | Configuration | NLS1 | Endonuclease | NLS2 | NLS3 | Scaffold |
| hU6c:hU6c | ← [symbol] → | hU6c-Cas9- | c-Myc- | SluCas9 | SV-40-GSGS | Nucleoplasmin- | V5 |
| | | hU6c | GSVD | | | GSGS | |
| | ← [symbol] → | hU6c-Cas9- | c-Myc- | SaCas9-KKH | SV-40-GSGS | Nucleoplasmin- | V2 |
| | | hU6c | GSVD | | | GSGS | |
| | ← [symbol] → | hU6c-Cas9- | c-Myc- | SluCas9 | SV-40-MCS- | Nucleoplasmin- | V5 |
| | | hU6c | GSVD | | GSGS | GSGS | |
| | ← [symbol] → | hU6c-Cas9- | c-Myc- | SaCas9-KKH | SV-40-MCS- | Nucleoplasmin- | V2 |
| | | hU6c | GSVD | | GSGS | GSGS | |
| hU6:7SK2 | ← [symbol] → | hU6c-Cas9- | c-Myc- | SluCas9 | SV-40-GSGS | Nucleoplasmin- | V5 |
| | | 7SK2 | GSVD | | | GSGS | |
| | ← [symbol] → | hU6c-Cas9- | c-Myc- | SaCas9-KKH | SV-40-GSGS | Nucleoplasmin- | V2 |
| | | 7SK2 | GSVD | | | GSGS | |
| | ← [symbol] → | hU6c-Cas9- | c-Myc- | SluCas9 | SV-40-MCS- | Nucleoplasmin- | V5 |
| | | 7SK2 | GSVD | | GSGS | GSGS | |
| | ← [symbol] → | hU6c-Cas9- | c-Myc- | SaCas9-KKH | SV-40-MCS- | Nucleoplasmin- | V2 |
| | | 7SK2 | GSVD | | GSGS | GSGS | |
| hU6c:H1m | ← [symbol] → | hU6c-Cas9- | c-Myc- | SluCas9 | SV-40-GSGS | Nucleoplasmin- | V5 |
| | | H1m | GSVD | | | GSGS | |
| | ← [symbol] → | hU6c-Cas9- | c-Myc- | SaCas9-KKH | SV-40-GSGS | Nucleoplasmin- | V2 |
| | | H1m | GSVD | | | GSGS | |
| 7SK2:H1m | ← [symbol] → | 7SK2-Cas9- | SV-40- | SluCas9 | Nucleoplasmin | N/A | V5 |
| | | H1m | GS | | | | |
| | ← [symbol] → | 7SK2-Cas9- | SV-40- | SaCas9-KKH | Nucleoplasmin | N/A | V2 |
| | | H1m | GS | | | | |
| | ← [symbol] → | 7SK2-Cas9- | SV-40 | SluCas9 | Nucleoplasmin | N/A | V5 |
| | | H1m | (+) | | | | |
| | ← [symbol] → | 7SK2-Cas9- | SV-40 | SaCas9-KKH | Nucleoplasmin | N/A | V2 |
| | | H1m | (+) | | | | |
| | ← [symbol] → | 7SK2-Cas9- | c-Myc- | SluCas9 | SV-40-MCS- | Nucleoplasmin- | V5 |
| | | H1m | GSVD | | GSGS | GSGS | |
| | ← [symbol] → | 7SK2-Cas9- | c-Myc- | SaCas9-KKH | SV-40-MCS- | Nucleoplasmin- | V2 |
| | | H1m | GSVD | | GSGS | GSGS | |
| | ← [symbol] → | 7SK2-Cas9- | c-Myc- | SluCas9 | SV-40-MCS- | Nucleoplasmin- | V2 |
| | | H1m | GSVD | | GSGS | GSGS | |
| H1m:M11 | ← [symbol] → | H1m-Cas9- | c-Myc- | SluCas9 | SV-40-MCS- | Nucleoplasmin- | V5 |
| | | M11 | GSVD | | GSGS | GSGS | |
| | ← [symbol] → | H1m-Cas9- | c-Myc- | SaCas9-KKH | SV-40-MCS- | Nucleoplasmin- | V5 |
| | | M11 | GSVD | | GSGS | GSGS | |

TABLE 8

| Promoter | Sequences |
|---|---|
| hU6c | GAGGGCCTATTTCCCATGATTCCTTCATATTT |
| | GCATATACGATACAAGGCTGTTAGAGAGATAA |
| | TTGGAATTAATTTGACTGTAAACACAAAGATA |
| | TTAGTACAAAATACGTGACGTAGAAAGTAATA |
| | ATTTCTTGGGTAGTTTGCAGTTTTAAAATTAT |
| | GTTTTAAAATGGACTATCATATGCTTACCGTA |
| | ACTTGAAAGTATTTCGATTTCTTGGCTTTATA |
| | TATCTTGTGGAAAGGACGAAACACC |
| | (SEQ ID NO: 705) |
| 7SK2 | CTGCAGTATTTAGCATGCCCCACCCATCTGCA |
| | AGGCATTCTGGATAGTGTCAAAACAGCCGGAA |
| | ATCAAGTCCGTTTATCTCAAACTTTAGCATTT |
| | TGGGAATAAATGATATTTGCTATGCTGGTTAA |
| | ATTAGATTTTAGTTAAATTTCCTGCTGAAGCT |
| | CTAGTACGATAAGCAACTTGACCTAAGTGTAA |
| | AGTTGAGACTTCCTTCAGGTTTATATAGCTTG |
| | TGCGCCGCTTGGGTACCTC |
| | (SEQ ID NO: 706) |
| H1m | AATATTTGCATGTCGCTATGTGTTCTGGGAAA |
| | TCACCATAAACGTGAAATGTCTTTGGATTTGG |
| | GAATCTTATAAGTTCTGTATGAGACCACTCTT |
| | TCCC |
| | (SEQ ID NO: 707) |
| M11 | ATATTTAGCATGTCGCTATGTGTTCTGGGAAA |
| | CTTGACCTAAGTGTAAAGTTGAGATTTCCTTC |
| | AGGTTTATATAGTTCTGTATGAGACCACTCTT |
| | TCCC |
| | (SEQ ID NO: 708) |
| NLS-linker | AA Sequences |
| c-Myc-GSVD | PAAKKKKLDGSVD (SEQ ID NO: 36) |
| SV-40-GS | PKKKRKVGS (SEQ ID NO: 37) |
| SV-40 (+) | PKKKRKVGIHGVPAA (SEQ ID NO: 38) |
| SV-40-GSGS | GSGSPKKKRKV (SEQ ID NO: 39) |
| SV-40- | TGGGPGGGAAAGSGSPKKKRKV |
| MCS-GSGS | (SEQ ID NO: 40) |
| Nucleoplasmin- | GSGSKRPAATKKAGQAKKKK |
| GSGS | (SEQ ID NO: 41) |
| Scaffold | Sequences |
| SluCas9 | GUUUCAGUACUCUGGAAACAGAAUCUACUGAA |
| scaffold V5 | ACAAGACAAUAUGUCGUGUUUAUCCCAUCAAU |
| | UUAUUGGUGGGAU |
| | (SEQ ID NO: 922) |
| SluCas9 | GTTTAAGTACTCTGTGCTGGAAACAGCACAGA |
| scaffold V2 | ATCTACTTAAACAAGGCAAAATGCCGTGTTTA |
| | TCTCGTCAACTTGTTGGCGAGA |
| | (SEQ ID NO: 500) |

Sequences of selected primers used for amplification of the specific human locus containing the sgRNA sites are shown in Table 9.

TABLE 9

| Name | Sequence |
|---|---|
| MiSeq_hE45_F | TCGTCGGCAGCGTCAGATGTGTATAAGAG |
| | ACAGGTCTTTCTGTCTTGTATCCTTTGG |
| | (SEQ ID NO: 724) |
| MiSeq_hE45_R | GTCTCGTGGGCTCGGAGATGTGTATAAGA |
| | GACAGAATGTTAGTGCCTTTCACCC |
| | (SEQ ID NO: 725) |
| TIDE_hE45_F* | GTCTTTCTGTCTTGTATCCTTTGG |
| | (SEQ ID NO: 726) |

*TIDE_hE45_F is used for Sanger sequencing.

5. Results

A set of sgRNAs found within the DMD gene was selected for evaluation of indel frequency and profile. Among this selection, 27 sgRNAs were located within exon 45, 39 sgRNAs were located within exon 51 and 29 sgRNAs were located within exon 53. To evaluate indel frequency and editing profiles, plasmid transfection was performed in HEK293FT (FIG. 6B) and Neuro-2a cell lines (FIG. 6D). The average indel frequency of sgRNAs targeting exon 45 was determined in HEK293FT cells (FIG. 6B) and in Neuro-2a cells (FIG. 6D), with a high-performing SpCas9 sgRNA (E45Sp52) included as a reference. Of these, 20 sgRNAs pairs showed an average total indel frequency higher than 60% and 9 sgRNA pairs showed an average precise segmental deletion frequency higher than 50% in 293FT cells. Five sgRNAs tested in the Neuro-2a cell line showed an average precise segmental deletion frequency higher than 40% (FIG. 6D). With regard to indel profile, sgRNA pairs E45SaKKH10/20, E45Slu21/7 and E45Slu18/4 were found to have the highest percent of precise segmental deletion that could lead to potential exon reframing in 293FT cells (FIG. 6B), and sgRNA pairs E45SaKKH10/23, E45Slu17/22, E45Slu17/23 and E45Slu19/21 were found to have the highest percent of precise segmental deletion that could lead to potential exon reframing in Neuro-2a cells (FIG. 6D). Table 10 shows the SEQ ID NOs corresponding to the sgRNA identifiers in FIGS. 6B and 6C.

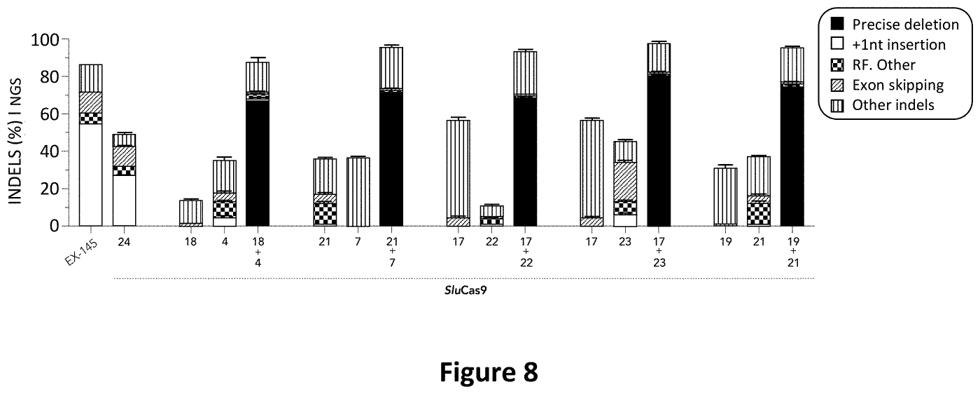

TABLE 10

| sgRNA identifier | |
|---|---|
| in certain | |
| Figures, including | |
| FIG. 6 and FIG. 8 | SEQ ID NO(s) |
| SaCas9: 2 and 7 | 10 and 15 |
| SaCas9: 2 and 8 | 10 and 16 |
| SaCas9: 4 and 8 | 12 and 16 |
| SaCas9-KKH: 2 and 9 | 1001 and 1005 |
| SaCas9-KKH: 2 and 26 | 1001 and 15 |
| SaCas9-KKH: 2 and 27 | 1001 and 16 |
| SaCas9-KKH: 7 and 9 | 1003 and 1005 |
| SaCas9-KKH: 27 and 7 | 16 and 1003 |
| SaCas9-KKH: 10 and 16 | 12 and 1010 |
| SaCas9-KKH: 10 and 18 | 12 and 1012 |
| SaCas9-KKH: 10 and 20 | 12 and 1013 |
| SaCas9-KKH: 10 and 23 | 10 and 1016 |
| SaCas9-KKH: 24 and 9 | 1017 and 1005 |
| SaCas9-KKH: 24 and 27 | 1017 and 16 |
| SaCas9-KKH: 25 and 27 | 1018 and 16 |
| SluCas9: 21 and 7 | 148 and 134 |
| SluCas9: 22 and 8 | 149 and 135 |
| SluCas9: 23 and 8 | 150 and 135 |
| SluCas9: 4 and 9 | 131 and 136 |
| SluCas9: 24 and 9 | 151 and 136 |
| SluCas9: 12 and 4 | 139 and 131 |
| SluCas9: 12 and 24 | 139 and 151 |
| SluCas9: 13 and 4 | 140 and 131 |
| SluCas9: 13 and 24 | 140 and 151 |
| SluCas9: 14 and 21 | 141 and 148 |
| SluCas9: 17 and 22 | 144 and 149 |
| SluCas9: 17 and 23 | 144 and 150 |
| SluCas9: 18 and 4 | 145 and 131 |
| SluCas9: 18 and 24 | 145 and 151 |
| SluCas9: 19 and 21 | 146 and 148 |
| SaCas9-4 | 12 |
| SluCas9-24 | 151 |
| EX-145 | SpCas9 control |
| | (ATCTTACAGGAACTCCAGGA) |
| | (SEQ ID NO: 727) |

### Example 5: Testing of sgRNA Scaffold Sequences

1. Materials and Methods

Primary human skeletal muscle myoblasts (HsMM; Lonza CC-2580: lot #20TL070666, P0) were recovered and passaged in SkBM®-2 Skeletal Muscle Myoblast Basal Medium plus SkGM®-2 SingleQuots (CC-3246, CC-3244; Lonza) in the incubator at 37° C. with 5% CO2. When HsMM culture reached approximately 80% to 90% confluence and were actively proliferating, the cells were harvested for SluCas9 ribonucleoprotein (RNP) delivery. After thawing, the cells were passaged once before SluCas9 RNP delivery.

To form SluCas9 RNPs, the appropriate amount of synthetic sgRNA (Synthego: SO #7292552) and recombinant SluCas9 protein (Aldevron: Lot #M22536-01) were mixed in supplemented P5 Primary Cell nucleofection solution (Lonza V4XP-5032). In total, three sgRNA:SluCas9 doses were tested, including a low dose with 37.5 pmol:6.25 pmol, a middle dose 75 pmol:12.5 pmol, and a high dose 150:25. The sgRNAs and SluCas9 proteins were incubated for at least 10 minutes at room temperature for Cas9-sgRNA RNP formation.

While the SluCas9 RNP was forming, HsMMs were rinsed with HEPES buffered saline solution, dissociated from tissue culture flasks by trypsin, and centrifuge at 90×g for 10 minutes. The cell pellets were resuspended in fresh, pre-warmed, complete growth medium. The number of cells were counted. Appropriate number of cells were transfer into a new centrifuge tube, pelleted by centrifugation at 90×g for 10 minutes, and resuspended in supplemented nucleofection solution. About 200,000 cells in 15 μl nucleofection solution were mixed with about 7 μl of preformed SluCas9:sgRNA RNP complex.

Approximately 20 μl of the cell and RNP mix were transferred into a 16-well nucleofection strip tube, and then nucleofected with the DS-158 program in a 4D-Nucleofector (Lonza) Immediately after nucleofection, about 80 μl pre-warmed media were added into the each nucleocuvette and incubated at 37 degrees for 10 minutes. The contents (100 μl) of each nucleocuvette were transferred into one well in a 12-well plate filled with 2 ml media and incubated at 37 degrees for 48 hours.

To determine cell viability 48 hours after nucleofection, the cells were stained with Hoechst and Propidium Iodide (Life Technologies). Cell viability was then assessed using ImageXpress Micro (Molecular Devices). In general, samples with an overall cell viability above 70% were harvested and analyzed for indel analysis.

To isolate genomic DNA from HsMMs, the cells were washed with saline buffer, trypsinized and centrifuged. The cell pellets were treated with lysis buffer from the Maxwell RSC Blood DNA Kit (Promega #AS1400), and genomic DNAs were extracted using a Maxwell® RSC48 instrument (Promega #AS8500) according to the manufacturer's instruction. The concentrations of genomic DNAs were determined using Qubit™ 1× dsDNA HS Assay Kit (Thermo Fisher Scientific Q33231) according to the manufacturer's instruction.

To determine the gene editing efficiency, the genomic DNAs were amplified using primers flanking the DMD exon 45 genomic region. The following primer sequences were used: MiSeq_hE45_F TCGTCGGCAGCGTCAGATGTGTATAAGAGACAGgtattctgtettgtatcctttgg (SEQ ID NO: 724) and MiSeq_hE45_R GTCTCGTGGGCTCGGAGATGTGTATAAGAGACAGaatgttagtgcctttcaccc (SEQ ID NO: 725). The size of the amplicons was verified by analyze a small amount of the PCR products on 2% E-gels (Thermo Fisher Scientific). A portion of the PCR product and the forward primer were then sent for sanger sequencing at Genewiz.

The sequencing results that pass the quality filter were used to determine editing efficiency and indel profile using the TIDE (Tracking of Indels by DEcomposition) algorithm. The following version of the algorithm was used for analysis https://shiny.vrtx.com/app/orrj/tide/ with the appropriate sgRNA sequence, the default analysis parameters, and a mock nucleofected sample as the control. Percentage of other insertions and deletions that have the potential to restore the reading frame of particular DMD patient mutations of interest were referred to as “RF other”. This represents the sum of 2, 5, 8, 11 bp deletions within the alignment window of −20 bp to +20 bp around the Cas9 cut site. The editing efficiency (% mutation) outputs for +1 bp insertion, RF. Other, and other indels from TIDE were then plotted using Prism 9.

2. Results

To optimize and improve gene editing efficiency of the top SluCas9 single-guide RNA (sgRNA) candidates, different sgRNA scaffold sequences were tested in primary human skeletal muscle myoblasts (HsMM) using synthetic sgRNAs as shown in Table 11 below. Two spacer sequences were tested: E45SL23 (SEQ ID NO: 150 (DNA); SEQ ID NO: 930 (RNA)) and E45SL24 (SEQ ID NO: 151 (DNA); SEQ ID NO: 931 (RNA)). Three scaffold sequences were tested: Slu-VCGT-4.5 (SEQ ID NO: 601 (DNA); SEQ ID NO: 918 (RNA)), Slu-VCGT-4 (SEQ ID NO: 917 (DNA); SEQ ID NO: 919 (RNA)), Slu-VCGT-5 (SEQ ID NO: 901 (DNA); SEQ ID NO: 920 (RNA)).

TABLE 11
Exemplary sgRNAs for testing

| | Scaffold | | Spacer | Spacer | SEQ ID | |
|---|---|---|---|---|---|---|
| | (SEQ ID | Scaffold | SEQ | (RNA | NO of | |
| sgRNA ID | NO of RNA) | (RNA version) | ID NO | version) | sgRNA | sgRNA sequence |
| E45SL23- | Slu-VCGT- | GUUUAAGUACUCU | 930 | GGUA | 924 | GGUAUCUUACAGGAA |
| VCGT-4.5 | 4.5 | GUGCUGGAAACAG | | UCUU | | CUCCAGGGUUUAAGU |
| | (SEQ ID | CACAGAAUCUACU | | ACAG | | ACUCUGUGCUGGAAA |
| | NO: 918) | GAAACAAGACAAU | | GAAC | | CAGCACAGAAUCUAC |
| | | AUGUCGUGUUUAU | | UCCA | | UGAAACAAGACAAUA |
| | | CCCAUCAAUUUAU | | GG | | UGUCGUGUUUAUCCC |
| | | UGGUGGGA | | | | AUCAAUUUAUUGGUG |
| | | | | | | GGA |
| E45SL24- | Slu-VCGT- | GUUUAAGUACUCU | 931 | UUUU | 925 | UUUUGGUAUCUUACA |
| VCGT-4.5 | 4.5 | GUGCUGGAAACAG | | GGUA | | GGAACUCGUUUAAGU |
| | (SEQ ID | CACAGAAUCUACU | | UCUU | | ACUCUGUGCUGGAAA |
| | NO: 919) | GAAACAAGACAAU | | ACAG | | CAGCACAGAAUCUAC |
| | | AUGUCGUGUUUAU | | GAAC | | UGAAACAAGACAAUA |
| | | CCCAUCAAUUUAU | | UC | | UGUCGUGUUUAUCCC |
| | | UGGUGGGA | | | | AUCAAUUUAUUGGUG |
| | | | | | | GGA |
| E45SL23- | Slu-VCGT- | GUUUCAGUACUCU | 930 | GGUA | 926 | GGUAUCUUACAGGAA |
| VCGT-4 | 4 | GUGCUGGAAACAG | | UCUU | | CUCCAGGGUUUCAGU |
| | (SEQ ID | CACAGAAUCUACU | | ACAG | | ACUCUGUGCUGGAAA |
| | NO: 920) | GAAACAAGACAAU | | GAAC | | CAGCACAGAAUCUAC |
| | | AUGUCGUGUUUAU | | UCCA | | UGAAACAAGACAAUA |
| | | CCCAUCAAUUUAU | | GG | | UGUCGUGUUUAUCCC |
| | | UGGUGGGAU | | | | AUCAAUUUAUUGGUG |
| | | | | | | GGAU |
| E45SL24- | Slu-VCGT- | GUUUCAGUACUCU | 931 | UUUU | 927 | UUUUGGUAUCUUACA |
| VCGT-4 | 4 | GUGCUGGAAACAG | | GGUA | | GGAACUCGUUUCAGU |
| | (SEQ ID | CACAGAAUCUACU | | UCUU | | ACUCUGUGCUGGAAA |
| | NO: 921) | GAAACAAGACAAU | | ACAG | | CAGCACAGAAUCUAC |
| | | AUGUCGUGUUUAU | | GAAC | | UGAAACAAGACAAUA |
| | | CCCAUCAAUUUAU | | UC | | UGUCGUGUUUAUCCC |
| | | UGGUGGGAU | | | | AUCAAUUUAUUGGUG |
| | | | | | | GGAU |
| E45SL23- | Slu-VCGT- | GUUUcAGUACUCU | 930 | GGUA | 928 | GGUAUCUUACAGGAA |
| VCGT-5 | 5 | GGAAACAGAAUCU | | UCUU | | CUCCAGGGUUUcAGU |
| | (SEQ ID | ACUGAAACAAGAC | | ACAG | | ACUCUGGAAACAGAA |
| | NO: 922) | AAUAUGUCGUGUU | | GAAC | | UCUACUGAAACAAGA |
| | | UAUCCCAUCAAUU | | UCCA | | CAAUAUGUCGUGUUU |
| | | UAUUGGUGGGAU | | GG | | AUCCCAUCAAUUUAU |
| | | | | | | UGGUGGGAU |
| E45SL24- | Slu-VCGT- | GUUUcAGUACUCU | 931 | UUUU | 929 | UUUUGGUAUCUUACA |
| VCGT-5 | 5 | GGAAACAGAAUCU | | GGUA | | GGAACUCGUUUcAGU |
| | (SEQ ID | ACUGAAACAAGAC | | UCUU | | ACUCUGGAAACAGAA |
| | NO: 923) | AAUAUGUCGUGUU | | ACAG | | UCUACUGAAACAAGA |
| | | UAUCCCAUCAAUU | | GAAC | | CAAUAUGUCGUGUUU |
| | | UAUUGGUGGGAU | | UC | | AUCCCAUCAAUUUAU |
| | | | | | | UGGUGGGAU |

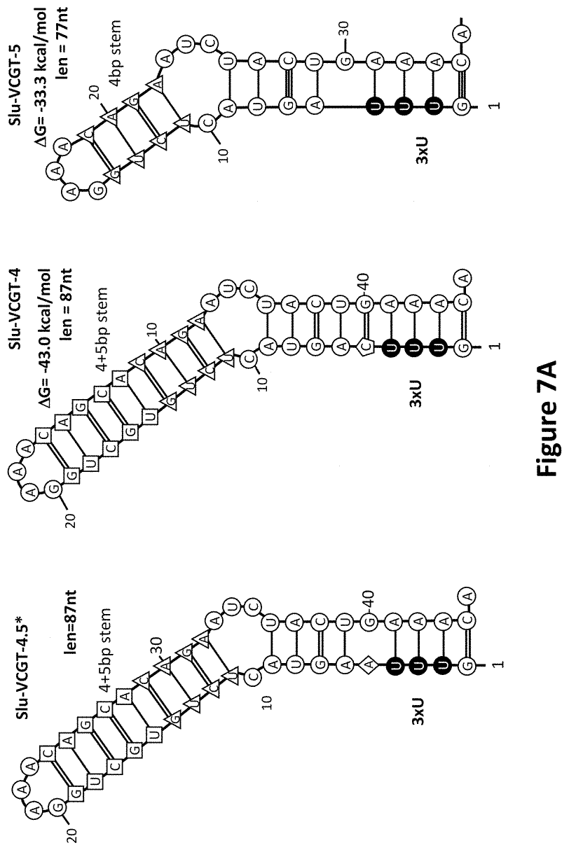

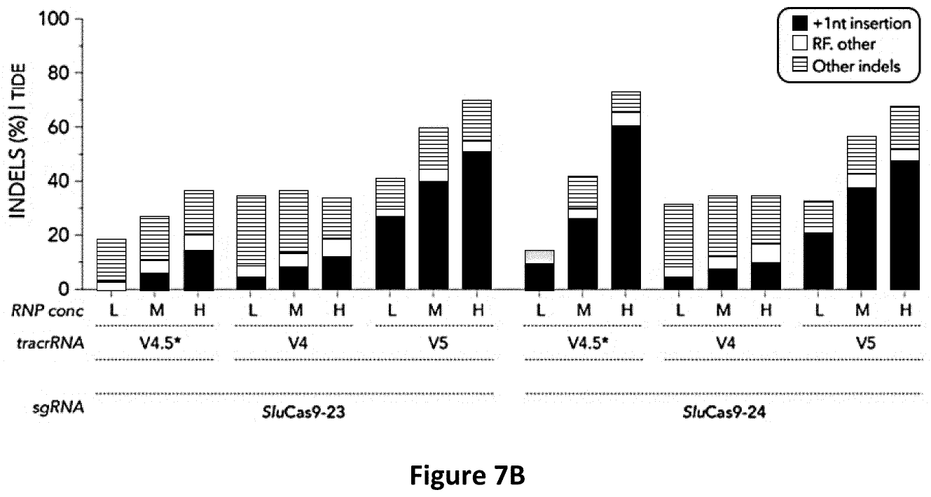

The three scaffold sequences differ by the nucleotide identity, and thus the stem-loop I in RNA secondary structure (FIG. 7A). In addition to differences in stem-loop I, Slu-VCGT-4.5 lacks the last nucleotide U at the 3′ end of Stem 3 (not shown). The results indicate that, Slu-VCGT-5 scaffold produces higher editing efficiency compared to guides with a V4 or V4.5 scaffold in most conditions tested (shown in FIG. 7B).

### Example 6

1. Materials and Methods

Transfection of HEK293FT Cells

293FT cells were transfected in 12-well plates with 750 ng plasmid+2.25 μL of Lipofectamine 2000. Three days after transfection, cells were trypsinized and sorted for GFP. GFP-positive cells were sorted directly into lysis buffer, and DNA extraction was performed using the Promega Maxwell RSC Blood DNA Kit. PCR was then performed on the DNA using exon-specific primers that targeted the relevant cut site.

Transfection of N2a Cells

N2a (Neuro2a) cells were transfected in 12-well plates after 24-hour of growth with 1000 ng plasmid+3 μL of Lipofectamine 2000. Three days after transfection, cell were trypsinized and sorted for GFP (green fluroescent protein). GFP-positive cells were sorted via FACS (flurorescence-activated cell sorting) directly into lysis buffer, and DNA extraction was performed using the Promega Maxwell RSC Blood DNA Kit. PCR was then performed on the DNA using exon-specific primers that targeted the relevant cut site.

Amplicon Deep Sequencing Library Preparation for HEK293FT Cells

Genomic DNA were extracted after sorting for 100 k GFP positive cells and were subjected for amplification by locus specific amplicon. The amplified products were purified by AMPure beads (0.8×) followed by QC with 1% E-gel and concentrations were measured using QuBiT for normalization of samples. Barcoding PCR was carried out with i5 and i7 indices and they were purified by 0.7× AMPure beads followed by QC with Tapestation or 1% E-gel. The barcoded samples were then measured by QuBiT and based on the concentration, the samples were pooled and subjected for library preparation for loading into MiSeq. 8 pM of library was loaded with 20% PhiX spike-in and the output were transferred to the Computational Genomics team for assessing the indel efficiencies for the guides.

Human Primary Skeletal Muscle Myoblasts (HsMMs) Culture

On day zero, two frozen vials of HsMMs (lot 20TL070666) was thawed and grown in complete growth media in a 37° C., 5% CO2, humidified incubator in two T75 flasks. The following day, on day 1, the media was changed. On day 3, the cells were passaged such that there were 9x10 cells seeded into 7 T175 flasks. On day 4, the media was changed in each flask. On day 6, RNP nucleofection was performed.

Nucleofection of HsMM Cells

HsMM cells were nucleofected with RNP using the Lonza 4D nucleofector. For each sample, 7 μL of RNP were combined with 0.3e6 cells in 15 μL P5 solution. RNP was prepared in a 6:1 gRNA:Cas9 ratio at various concentrations. For dual-cut samples that contain 2 gRNAs, RNP was pre-formed with a single gRNA first. RNP was formed by incubating gRNA and protein for 20 min at room temperature. After electroporation, 80 μL of complete growth media was added to each sample and samples were incubated in a 37° C., 5% CO2, humidified incubator. After 10 minutes, the samples were transferred to 12-well plates containing 2 mL of complete growth media that had been previously equilibrated in a 37° C., 5% CO2, humidified incubator.

Cell Harvesting and gDNA Extraction

To determine cell viability 48 hours after nucleofection, the cells were stained with Hoechst and Propidium Iodide (Life Technologies). Cell viability was then assessed using ImageXpress Micro (Molecular Devices). In general, samples with an overall cell viability above 70% were harvested and analyzed for indel analysis. To isolate genomic DNA from HsMMs, the cells were washed with saline buffer, trypsinized and centrifuged. The cell pellets were treated with lysis buffer from the Maxwell RSC Blood DNA Kit (Promega #AS1400), and genomic DNAs were extracted using a Maxwell® RSC48 instrument (Promega #AS8500) according to the manufacturer's instruction. The concentrations of genomic DNAs were determined using Qubit™ 1× dsDNA HS Assay Kit (Thermo Fisher Scientific Q33231) according to the manufacturer's instruction.

Amplicon Deep Sequencing Library Preparation for HsMM

To determine the gene editing efficiency, the genomic DNAs were amplified using primers flanking the DMD exon 45 genomic region. The following primer sequences were used: MiSeq_hE45_F TCGTCGGCAGCGTCAGATGTGTATAAGAGACAGgtctttctgtcttgtatcctttgg (SEQ ID NO: 724) and MiSeq_hE45_R GTCTCGTGGGCTCGGAGATGTGTATAAGAGACAGaatgttagtgcctttcaccc (SEQ ID NO: 725).

The size of the amplicons was verified by analyze a small amount of the PCR products on 2% E-gels (Thermo Fisher Scientific). The PCR product was purified by AMPure XP beads (A63881). The purified PCR product was amplified again with primers that contain barcodes and Illumina adaptors. Multiple barcoded samples were pooled, combined with PhiX library, and loaded onto the Illumina Mi-Seq platform. MiSeq Reagent Kit v3 (MS-102-3003) was used to produce a 600-cycle run.

Maintenance and Differentiation of C2C12 Myotubes

C2C12 are maintained in DMEM supplemented with 10% FBS (Fetal Bovine Serum) and 1% Pen/Strep. For culture purposes, cells should not be allowed to reach >75% confluency. C2C12 myoblasts are differentiated in DMEM supplemented with 2% HS and 1% Pen/Strep. Briefly, cells are seeded at 42-45 k/cm² and differentiation is started 24 hr after seeding (once cells reach ˜90-95% confluency). Differentiation medium is changed on day 1 and then refreshed every other day.

Transduction of C2C12 Myotubes

C2C12 myoblasts were allowed to differentiate to myotubes in differentiation medium (DMEM with 2% horse serum and 1% Pen/Strep) for 6 days. Two hours before viral transduction, myotubes were treated with Neuraminidase type III (Sigma-Aldrich, 50 mU/ml), followed by washing with differentiation medium twice. Myotubes were incubated with AAV (MOI 1.0E7) and centrifuged at 1000×g at 4 C for 1.5 hours. After spin transduction, the virus is aspirated, and the myotubes are washed with cold PBS once followed by two washes with differentiation medium. The myotubes are cultured in differentiation medium for an additional week (7 days) before being harvested for INDEL analysis (ICE/NGS) or fixed for immunohistochemistry analysis (IHC). DNA/RNA extraction was performed using the Qiagen AllPrep DNA/RNA Minikit

Cas9 Nuclear Localization by Immunofluorescence Staining of C2C12 Myotubes

7 days post AAV transduction (day 12 of myotube differentiation) C2C12 myotubes were fixed with 4% paraformaldehyde (PFA) for 20 mins at room temperature (RT). After fixation, cells were washed twice with PBS followed by permeabilization with PBS-0.5% Triton for 15 mins at RT. Prior to incubation with primary antibodies cells were incubated with a blocking buffer for 30 mins at RT (PBS+10% FBS+0.1% Triton). Cell were then incubated with primary antibodies in block for 2 hours at RT (or overnight at 4° C.) followed by 3 washes with PBS-Tween 0.1%. Secondary antibodies are added for 1.5-2 hours at RT. Antibodies for these studies are listed here at Table 12:

TABLE 12

| | Host | Dilution |
|---|---|---|
| Primary Antibodies | | |
| SluCas9 (GenScript 11E3A9-1) | Mouse | 1:100 |
| SaCas9 (Diagenode C15200230-100) | Mouse | 1:400 |
| MyoG | Rabbit | 1:200 |
| Secondary Antibodies | | |
| Anti-mouse 488 | | 1:500 |
| Anti-Rabbit 594 | | 1:500 |

Vector Genome Quantitation

Quantitative polymerase chain reaction (qPCR) was used to quantify levels of AAV9 DNA in C2C12 differentiated cells. gDNA was extracted using the Qiagen AllPrep kit and quantified using the Qubit 4 Fluorometer and diluted to a final concentration of 2.5 ng/μL.

Absolute quantification of AAV9 vectors was performed by constructing a standard curve prepared from known quality of linearized plasmids encoding the region of interest. A set of Quality control (QC) were included to validate the methods of quantification. A set of non-template control (NTC) samples were included to confirm the specificity of reactions.

qPCR reactions were conducted in triplicate using a QuantStudio 6 Flex Real-Time PCR System (Thermo Fisher). A linear regression analysis was performed using threshold cycle (Ct) values of the standard curve. From this linear regression, the Ct values of the samples were used to quantify the number of copies per μg of gDNA of AAV9 present in each sample.

Cas9 and sgRNA Transgene Expression

Reverse transcription polymerase chain reaction (RT-qPCR) was used to quantify levels of Cas9 mRNA and the expression of gRNA in C2C12 differentiated cells. RNA was extracted using the Qiagen AllPrep kit and extracted RNA were quantified using the Qubit 4 Fluorometer and diluted to a final concentration of 20 ng/μL. Absolute quantification of gRNA and Cas9 mRNA were performed by constructing a standard curve prepared from known quality of a T7 transcript encoding the corresponding region of interest. A set of Quality control (QC) were included to validate the methods of quantification. A set of non-template control (NTC) samples were included to confirm the specificity of reactions.

RT-qPCR reactions were conducted in triplicate using a QuantStudio 6 Flex Real-Time PCR System (Thermo Fisher). A linear regression analysis was performed using threshold cycle (Ct) values of the standard curve. From this linear regression, the Ct values of the samples were used to quantify the number of copies per μg of RNA of either Cas9 mRNA or gRNA present in samples.

Amplicon Deep Sequencing Library Preparation for C2C12

gDNA was extracted using the Qiagen AllPrep kit. The relevant locus for exon 45 of mouse Dmd was amplified by PCR and amplified products were purified by AMPure beads (0.8×) followed by QC with 1% E-gel. DNA concentrations were measured using QuBiT for normalization of samples and Illumina sequencing libraries were created from these PCR products using a MiSeq Reagent Kit v3 (600-cycle).

Next Generation Sequencing Data Analysis

A custom bioinformatic workflow was used to process the Illumina sequencing data. First, poor quality reads were removed. Reads that passed the quality filter were trimmed using Trimmomatic to remove adapters and low quality bases. Then reads mapping to the PhiX genome were removed, and paired end reads were merged using PEAR. Based on the expected cut sites corresponding to the two guides, three reference amplicon sequences were created—wildtype amplicon, deletion amplicon with a deletion between the cutsites for the two guides, and inversion amplicon with an inversion of the sequence between the two the cutsites for the two guides. The merged reads were assigned to one of three amplicons (wild-type, deletion, inversion) based on the alignment score provided by the Needleman Wunsch algorithm as implemented in ParasailNeedle. The CIGAR string provided by the alignment algorithm was parsed to identify the sequence of indel events. The summarized table of indel events and their frequencies was overlaid with information on exon length, exon frame, and position of the premature stop codon introduced as a result of the DMD disease causing mutation, to characterize Cas9-associated indel events as productive (e.g., precise deletion, RF+1, RF Other, Exon skipping) or non-productive (e.g., OE) (Table 13). Throughout this workflow, a stringent set of QQC criteria was applied to filter poor quality samples. (Table 13).

TABLE 13

| | | |
|---|---|---|
| Indel | Ranking | |
| group | Order | Definition |

Wild-type amplicon

| | | |
|---|---|---|
| NE | 1 | insertion = 0 & deletion = 0 |
| RF + 1 | 2 | 1-nt insertion at a reframe guide |
| RF Other | 3 | 3n + 1 deletion within the exon boundary at the |
| | | only reframe guide \| no edits at the exon guide |
| | | aggregated 3n + 1 deletion within the exon |
| | | boundary at the two reframe guides |
| | | <17-nt insertion at the only reframe guide \| no |
| | | edits at the exon gate |
| | | <17-nt aggregated 3n +1 insertion at the two |
| | | reframe guides |
| Exon | 4 | indels overlap with the splice acceptor (AG) |
| skipping | | (5′-end of the target exon) |
| | | deletion with ≥ 9-nt overlap with the splicing |
| | | window at one of the two guides |
| | | insertion at the exact GT/AG splicing sites and |
| | | with length ≥ 9-nt at one of the two guides |
| OE | 5 | Other indels |

Deletion amplicon

| | | |
|---|---|---|
| Precise | 1 | segmental deletion between the two cut sites |
| deletion | | |
| (Rest) | 2 | SingleCut indel profiling with 3n phase |

Inversion amplicon

| | | |
|---|---|---|
| OE | 1 | all indels |

Animal and Study Design

This study was designed to evaluate all-in-one gene editing vector candidates that have been created to include both the sgRNA and a Cas9 endonuclease to mediate gene editing at the DMD locus. In this study, the efficacy of the one-vector gene editing candidates was assessed in vivo by measuring dystrophin restoration, on-target gene editing, tissue vector genomes, and Cas9 and sgRNA transgene expression following an intraperitoneal administration to dEx44 mice at postnatal day 4 or 5.

Table 14A describes the in-vivo study design:

| Group | Mouse | | | Test Article Vector | # of | Dose | Sample Collection and |
|---|---|---|---|---|---|---|---|
| # | Strain | Age | Test Article | Configuration | Animals | (vg/kg) | Analysis |
| 1 | WT | PND4 to | Vehicle | N/A | 6 | N/A | Tissue collection at 4 |
| 2 | ΔEx44 | PND5 | | | 6 | N/A | weeks post-dose |
| 3 | | | EX 145 | AAV9-Cas9:AAV9-M-sgRNA- | 8 | 1 × 10¹⁴: | Collect: heart, TA, |
| | | | | 145 | | 1 × 10¹⁴ | quadriceps, triceps, |
| 4 | | | AAV9-E45S124 | Slu24:2XNLS:7SK-Hlm | 8 | 2 × 10¹⁴ | diaphragm, and liver |
| 5 | | | AAV9-vVT046 | Slu18/4:2XNLS+:hU6-hU6 | 8 | 2 × 10¹⁴ | Analyses: Dystrophin |
| 6 | | | AAV9-vVT047 | Slu18/4:3XNLS:hU6-hU6 | 8 | 2 × 10¹⁴ | restoration, on-target gene |
| 7 | | | AAV9-vVT048 | Slu18/4:3XNLS:hU6-7SK | 8 | 2 × 10¹⁴ | editing, Cas9 protein |
| 8 | | | AAV9-vVT052 | Slu21/7:2XNLS+:7SK-H1m | 8 | 2 × 10¹⁴ | expression, vector |
| 9 | | | AAV9-vVT054 | Slu24:3XNLS:7SK-H1m | 8 | 2 × 10¹⁴ | genome copy number, and |
| 10 | | | AAV9-vVT049 | Slu18/4:3XNLS:H1m-M11 | 8 | 2 × 10¹⁴ | transgene expression on |
| 11 | | | AAV9-vVT050B | Slu18/4:3XNLS:7SK-H1m | 8 | 2 × 10¹⁴ | select tissues |
| 13 | | | AAV9-vVT051 | Slu18/4:2XNLS+:7SK-H1m | 8 | 2 × 10¹⁴ | |
| 14 | | | AAV9-vVT053B | SaKKH10/20:2XNLS+:7SK- | 8 | 2 × 10¹⁴ | |
| | | | | H1m | | | |

WT = wildtype
PND4 to PND5 = postnatal day 4 to postnatal day 5
N/A = no applicable

Amplicon Deep Sequencing Library Preparation for In Vivo Study

gDNA was extracted from mouse heart, quadricep, and triceps tissue using the Maxwell RSC Tissue DNA Kit and quantified via Qubit. The relevant locus of exon 45 of the mouse DMD gene was amplified using PCR. PCR products were visualized using both E-gel and TapeStation to confirm proper size and were purified using AMPure XP beads. PCR products then underwent a second PCR reaction to add unique 5′ and 3′ barcodes corresponding to each sample. These PCR products were quantified via Qubit and normalized to 4 nM each. The normalized products were then pooled, combined with a PhiX library to increase diversity, and loaded onto a MiSeq instrument. The library was sequenced using a MiSeq Reagent Kit v3 (600-cycle) and raw data was transferred to the DCS team.

Next Generation Sequencing Data Analysis for In Vivo Study

A custom bioinformatic workflow was used to process the Illumina sequencing data. First, poor quality reads (below Q30) were removed. The surviving reads were trimmed using Trimmomatic Trimmomatic to remove adapters and low quality bases. Then reads mapping to the PhiX genome were removed, and paired end reads were merged using PEAR. Based on the expected cut sites corresponding to the two guides, three reference amplicon sequences were created—wildtype amplicon, deletion amplicon with a deletion between the cut sites for the two guides, and inversion amplicon with an inversion of the sequence between the two the cut sites for the two guides. The merged reads were assigned to one of three amplicons (wild-type, deletion, inversion) based on the alignment score provided by the Needleman Wunsch algorithm as implemented in ParasailNeedle. The CIGAR string provided by the alignment algorithm was parsed to identify the sequence of indel events. The summarized table of indel events and their frequencies was overlaid with information on exon length, exon frame, and position of the premature stop codon introduced as a result of the DMD disease causing mutation, to characterize Cas9-associated indel events as productive (e.g., precise deletion, RF+1, RF Other, Exon skipping) or non-productive (e.g., OE) (Table 1). Throughout this workflow, a stringent set of QC criteria was applied to filter poor quality samples and all samples are checked for potential contaminations from other treatment groups in the same study.

2. Results

Dual Cut sgRNA In Vitro Screening

The average indel frequency of sgRNAs targeting exon 45 was tested in primary human skeletal muscle myoblasts (HsMM) (FIG. 8), with a high-performing SpCas9 sgRNA (E45Sp52) included as a reference. Each of the five SluCas9 sgRNA pairs evaluated showed an average precise segmental deletion frequency higher than 60% (FIG. 8). The sequences of the spacers for the guide RNAs used in FIG. 8 (i.e., 18, 4, and 18+4; 21, 7, and 21+7; 17, 22, and 17+22; 17, 23 and 17+23; and 19, 21, and 19+21) are shown in Table 10.

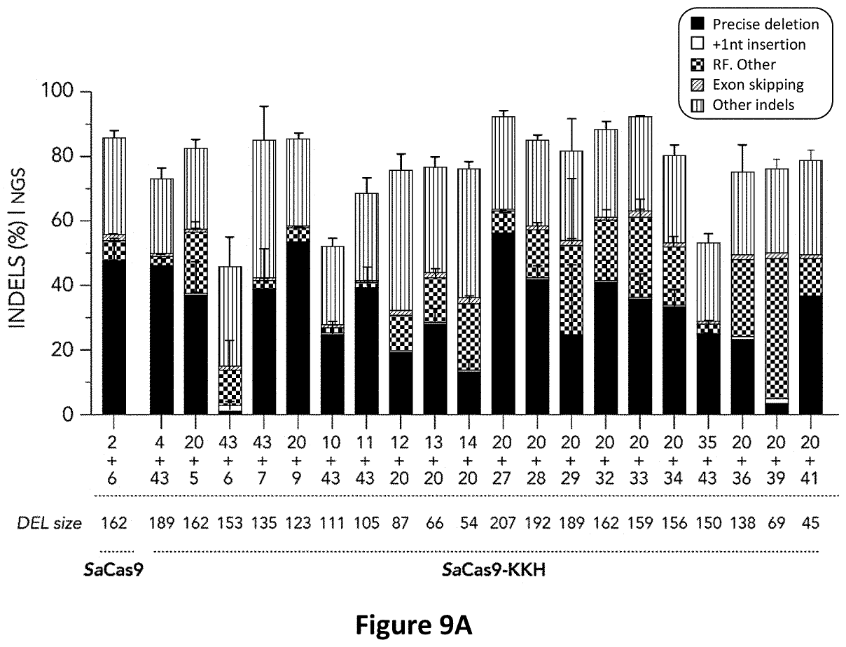

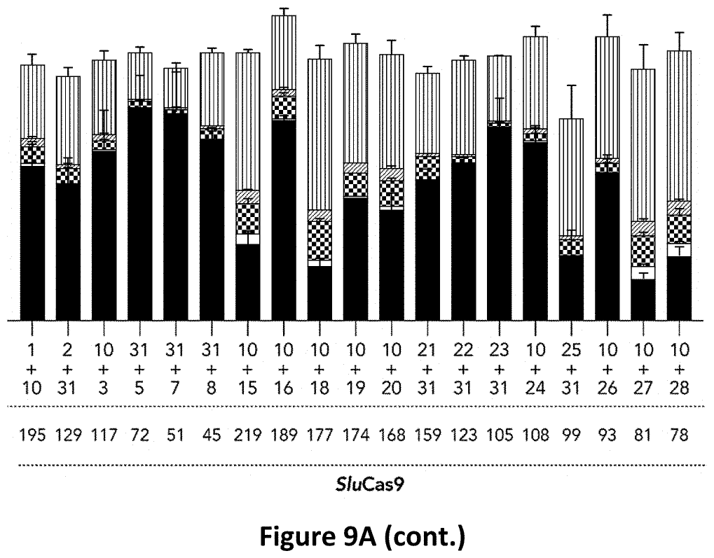

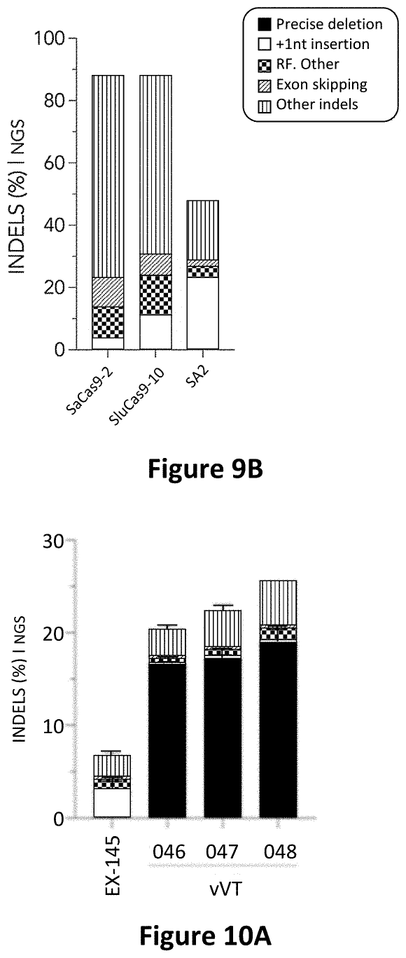

The average indel frequency of sgRNAs targeting exon 51 was determined in HEK293FT cells (FIG. 9), which shows an indel frequency higher than 50% and an average precise segmental deletion frequency above 50% for E51SaKKH20/9, E51SaKKH20/27, E51SL10/3, E51SL31/5, E51SL31/7, E51SL31/8, E51SL10/16, E51SL23/31 and E51SL10/24 in HEK293FT cells. Table 14B shows the sgRNA ID and the spacer sequence for each sgRNA pairing shown in FIG. 9, where the pairings shown on the X-axis in FIG. 9 (e.g., 2+6) are within the sgRNA ID as the last character of each term (e.g., E51Sa2_E51Sa6).

TABLE 14B

| | Protospacer sequence |
|---|---|
| sgRNA ID | (a comma separates the two spacers) |
| E51Sa2_E51Sa6 | GTTGTGTCACCAGAGTAACAG (SEQ ID NO: 20), |
| | TTGATCAAGTTATAAAATCACA (SEQ ID NO: 24) |
| E51SaCas9KKH4_E51SaCas9KKH43 | ATGATCATCTCGTTGATATCCT (SEQ ID NOS: 1022, 2031), |
| | TAGCTCCTACTCAGACTGTTAC (SEQ ID NO: 1055) |
| E51SaCas9KKH20_E51SaCas9KKH5 | CCACAGGTTGTGTCACCAGAGT (SEQ ID NO: 1037), |
| | AAGGTCACCCACCATCACCCTC (SEQ ID NO: 1023) |
| E51SaCas9KKH43_E51SaCas9KKH6 | TAGCTCCTACTCAGACTGTTAC (SEQ ID NO: 1055), |
| | ATCACCCTCTGTGATTTTATAA (SEQ ID NO: 1024) |
| E51SaCas9KKH43_E51SaCas9KKH7 | TAGCTCCTACTCAGACTGTTAC (SEQ ID NO: 1055), |
| | ATAACTTGATCAAGCAGAGAAA (SEQ ID NO: 1025) |
| E51SaCas9KKH20_E51SaCas9KKH9 | CCACAGGTTGTGTCACCAGAGT (SEQ ID NO: 1037), |
| | ATCAAGCAGAGAAAGCCAGTCG (SEQ ID NO: 1027) |
| E51SaCas9KKH10_E51SaCas9KKH43 | CAGTCGGTAAGTTCTGTCCAAG (SEQ ID NO: 1028), |
| | TAGCTCCTACTCAGACTGTTAC (SEQ ID NO: 1055) |
| E51SaCas9KKH11_E51SaCas9KKH43 | GTAAGTTCTGTCCAAGCCCGGT (SEQ ID NO: 1029), |
| | TAGCTCCTACTCAGACTGTTAC (SEQ ID NO: 1055) |
| E51SaCas9KKH12_E51SaCas9KKH20 | AGCCCGGTTGAAATCTGCCAGA (SEQ ID NOS: 1030, 2041), |
| | CCACAGGTTGTGTCACCAGAGT (SEQ ID NO: 1037) |
| E51SaCas9KKH13_E51SaCas9KKH20 | AGCAGGTACCTCCAACATCAAG (SEQ ID NOS: 1031, 2043), |
| | CCACAGGTTGTGTCACCAGAGT (SEQ ID NO: 1037) |
| E51SaCas9KKH14_E51SaCas9KKH20 | CAACATCAAGGAAGATGGCATT (SEQ ID NO: 1032), |
| | CCACAGGTTGTGTCACCAGAGT (SEQ ID NO: 1037) |
| E51SaCas9KKH20_E51SaCas9KKH27 | CCACAGGTTGTGTCACCAGAGT (SEQ ID NO: 1037), |
| | TCAACGAGATGATCATCAAGCA (SEQ ID NOS: 1042, 2060) |
| E51SaCas9KKH20_E51SaCas9KKH28 | CCACAGGTTGTGTCACCAGAGT (SEQ ID NO: 1037), |
| | GTGACCTTGAGGATATCAACGA (SEQ ID NO: 1043) |
| E51SaCas9KKH20_E51SaCas9KKH29 | CCACAGGTTGTGTCACCAGAGT (SEQ ID NO: 1037), |
| | TGGGTGACCTTGAGGATATCAA (SEQ ID NOS: 1044, 2063) |
| E51SaCas9KKH20_E51SaCas9KKH32 | CCACAGGTTGTGTCACCAGAGT (SEQ ID NO: 1037), |
| | AAGTTATAAAATCACAGAGGGT (SEQ ID NO: 1045) |
| E51SaCas9KKH20_E51SaCas9KKH33 | CCACAGGTTGTGTCACCAGAGT (SEQ ID NO: 1037), |
| | ATCAAGTTATAAAATCACAGAG (SEQ ID NO: 1046) |
| E51SaCas9KKH20_E51SaCas9KKH34 | CCACAGGTTGTGTCACCAGAGT (SEQ ID NO: 1037), |
| | TTGATCAAGTTATAAAATCACA (SEQ ID NO: 24) |
| E51SaCas9KKH35_E51SaCas9KKH43 | CTTTCTCTGCTTGATCAAGTTA (SEQ ID NO: 1047), |
| | TAGCTCCTACTCAGACTGTTAC (SEQ ID NO: 1055) |
| E51SaCas9KKH20_E51SaCas9KKH36 | CCACAGGTTGTGTCACCAGAGT (SEQ ID NO: 1037), |
| | CCGACTGGCTTTCTCTGCTTGA (SEQ ID NO: 1048) |
| E51SaCas9KKH20_E51SaCas9KKH39 | CCACAGGTTGTGTCACCAGAGT (SEQ ID NO: 1037), |
| | AAATGCCATCTTCCTTGATGTT (SEQ ID NOS: 1051, 2070) |
| E51SaCas9KKH20_E51SaCas9KKH41 | CCACAGGTTGTGTCACCAGAGT (SEQ ID NO: 1037), |
| | AGGAAACTGCCATCTCCAAACT (SEQ ID NO: 1053) |
| E51SL1_E51SL10 | TGATCATCTCGTTGATATCCTC (SEQ ID NO: 170), |
| | GTCACCAGAGTAACAGTCTGAG (SEQ ID NO: 179) |
| E51SL2_E51SL31 | TTGATCAAGCAGAGAAAGCCAG (SEQ ID NO: 171), |
| | AGCTCCTACTCAGACTGTTACT (SEQ ID NO: 200) |
| E51SL10_E51SL3 | GTCACCAGAGTAACAGTCTGAG (SEQ ID NO: 179), |
| | AGTCGGTAAGTTCTGTCCAAGC (SEQ ID NO: 172) |
| E51SL31_E51SL5 | AGCTCCTACTCAGACTGTTACT (SEQ ID NO: 200), |
| | CAGAGCAGGTACCTCCAACATC (SEQ ID NO: 174) |
| E51SL31_E51SL7 | AGCTCCTACTCAGACTGTTACT (SEQ ID NO: 200), |
| | CAAGGAAGATGGCATTTCTAGT (SEQ ID NO: 176) |
| E51SL31_E51SL8 | AGCTCCTACTCAGACTGTTACT (SEQ ID NO: 200), |
| | AGATGGCATTTCTAGTTTGGAG (SEQ ID NO: 177) |
| E51SL10_E51SL15 | GTCACCAGAGTAACAGTCTGAG (SEQ ID NO: 179), |
| | CAACGAGATGATCATCAAGCAG (SEQ ID NO: 184) |
| E51SL10_E51SL16 | GTCACCAGAGTAACAGTCTGAG (SEQ ID NO: 179), |
| | GAGGGTGATGGTGGGTGACCTT (SEQ ID NOS: 22, 185) |
| E51SL10_E51SL18 | GTCACCAGAGTAACAGTCTGAG (SEQ ID NO: 179), |
| | TATAAAATCACAGAGGGTGATG (SEQ ID NOS: 23, 187) |
| E51SL10_E51SL19 | GTCACCAGAGTAACAGTCTGAG (SEQ ID NO: 179), |
| | AGTTATAAAATCACAGAGGGTG (SEQ ID NO: 188) |
| E51SL10_E51SL20 | GTCACCAGAGTAACAGTCTGAG (SEQ ID NO: 179), |
| | TGATCAAGTTATAAAATCACAG (SEQ ID NO: 189) |
| E51SL21_E51SL31 | TTGATCAAGTTATAAAATCACA (SEQ ID NO: 24), |
| | AGCTCCTACTCAGACTGTTACT (SEQ ID NO: 200) |
| E51SL22_E51SL31 | GGGCTTGGACAGAACTTACCGA (SEQ ID NO: 191), |
| | AGCTCCTACTCAGACTGTTACT (SEQ ID NO: 200) |
| E51SL23_E51SL31 | CTCTGGCAGATTTCAACCGGGC (SEQ ID NO: 192), |
| | AGCTCCTACTCAGACTGTTACT (SEQ ID NO: 200) |
| E51SL10_E51SL24 | GTCACCAGAGTAACAGTCTGAG (SEQ ID NO: 179), |
| | ACCTGCTCTGGCAGATTTCAAC (SEQ ID NO: 193) |
| E51SL25_E51SL31 | TACCTGCTCTGGCAGATTTCAA (SEQ ID NO: 194), |
| | AGCTCCTACTCAGACTGTTACT (SEQ ID NO: 200) |
| E51SL10_E51SL26 | GTCACCAGAGTAACAGTCTGAG (SEQ ID NO: 179), |
| | CTTGATGTTGGAGGTACCTGCT (SEQ ID NO: 195) |
| E51SL10_E51SL27 | GTCACCAGAGTAACAGTCTGAG (SEQ ID NO: 179), |
| | AATGCCATCTTCCTTGATGTTG (SEQ ID NO: 196) |
| E51SL10_E51SL28 | GTCACCAGAGTAACAGTCTGAG (SEQ ID NO: 179), |
| | AGAAATGCCATCTTCCTTGATG (SEQ ID NO: 197) |
| E51SLCas9KH26_E51SLCas9KH73 | GATGGCAGTTTCCTTAGTAACC (SEQ ID NOS: 1036, 2048), |
| | AGCTCCTACTCAGACTGTTACT (SEQ ID NO: 200) |
| E51SLCas9KH25_E51SLCas9KH73 | TAGTTTGGAGATGGCAGTTTCC (SEQ ID NO: 2047), |
| | AGCTCCTACTCAGACTGTTACT (SEQ ID NO: 200) |
| E51SLCas9KH24_E51SLCas9KH73 | TGGCATTTCTAGTTTGGAGATG (SEQ ID NO: 2046), |
| | AGCTCCTACTCAGACTGTTACT (SEQ ID NO: 200) |
| E51SLCas9KH21_E51SLCas9KH73 | CAAGGAAGATGGCATTTCTAGT (SEQ ID NO: 176), |
| | AGCTCCTACTCAGACTGTTACT (SEQ ID NO: 200) |
| E51SLCas9KH20_E51SLCas9KH30 | AACATCAAGGAAGATGGCATTT (SEQ ID NO: 2044), |
| | CACAGGTTGTGTCACCAGAGTA (SEQ ID NO: 2051) |
| E51SLCas9KH19_E51SLCas9KH30 | GGTACCTCCAACATCAAGGAAG (SEQ ID NO: 175), |
| | CACAGGTTGTGTCACCAGAGTA (SEQ ID NO: 2051) |
| E51SLCas9KH20_E51SLCas9KH32 | AACATCAAGGAAGATGGCATTT (SEQ ID NO: 2044), |
| | TGTCACCAGAGTAACAGTCTGA (SEQ ID NO: 2053) |
| E51SLCas9KH20_E51SLCas9KH34 | AACATCAAGGAAGATGGCATTT (SEQ ID NO: 2044), |
| | CACCAGAGTAACAGTCTGAGTA (SEQ ID NO: 2054) |
| E51SLCas9KH18_E51SLCas9KH73 | AGCAGGTACCTCCAACATCAAG (SEQ ID NOS: 1031, 2043), |
| | AGCTCCTACTCAGACTGTTACT (SEQ ID NO: 200) |
| E51SLCas9KH17_E51SLCas9KH73 | CAGAGCAGGTACCTCCAACATC (SEQ ID NO: 174), |
| | AGCTCCTACTCAGACTGTTACT (SEQ ID NO: 200) |
| E51Sa2 | GTTGTGTCACCAGAGTAACAGT (SEQ ID NO: 20) |
| E51SL10 | GTCACCAGAGTAACAGTCTGAG (SEQ ID NO: 179) |
| E51SluCas9-2 | TTGATCAAGCAGAGAAAGCCAG (SEQ ID NO: 171) |
| E51SluCas9-10 | GTCACCAGAGTAACAGTCTGAG (SEQ ID NO: 179) |

AAV Configurations for the Dual Cut Single Vector Candidates

A combination of promoter orientations, and configurations, NLS sequences and sgRNA scaffolds were selected for generating AAV plasmids and evaluation on sgRNA transgene expression, AAV manufacturability, and editing efficiency in vitro and in vivo. AAV plasmid configurations are listed in Table 15 and Table 16. Promoter, NLS and scaffold sequences are listed in Table 17.

Guide RNA were prepared according to standard methods in a single guide (sgRNA) format. A single AAV vector was prepared that expresses the guide RNA pair and a variant SaCas9 or the guide RNA pair and a variant SluCas9. The AAV vectors were administered to C2C12 cells in vitro and to mice (e.g., dEx44 mice) in vivo to assess the ability of the AAV to express the guide RNA and Cas9, edit the targeted exon, and reframe the dystrophin gene (in vivo studies only).

TABLE 15

| Pol III | | | | | | | |
|---|---|---|---|---|---|---|---|
| Promoter | Orientation | Configuration | NLS1 | Endonuclease | NLS2 | NLS3 | Scaffold |
| hU6c:hU6c | ← [symbol] → | hU6c-Cas9- | c-Myc- | SluCas9 | SV-40-GSGS | Nucleoplasmin- | V5 |
| | | hU6c | GSVD | | | GSGS | |
| | ← [symbol] → | hU6c-Cas9- | c-Myc- | SaCas9-KKH | SV-40-GSGS | Nucleoplasmin- | V2 |
| | | hU6c | GSVD | | | GSGS | |
| | ← [symbol] → | hU6c-Cas9- | c-Myc- | SluCas9 | SV-40-MCS- | Nucleoplasmin- | V5 |
| | | hU6c | GSVD | | GSGS | GSGS | |
| | ← [symbol] → | hU6c-Cas9- | c-Myc- | SaCas9-KKH | SV-40-MCS- | Nucleoplasmin- | V2 |
| | | hU6c | GSVD | | GSGS | GSGS | |
| | ← [symbol] → | hU6c-Cas9- | SV-40 | SluCas9 | Nucleoplasmin | N/A | V5 |
| | | hU6c | (+) | | | | |
| | ← [symbol] → | hU6c-Cas9- | SV-40 | SaCas9-KKH | Nucleoplasmin | N/A | V2 |
| | | hU6c | (+) | | | | |
| | ← ← [symbol] | hU6c-hU6c- | c-Myc- | SluCas9 | SV-40-GSGS | Nucleoplasmin- | V5 |
| | | Cas9 | GSVD | | | GSGS | |
| | ← ← [symbol] | hU6c-hU6c- | c-Myc- | SaCas9-KKH | SV-40-GSGS | Nucleoplasmin- | V2 |
| | | Cas9 | GSVD | | | GSGS | |
| | ← ← [symbol] | hU6c-hU6c- | c-Myc- | SluCas9 | SV-40-MCS- | Nucleoplasmin- | V5 |
| | | Cas9 | GSVD | | GSGS | GSGS | |
| | ← ← [symbol] | hU6c-hU6c- | c-Myc- | SaCas9-KKH | SV-40-MCS- | Nucleoplasmin- | V2 |
| | | Cas9 | GSVD | | GSGS | GSGS | |
| | ← ← [symbol] | hU6c-hU6c- | SV-40 | SluCas9 | Nucleoplasmin | N/A | V5 |
| | | Cas9 | (+) | | | | |
| | ← ← [symbol] | hU6c-hU6c- | SV-40 | SaCas9-KKH | Nucleoplasmin | N/A | V2 |
| | | Cas9 | (+) | | | | |
| | → → [symbol] | hU6c-hU6c- | c-Myc- | SluCas9 | SV-40-GSGS | Nucleoplasmin- | V5 |
| | | Cas9 | GSVD | | | GSGS | |
| | → → [symbol] | hU6c-hU6c- | c-Myc- | SaCas9-KKH | SV-40-GSGS | Nucleoplasmin- | V2 |
| | | Cas9 | GSVD | | | GSGS | |
| | → → [symbol] | hU6c-hU6c- | c-Myc- | SluCas9 | SV-40-MCS- | Nucleoplasmin- | V5 |
| | | Cas9 | GSVD | | GSGS | GSGS | |
| | → → [symbol] | hU6c-hU6c- | c-Myc- | SaCas9-KKH | SV-40-MCS- | Nucleoplasmin- | V2 |
| | | Cas9 | GSVD | | GSGS | GSGS | |
| | → → [symbol] | hU6c-hU6c- | SV-40 | SluCas9 | Nucleoplasmin | N/A | V5 |
| | | Cas9 | (+) | | | | |
| | → → [symbol] | hU6c-hU6c- | SV-40 | SaCas9-KKH | Nucleoplasmin | N/A | V2 |
| | | Cas9 | (+) | | | | |
| hU6c:7SK2 | ← [symbol] → | hU6c-Cas9- | c-Myc- | SluCas9 | SV-40-GSGS | Nucleoplasmin- | V5 |
| | | 7SK2 | GSVD | | | GSGS | |
| | ← [symbol] → | hU6c-Cas9- | c-Myc- | SaCas9-KKH | SV-40-GSGS | Nucleoplasmin- | V2 |
| | | 7SK2 | GSVD | | | GSGS | |
| | ← [symbol] → | hU6c-Cas9- | c-Myc- | SluCas9 | SV-40-MCS- | Nucleoplasmin- | V5 |
| | | 7SK2 | GSVD | | GSGS | GSGS | |
| | ← [symbol] → | hU6c-Cas9- | c-Myc- | SaCas9-KKH | SV-40-MCS- | Nucleoplasmin- | V2 |
| | | 7SK2 | GSVD | | GSGS | GSGS | |
| | ← ← [symbol] | hU6c-7SK2- | c-Myc- | SluCas9 | SV-40-MCS- | Nucleoplasmin- | V5 |
| | | Cas9 | GSVD | | GSGS | GSGS | |
| | ← ← [symbol] | hU6c-7SK2- | c-Myc- | SaCas9-KKH | SV-40-MCS- | Nucleoplasmin- | V2 |
| | | Cas9 | GSVD | | GSGS | GSGS | |
| | ← ← [symbol] | hU6c-7SK2- | c-Myc- | SluCas9 | SV-40-GSGS | Nucleoplasmin- | V5 |
| | | Cas9 | GSVD | | | GSGS | |
| | ← ← [symbol] | hU6c-7SK2- | c-Myc- | SaCas9-KKH | SV-40-GSGS | Nucleoplasmin- | V2 |
| | | Cas9 | GSVD | | | GSGS | |
| | ← ← [symbol] | hU6c-7SK2- | SV-40 | SluCas9 | Nucleoplasmin | N/A | V5 |
| | | Cas9 | (+) | | | | |
| | ← ← [symbol] | hU6c-7SK2- | SV-40 | SaCas9-KKH | Nucleoplasmin | N/A | V2 |
| | | Cas9 | (+) | | | | |
| | → → [symbol] | hU6c-7SK2- | c-Myc- | SluCas9 | SV-40-MCS- | Nucleoplasmin- | V5 |
| | | Cas9 | GSVD | | GSGS | GSGS | |
| | → → [symbol] | hU6c-7SK2- | c-Myc- | SaCas9-KKH | SV-40-MCS- | Nucleoplasmin- | V2 |
| | | Cas9 | GSVD | | GSGS | GSGS | |
| | → → [symbol] | hU6c-7SK2- | c-Myc- | SluCas9 | SV-40-GSGS | Nucleoplasmin- | V5 |
| | | Cas9 | GSVD | | | GSGS | |
| | → → [symbol] | hU6c-7SK2- | c-Myc- | SaCas9-KKH | SV-40-GSGS | Nucleoplasmin- | V2 |
| | | Cas9 | GSVD | | | GSGS | |
| | → → [symbol] | hU6c-7SK2- | SV-40 | SluCas9 | Nucleoplasmin | N/A | V5 |
| | | Cas9 | (+) | | | | |
| | → → [symbol] | hU6c-7SK2- | SV-40 | SaCas9-KKH | Nucleoplasmin | N/A | V2 |
| | | Cas9 | (+) | | | | |
| 7SK2:hU6c | ← [symbol] → | 7SK2-Cas9- | c-Myc- | SluCas9 | SV-40-GSGS | Nucleoplasmin- | V5 |
| | | hU6c | GSVD | | | GSGS | |
| | ← [symbol] → | 7SK2-Cas9- | c-Myc- | SaCas9-KKH | SV-40-GSGS | Nucleoplasmin- | V2 |
| | | hU6c | GSVD | | | GSGS | |
| | ← [symbol] → | 7SK2-Cas9- | c-Myc- | SluCas9 | SV-40-MCS- | Nucleoplasmin- | V5 |
| | | hU6c | GSVD | | GSGS | GSGS | |
| | ← [symbol] → | 7SK2-Cas9- | c-Myc- | SaCas9-KKH | SV-40-MCS- | Nucleoplasmin- | V2 |
| | | hU6c | GSVD | | GSGS | GSGS | |
| | ← ← [symbol] | 7SK2-hU6c- | c-Myc- | SluCas9 | SV-40-MCS- | Nucleoplasmin- | V5 |
| | | Cas9 | GSVD | | GSGS | GSGS | |
| | ← ← [symbol] | 7SK2-hU6c- | c-Myc- | SaCas9-KKH | SV-40-MCS- | Nucleoplasmin- | V2 |
| | | Cas9 | GSVD | | GSGS | GSGS | |
| | ← ← [symbol] | 7SK2-hU6c- | c-Myc- | SluCas9 | SV-40-GSGS | Nucleoplasmin- | V5 |
| | | Cas9 | GSVD | | | GSGS | |
| | ← ← [symbol] | 7SK2-hU6c- | c-Myc- | SaCas9-KKH | SV-40-GSGS | Nucleoplasmin- | V2 |
| | | Cas9 | GSVD | | | GSGS | |
| | ← ← [symbol] | 7SK2-hU6c- | SV-40 | SluCas9 | Nucleoplasmin | N/A | V5 |
| | | Cas9 | (+) | | | | |
| | ← ← [symbol] | 7SK2-hU6c- | SV-40 | SaCas9-KKH | Nucleoplasmin | N/A | V2 |
| | | Cas9 | (+) | | | | |
| | → → [symbol] | 7SK2-hU6c- | c-Myc- | SluCas9 | SV-40-MCS- | Nucleoplasmin- | V5 |
| | | Cas9 | GSVD | | GSGS | GSGS | |
| | → → [symbol] | 7SK2-hU6c- | c-Myc- | SaCas9-KKH | SV-40-MCS- | Nucleoplasmin- | V2 |
| | | Cas9 | GSVD | | GSGS | GSGS | |
| | → → [symbol] | 7SK2-hU6c- | c-Myc- | SluCas9 | SV-40-GSGS | Nucleoplasmin- | V5 |
| | | Cas9 | GSVD | | | GSGS | |
| | → → [symbol] | 7SK2-hU6c- | c-Myc- | SaCas9-KKH | SV-40-GSGS | Nucleoplasmin- | V2 |
| | | Cas9 | GSVD | | | GSGS | |
| | → → [symbol] | 7SK2-hU6c- | SV-40 | SluCas9 | Nucleoplasmin | N/A | V5 |
| | | Cas9 | (+) | | | | |
| | → → [symbol] | 7SK2-hU6c- | SV-40 | SaCas9-KKH | Nucleoplasmin | N/A | V2 |
| | | Cas9 | (+) | | | | |
| hU6c:H1m | ← [symbol] → | hU6c-Cas9- | c-Myc- | SluCas9 | SV-40-GSGS | Nucleoplasmin- | V5 |
| | | H1m | GSVD | | | GSGS | |
| | ← [symbol] → | hU6c-Cas9- | c-Myc- | SaCas9-KKH | SV-40-GSGS | Nucleoplasmin- | V2 |
| | | H1m | GSVD | | | GSGS | |
| 7SK2:H1m | ← [symbol] → | 7SK2-Cas9- | SV-40- | SluCas9 | Nucleoplasmin | N/A | V5 |
| | | H1m | GS | | | | |
| | ← [symbol] → | 7SK2-Cas9- | SV-40- | SaCas9-KKH | Nucleoplasmin | N/A | V2 |
| | | H1m | GS | | | | |
| | ← [symbol] → | 7SK2-Cas9- | SV-40 | SluCas9 | Nucleoplasmin | N/A | V5 |
| | | H1m | (+) | | | | |
| | ← [symbol] → | 7SK2-Cas9- | SV-40 | SaCas9-KKH | Nucleoplasmin | N/A | V2 |
| | | H1m | (+) | | | | |
| | ← [symbol] → | 7SK2-Cas9- | c-Myc- | SluCas9 | SV-40-MCS- | Nucleoplasmin- | V5 |
| | | H1m | GSVD | | GSGS | GSGS | |
| | ← [symbol] → | 7SK2-Cas9- | c-Myc- | SaCas9-KKH | SV-40-MCS- | Nucleoplasmin- | V2 |
| | | H1m | GSVD | | GSGS | GSGS | |
| | ← [symbol] → | 7SK2-Cas9- | c-Myc- | SaCas9 | SV-40-MCS- | Nucleoplasmin- | V2 |
| | | H1m | GSVD | | GSGS | GSGS | |
| H1m:M11 | ← [symbol] → | H1m-Cas9- | c-Myc- | SluCas9 | SV-40-MCS- | Nucleoplasmin- | V5 |
| | | M11 | GSVD | | GSGS | GSGS | |
| | ← [symbol] → | H1m-Cas9- | c-Myc- | SaCas9-KKH | SV-40-MCS- | Nucleoplasmin- | V2 |
| | | M11 | GSVD | | GSGS | GSGS | |

TABLE 16

| | Pol III Promoter | Orientation | Configuration |
|---|---|---|---|
| Self-complementary AAV | hU6:H1:7SK | → → → | hU6-H1-7SK1 |
| | hU6:7SK | → □ → | hU6-Stuffer-7SK1 |
| | hU6:7SK | → → □ | hU6-7SK-Stuffer |
| | hU6c:hU6c | ← □ → | hU6c-Stuffer-hU6c |
| | hU6c:mU6 | ← □ → | hU6c-Stuffer-mU6 |
| | 7SK2:hU6c:hU6c:7SK2 | ← □ → | 7SK2-hU6c-Stuffer-hU6c-7SK2 |
| | 7SK2:hU6c:mU6:7SK2 | ← □ → | 7SK2-hU6c-Stuffer-mU6c-7SK2 |
| | 7SK2:hU6c:hU6c:7SK2 | ← □ → | 7SK2-hU6c-Stuffer-hU6c-7SK2 |
| | 7SK2:hU6c:mU6:7SK2 | ← □ → | 7SK2-hU6c-Stuffer-mU6-7SK2 |
| Single-stranded AAV | hU6c:hU6c | ← □ → | hU6c-Stuffer-hU6c |
| | hU6c:mU6 | ← □ → | hU6c-Stuffer-mU6 |
| | 7SK2:hU6c:hU6c:7SK2 | ← □ → | 7SK2-hU6c-Stuffer-hU6c-7SK2 |
| | 7SK2:hU6c:mU6:75K2 | ← □ → | 7SK2-hU6c-Stuffer-mU6c-7SK2 |
| | 7SK2:hU6c:hU6c:75K2 | ← □ → | 7SK2-hU6c-Stuffer-hU6c-7SK2 |
| | 7SK2:hU6c:mU6:75K2 | ← □ → | 7SK2-hU6c-Stuffer-mU6-7SK2 |

TABLE 17

| Promoter | Sequences |
|---|---|
| hU6c | GAGGGCCTATTTCCCATGATTCCTTCATATTT |
| | GCATATACGATACAAGGCTGTTAGAGAGATAA |
| | TTGGAATTAATTTGACTGTAAACACAAAGATA |
| | TTAGTACAAAATACGTGACGTAGAAAGTAATA |
| | ATTTCTTGGGTAGTTTGCAGTTTTAAAATTAT |
| | GTTTTAAAATGGACTATCATATGCTTACCGTA |
| | ACTTGAAAGTATTTCGATTTCTTGGCTTTATA |
| | TATCTTGTGGAAAGGACGAAACACC |
| | (SEQ ID NO: 705) |
| 7SK2 | CTGCAGTATTTAGCATGCCCCACCCATCTGCA |
| | AGGCATTCTGGATAGTGTCAAAACAGCCGGAA |
| | ATCAAGTCCGTTTATCTCAAACTTTAGCATTT |
| | TGGGAATAAATGATATTTGCTATGCTGGTTAA |
| | ATTAGATTTTAGTTAAATTTCCTGCTGAAGCT |
| | CTAGTACGATAAGCAACTTGACCTAAGTGTAA |
| | AGTTGAGACTTCCTTCAGGTTTATATAGCTTG |
| | TGCGCCGCTTGGGTACCTC |
| | (SEQ ID NO: 706) |
| H1m | AATATTTGCATGTCGCTATGTGTTCTGGGAAA |
| | TCACCATAAACGTGAAATGTCTTTGGATTTGG |
| | GAATCTTATAAGTTCTGTATGAGACCACTCTT |
| | TCCC |
| | (SEQ ID NO: 707) |
| M11 | ATATTTAGCATGTCGCTATGTGTTCTGGGAAA |
| | CTTGACCTAAGTGTAAAGTTGAGATTTCCTTC |
| | AGGTTTATATAGTTCTGTATGAGACCACTCTT |
| | TCCC |
| | (SEQ ID NO: 708) |
| hU6 | ccCGAGTCCAACACCCGTGGGAATCCCATGGG |
| | CACCATGGCCCCTCGCTCCAAAAATGCTTTCG |
| | CGTCGCGCAGACACTGCTCGGTAGTTTCGGGG |
| | ATCAGCGTTTGAGTAAGAGCCCGCGTCTGAAC |
| | CCTCCGCGCCGCCCCGGCCCCAGTGGAAAGAC |
| | GCGCAGGCAAAACGCACCACGTGACGGAGCGT |
| | GACCGCGCGCCGAGCGCGCGCCAAGGTCGGGC |
| | AGGAAGAGGGCCTATTTCCCATGATTCCTTCA |
| | TATTTGCATATACGATACAAGGCTGTTAGAGA |
| | GATAATTAGAATTAATTTGACTGTAAACACAA |
| | AGATATTAGTACAAAATACGTGACGTAGAAAG |
| | TAATAATTTCTTGGGTAGTTTGCAGTTTTAAA |
| | ATTATGTTTTAAAATGGACTATCATATGCTTA |
| | CCGTAACTTGAAAGTATTTCGATTTCTTGGCT |
| | TTATATATCTTGTGGAAAGGACGAAACACC |
| | (SEQ ID NO: 42) |
| mU6 | Gatccgacgccgccatctctaggcccgcgccg |
| | gccccctcgcacagacttgtgggagaagctcg |
| | gctactcccctgccccggttaatttgcatata |
| | atatttcctagtaactatagaggcttaatgtg |
| | cgataaaagacagataatctgttctttttaat |
| | actagctacattttacatgataggcttggatt |
| | tctataagagatacaaatactaaattattatt |
| | ttaaaaaacagcacaaaaggaaactcacccta |
| | actgtaaagtaattgtgtgttttgagactata |
| | aatatcccttggagaaaagccttgtt |
| | (SEQ ID NO: 43) |
| H1 | CATATTTGCATGTCGCTATGTGTTCTGGGAAA |
| | TCACCATAAACGTGAAATGTCTTTGGATTTGG |
| | GAATCTTATAAGTTCTGTATGAGACCACGGTA |
| | CACC |
| | (SEQ ID NO: 44) |
| 7SK1 | CTGCAGTATTTAGCATGCCCCACCCATCTGCA |
| | AGGCATTCTGGATAGTGTCAAAACAGCCGGAA |
| | ATCAAGTCCGTTTATCTCAAACTTTAGCATTT |
| | TGGGAATAAATGATATTTGCTATGCTGGTTAA |
| | ATTAGATTTTAGTTAAATTTCCTGCTGAAGCT |
| | CTAGTACGATAAGTAACTTGACCTAAGTGTAA |
| | AGTTGAGATTTCCTTCAGGTTTATATAGCTTG |
| | TGCGCCGCCTGGGTAC |
| | (SEQ ID NO: 45) |
| NLS-linker | AA Sequences |
| c-Myc-GSVD | PLAKKKKLDGSVD (SEQ ID NO: 36) |
| SV-40-GS | PKKKRKVGS (SEQ ID NO: 37) |
| SV-40 (+) | PKKKRKVGIHGVPAA (SEQ ID NO: 38) |
| SV-40-GSGS | GSGSPKKKRKV (SEQ ID NO: 39) |
| SV-40- | TGGGPGGGAAAGSGSPKKKRKV |
| MCS-GSGS | (SEQ ID NO: 40) |
| Nucleoplasmin- | GSGSKRPAATKKAGQAKKKK |
| GSGS | (SEQ ID NO: 41) |
| Scaffold | Sequences |
| SluCas9 | GUUUCAGUACUCUGGAAACAGAAUCUACUGAA |
| scaffold V5 | ACAAGACAAUAUGUCGUGUUUAUCCCAUCA |
| | AUUUAUUGGUGGGAU |
| | (SEQ ID NO: 922) |
| SaCas9 | GTTTAAGTACTCTGTGCTGGAAACAGCACAGA |
| scaffold V2 | ATCTACTTAAACAAGGCAAAATGCCGTGTTTA |
| | TCTCGTCAACTTGTTGGCGAGA |
| | (SEQ ID NO: 500) |

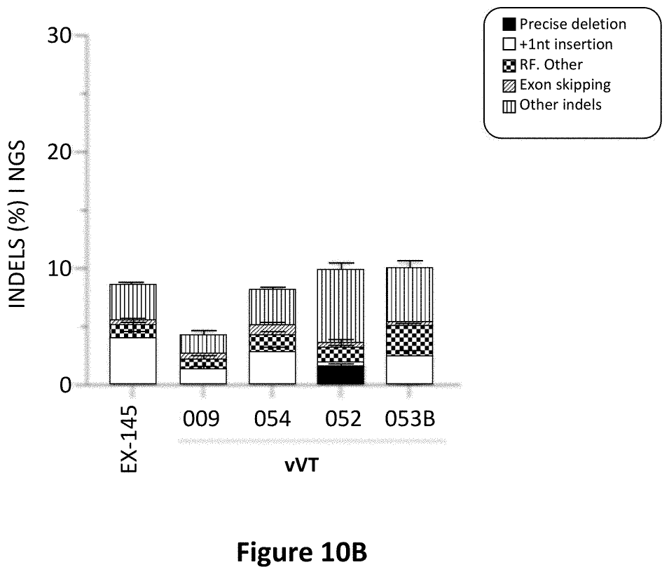

In particular, the ability of in vitro single AAV-mediated delivery of gene-editing components to introduce productive editing and precise segmental deletion in the C2C12 mouse myotubes was tested (Table 18B, FIG. 10). In FIGS. 10-12, and 14-17, vector IDs vVT 046, 047, 048, 054, and 052 are shown in Table 18B with their associated sgRNA pairs. vVT009 has a 7SK2-SluCas9-H1m configuration, with a SluV2 scaffold, and a SV40 NLS on the N-terminus and nucleoplasmin NLS on the C-terminus. vVT053B has the same configuration and sequences as vVT053. The B stands for an A deletion on the backbone of pVT053B (outside of ITRs) that was introduced during cloning. The spacer sequences used in these sgRNA pairs are as follows (Table 18A):

TABLE 18A

| | Protospacer sequence |
|---|---|
| sgRNA ID | (a comma separates the two protospacer sequences) |
| E45SL18_E45SL4 | TTGTCAGAACAcTGAATGCAAC (SEQ ID NO: 553), |
| | cTTGaCGCTGCCCAATGCCATC (SEQ ID NO: 300) |
| E45SL21_E45SL7 | TACAGGAACTCCAGGATGGCAT (SEQ ID NO: 148/306), |
| | CTGgCAGAaAGgAgAAAGAGGT (SEQ ID NO: 301) |
| E45SaCas9KKH10_E45SaCas9KKH20 | TTGaCGCTGCCCAATGCCATCC (SEQ ID NO: 317), |
| | ACAGATGICAaTATTCTACAaG (SEQ ID NO: 554) |

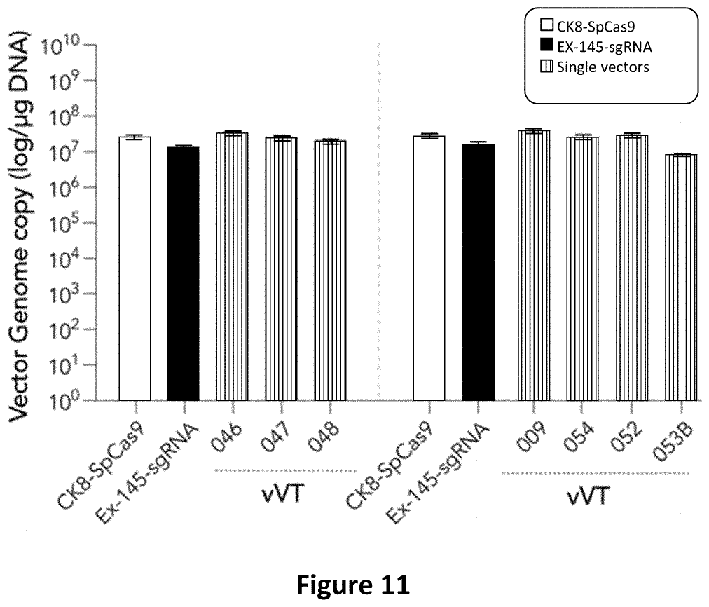

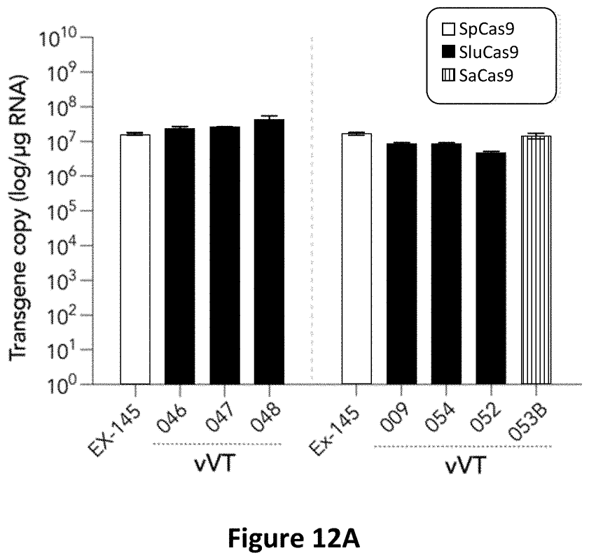

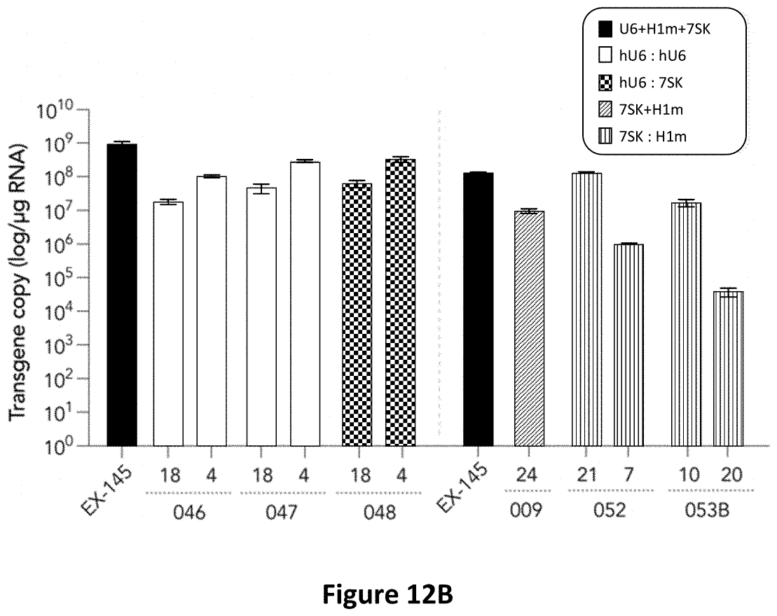

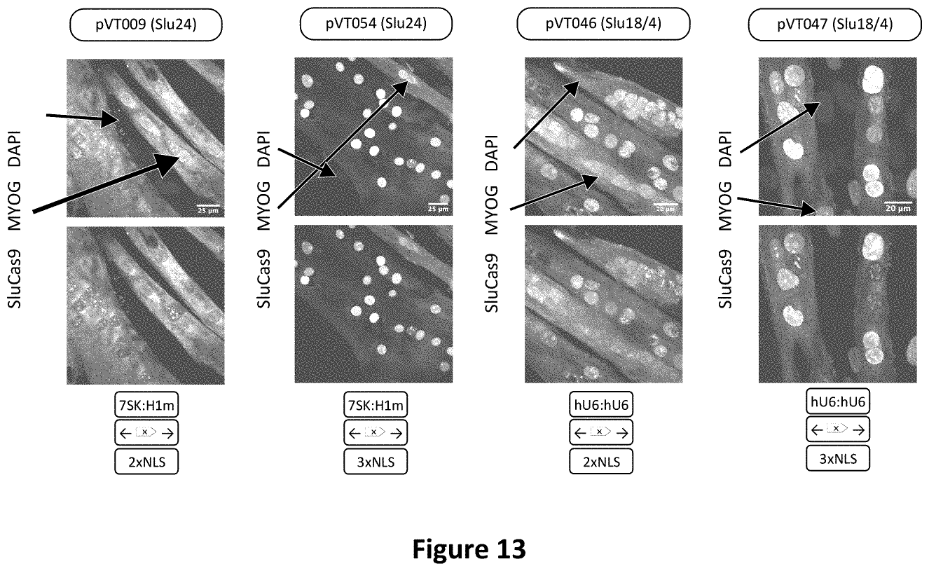

Selected AAV configurations were evaluated for vector genome quantitation (FIG. 11) and transgene expression (FIG. 12), and immunofluorescence in C2C12 mouse myotubes (FIG. 13). Three AAV vectors, vVT046, vVT047, and vVT048, showed an average precise segmental deletion frequency higher than 15% in C2C12 mouse myotubes (FIG. 10). Equivalent vector genome copy number (FIG. 11) and Cas9 transgene expression (FIG. 12A) were observed between all AAV vectors in C2C12 mouse myotubes. Higher sgRNA expression was observed with hU6-H1m-7SK, hU6c-hU6c and hU6c-751(2. PolIII promoter combinations (FIG. 12B). Slightly lower sgRNA expression was observed with 7SK2-H1m promoter combination (FIG. 12B). sgRNA located upstream of Cas9 demonstrated overall higher expression in 7SK2-H1m promoter combination (FIG. 12B). Cas9 with 2×NLS+ and 3×NLS results in superior Cas9 localization to myonuclei (FIG. 13).

TABLE 18B

| Orientation | Configuration | NLS1 | Endonuclease | NLS2 | NLS3 | sgRNA pair | Scaffold | Vector ID |
|---|---|---|---|---|---|---|---|---|
| ← [symbol] → | hU6c-Cas9- | SV-40 | SluCas9 | Nucleoplasmin | N/A | E45Slu18/4 | V5 | vVT046 |
| | hU6c | (+) | | | | | | |
| ← [symbol] → | hU6c-Cas9- | c-Myc- | SluCas9 | SV-40-GSGS | Nucleoplasmin- | E45Slu18/4 | V5 | vVT047 |
| | hU6c | GSVD | | | GSGS | | | |
| ← [symbol] → | hU6c-Cas9- | c-Myc- | SluCas9 | SV-40-GSGS | Nucleoplasmin- | E45Slu18/4 | V5 | vVT048 |
| | 7SK2 | GSVD | | | GSGS | | | |
| ← [symbol] → | H1m-Cas9- | c-Myc- | SluCas9 | SV-40-GSGS | Nucleoplasmin- | E45Slu18/4 | V5 | vVT049 |
| | M11 | GSVD | | | GSGS | | | |
| ← [symbol] → | 7SK2-Cas9- | c-MYc- | SluCas9 | SV-40-GSGS | Nucleoplasmin- | E45Slu18/4 | V5 | vVT050 |
| | H1m | GSVD | | | GSGS | | | |
| ← [symbol] → | 7SK2-Cas9- | SV-40 | SluCas9 | Nucleoplasmin | N/A | E45Slu18/4 | V5 | vVT051 |
| | H1m | (+) | | | | | | |
| ← [symbol] → | 7SK2-Cas9- | SV-40 | SluCas9 | Nucleoplasmin | N/A | E45Slu21/7 | V5 | vVT052 |
| | H1m | (+) | | | | | | |
| ← [symbol] → | 7SK2-Cas9- | SV-40 | SaCas9-KKH | Nucleoplasmin | N/A | E45SaKKH | V2 | vVT053 |
| | H1m | (+) | | | | 10/20 | | |
| ← [symbol] → | 7SK2-Cas9- | c-Myc- | SluCas9 | SV-40-GSGS | Nucleoplasmin- | E45Slu24 | V5 | vVT054 |
| | H1m | GSVD | | | GSGS | | | |

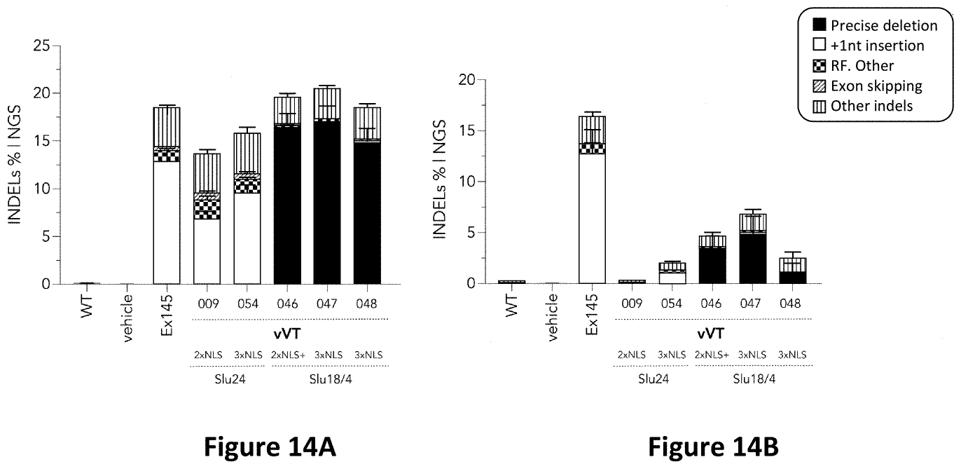

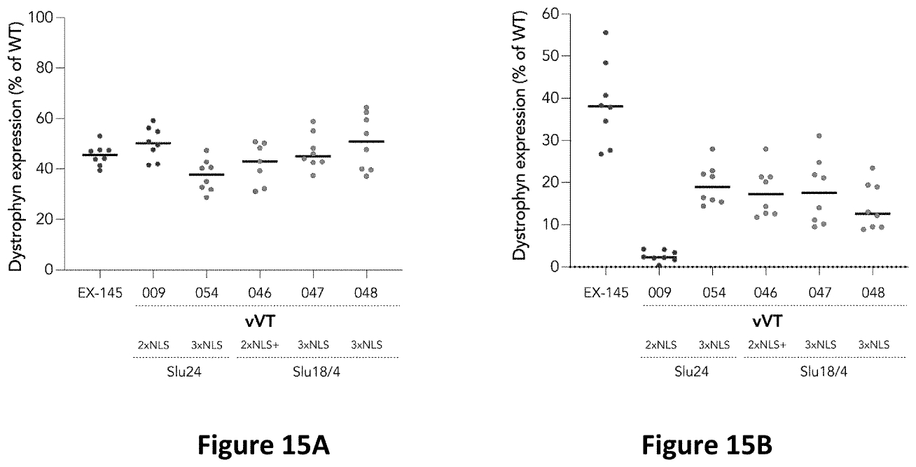

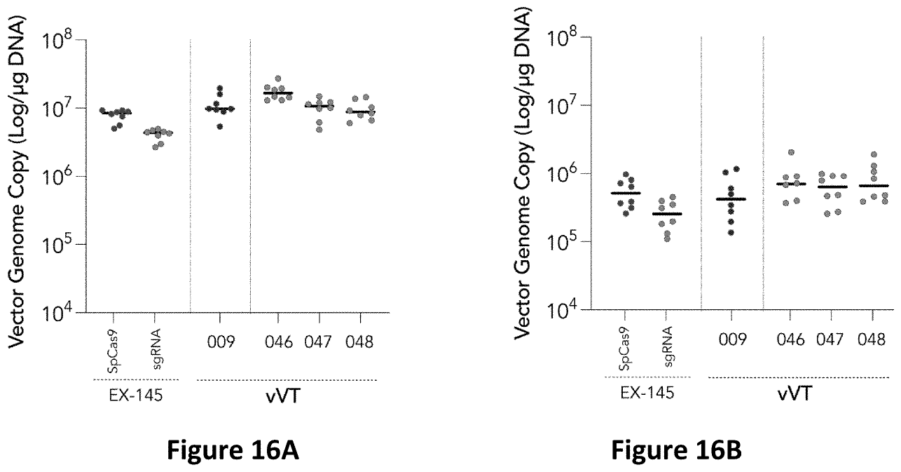

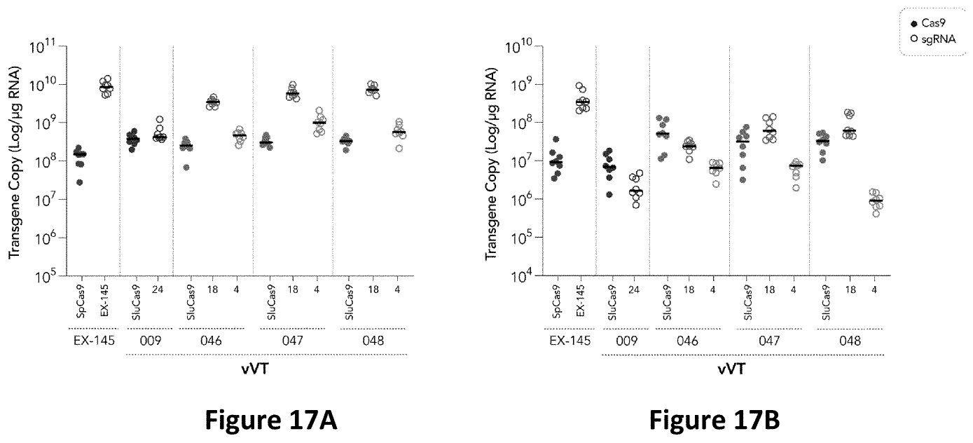

The ability of in vivo single AAV-mediated delivery of gene-editing components to successfully reframe exon 45 in the cardiac and skeletal muscle of dEx44 mice was tested. All selected AAV configurations showed an average total indel frequency higher than 12.5% and E45Slu18/4 sgRNA pair showed an average precise segmental deletion frequency higher than 15% in the heart (FIG. 14A). vVT047 with E45Slu18/4 sgRNA pair showed an average precise segmental deletion frequency higher than 5% in quadriceps (FIG. 14B). All selected AAV configurations showed an average total dystrophin protein restoration higher than 30% of WT in hearts of dEx44 DMD mice (FIG. 15A). vVT054, vVT046, vVT047 and vVT048 showed an average total dystrophin protein restoration higher than 10% of WT in quadriceps of dEx44 DMD mice (FIG. 15B). AAV vectors with 2×NLS+ and 3×NLS enhances editing efficiency and dystrophin restoration in skeletal muscles (FIG. 15B). Equivalent AAV vector genome was observed in all groups in heart and skeletal muscles. Higher overall AAV vector copy was observed in heart than compared to skeletal muscle (FIGS. 16A and 16B). The upstream sgRNA was more efficiently expressed than the downstream cassette in vVT046, vVT047, vVT048 AAV vectors (FIGS. 17A and 17B). Both upstream and downstream sgRNA expression levels were decreased relative to Cas9 levels in muscle compared to heart tissue (FIGS. 17A and 17B).

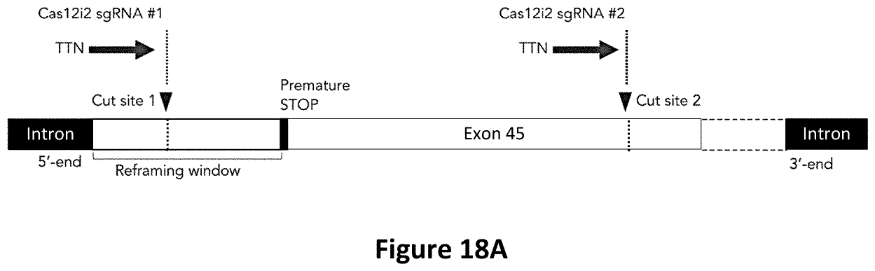

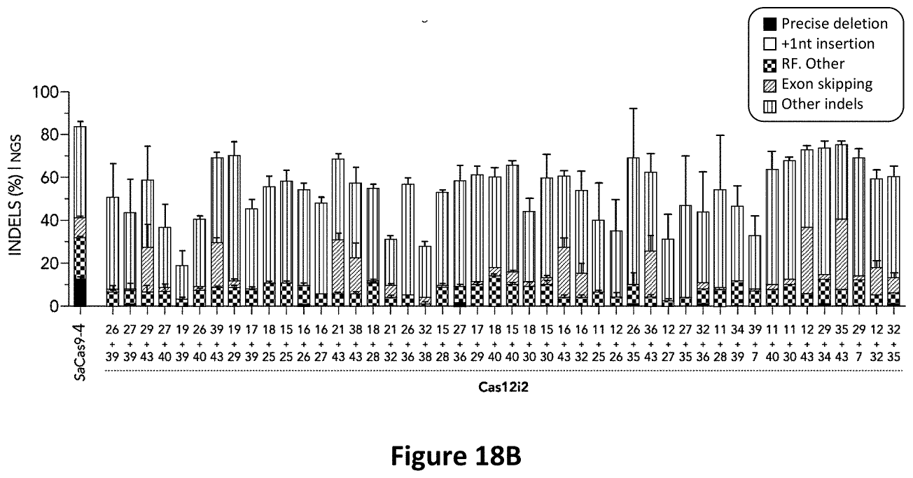

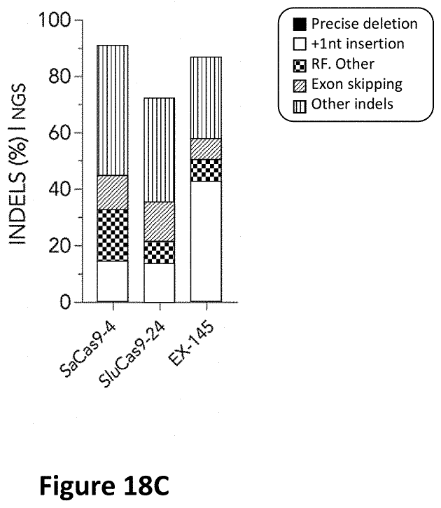

Similar experiments were performed with a variety of sgRNAs (see spacer sequences in Table 19; FIGS. 18A-18C) and Cas12i2 endonuclease in exon 45 in HEK293FT cells.

TABLE 19

| | Protospacer sequences (a comma |
|---|---|
| sgRNA ID | separates the two protospacer sequences) |
| E45CAS12I2TTN26_E45CAS12I2TTN39 | TTCCCCAGTTGCATTCAATG (SEQ ID NO: 555), |
| | GGCAGCGGCAAACTGTTGTC (SEQ ID NO: 556) |
| E45CAS12I2TTN27_E45CAS12I2TTN39 | CCCAGTTGCATTCAATGTTC (SEQ ID NO: 557), |
| | GGCAGCGGCAAACTGTTGTC (SEQ ID NO: 556) |
| E45CAS12I2TTN29_E45CA512I2TTN43 | AATGTTCTGACAACAGTTTG (SEQ ID NO: 558), |
| | TGGTATCTTACAGGAACTCC (SEQ ID NO: 559) |
| E45CAS12I2TTN27_E45CAS12I2TTN40 | CCCAGTTGCATTCAATGTTC (SEQ ID NO: 557), |
| | CAGGAACTCCAGGATGGCAT (SEQ ID NO: 560) |
| E45CAS12I2TTN19_E45CAS12I2TTN39 | TTGAGGATTGCTGAATTATT (SEQ ID NO: 561), |
| | GGCAGCGGCAAACTGTTGTC (SEQ ID NO: 556) |
| E45CAS12I2TTN26_E45CAS12I2TTN40 | TTCCCCAGTTGCATTCAATG (SEQ ID NO: 555), |
| | CAGGAACTCCAGGATGGCAT (SEQ ID NO: 560) |
| E45CAS12I2TTN39_E45CAS12I2TTN43 | GGCAGCGGCAAACTGTTGTC (SEQ ID NO: 556), |
| | TGGTATCTTACAGGAACTCC (SEQ ID NO: 559) |
| E45CAS12I2TTN19_E45CAS12I2TTN29 | TTGAGGATTGCTGAATTATT (SEQ ID NO: 561), |
| | AATGTTCTGACAACAGTTTG (SEQ ID NO: 558) |
| E45CAS12I2TTN17_E45CAS12I2TTN39 | CCTGTAGAATACTGGCATCT (SEQ ID NO: 562), |
| | GGCAGCGGCAAACTGTTGTC (SEQ ID NO: 556) |
| E45CAS12I2TTN18_E45CAS12I2TTN25 | CTGTAGAATACTGGCATCTG (SEQ ID NO: 563), |
| | CTTCCCCAGTTGCATTCAAT (SEQ ID NO: 564) |
| E45CAS12I2TTN15_E45CAS12I2TTN25 | TTCCTGTAGAATACTGGCAT (SEQ ID NO: 565), |
| | CTTCCCCAGTTGCATTCAAT (SEQ ID NO: 564) |
| E45CAS12I2TTN16_E45CAS12I2TTN26 | TCCTGTAGAATACTGGCATC (SEQ ID NO: 566), |
| | TTCCCCAGTTGCATTCAATG (SEQ ID NO: 555) |
| E45CAS12I2TTN16_E45CAS12I2TTN27 | TCCTGTAGAATACTGGCATC (SEQ ID NO: 566), |
| | CCCAGTTGCATTCAATGTTC (SEQ ID NO: 557) |
| E45CAS12I2TTN21_E45CAS12I2TTN43 | GAGGATTGCTGAATTATTTC (SEQ ID NO: 567), |
| | TGGTATCTTACAGGAACTCC (SEQ ID NO: 559) |
| E45CAS12I2TTN38_E45CAS12I2TTN43 | TCAGAACATTGAATGCAACT (SEQ ID NO: 568), |
| | TGGTATCTTACAGGAACTCC (SEQ ID NO: 559) |
| E45CAS12I2TTN18_E45CAS12I2TTN28 | CTGTAGAATACTGGCATCTG (SEQ ID NO: 563), |
| | CATTCAATGTTCTGACAACA (SEQ ID NO: 569) |
| E45CAS12I2TTN21_E45CAS12I2TTN32 | GAGGATTGCTGAATTATTTC (SEQ ID NO: 567), |
| | CCGCTGCCCAATGCCATCCT (SEQ ID NO: 570) |
| E45CAS12I2TTN26_E45CAS12I2TTN36 | TTCCCCAGTTGCATTCAATG (SEQ ID NO: 555), |
| | AGCAATCCTCAAAAACAGAT (SEQ ID NO: 571) |
| E45CAS12I2TTN32_E45CAS12I2TTN38 | CCGCTGCCCAATGCCATCCT (SEQ ID NO: 570), |
| | TCAGAACATTGAATGCAACT (SEQ ID NO: 568) |
| E45CAS12I2TTN15_E45CAS12I2TTN28 | TTCCTGTAGAATACTGGCAT (SEQ ID NO: 565), |
| | CATTCAATGTTCTGACAACA (SEQ ID NO: 569) |
| E45CAS12I2TTN27_E45CAS12I2TTN36 | CCCAGTTGCATTCAATGTTC (SEQ ID NO: 557), |
| | AGCAATCCTCAAAAACAGAT (SEQ ID NO: 571) |
| E45CAS12I2TTN17_E45CAS12I2TTN29 | CCTGTAGAATACTGGCATCT (SEQ ID NO: 562), |
| | AATGTTCTGACAACAGTTTG (SEQ ID NO: 558) |
| E45CAS12I2TTN18_E45CAS12I2TTN40 | CTGTAGAATACTGGCATCTG (SEQ ID NO: 563), |
| | CAGGAACTCCAGGATGGCAT (SEQ ID NO: 560) |
| E45CAS12I2TTN15_E45CAS12I2TTN40 | TTCCTGTAGAATACTGGCAT (SEQ ID NO: 565), |
| | CAGGAACTCCAGGATGGCAT (SEQ ID NO: 560) |
| E45CAS12I2TTN18_E45CAS12I2TTN30 | CTGTAGAATACTGGCATCTG (SEQ ID NO: 563), |
| | TGACAACAGTTTGCCGCTGC (SEQ ID NO: 572) |
| E45CAS12I2TTN15_E45CAS12I2TTN30 | TTCCTGTAGAATACTGGCAT (SEQ ID NO: 565), |
| | TGACAACAGTTTGCCGCTGC (SEQ ID NO: 572) |
| E45CAS12I2TTN16_E45CAS12I2TTN43 | TCCTGTAGAATACTGGCATC (SEQ ID NO: 566), |
| | TGGTATCTTACAGGAACTCC (SEQ ID NO: 559) |
| E45CAS12I2TTN16_E45CAS12I2TTN32 | TCCTGTAGAATACTGGCATC (SEQ ID NO: 566), |
| | CCGCTGCCCAATGCCATCCT (SEQ ID NO: 570) |
| E45CAS12I2TTN11_E45CAS12I2TTN25 | GCAGACCTCCTGCCACCGCA (SEQ ID NO: 573), |
| | CTTCCCCAGTTGCATTCAAT (SEQ ID NO: 564) |
| E45CAS12I2TTN12_E45CAS12I2TTN26 | CAGACCTCCTGCCACCGCAG (SEQ ID NO: 574), |
| | TTCCCCAGTTGCATTCAATG (SEQ ID NO: 555) |
| E45CAS12I2TTN26_E45CAS12I2TTN35 | TTCCCCAGTTGCATTCAATG (SEQ ID NO: 555), |
| | TACAGGAAAAATTGGGAAGC (SEQ ID NO: 575) |
| E45CAS12I2TTN36_E45CA512I2TTN43 | AGCAATCCTCAAAAACAGAT (SEQ ID NO: 571), |
| | TGGTATCTTACAGGAACTCC (SEQ ID NO: 559) |
| E45CAS12I2TTN12_E45CAS12I2TTN27 | CAGACCTCCTGCCACCGCAG (SEQ ID NO: 574), |
| | CCCAGTTGCATTCAATGTTC (SEQ ID NO: 557) |
| E45CAS12I2TTN27_E45CAS12I2TTN35 | CCCAGTTGCATTCAATGTTC (SEQ ID NO: 557), |
| | TACAGGAAAAATTGGGAAGC (SEQ ID NO: 575) |
| E45CAS12I2TTN32_E45CAS12I2TTN36 | CCGCTGCCCAATGCCATCCT (SEQ ID NO: 570), |
| | AGCAATCCTCAAAAACAGAT (SEQ ID NO: 571) |
| E45CAS12I2TTN11_E45CAS12I2TTN28 | GCAGACCTCCTGCCACCGCA (SEQ ID NO: 573), |
| | CATTCAATGTTCTGACAACA (SEQ ID NO: 569) |
| E45CAS12I2TTN34_E45CAS12I2TTN39 | GGAAGCCTGAATCTGCGGTG (SEQ ID NO: 576), |
| | GGCAGCGGCAAACTGTTGTC (SEQ ID NO: 556) |
| E45CAS12I2TTN39_E45CAS12I2TTN7 | GGCAGCGGCAAACTGTTGTC (SEQ ID NO: 556), |
| | TTCTGTCTGACAGCTGTTTG (SEQ ID NO: 577) |
| E45CAS12I2TTN11_E45CAS12I2TTN40 | GCAGACCTCCTGCCACCGCA (SEQ ID NO: 573), |
| | CAGGAACTCCAGGATGGCAT (SEQ ID NO: 560) |
| E45CAS12I2TTN11_E45CAS12I2TTN30 | GCAGACCTCCTGCCACCGCA (SEQ ID NO: 573), |
| | TGACAACAGTTTGCCGCTGC (SEQ ID NO: 572) |
| E45CAS12I2TTN12_E45CAS12I2TTN43 | CAGACCTCCTGCCACCGCAG (SEQ ID NO: 574), |
| | TGGTATCTTACAGGAACTCC (SEQ ID NO: 559) |
| E45CAS12I2TTN29_E45CAS12I2TTN34 | AATGTTCTGACAACAGTTTG (SEQ ID NO: 558), |
| | GGAAGCCTGAATCTGCGGTG (SEQ ID NO: 576) |
| E45CAS12I2TTN35_E45CAS12I2TTN43 | TACAGGAAAAATTGGGAAGC (SEQ ID NO: 575), |
| | TGGTATCTTACAGGAACTCC (SEQ ID NO: 559) |
| E45CAS12I2TTN29_E45CAS12I2TTN7 | AATGTTCTGACAACAGTTTG (SEQ ID NO: 558), |
| | TTCTGTCTGACAGCTGTTTG (SEQ ID NO: 577) |
| E45CAS12I2TTN12_E45CAS12I2TTN32 | CAGACCTCCTGCCACCGCAG (SEQ ID NO: 574), |
| | CCGCTGCCCAATGCCATCCT (SEQ ID NO: 570) |
| E45CAS12I2TTN32_E45CAS12I2TTN35 | CCGCTGCCCAATGCCATCCT (SEQ ID NO: 570), |
| | TACAGGAAAAATTGGGAAGC (SEQ ID NO: 575) |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

1. A composition comprising:
a. a single nucleic acid molecule comprising:
i. a nucleic acid encoding Staphylococcus aureus Cas9 (SaCas9) or Staphylococcus lugdunensis (SluCas9) and at least one, at least two, or at least three guide RNAs; or
ii. a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or
iii. a nucleic acid encoding Staphylococcus aureus Cas9 (SaCas9) or Staphylococcus lugdunensis (SluCas9) and 1, 2, or 3 guide RNAs; or
b. two nucleic acid molecules comprising:
i. a first nucleic acid encoding Staphylococcus aureus Cas9 (SaCas9) or Staphylococcus lugdunensis (SluCas9); and
a second nucleic acid that does not encode a SaCas9 or SluCas9 and encodes any one of the following:
1. at least one, at least two, at least three, at least four, at least five, or at least six guide RNAs; or
2. from one to n guide RNAs, wherein n is no more than the maximum number of guide RNAs that can be expressed from said nucleic acid; or
3. from one to six guide RNAs; or
ii. a first nucleic acid encoding Staphylococcus aureus Cas9 (SaCas9) or Staphylococcus lugdunensis (SluCas9) and
1. at least one, at least two, or at least three guide RNAs; or
2. from one to n guide RNAs, wherein n is no more than the maximum number of guide RNAs that can be expressed from said nucleic acid; or
3. 1, 2, or 3 guide RNAs; and
a second nucleic acid that does not encode a SaCas9 or SluCas9, optionally wherein the second nucleic acid comprises any one of the following:
1. at least one, at least two, at least three, at least four, at least five, or at least six guide RNAs; or
2. from one to n guide RNAs, wherein n is no more than the maximum number of guide RNAs that can be expressed from said nucleic acid; or
3. from one to six guide RNAs; or
iii. a first nucleic acid encoding Staphylococcus aureus Cas9 (SaCas9) or Staphylococcus lugdunensis (SluCas9) and at least one, at least two, or at least three guide RNAs; and
a second nucleic acid that does not encode a SaCas9 or SluCas9 and encodes from one to six guide RNAs; or
iv. a first nucleic acid encoding Staphylococcus aureus Cas9 (SaCas9) or Staphylococcus lugdunensis (SluCas9) and at least two guide RNAs, wherein at least one guide RNA binds upstream of a target sequence and at least one guide RNA binds downstream of the target sequence; and
a second nucleic acid that does not encode a SaCas9 or SluCas9 and encodes at least one additional copy of each of the guide RNAs encoded in the first nucleic acid,
wherein the guide RNA(s) target a region in the dystrophin gene.

2. A composition comprising two nucleic acid molecules comprising i) a first nucleic acid encoding Staphylococcus aureus Cas9 (SaCas9) or Staphylococcus lugdunensis Cas9 (SluCas9); and ii) a second nucleic acid encoding at least 2 or at least 3 copies of a first guide RNA and at least 2 or at least 3 copies of a second guide RNA, wherein the first and second guide RNA function to excise a portion of a DMD gene.

3. A composition comprising one or more nucleic acid molecules encoding an Staphylococcus aureus Cas9 (SaCas9) or Staphylococcus lugdunensis Cas9 (SluCas9) and a pair of guide RNAs, wherein each guide RNA targets a different sequence in a DMD gene, wherein the endonuclease and pair of guide RNAs are capable of excising a DNA fragment from the DMD gene; wherein the DNA fragment is between 5-250 nucleotides in length.

4-6. (canceled)

7. The composition of claim 3, wherein the excised DNA fragment comprises a splice acceptor site, splice donor site, a premature stop codon, or wherein the excised DNA fragment does not comprise an entire exon of the DMD gene.

8-10. (canceled)

11. The composition of claim 1, comprising a single nucleic acid molecule encoding one or more guide RNAs and a Cas9, wherein the single nucleic acid molecule comprises:
a. a first nucleic acid encoding one or more spacer sequences selected from any one of SEQ ID NOs: 1-35, 1000-1078, or 3000-3069 and a second nucleic acid encoding a Staphylococcus aureus Cas9 (SaCas9); or
b. a first nucleic acid encoding one or more spacer sequences selected from any one of SEQ ID NOs: 100-225, 2000-2116, or 4000-4251 and a second nucleic acid encoding a Staphylococcus lugdunensis Cas9 (SluCas9); or
c. a first nucleic acid encoding one or more spacer sequences comprising at least 20 contiguous nucleotides of a spacer sequence selected from any one of SEQ ID NOs: 1-35, 1000-1078, or 3000-3069 and a second nucleic acid encoding a Staphylococcus aureus Cas9 (SaCas9); or
d. a first nucleic acid encoding one or more spacer sequences comprising at least 20 contiguous nucleotides of a spacer sequence selected from any one of SEQ ID NOs: 100-225, 2000-2116, or 4000-4251 and a second nucleic acid encoding a Staphylococcus lugdunensis Cas9 (SluCas9); or
e. a first nucleic acid encoding one or more spacer sequences that is at least 90% identical to any one of SEQ ID NOs: 1-35, 1000-1078, or 3000-3069 and a second nucleic acid encoding a Staphylococcus aureus Cas9 (SaCas9); or
f. a first nucleic acid encoding one or more spacer sequences that is at least 90% identical to any one of SEQ ID NOs: 100-225, 2000-2116, or 4000-4251 and a second nucleic acid encoding a Staphylococcus lugdunensis Cas9 (SluCas9); or
g. a first nucleic acid encoding a pair of guide RNAs comprising a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
h. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
i. a first nucleic acid encoding a pair of guide RNAs that is at least 90% identical to a first and second spacer sequence selected from any one of the following pairs of spacer sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9); or
j. a first nucleic acid encoding a pair of guide RNAs comprising a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis Cas9 (SluCas9); or
k. a first nucleic acid encoding a pair of guide RNAs comprising at least 17, 18, 19, 20, or 21 contiguous nucleotides of a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis Cas9 (SluCas9); or
l. a first nucleic acid encoding a pair of guide RNAs that is at least 90% identical to a first and second spacer sequence selected from any one of the following pairs of spacer sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis Cas9 (SluCas9); or
m. a first nucleic acid encoding a pair of guide RNAs that is at least 90% identical to a first and second spacer sequence selected from any one of the following pairs of spacer sequences:
i. SEQ ID NOS: 148 and 134,
ii. SEQ ID Nos: 145 and 131,
iii. SEQ ID Nos: 144 and 149;
iv. SEQ ID Nos: 144 and 150;
v. SEQ ID Nos: 146 and 148;
and a second nucleic acid encoding a Staphylococcus lugdunensis Cas9 (SluCas9); or
n. a first nucleic acid encoding a pair of guide RNAs that is at least 90% identical to a first and second spacer sequence selected from any one of the following pairs of spacer sequences:
i. SEQ ID NOs: 12 and 1013; and
ii. SEQ ID Nos: 12 and 1016;
and a second nucleic acid encoding a SaCas9-KKH.

12. The composition of claim 11, comprising one or more nucleic acid molecules encoding a Staphylococcus lugdunensis Cas9 (SluCas9) and at least two guide RNAs, wherein a first and second guide RNA target different sequences in a DMD gene, wherein the first and a second guide RNA comprise a sequence that is at least 90% identical to a first and second spacer sequence selected from any one of the following pairs of spacer sequences:
i. SEQ ID NOS: 148 and 134,
ii. SEQ ID Nos: 145 and 131,
iii. SEQ ID Nos: 144 and 149;
iv. SEQ ID Nos: 144 and 150;
v. SEQ ID Nos: 146 and 148.

13. The composition of claim 11, comprising one or more nucleic acid molecules encoding an endonuclease and at least two guide RNAs, wherein the guide RNAs each target a different sequence in a DMD gene, wherein the guide RNAs each comprise a sequence that is at least 90% identical to a first and second spacer sequence selected from any one of the following pairs of spacer sequences:
i. SEQ ID NOs: 12 and 1013; and
ii. SEQ ID Nos: 12 and 1016; and
a second nucleic acid encoding a SaCas9-KKH.

14. The composition of claim 1, wherein the first and/or the second nucleic acid, if present, encodes at least two, at least three, at least four, at least five, or at least six guide RNAs.

15-24. (canceled)

25. The composition of claim 1, wherein the second nucleic acid, if present, encodes 1) from one to n guide RNAs, wherein n is no more than the maximum number of guide RNAs that can be expressed from said nucleic acid; 2) from one to six guide RNAs; 3) 2-10, 2-9, 2-8, 2-7, 2-6, 2-5, 2-4, or 2-3 guide RNAs; or 4) 2, 3, 4, 5, or 6 guide RNAs.

26-41. (canceled)

42. The composition of claim 1, wherein the composition comprises at least two nucleic acid molecules, wherein the first nucleic acid molecule comprises a sequence encoding an endonuclease, wherein the second nucleic acid molecule encodes a first guide RNA and a second guide RNA, wherein the first guide RNA and the second guide RNA are not the same sequence, and wherein the second nucleic acid molecule does not encode an endonuclease, wherein the first nucleic acid molecule encodes a Staphylococcus aureus Cas9 (SaCas9) endonuclease, and wherein the first guide RNA comprises the first sequence and the second guide RNAs comprises the second sequence from any one the following pairs of sequences: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018.

43. (canceled)

44. The composition of claim 1, wherein the composition comprises at least two nucleic acid molecules, wherein the first nucleic acid molecule comprises a sequence encoding an endonuclease, wherein the second nucleic acid molecule encodes a first guide RNA and a second guide RNA, wherein the first guide RNA and the second guide RNA are not the same sequence, and wherein the second nucleic acid molecule does not encode an endonuclease, wherein the first nucleic acid molecule encodes a Staphylococcus lugdunensis Cas9 (SluCas9) endonuclease, and wherein the first guide RNA comprises the first sequence and the second guide RNAs comprises the second sequence from any one the following pairs of sequences: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146.

45-94. (canceled)

95. The composition of claim 1, wherein the single nucleic acid molecule or the first nucleic acid comprises from 5′ to 3′ with respect to the plus strand: the reverse complement of a nucleotide sequence encoding a first guide RNA scaffold sequence, the reverse complement of a nucleotide sequence encoding the first guide RNA sequence, the reverse complement of a promoter for expression of the nucleotide sequence encoding the first guide RNA sequence, a promoter for expression of a nucleotide sequence encoding Staphylococcus aureus Cas9 (SaCas9) or Staphylococcus lugdunensis Cas9 (SluCas9), a nucleotide sequence encoding the SaCas9 or SluCas9, a polyadenylation sequence, a promoter for expression of the second guide RNA in the same direction as the promoter for the SaCas9 or SluCas9, a nucleotide sequence encoding the second guide RNA sequence, and a nucleotide sequence encoding the second guide RNA scaffold sequence.

96-101. (canceled)

102. The composition of claim 95, wherein the scaffold sequence for the first and/or second guide RNA comprises the sequence of SEQ ID NO: 901.

103. (canceled)

104. An AAV9 vector comprising any of the nucleic acids recited in claim 1, wherein the AAV9 vector is less than 5 kb, less than 4.9 kb, 4.85, 4.8, or 4.75 kb in size from ITR to ITR, inclusive of both ITRs.

105. The AAV9 vector of claim 104, wherein the AAV9 vector is between 3.9-5 kb, 4-5 kb, 4.2-5 kb, 4.4-5 kb, 4.6-5 kb, 4.7-5 kb, 3.9-4.9 kb, 4.2-4.9 kb, 4.4-4.9 kb, 4.7-4.9 kb, 3.9-4.85 kb, 4.2-4.85 kb, 4.4-4.85 kb, 4.6-4.85 kb, 4.7-4.85 kb, 4.7-4.9 kb, 3.9-4.8 kb, 4.2-4.8 kb, 4.4-4.8 kb or 4.6-4.8 kb in size from ITR to ITR, inclusive of both ITRs.

106. (canceled)

107. A composition comprising a guide RNA encoded by a sequence comprising any one of:
a. SEQ ID NOs: 1-24, 1000-1078, or 3000-3048 or complements thereof; or
b. SEQ ID NOs: 100-200, 2000-2116, or 4000-4201 or complements thereof.

108. A composition comprising a guide RNA encoded by a sequence comprising any one of:
a. SEQ ID NOs: 25-35 or 201-225, or complements thereof;
b. SEQ ID NOs: 201-225, or 4202-4251 or complements thereof.

109-110. (canceled)

111. A method of treating Duchenne Muscular Dystrophy (DMD), the method comprising delivering to a cell the composition of claim 1.

112-115. (canceled)

116. The method of claim 111, wherein the single nucleic acid molecule is delivered to the cell on a single vector.

117. The method of claim 111, comprising a nucleic acid molecule encoding SaCas9, wherein the spacer sequence is selected from any one of SEQ ID NOs: 12, 15, 16, 20, 27, 28, 32, 33, and 35.

118. The method of claim 111, comprising a nucleic acid molecule encoding SaCas9, wherein the SaCas9 comprises an amino acid sequence that is at least 80%, 85%, 90%, 91%, 92%, 93%, 94%, 95%, 96%, 97%, 98%, 99% or 100% identical to the amino acid sequence of SEQ ID NO: 711.

119. (canceled)

120. The method of claim 111, comprising a nucleic acid molecule encoding SaCas9, wherein the SaCas9 comprises an amino acid sequence that is at least 80%, 85%, 90%, 91%, 92%, 93%, 94%, 95%, 96%, 97%, 98%, 99% or 100% identical to an amino acid sequence selected from any one of SEQ ID NOs: 715-717.

121. The method of claim 111, comprising a nucleic acid molecule encoding SluCas9, wherein the spacer sequence is selected from any one of SEQ ID NOs: 131, 134, 135, 136, 139, 144, 148, 149, 150, 151, 179, 184, 201, 210, 223, 224, and 225.

122. The method of claim 111, comprising a nucleic acid molecule encoding SluCas9, wherein the SluCas9 comprises an amino acid sequence that is at least 80%, 85%, 90%, 91%, 92%, 93%, 94%, 95%, 96%, 97%, 98%, 99% or 100% identical to the amino acid sequence of SEQ ID NO: 712.

123. (canceled)

124. The method of claim 111, comprising a nucleic acid molecule encoding SluCas9, wherein the SluCas9 comprises an amino acid sequence that is at least 80%, 85%, 90%, 91%, 92%, 93%, 94%, 95%, 96%, 97%, 98%, 99% or 100% identical to an amino acid sequence selected from any one of SEQ ID NOs: 718-720.

125. A method of excising a portion of an exon with a premature stop codon in a subject with Duchenne Muscular Dystrophy (DMD), the method comprising delivering to a cell a single nucleic acid molecule comprising:
i) a nucleic acid encoding a pair of guide RNAs wherein the first guide RNA binds to a target sequence within the exon that is upstream of the premature stop codon, and wherein the second guide RNA binds to a sequence that is downstream of the premature stop codon and downstream of the sequence bound by the first guide RNA; and
a. a nucleic acid encoding a Staphylococcus aureus Cas9 (SaCas9); or
b. a nucleic acid encoding a Staphylococcus lugdunensis Cas9 (SluCas9);
wherein the pair of guide RNAs and Cas9 excise a portion of the exon.

126-131. (canceled)

132. The method of claim 125, wherein a portion of a DMD gene is excised, wherein the portion is within exon 43, 44, 45, 50, 51, or 53.

133. The method of claim 125, comprising a pair of guide RNAs, wherein the pair of guide RNA comprise a first and second spacer sequence selected from any one of SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146.

134-158. (canceled)

159. The composition of claim 2, wherein the first nucleic acid does not encode a guide RNA.

160. The composition of claim 2, wherein the first nucleic acid encodes a copy of the first guide RNA and a copy of the second guide RNA.
